SEQUENCE LISTING (69 sequences)

SEQ ID NO: 1 - DNA, 64 bp, Artificial Sequence
  5 ssODN (rs76763715)
ttccagtcggtccagccaaccacatggtacaggaggttctagggtaaggacaaaggcaaagaga

SEQ ID NO: 2 - DNA, 36 bp, Artificial Sequence
  3 ssODN (rs76763715)
caaaggctcaacactgggggtccccagagagtgtag

SEQ ID NO: 3 - DNA, 100 bp, Artificial Sequence
  ssODN (rs76763715)
ttccagtcggtccagccaaccacatggtacaggaggttctagggtaaggacaaaggcaaagagacaaaggctcaacactgggggtccccagagagtgtag

SEQ ID NO: 4 - DNA, 64 bp, Artificial Sequence
  5 ssODN (rs76763715)
ttccagtcggtccagccgaccacatggtacaggaggttctagggtaaggacaaaggcaaagaga

SEQ ID NO: 5 - DNA, 100 bp, Artificial Sequence
  ssODN (rs76763715)
ttccagtcggtccagccgaccacatggtacaggaggttctagggtaaggacaaaggcaaagagacaaaggctcaacactgggggtccccagagagtgtag

SEQ ID NO: 6 - protein, 497 aa, Homo sapiens
  Human GBA1 (wildtype)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 7 - protein, 497 aa, Homo sapiens
  Human GBA1 (N370S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITSLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 8 - DNA, 20 bp, Artificial Sequence
  crRNA (rs76763715)
ggacaaaggcaaagagacaa

SEQ ID NO: 9 - protein, 497 aa, Homo sapiens
  Human GBA1 (L444P mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDPDAVALMHPDGSAVVVVLNRSSKDVPLTIKDPAVGFLETISPGYSIHTYLWRRQ

SEQ ID NO: 10 - protein, 497 aa, Homo sapiens
  Human GBA1 (E326K mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGKTHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 11 - DNA, 13625 bp, Homo sapiens
  Human GBA1 (wildtype)
acagaagtacaatgctgattatgatctgtcagctcagcaaggggcagacaccctggccttcatgtctctcctggaggagaagttgatcccggtgctggtgagtgtgcccagacctcccagcatccatggccagccggggaggggacgggaacacacagacccacacagagactcaggagagcatggaggtcagaagcccaccttgaatcagacaggtgcactggctcagacctgcctgtttcttcctgcccacccaatccaggtacatactttttggatagacaccaagaactacgtagaagtgacccggaagtggtatgcagaggctatgccctttcccctcaacttcttcctgcctggccgcatgcagcggcagtacatggaacggctacagctgctgactggggagcacaggcctgaggacgaggaagagctggagaaggaggtagctctgagaccgggggctattgtatgagatgagccccaaggatgctggccaggaatgggagtgcttaggtgcggaggtggcactgttcccgcagctgcaagcctacctgtgtcgcccctacagctgtaccgagaggctcgggagtgtctgaccctgctctctcagcgcctgggctctcaaaagttcttctttggagatgcgtgagtctgactccaagagggtaatgggtggcttggaagaagatacaggttcagatggagcagctggagctggggctggggctggggctggctcaggctctggataggaggtccctgagacagatactggccctggtgacagtggggctgtgcgtggggccagagccttctcagaggtacaaaagggtagggtgggagggcagccaggcacaggaagggcctgaagagctgtggggcactgagtgtgccctttatgcagccctgggatagagccctattcagggccaggctggcgccacctggggatctctccccataccaggtctagaactgtgtgtcctgtccttccctggtggccgcctgctgcccagagcccacctcccaaggctgactcttcctccagctccatctttaccccttctaccccagtggttctcctccatcccacccttctctctctgctccagccctgcctccttggacgccttcgtcttcagctacttggccctgctgctgcaggcaaagctgcccagtgggaagctgcaggtccacctgcgtgggctgcacaacctctgtgcctattgtacccacattctcagtctctacttcccctgggatggaggtaaggggcagatgggaggggcagccctggggagagtgggcagggatccaagaactagttctcctaacacaccttccttccttgaccctcagctgaggtaccaccgcaacgccagacaccagcaggcccagagactgaggaggagccataccggcgccggaaccagatcctatctgtgctggcaggactggcagccatggtgggctacgccttgctcagcggcattgtctccatccagcgggcaacgcctgctcgggccccaggcacccggaccctgggcatggctgaggaggatgaagaggaatgatttgtcctcacgctcccaagactggtttttctactctcatgcattccagaggcccccgtgcctcctcgttgttggtacagccggacacggggtgctgccacccagaataaagccactcacactgactgggctcaaacattttctcctttaagagctgccatttttcctggctggtgccataggaatcatctgggtgcctgggcacacccgctgctgctttaaggcttccgccctgatgctgacactgctgctccacgggcccagttctgcatctccaggaaagacaaacagtctccagttttgggcccagctttcctagtctcttcttttccttaccctcagccctgatcttgtgtttgtacggacagtgagctcaccctaggcctggacccaggcccagtttccaaagcaagcagcacacagcgcatgttcacataagcatgggggctggggggacactggggcttactgatctttttctaggggcctccagcccctggcaccacctagaggggaaagtgagtcacccaaaccattgcccctgggcttacgtcgctgtaagctcacactggccctgctgtgccctctttagtcacagacagcgtgtgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagctagggagcagggaggagaagctgagagtgtgatcctgccaaggcccccaacgctgtcttcagcccacttcccagacctcaccattgccctcaccggtttagcacgaccacaacagcagagccatcgggatgcatcagtgccactgcgtccaggtcgttcttctgactggcaaccagccccactctctgggagccctcaggaatgaacttgctgaatgtgggaacagatggtcagagtccctcggggtacctcccatgaaaccctcatctaagaagtcacccacccacggacccaccccataactcctgcagaggctctgccctggctctctaggcctggagccatgctgctgggcactgaccctgcttttctgcatcgcagtccagcctcaggcattggggttttctgttgctacctagtcacttcctgcctccatggtgcaaaaggggatgggtgtgcctcttccgaggttccaccctgaacaccttcctgctccctcgtggtgtagagtgatgtaagccatccgatgtaggagatgataggcctggtatggaatgggggtgcccgccctccactcacctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggttctagggtaaggacaaaggcaaagagacaaaggcgcaacactgggggtccccagagagtgtaggtaagggtcacatgtgggagaggcagctgtgggtaggtcagccctgtgaggggcacattccttagtagctaaggagttgggggtgtgaagatccaggcatctcaaggggagctgagaagtctgaggcagctgcaagtgcctcagtagttgcaaaaggggcaatgaggtgtgcagacctgtgaaggaaagggaagatagggaatcatggttccccagagttgctcaaaagggcaggctagctggggaaagctggacaggaagggcttctgtcagtctttggtgaaactagtaagaggtctgaggtctgctttgcaggaagggagactggggtggcttaccgtgatgatgctgtggctgtactgcatccctcgatcccaggagcctagccgcacactctgctcccagaacttggagcccacacaggcctctgaggcaaagagcatggtgttggggaacaggcggtgtgtctcccctagggtggctttggctggagccagaaagtccaggtaccaatgtacagcaatgccatgaacatatttagctgcttctgggtctgtcagtacctgcaaaggaagagcaactgatcctggaccttgcacacaggcttctggaacttctagttcctgttgtaggaatcctggagttgggtgacgggaagaatgcaactagagaggtttggggagatttttttttgtttttgagacaggatgtcactctgtcactcgggctggagtacagtggcgcaatcacgactcactgcagccttgacttcctagggtcaactgatcctcccacattagcctcctgagtagctgggactacacgggtgccacacccagctaatttgtgtgtgtatgtgtgtgtatgtatgtgtgtgtgtgtatatatatatacatataaacacatatatatgtatatacacatacatacatgaaccaccacccccagcctagatagttttgttttgttttgtttcgagatggagtctcgctcagcctcccaggctggagtgcagtggcgcgatctcggctcaccgcaaccaccatctcccgggttcaactgattctcccttctcagcctcctgagtagctgggattacagacacccaccatcatgcccagataattttttttttttgtattttagtagacacagggtttcaacacgttggccagggtggtcttaaactcctgacctcaggtgatcacccgcctcggcttcccaaagtgctgggtttgcatgagtgagccacctcgcccagcccctagaaaggtttcaagcgacaactgtgggatccatggcaccctggaggtccaggggaatggtgctctaggaatccatagttgggtagagaaatcgctctaagtttgggagccagtcatttggatgctggatttgaaggtcactggagcaccatggaggtccaggccttaccacctttgcccagtggggcagcagcaagcgttggtcatccagcatgagtaggcggacattgtggtgagtactgttggcgagggtaggacctaggtcacgggcaatgaagtctcgctgatgttcaggggtgaagcccaggcactggaaggggtatccactcaacagcccagcagaaggctcattttcagctgtcactgcccagaactgtaacttgtgctcagcataggcatccaggaacctggcaagagaaaggtcatgaatgatccggccaagaaagtggaccagaccagctgggtgtggtggctcacacctgtaatcccagcactttgggaagccgaggcaggtggatcacttgagttcaggagttcgagaacagcctggcgaaaccccgtctctactaaaaatagaaaaatcagctgggcctggtggcaggcgcctataatcccagctacttggaaggctgaggcaggagaattgcttgaactcaggaggcagaggttacagtgagtgaagatggcgccacagcactccagcctgggtgacagagagagagactccttctcaaaaaaaaaaaaaagaagaaaaataaaaagaaagtgggccagaccgagagaacaggaagcctgatggagtgggcaagattgacaggcccaaggctgaaaggcccagaaggtagaaaggtgagctgaggacaggcagatctggaagtggaactaggttgagggttgggacacagatcagcatggctaaatgggaggccagtcctgatcccacatccttgctgatcccttacttcacaaagtatctggcccaggtctggtggtagatgtctccgggctgtcccttgagtgaccccttcccattcaccgctccattggtcttgagccaagtgggtgatgtccaggggctggcaaggagtgaaacgggacgctgggccaactgcagggctcggtgaatcaggggtatctagagacaaaggtagtgaagagagaagcacccagagttggaacacatactagcccaaccagtgcatccggttcagccattagcctccaccctcccacccccaggacaaaacagcaggggacaaaatgtctgtacaagcagacctaccctacagtttctcaacccccagacatcagggccctcagggcctgaaaaagctagaatgcctaccttgagcttggtatcttcctctgggaggctgaagttgtgcaactggaaatcatcaggggtgtctgcataggtgtaggtgcggatggagaagtcacagctggccatgggtacccggatgatgttatatccgattcctacagaaaaggatgatcaagatatggtagtccgagtcaataggagagtatgggactctgcttatcacttgccagtcctaatagtgtctgagtcagggccaaagggaacttgggctcctgggttggaacctgtggaggctggcacctgggtgaagcgcaggcctttctgagcctgagtccgtagcagttagcagatgataggcggtgaaatcttatttcacagggcattaaaacaggaaccaaatgtcagggatgggcagaagtcagggtccaaagaaagggcaaagaaaagtgtcagtggctcacgcatgtaatcccagcactttgggaggccgatgtgggcagatcacgaggtcaggagttcgcaatcatcctggccaacatagtgaaaccctgtctctactaaaaatacaaaaaattagccgggcgtggtggcaggcacctgtaatcccaggtactcgagaggctgagacaggagaatcacctgaacccgggaggtggaggttgcagtgagctgagattgtgccactgcactccagcctgggtgacagtgcgagactctgtctcaaaaaaaaaaaaagaaaaaagaaaagtgtctgctgggctcggtggctcacacttgtaattccagcacttcgggaggccaaggcaggtatatcatttgaggtcaggagtttaagactagcctggtcaacatgatgaaaccctgtctctactaaaaatacaaaaattagccaggtggtagtggcgcacgcctataattccagctactcaggaggctgaggcaggagaatcactatagcctgggaggcagaggttgctgtgaggggagatcacaccactgcactcctgtctcgccgacagaatgggcagagtgagattctgcctcaaaaaaattttaaaaaaagaaaagaaaaacgaaaagtttcaatggctctatgtcatcttgtccccttcctcctcaccttcttcagagaagtacgatttaagtagcaaattttgggcagggggtgacagggcaaggatgttgagagcagcagcatctgtcatggcccctccaaatcccttcactttctggaacttctgttctggctgcagggtcagtagcaggcctgaggacatccacagggaataagggtatcagtacccagcgggaaactccatggtgatcactgacaccatttacctctaggaggacccagcctggcccagggggtgaggggtgtaatggttacctgtgcccgtgtgattagcctggatgggccccatactcagctccatccgtcgcccactgcgtgtactctcatagcggctgaaggtaccaagggcaggaaaggtcggggggtcaaaggagtcacagtatgtggcattgcagacacacaccaccgagctgtagccgaagcttttagggatgcaggggcgggcacctgggagggagggagtacaagcagagtgaggtctgatgaagacatggagaatggacacatctgctaggagagactgaacacggtttcaaaattcctcaccccttggccgggcgcaggggctcacacctgtaatcctagcactttaggaggccgaggtgggcggatcacctgaggtgaggagtttgagacctgccttgccagcatggtgaaaccccgtctctactaaaaatacaaaaattagctgggcgtagtggtggccgcctgtaatcccagctacctgggaagctgaagcaagagaatcgcttgaatctgggaggcagaggttggaatgagccaaaattgcaccactgcactccagcccaggcaacagagtaagactctgtttcaaaagaaaaaaaacaaaatcctcaccccaaagttggtctcagtcactcaaaaggatttattgagcacctactaaaagtctaccaccagcttactggaaggctaccaaaggactatgaggcagaagggaggctctgtgctacctccccactgccttgactcactcacctgatgcccacgacactgcctgaagtagaagcaatcctgtgaggctgccagccatgatgcttaccctactcaaaggcttgggacattcctgaggacagaatgaggaatgactgaaaagcaagcccctctccacccctcaactactctcctgggcagggcttagctgcctttgggtgcccatggcccggtctcccacattcattaggacaaggcccacagaaacctgggtgcagctctccctgcaacccttctgatgacaactctctgcccaccccaaatcaggatgctccccaccactcttcccacccatttcaacttcgacccctccctccatctgtgccttgctcaaagagccatgatggccctggattcaaagagagtctgtcattcattaaattccagtgccaggattccagaagcactgtgacaatgctgattgggagctctctctcttacctctctggaaggacttgaaaactccatcccctcagggtcattagatgaagagaagaccacaggggttccagagtctctgaaggatagaggatccactaaacaaaaacaaggatgcaggtacctgccttagctataggcactaggttagccctgcaagtaattccggcttcccgatgtggatgggtcatgtgatgactaggagcgtcacatgacacaggaagtgaggcaatcacagccatatttctaaagggcaattggcttcctctcatctgttacagattatatgccctataaaactctggagggcatgtatgggtgacaactttaggaagagcctagaacacagatttgcatggagaatggggtagaggctcctaaatcccagaggatgggaaccacagcaggcacacttcttttttgatagaaatgtcaaaaaggtacaaataaagctgatataatttaaaaaaaaaaacttcactgtgcaaaatactgacactaccaagatgtttaagacagtctttgccctatagaggtgtgtgaggcatggaagtcagacacacagatatttacagataaagacagaaacggtaacaggtgtgcatggagagctctctgagatgaggagggaccattcgtgggtggggaattatccaggatggcttcacagaggaaaaagtgaggggggttattagccagtgaagtgcagggcacaagagggtgggacactggcagtgggatgacaggactggagggagggagtggtcagtgcggctcctctgcagcgtcccttgtttcatcatcagatgcagatggtaataactgttcttccttcctcacaagacagggaggtttgtaaagtcgttcaaaaaccaaagtgttgtacaaagccatatcctcagtggacacaaggaggaagctgtccatggtgtggcctcatgaaccacatcaaatgagatttagcgggagtggcacacacagtcatgacctgactaatcccagctctcagcccatttccttgcctggaaaatggaggcaatgccacaacctcaaagggtggttactgcagtcagtgaggtaagtgcagtgcctgaccacttggtaggcacctgggaactacttgtctcttgtttgtattttttgttgttgttgttttttgagacagagtctcactctgttgcccaggctggagtgcagtggcgtgatcttggctcactgcaacctccacctcctgggttcgagtgattctcctgcctcagcctcccaagtagctgggattacaggtgccctccaacatgctcagctaatttttttatttttagtagagatggggtttcactatgttggccaggctggtctgaaactctggacctcaggtgatctgctgccttggtatccgaaagtgctgggattacaggcatgagccactgcgctggaccccagccatcctttttgttctttaaatgatctcagtgaagtctttctagatacactccaagcaaaattgatcatttcttcttctaggttcctccagtaatttttttttttttggttttgagacagagtcttgccctgttgcccaggctggagtgcagtgtgatctcggctcaagcaattgccctgcctcagcctcctgagtagctgggattacaggagcccaccaccatgcccagctaatttttgtatttttagtagagatggggtttcaccatgttggccaggctggtctcgaacccctgacctcaggtgatccaccagcctcagcctcccaaggtgttgggattatagcaatgagccaccacacccggtccctccagtaattaagtacagtctcagtgagacagcaagtttggaatcctggctacaccatttactggctgtttgactttcaataaatcaatcactctaagcctctgtttcatctataaaaggggagtgataactcctacctcacagaattgttgtgaggtttgagtgtgataatgtgtctctagtacactgcttgacactaaacattgcaacatgtcaggcctctgttcctggagttccttgaaggaatgtcttatgcattctaagtatcctcgtagatagcctggcacaggggtagctgtcaagttgtagaattgaactagttgcttcactatgtcagtagccaccccttccagacttctcacttttcaaggaaacattgcacaggatttgttctgggtagggcaggtaatatctagtaccttacttccctcaagttcattcatctcacagatatttcctgagcacattctacatttgcctctcctgctctattgagtttagaagtccaaacatctccttccacttcccctctgcatagtgagcctcttcttttttggccaggtactgagcaattttttgtttgtttgtttgagacagggtctccgtatgttgccctggatggagtgtagtagcttgatttcggctcagtgcaacctctgcctccagaataagcaatcctcccacctcagcctccagaatagctgggactataggcgcacaccaccacacctggctaatttctgtattttttgtagagacggggtcttactgtgttgcgcaggttggtctcaaactcctaaactcaaaggatcctcccgccttggctttgcaaagtgctgggattacagggtgaggcactgcgcccagtccgcttgttttttgttttgtttttttttttgagacggagtatctctgtcacccaggctggagtgcagtggcacgatctcggttcactgcaacctccgcctcctaggttcaagcggagggtagggaccagtccatcctggcacccatctgcagctccagtgcgaatcccaaccccgacgctcgtcgccgggctccgtgaatgtttgtcacatgtctgaagaacgtatgaattacataaccttcttcccactccacccctcaaaaagcaagtggatataaagacttgaagattttataatctcttcttcattagtaaaatctgaccatccttaccgttaaaaataataatgatggttggccgggcgcggtggctcacgcctataatcacagctgtttggcaggccgaggcgggcggatcacgaggtcaggaattggagaccagcctgaccaacatggtgaaaccccgtctctactaaaagatacaaaaaattagccgtgcgtggtggcgggtgcctgtaatcccacgtacttgggaggctgaggtcggagaatcgcctgaacccgggaggcggaggttgcagtgagccaagaaggcgccattacactccagcctgggcgacagggcgagactccctcaaaaaataataataataacaataataataataatggtgaaaaaggttaagtgcgaacgcagggaggggacagctaagatccaaaggtcgaaatattcattacgcctaccgtcggcgaagagaaacagcagccccaaccggaggcaaaacgaaatcccaccgcagcctgcaaaggcgcctgggcgggactggagactggggccccgcgcagtaagactctgaaggcaggatgcagcccgaccacccgcagccgcgagaaaagcagccctggggagtcggggcgggacctggattggaaaagagacggtcactcatgcagaggcgggactcagagcccttcctcaagtctcattggtcaagttgaacgaacaagtgtcgctggcgagccggagagagagagagagcgagagcgagagagcgagcgagagagagcgagagagcgagagagcgagcgagagagagagagagagagagaggagccggcgcgagaactacgcatgcgtgtcggcgttttcccgccagcacactgttggtggatgggggcgattgaattcccacagtgagtccagcccaccgaagctcagaggattcctaaccttcctctttagagagcctcaggttagggaacgtccagtgcgcagaagctgccccgtgggaatcccattgtccatcgcctcctaatgtttcgtcctggccgtgccctatcccttcctgaggctgggttgttatgatgctgaattattcaagaagtcttgcagcctgacgccatctctgggcagtgctcctcccacctccctgtcttccttggagggcaccacgttgcccctacaagcacagggtcctgaagctgtttacagggccccaccctgccactttagtatcttaacgaatgtttgttgaggacctactgagtgttagacccactgcaatgagcaagtttctgatcttgggaccttaacattctatgggacgacacagatatataaacaaaaataataattttagatagtgatacatgctgaaaccatgggtatgtggccaggcgcggtgagtcacacctgtaatcccagcactttgggagaccgaggcaggcggattacttgaggtcaggagtttgagacccgcctggccaacatggtgaaaccctatctcttctaaaaatacaaaatttagctgggcgggcatggggacgggcgcctgtaatcccagctactcgggaggctgaggcaggagaattgcttgaacccaggaggtggaaggttgcagtgagcccaaatcacaccactgcactccagcctgggtaacaaagcaagaccctgtctcaaaaaaagacaaaatgaaacaaacaaacaaaaaaaaacatggctatgtgatagaatgactgcgatggggattgggtgctcactgaaatggtgagaatggagctaaaacctgatattaggtgatagcaccagatataggggccaagggtttcatgcttggaaaacagcaggtgcaaacgtccttgtgctgtgaggaaatttggctggaacagaaggtgggccagattgtagctgaccatagtctctgggagtttggagatgattctaaatacaatgggaagccattaaattgaggcagagccttgatgtgacgtgctttatcatcattttgctgagtggagaatggattgctgggaaaagtgaaagactgattcagaggttgtggagagaggatggctggagctaggtagctggggaggtggtgggatgtggatgaattcaggataggattggttgtgttggatatgggaaaagaagtcaccatggttcctgagcttttggaatgtgcaaatgggtaatggaggtgccattgagatgggaacagtggaaaaataaccggtgctggggtgggaatcaagagttcagttgtggccatgttgaatctgaaatgcccattagacatccaagcagacatgctgagttggatggcgctcat

SEQ ID NO: 12 - DNA, 13625 bp, Homo sapiens
  Human GBA1 (N370S mutant)
acagaagtacaatgctgattatgatctgtcagctcagcaaggggcagacaccctggccttcatgtctctcctggaggagaagttgatcccggtgctggtgagtgtgcccagacctcccagcatccatggccagccggggaggggacgggaacacacagacccacacagagactcaggagagcatggaggtcagaagcccaccttgaatcagacaggtgcactggctcagacctgcctgtttcttcctgcccacccaatccaggtacatactttttggatagacaccaagaactacgtagaagtgacccggaagtggtatgcagaggctatgccctttcccctcaacttcttcctgcctggccgcatgcagcggcagtacatggaacggctacagctgctgactggggagcacaggcctgaggacgaggaagagctggagaaggaggtagctctgagaccgggggctattgtatgagatgagccccaaggatgctggccaggaatgggagtgcttaggtgcggaggtggcactgttcccgcagctgcaagcctacctgtgtcgcccctacagctgtaccgagaggctcgggagtgtctgaccctgctctctcagcgcctgggctctcaaaagttcttctttggagatgcgtgagtctgactccaagagggtaatgggtggcttggaagaagatacaggttcagatggagcagctggagctggggctggggctggggctggctcaggctctggataggaggtccctgagacagatactggccctggtgacagtggggctgtgcgtggggccagagccttctcagaggtacaaaagggtagggtgggagggcagccaggcacaggaagggcctgaagagctgtggggcactgagtgtgccctttatgcagccctgggatagagccctattcagggccaggctggcgccacctggggatctctccccataccaggtctagaactgtgtgtcctgtccttccctggtggccgcctgctgcccagagcccacctcccaaggctgactcttcctccagctccatctttaccccttctaccccagtggttctcctccatcccacccttctctctctgctccagccctgcctccttggacgccttcgtcttcagctacttggccctgctgctgcaggcaaagctgcccagtgggaagctgcaggtccacctgcgtgggctgcacaacctctgtgcctattgtacccacattctcagtctctacttcccctgggatggaggtaaggggcagatgggaggggcagccctggggagagtgggcagggatccaagaactagttctcctaacacaccttccttccttgaccctcagctgaggtaccaccgcaacgccagacaccagcaggcccagagactgaggaggagccataccggcgccggaaccagatcctatctgtgctggcaggactggcagccatggtgggctacgccttgctcagcggcattgtctccatccagcgggcaacgcctgctcgggccccaggcacccggaccctgggcatggctgaggaggatgaagaggaatgatttgtcctcacgctcccaagactggtttttctactctcatgcattccagaggcccccgtgcctcctcgttgttggtacagccggacacggggtgctgccacccagaataaagccactcacactgactgggctcaaacattttctcctttaagagctgccatttttcctggctggtgccataggaatcatctgggtgcctgggcacacccgctgctgctttaaggcttccgccctgatgctgacactgctgctccacgggcccagttctgcatctccaggaaagacaaacagtctccagttttgggcccagctttcctagtctcttcttttccttaccctcagccctgatcttgtgtttgtacggacagtgagctcaccctaggcctggacccaggcccagtttccaaagcaagcagcacacagcgcatgttcacataagcatgggggctggggggacactggggcttactgatctttttctaggggcctccagcccctggcaccacctagaggggaaagtgagtcacccaaaccattgcccctgggcttacgtcgctgtaagctcacactggccctgctgtgccctctttagtcacagacagcgtgtgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagctagggagcagggaggagaagctgagagtgtgatcctgccaaggcccccaacgctgtcttcagcccacttcccagacctcaccattgccctcaccggtttagcacgaccacaacagcagagccatcgggatgcatcagtgccactgcgtccaggtcgttcttctgactggcaaccagccccactctctgggagccctcaggaatgaacttgctgaatgtgggaacagatggtcagagtccctcggggtacctcccatgaaaccctcatctaagaagtcacccacccacggacccaccccataactcctgcagaggctctgccctggctctctaggcctggagccatgctgctgggcactgaccctgcttttctgcatcgcagtccagcctcaggcattggggttttctgttgctacctagtcacttcctgcctccatggtgcaaaaggggatgggtgtgcctcttccgaggttccaccctgaacaccttcctgctccctcgtggtgtagagtgatgtaagccatccgatgtaggagatgataggcctggtatggaatgggggtgcccgccctccactcacctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggctctagggtaaggacaaaggcaaagagacaaaggctcaacactgggggtccccagagagtgtaggtaagggtcacatgtgggagaggcagctgtgggtaggtcagccctgtgaggggcacattccttagtagctaaggagttgggggtgtgaagatccaggcatctcaaggggagctgagaagtctgaggcagctgcaagtgcctcagtagttgcaaaaggggcaatgaggtgtgcagacctgtgaaggaaagggaagatagggaatcatggttccccagagttgctcaaaagggcaggctagctggggaaagctggacaggaagggcttctgtcagtctttggtgaaactagtaagaggtctgaggtctgctttgcaggaagggagactggggtggcttaccgtgatgatgctgtggctgtactgcatccctcgatcccaggagcctagccgcacactctgctcccagaacttggagcccacacaggcctctgaggcaaagagcatggtgttggggaacaggcggtgtgtctcccctagggtggctttggctggagccagaaagtccaggtaccaatgtacagcaatgccatgaacatatttagctgcttctgggtctgtcagtacctgcaaaggaagagcaactgatcctggaccttgcacacaggcttctggaacttctagttcctgttgtaggaatcctggagttgggtgacgggaagaatgcaactagagaggtttggggagatttttttttgtttttgagacaggatgtcactctgtcactcgggctggagtacagtggcgcaatcacgactcactgcagccttgacttcctagggtcaactgatcctcccacattagcctcctgagtagctgggactacacgggtgccacacccagctaatttgtgtgtgtatgtgtgtgtatgtatgtgtgtgtgtgtatatatatatacatataaacacatatatatgtatatacacatacatacatgaaccaccacccccagcctagatagttttgttttgttttgtttcgagatggagtctcgctcagcctcccaggctggagtgcagtggcgcgatctcggctcaccgcaaccaccatctcccgggttcaactgattctcccttctcagcctcctgagtagctgggattacagacacccaccatcatgcccagataattttttttttttgtattttagtagacacagggtttcaacacgttggccagggtggtcttaaactcctgacctcaggtgatcacccgcctcggcttcccaaagtgctgggtttgcatgagtgagccacctcgcccagcccctagaaaggtttcaagcgacaactgtgggatccatggcaccctggaggtccaggggaatggtgctctaggaatccatagttgggtagagaaatcgctctaagtttgggagccagtcatttggatgctggatttgaaggtcactggagcaccatggaggtccaggccttaccacctttgcccagtggggcagcagcaagcgttggtcatccagcatgagtaggcggacattgtggtgagtactgttggcgagggtaggacctaggtcacgggcaatgaagtctcgctgatgttcaggggtgaagcccaggcactggaaggggtatccactcaacagcccagcagaaggctcattttcagctgtcactgcccagaactgtaacttgtgctcagcataggcatccaggaacctggcaagagaaaggtcatgaatgatccggccaagaaagtggaccagaccagctgggtgtggtggctcacacctgtaatcccagcactttgggaagccgaggcaggtggatcacttgagttcaggagttcgagaacagcctggcgaaaccccgtctctactaaaaatagaaaaatcagctgggcctggtggcaggcgcctataatcccagctacttggaaggctgaggcaggagaattgcttgaactcaggaggcagaggttacagtgagtgaagatggcgccacagcactccagcctgggtgacagagagagagactccttctcaaaaaaaaaaaaaagaagaaaaataaaaagaaagtgggccagaccgagagaacaggaagcctgatggagtgggcaagattgacaggcccaaggctgaaaggcccagaaggtagaaaggtgagctgaggacaggcagatctggaagtggaactaggttgagggttgggacacagatcagcatggctaaatgggaggccagtcctgatcccacatccttgctgatcccttacttcacaaagtatctggcccaggtctggtggtagatgtctccgggctgtcccttgagtgaccccttcccattcaccgctccattggtcttgagccaagtgggtgatgtccaggggctggcaaggagtgaaacgggacgctgggccaactgcagggctcggtgaatcaggggtatctagagacaaaggtagtgaagagagaagcacccagagttggaacacatactagcccaaccagtgcatccggttcagccattagcctccaccctcccacccccaggacaaaacagcaggggacaaaatgtctgtacaagcagacctaccctacagtttctcaacccccagacatcagggccctcagggcctgaaaaagctagaatgcctaccttgagcttggtatcttcctctgggaggctgaagttgtgcaactggaaatcatcaggggtgtctgcataggtgtaggtgcggatggagaagtcacagctggccatgggtacccggatgatgttatatccgattcctacagaaaaggatgatcaagatatggtagtccgagtcaataggagagtatgggactctgcttatcacttgccagtcctaatagtgtctgagtcagggccaaagggaacttgggctcctgggttggaacctgtggaggctggcacctgggtgaagcgcaggcctttctgagcctgagtccgtagcagttagcagatgataggcggcgaaatcttatttcacagggcattaaaacaggaaccaaatgtcagggatgggcagaagtcagggtccaaagaaagggcaaagaaaagtgtcagtggctcacgcatgtaatcccagcactttgggaggccgatgtgggcagatcacgaggtcaggagttcgcaatcatcctggccaacatagtgaaaccctgtctctactaaaaatacaaaaaattagccgggcgtggtggcaggcacctgtaatcccaggtactcgagaggctgagacaggagaatcacctgaacccgggaggtggaggttgcagtgagctgagattgtgccactgcactccagcctgggtgacagtgcgagactctgtctcaaaaaaaaaaaaagaaaaaagaaaagtgtctgctgggctcggtggctcacacttgtaattccagcacttcgggaggccaaggcaggtatatcatttgaggtcaggagtttaagactagcctggtcaacatgatgaaaccctgtctctactaaaaatacaaaaattagccaggtggtagtggcgcacgcctataattccagctactcaggaggctgaggcaggagaatcactatagcctgggaggcagaggttgctgtgaggggagatcacaccactgcactcctgtctcgccgacagaatgggcagagtgagattctgcctcaaaaaaattttaaaaaaagaaaagaaaaacgaaaagtttcaatggctctatgtcatcttgtccccttcctcctcaccttcttcagagaagtacgatttaagtagcaaattttgggcagggggtgacagggcaaggatgttgagagcagcagcatctgtcatggcccctccaaatcccttcactttctggaacttctgttctggctgcagggtcagtagcaggcctgaggacatccacagggaataagggtatcagtacccagcgggaaactccatggtgatcactgacaccatttacctctaggaggacccagcctggcccagggggtgaggggtgtaatggttacctgtgcccgtgtgattagcctggatgggccccatactcagctccatccgtcgcccactgcgtgtactctcatagcggctgaaggtaccaagggcaggaaaggtcggggggtcaaaggagtcacagtatgtggcattgcagacacacaccaccgagctgtagccgaagcttttagggatgcaggggcgggcacctgggagggagggagtacaagcagagtgaggtctgatgaagacatggagaatggacacatctgctaggagagactgaacacggtttcaaaattcctcaccccttggccgggcgcaggggctcacacctgtaatcctagcactttaggaggccgaggtgggcggatcacctgaggtgaggagtttgagacctgccttgccagcatggtgaaaccccgtctctactaaaaatacaaaaattagctgggcgtagtggtggccgcctgtaatcccagctacctgggaagctgaagcaagagaatcgcttgaatctgggaggcagaggttggaatgagccaaaattgcaccactgcactccagcccaggcaacagagtaagactctgtttcaaaagaaaaaaaacaaaatcctcaccccaaagttggtctcagtcactcaaaaggatttattgagcacctactaaaagtctaccaccagcttactggaaggctaccaaaggactatgaggcagaagggaggctctgtgctacctccccactgccttgactcactcacctgatgcccacgacactgcctgaagtagaagcaatcctgtgaggctgccagccatgatgcttaccctactcaaaggcttgggacattcctgaggacagaatgaggaatgactgaaaagcaagcccctctccacccctcaactactctcctgggcagggcttagctgcctttgggtgcccatggcccggtctcccacattcattaggacaaggcccacagaaacctgggtgcagctctccctgcaacccttctgatgacaactctctgcccaccccaaatcaggatgctccccaccactcttcccacccatttcaacttcgacccctccctccatctgtgccttgctcaaagagccatgatggccctggattcaaagagagtctgtcattcattaaattccagtgccaggattccagaagcactgtgacaatgctgattgggagctctctctcttacctctctggaaggacttgaaaactccatcccctcagggtcattagatgaagagaagaccacaggggttccagagtctctgaaggatagaggatccactaaacaaaaacaaggatgcaggtacctgccttagctataggcactaggttagccctgcaagtaattccggcttcccgatgtggatgggtcatgtgatgactaggagcgtcacatgacacaggaagtgaggcaatcacagccatatttctaaagggcaattggcttcctctcatctgttacagattatatgccctataaaactctggagggcatgtatgggtgacaactttaggaagagcctagaacacagatttgcatggagaatggggtagaggctcctaaatcccagaggatgggaaccacagcaggcacacttcttttttgatagaaatgtcaaaaaggtacaaataaagctgatataatttaaaaaaaaaaacttcactgtgcaaaatactgacactaccaagatgtttaagacagtctttgccctatagaggtgtgtgaggcatggaagtcagacacacagatatttacagataaagacagaaacggtaacaggtgtgcatggagagctctctgagatgaggagggaccattcgtgggtggggaattatccaggatggcttcacagaggaaaaagtgaggggggttattagccagtgaagtgcagggcacaagagggtgggacactggcagtgggatgacaggactggagggagggagtggtcagtgcggctcctctgcagcgtcccttgtttcatcatcagatgcagatggtaataactgttcttccttcctcacaagacagggaggtttgtaaagtcgttcaaaaaccaaagtgttgtacaaagccatatcctcagtggacacaaggaggaagctgtccatggtgtggcctcatgaaccacatcaaatgagatttagcgggagtggcacacacagtcatgacctgactaatcccagctctcagcccatttccttgcctggaaaatggaggcaatgccacaacctcaaagggtggttactgcagtcagtgaggtaagtgcagtgcctgaccacttggtaggcacctgggaactacttgtctcttgtttgtattttttgttgttgttgttttttgagacagagtctcactctgttgcccaggctggagtgcagtggcgtgatcttggctcactgcaacctccacctcctgggttcgagtgattctcctgcctcagcctcccaagtagctgggattacaggtgccctccaacatgctcagctaatttttttatttttagtagagatggggtttcactatgttggccaggctggtctgaaactctggacctcaggtgatctgctgccttggtatccgaaagtgctgggattacaggcatgagccactgcgctggaccccagccatcctttttgttctttaaatgatctcagtgaagtctttctagatacactccaagcaaaattgatcatttcttcttctaggttcctccagtaatttttttttttttggttttgagacagagtcttgccctgttgcccaggctggagtgcagtgtgatctcggctcaagcaattgccctgcctcagcctcctgagtagctgggattacaggagcccaccaccatgcccagctaatttttgtatttttagtagagatggggtttcaccatgttggccaggctggtctcgaacccctgacctcaggtgatccaccagcctcagcctcccaaggtgttgggattatagcaatgagccaccacacccggtccctccagtaattaagtacagtctcagtgagacagcaagtttggaatcctggctacaccatttactggctgtttgactttcaataaatcaatcactctaagcctctgtttcatctataaaaggggagtgataactcctacctcacagaattgttgtgaggtttgagtgtgataatgtgtctctagtacactgcttgacactaaacattgcaacatgtcaggcctctgttcctggagttccttgaaggaatgtcttatgcattctaagtatcctcgtagatagcctggcacaggggtagctgtcaagttgtagaattgaactagttgcttcactatgtcagtagccaccccttccagacttctcacttttcaaggaaacattgcacaggatttgttctgggtagggcaggtaatatctagtaccttacttccctcaagttcattcatctcacagatatttcctgagcacattctacatttgcctctcctgctctattgagtttagaagtccaaacatctccttccacttcccctctgcatagtgagcctcttcttttttggccaggtactgagcaattttttgtttgtttgtttgagacagggtctccgtatgttgccctggatggagtgtagtagcttgatttcggctcagtgcaacctctgcctccagaataagcaatcctcccacctcagcctccagaatagctgggactataggcgcacaccaccacacctggctaatttctgtattttttgtagagacggggtcttactgtgttgcgcaggttggtctcaaactcctaaactcaaaggatcctcccgccttggctttgcaaagtgctgggattacagggtgaggcactgcgcccagtccgcttgttttttgttttgtttttttttttgagacggagtatctctgtcacccaggctggagtgcagtggcacgatctcggttcactgcaacctccgcctcctaggttcaagcggagggtagggaccagtccatcctggcacccatctgcagctccagtgcgaatcccaaccccgacgctcgtcgccgggctccgtgaatgtttgtcacatgtctgaagaacgtatgaattacataaccttcttcccactccacccctcaaaaagcaagtggatataaagacttgaagattttataatctcttcttcattagtaaaatctgaccatccttaccgttaaaaataataatgatggttggccgggcgcggtggctcacgcctataatcacagctgtttggcaggccgaggcgggcggatcacgaggtcaggaattggagaccagcctgaccaacatggtgaaaccccgtctctactaaaagatacaaaaaattagccgtgcgtggtggcgggtgcctgtaatcccacgtacttgggaggctgaggtcggagaatcgcctgaacccgggaggcggaggttgcagtgagccaagaaggcgccattacactccagcctgggcgacagggcgagactccctcaaaaaataataataataacaataataataataatggtgaaaaaggttaagtgcgaacgcagggaggggacagctaagatccaaaggtcgaaatattcattacgcctaccgtcggcgaagagaaacagcagccccaaccggaggcaaaacgaaatcccaccgcagcctgcaaaggcgcctgggcgggactggagactggggccccgcgcagtaagactctgaaggcaggatgcagcccgaccacccgcagccgcgagaaaagcagccctggggagtcggggcgggacctggattggaaaagagacggtcactcatgcagaggcgggactcagagcccttcctcaagtctcattggtcaagttgaacgaacaagtgtcgctggcgagccggagagagagagagagcgagagcgagagagcgagcgagagagagcgagagagcgagagagcgagcgagagagagagagagagagagaggagccggcgcgagaactacgcatgcgtgtcggcgttttcccgccagcacactgttggtggatgggggcgattgaattcccacagtgagtccagcccaccgaagctcagaggattcctaaccttcctctttagagagcctcaggttagggaacgtccagtgcgcagaagctgccccgtgggaatcccattgtccatcgcctcctaatgtttcgtcctggccgtgccctatcccttcctgaggctgggttgttatgatgctgaattattcaagaagtcttgcagcctgacgccatctctgggcagtgctcctcccacctccctgtcttccttggagggcaccacgttgcccctacaagcacagggtcctgaagctgtttacagggccccaccctgccactttagtatcttaacgaatgtttgttgaggacctactgagtgttagacccactgcaatgagcaagtttctgatcttgggaccttaacattctatgggacgacacagatatataaacaaaaataataattttagatagtgatacatgctgaaaccatgggtatgtggccaggcgcggtgagtcacacctgtaatcccagcactttgggagaccgaggcaggcggattacttgaggtcaggagtttgagacccgcctggccaacatggtgaaaccctatctcttctaaaaatacaaaatttagctgggcgggcatggggacgggcgcctgtaatcccagctactcgggaggctgaggcaggagaattgcttgaacccaggaggtggaaggttgcagtgagcccaaatcacaccactgcactccagcctgggtaacaaagcaagaccctgtctcaaaaaaagacaaaatgaaacaaacaaacaaaaaaaaacatggctatgtgatagaatgactgcgatggggattgggtgctcactgaaatggtgagaatggagctaaaacctgatattaggtgatagcaccagatataggggccaagggtttcatgcttggaaaacagcaggtgcaaacgtccttgtgctgtgaggaaatttggctggaacagaaggtgggccagattgtagctgaccatagtctctgggagtttggagatgattctaaatacaatgggaagccattaaattgaggcagagccttgatgtgacgtgctttatcatcattttgctgagtggagaatggattgctgggaaaagtgaaagactgattcagaggttgtggagagaggatggctggagctaggtagctggggaggtggtgggatgtggatgaattcaggataggattggttgtgttggatatgggaaaagaagtcaccatggttcctgagcttttggaatgtgcaaatgggtaatggaggtgccattgagatgggaacagtggaaaaataaccggtgctggggtgggaatcaagagttcagttgtggccatgttgaatctgaaatgcccattagacatccaagcagacatgctgagttggatggcgctcat

SEQ ID NO: 13 - DNA, 20 bp, Artificial Sequence
  crRNA (rs76763715)
agccgaccacatggtacagg

SEQ ID NO: 14 - DNA, 20 bp, Artificial Sequence
  crRNA (rs76763715)
gtacaggaggctctagggta

SEQ ID NO: 15 - DNA, 20 bp, Artificial Sequence
  crRNA (rs76763715)
gagacaaaggctcaacactg

SEQ ID NO: 16 - DNA, 20 bp, Artificial Sequence
  crRNA (rs76763715)
aagagacaaaggctcaacac

SEQ ID NO: 17 - DNA, 20 bp, Artificial Sequence
  crRNA (rs76763715)
acatggtacaggaggctcta

SEQ ID NO: 18 - DNA, 20 bp, Artificial Sequence
  crRNA (rs76763715)
aggctctagggtaaggacaa

SEQ ID NO: 19 - DNA, 20 bp, Artificial Sequence
  crRNA (rs76763715)
ctagagcctcctgtaccatg

SEQ ID NO: 20 - DNA, 20 bp, Artificial Sequence
  crRNA (rs76763715)
cacatggtacaggaggctct

SEQ ID NO: 21 - DNA, 20 bp, Artificial Sequence
  crRNA (rs76763715)
agcctcctgtaccatgtggt

SEQ ID NO: 22 - DNA, 20 bp, Artificial Sequence
  crRNA (rs76763715)
agtcggtccagccgaccaca

SEQ ID NO: 23 - DNA, 20 bp, Artificial Sequence
  crRNA (rs76763715)
atgtggtcggctggaccgac

SEQ ID NO: 24 - DNA, 20 bp, Artificial Sequence
  crRNA (rs76763715)
tccagccgaccacatggtac

SEQ ID NO: 25 - DNA, 40 bp, Artificial Sequence
  5 ssODN (rs76763715)
cagggcaaggttccagtcggtccagccgaccacatggtac

SEQ ID NO: 26 - DNA, 45 bp, Artificial Sequence
  3 ssODN (rs76763715)
agcaagttctagggtaaggacaaaggcaaagagacaaaggctcaa

SEQ ID NO: 27 - DNA, 85 bp, Artificial Sequence
  ssODN (rs76763715)
cagggcaaggttccagtcggtccagccgaccacatggtacagcaagttctagggtaaggacaaaggcaaagagacaaaggctcaa

SEQ ID NO: 28 - DNA, 47 bp, Artificial Sequence
  5 ssODN (rs76763715)
caaggttccagtcggtccagccgaccacatggtacaggaggttctag

SEQ ID NO: 29 - DNA, 41 bp, Artificial Sequence
  3 ssODN (rs76763715)
ggtaagcacaaaggcaaagagacaaaggcgcaacactgggg

SEQ ID NO: 30 - DNA, 88 bp, Artificial Sequence
  ssODN (rs76763715)
caaggttccagtcggtccagccgaccacatggtacaggaggttctagggtaagcacaaaggcaaagagacaaaggcgcaacactgggg

SEQ ID NO: 31 - DNA, 68 bp, Artificial Sequence
  5 ssODN (rs76763715)
aaggttccagtcggtccagccgaccacatggtacaggaggttctagggtaaggacaaaggcaaagaga

SEQ ID NO: 32 - DNA, 40 bp, Artificial Sequence
  3 ssODN (rs76763715)
caaacgctcaacactgggggtccccagagagtgtaggtaa

SEQ ID NO: 33 - DNA, 108 bp, Artificial Sequence
  ssODN (rs76763715)
aaggttccagtcggtccagccgaccacatggtacaggaggttctagggtaaggacaaaggcaaagagacaaacgctcaacactgggggtccccagagagtgtaggtaa

SEQ ID NO: 34 - DNA, 81 bp, Artificial Sequence
  5 ssODN (rs76763715)
aaggttccagtcggtccagccgaccacatggtacaggaggttctagggtaaggacaaaggcaaagagacaaaggctcaaca

SEQ ID NO: 35 - DNA, 40 bp, Artificial Sequence
  3 ssODN (rs76763715)
ctggccgtccccagagagtgtaggtaagggtcacatgtgg

SEQ ID NO: 36 - DNA, 121 bp, Artificial Sequence
  ssODN (rs76763715)
aaggttccagtcggtccagccgaccacatggtacaggaggttctagggtaaggacaaaggcaaagagacaaaggctcaacactggccgtccccagagagtgtaggtaagggtcacatgtgg

SEQ ID NO: 37 - DNA, 80 bp, Artificial Sequence
  5 ssODN (rs76763715)
caaggttccagtcggtccagccgaccacatggtacaggaggttctagggtaaggacaaaggcaaagagacaaaggctcaa

SEQ ID NO: 38 - DNA, 40 bp, Artificial Sequence
  3 ssODN (rs76763715)
cactccgggtccccagagagtgtaggtaagggtcacatgt

SEQ ID NO: 39 - DNA, 120 bp, Artificial Sequence
  ssODN (rs76763715)
caaggttccagtcggtccagccgaccacatggtacaggaggttctagggtaaggacaaaggcaaagagacaaaggctcaacactccgggtccccagagagtgtaggtaagggtcacatgt

SEQ ID NO: 40 - DNA, 43 bp, Artificial Sequence
  5 ssODN (rs76763715)
caaggttccagtcggtccagccgaccacatggtacaggaggtt

SEQ ID NO: 41 - DNA, 40 bp, Artificial Sequence
  3 ssODN (rs76763715)
ctaggctaaggacaaaggcaaagagacaaaggctcaacac

SEQ ID NO: 42 - DNA, 83 bp, Artificial Sequence
  ssODN (rs76763715)
caaggttccagtcggtccagccgaccacatggtacaggaggttctaggctaaggacaaaggcaaagagacaaaggctcaacac

SEQ ID NO: 43 - DNA, 55 bp, Artificial Sequence
  5 ssODN (rs76763715)
caaggttccagtcggtccagccgaccacatggtacaggaggttctagggtaagga

SEQ ID NO: 44 - DNA, 40 bp, Artificial Sequence
  3 ssODN (rs76763715)
caaagccaaagagacaaaggctcaacactgggggtcccca

SEQ ID NO: 45 - DNA, 95 bp, Artificial Sequence
  ssODN (rs76763715)
caaggttccagtcggtccagccgaccacatggtacaggaggttctagggtaaggacaaagccaaagagacaaaggctcaacactgggggtcccca

SEQ ID NO: 46 - DNA, 29 bp, Artificial Sequence
  5 ssODN (rs76763715)
aaggttccagtcggtccagccgacgacat

SEQ ID NO: 47 - DNA, 52 bp, Artificial Sequence
  3 ssODN (rs76763715)
ggtacaggaggttctagggtaaggacaaaggcaaagagacaaaggctcaaca

SEQ ID NO: 48 - DNA, 81 bp, Artificial Sequence
  ssODN (rs76763715)
aaggttccagtcggtccagccgacgacatggtacaggaggttctagggtaaggacaaaggcaaagagacaaaggctcaaca

SEQ ID NO: 49 - DNA, 41 bp, Artificial Sequence
  5 ssODN (rs76763715)
aaggttccagtcggtccagccgaccacatggtacaggaggt

SEQ ID NO: 50 - DNA, 40 bp, Artificial Sequence
  3 ssODN (rs76763715)
tctaccgtaaggacaaaggcaaagagacaaaggctcaaca

SEQ ID NO: 51 - DNA, 81 bp, Artificial Sequence
  ssODN (rs76763715)
aaggttccagtcggtccagccgaccacatggtacaggaggttctaccgtaaggacaaaggcaaagagacaaaggctcaaca

SEQ ID NO: 52 - DNA, 25 bp, Artificial Sequence
  5 ssODN (rs76763715)
aaggttccagtcggtccagccgacc

SEQ ID NO: 53 - DNA, 56 bp, Artificial Sequence
  3 ssODN (rs76763715)
acatggtacaggaggttctagggtaaggacaaaggcaaagagacaaaggctcaaca

SEQ ID NO: 54 - DNA, 81 bp, Artificial Sequence; identical to SEQ ID NO: 34
  ssODN (rs76763715)

SEQ ID NO: 55 - DNA, 40 bp, Artificial Sequence
  5 ssODN (rs76763715)
ttcggggttcagggcaaggttccagtcggtccagccgacc

SEQ ID NO: 56 - DNA, 56 bp, Artificial Sequence
  3 ssODN (rs76763715)
acatgatacaggaggttctagggtaaggacaaaggcaaagagacaaaggctcaaca

SEQ ID NO: 57 - DNA, 96 bp, Artificial Sequence
  ssODN (rs76763715)
ttcggggttcagggcaaggttccagtcggtccagccgaccacatgatacaggaggttctagggtaaggacaaaggcaaagagacaaaggctcaaca

SEQ ID NO: 58 - DNA, 49 bp, Artificial Sequence
  5 ssODN (rs76763715)
aattgggtcctccttcggggttcagggcaaggttccagtcggtccagcc

SEQ ID NO: 59 - DNA, 60 bp, Artificial Sequence
  3 ssODN (rs76763715)
gaccacatggtacaggaggttctagggtaaggacaaaggcaaagagacaaaggctcaaca

SEQ ID NO: 60 - DNA, 109 bp, Artificial Sequence
  ssODN (rs76763715)
aattgggtcctccttcggggttcagggcaaggttccagtcggtccagccgaccacatggtacaggaggttctagggtaaggacaaaggcaaagagacaaaggctcaaca

SEQ ID NO: 61 - DNA, 40 bp, Artificial Sequence
  5 ssODN (rs76763715)
gttcagggcaaggttccagtcggtccagccgaccacatgg

SEQ ID NO: 62 - DNA, 50 bp, Artificial Sequence
  3 ssODN (rs76763715)
tacagaaggttctagggtaaggacaaaggcaaagagacaaaggctcaaca

SEQ ID NO: 63 - DNA, 90 bp, Artificial Sequence
  ssODN (rs76763715)
gttcagggcaaggttccagtcggtccagccgaccacatggtacagaaggttctagggtaaggacaaaggcaaagagacaaaggctcaaca

SEQ ID NO: 64 - DNA, 3 bp, Artificial Sequence
  variation (1)...(1): N is A, G, T, or C
  PAM sequence
ngg

SEQ ID NO: 65 - DNA, 5 bp, Artificial Sequence
  variation (1)...(1): N is A, G, T, or C
  variation (3)...(3): R is G or A
  variation (4)...(4): R is G or A
  PAM Sequence
ngrrt

SEQ ID NO: 66 - DNA, 5 bp, Artificial Sequence
  variation (1)...(1): N is A, G, T, or C
  variation (3)...(3): R is G or A
  variation (4)...(4): R is G or A
  variation (5)...(5): N is A, G, T, or C
  PAM Sequence
ngrrn

SEQ ID NO: 67 - DNA, 8 bp, Artificial Sequence
  variation (1)...(1): N is A, G, T, or C
  variation (2)...(2): N is A, G, T, or C
  variation (3)...(3): N is A, G, T, or C
  variation (4)...(4): N is A, G, T, or C
  PAM Sequence
nnnngatt

SEQ ID NO: 68 - DNA, 8 bp, Artificial Sequence
  variation (1)...(1): N is A, G, T, or C
  variation (2)...(2): N is A, G, T, or C
  variation (3)...(3): N is A, G, T, or C
  variation (4)...(4): N is A, G, T, or C
  variation (5)...(5): R is G or A
  variation (6)...(6): Y is C or T
  PAM Sequence
nnnnryac

SEQ ID NO: 69 - DNA, 7 bp, Artificial Sequence
  variation (1)...(1): N is A, G, T, or C
  variation (2)...(2): N is A, G, T, or C
  variation (7)...(7): W is A or T
  PAM Sequence
nnagaaw

(19) United States
(12) Patent Application Publication
(10) Pub. No.: US 2025/0263752 A1
(43) Pub. Date: Aug. 21, 2025

(54) GENE EDITING OF GBA1 IN STEM CELLS AND METHOD OF USE OF CELLS DIFFERENTIATED THEREFROM

(71) Applicant: Aspen Neuroscience, Inc., San Diego, CA (US)

(72) Inventors: Ai ZHANG, San Diego, CA (US); Andres BRATT-LEAL, San Jose, CA (US); Jeanne LORING, San Diego, CA (US); Howard FEDEROFF, San Diego, CA (US)

(73) Assignee: Aspen Neuroscience, Inc., San Diego, CA (US)

(21) Appl. No.: 17/920,705

(22) PCT Filed: Apr. 20, 2021
(86) PCT No.: PCT/US2021/028255

§ 371 Date: Oct. 21, 2022

Related U.S. Application Data
(60) Provisional application No. 63/013,444, filed on Apr. 21, 2020.

(51) Int. Cl. C12N 15/90; A61K 35/30; C12N 5/074; C12N 9/22; C12N 15/11; C12Q 1/6888

(52) U.S. Cl. CPC C12N 15/907; A61K 35/30; C12N 5/0696; C12N 9/22; C12N 15/111; C12Q 1/6888; C12N 2310/20; C12N 2501/999; C12N 2506/08; C12N 2510/00; C12N 2513/00

(57) ABSTRACT

The present disclosure provides methods of correcting gene variants associated with Parkinson's Disease in pluripotent stem cells, and methods of lineage specific differentiation of such corrected pluripotent stem cells into floor plate midbrain progenitor cells, determined dopamine (DA) neuron progenitor cells, and/or DA neurons, or into glial cells, such as microglial cells, astrocytes, oligodendrocytes, or ependymocytes. Also provided are compositions uses thereof, such as for treating neurodegenerative diseases and conditions, including Parkinson's disease.

### CROSS-REFERENCE TO RELATED APPLICATION

This application is a national stage application under 35 U.S.C. § 371 of International Application No. PCT/US2021/028255, filed internationally on Apr. 20, 2021, which This application claims priority from U.S. provisional application 63/013,444, filed Apr. 21, 2020, entitled ‘GENE EDITING OF GBA1 IN STEM CELLS AND METHOD OF USE OF CELLS DIFFERENTIATED THEREFROM,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 165622000200SubSeqList.TXT, created Apr. 21, 2023, which is 64,521 bytes in size. The information in the electronic format of the Sequence Listing is incorporated by reference in its entirety.

### FIELD

The present disclosure relates to methods of genetically editing pluripotent stem cells, including induced pluripotent stem cells, prior to their differentiation into floor plate midbrain progenitor cells, determined dopamine (DA) neuron progenitor cells, and/or dopamine (DA) neurons, or into glial cells, such as microglia, astrocytes, oligodendrocytes, or ependymocytes. Also provided are compositions of the differentiated cells and therapeutic uses thereof, such as for treating neurodegenerative conditions and diseases, including Parkinson's disease.

### BACKGROUND

Genetic variants in certain genes, such as single nucleotide polymorphisms (SNPs) in the glucosylceramidase beta (GBA1) gene, have been associated with an increased risk of developing certain neurodegenerative diseases or disorders, such as Parkinson's Disease (PD). Various methods for differentiating pluripotent stem cells into lineage specific cell populations and the resulting cellular compositions are contemplated to find use in cell replacement therapies for patients with diseases resulting in a loss of function of a defined cell population. However, in some cases, such methods are limited in their ability to produce cells with consistent physiological characteristics, and cells resulting from such methods may be limited in their ability to engraft and innervate other cells in vivo. Moreover, in some cases, such methods involve the use of cells that retain a gene variant, e.g., a SNP, that is associated with an increased risk of developing PD. Improved methods and cellular compositions thereof are needed, including to provide for improved methods for correcting gene variants, e.g., SNPs, that are associated with PD in cells, and for differentiating such cells, such as to produce physiologically consistent cells.

### SUMMARY

Provided herein are methods of correcting a gene variant associated with Parkinson's Disease in a cell, such as in connection with preparing cell for replacement cell therapy for treating Parkinson's Disease. In particular embodiments, the gene variant is a variant of human GBA1.

Provided herein are method of correcting a GBA1 gene variant that includes: introducing, into a cell, one or more agents comprising a recombinant nuclease for inducing a DNA break within an endogenous target gene in the cell, wherein the target gene is human GBA1 and includes a single nucleotide polymorphism (SNP) that is associated with Parkinson's Disease; and introducing, into the cell, a single-stranded DNA oligonucleotide (ssODN), wherein the ssODN is homologous to the target gene and comprises a corrected form of the SNP, wherein the introducing of the one or more agents and the ssODN results in homology-directed repair (HDR) and integration of the ssODN into the target gene.

Also provided herein is a method of correcting a gene variant associated with Parkinson's Disease that includes: introducing into an induced pluripotent stem cell (iPSC), one or more agents comprising a recombinant nuclease for inducing a DNA break within an endogenous target gene in the cell, wherein the target gene is human GBA1 and includes a single nucleotide polymorphism (SNP) that is associated with Parkinson's Disease; and introducing, into the cell, a single-stranded DNA oligonucleotide (ssODN), wherein the ssODN is homologous to the target gene and includes a corrected form of the SNP, wherein (i) the introducing of the one or more agents and the ssODN results in homology-directed repair (HDR) and integration of the ssODN into the target gene; and (ii) after the integration of the ssODN into the target gene, the target gene comprises the corrected form of the SNP instead of the SNP.

Also provided herein is a method of correcting a GBA1 gene variant the method comprising: introducing, into a cell, a single-stranded DNA oligonucleotide (ssODN); wherein the cell comprises a DNA break within an endogenous target gene in the cell, wherein the target gene is human GBA1 and includes a single nucleotide polymorphism (SNP) that is associated with Parkinson's Disease, wherein the ssODN is homologous to the target gene and comprises a corrected form of the SNP, and wherein the introducing results in HDR and integration of the ssODN into the target gene.

In some of any such embodiments, the DNA break is a double strand break (DSB) at a cleavage site within the endogenous target gene. In some of any such embodiments, the DSB is induced by one or more agents comprising a recombinant nuclease.

In some of any such embodiments, the recombinant nuclease is capable of cleaving both strands of double stranded DNA. In some of any such embodiments, the recombinant nuclease is selected from the group consisting of a Cas nuclease, a transcription activator-like effector nuclease (TALEN), and a zinc finger nuclease (ZFN). In some of any such embodiments, the recombinant nuclease is a Cas nuclease.

In some of any such embodiments, the one or more agents comprises the Cas nuclease and a single guide RNA (sgRNA). In some of any such embodiments, the Cas nuclease and the sgRNA are in a complex when they are introduced into the cell. In some of any such embodiments, the Cas nuclease and the sgRNA are introduced as a ribonucleoprotein (RNP) complex. In some of any such embodiments, the Cas nuclease is introduced into the cell by introducing a nucleic acid encoding the Cas nuclease into the cell. In some of any such embodiments, the nucleic acid encoding the Cas nuclease is DNA. In some of any such embodiments, the nucleic acid encoding the Cas nuclease is RNA.

In some of any such embodiments, the Cas nuclease is selected from the group consisting of Cas3, Cas9, Cas10, Cas12, and Cas13. In some of any such embodiments, the Cas nuclease is Cas9. In some of any such embodiments, the Cas nuclease is Cas9 or a variant thereof. In some embodiments, the Cas nuclease is an enhanced specificity Cas9 (eSpCas9). In some embodiments, the Cas nuclease is a high fidelity Cas9 (HiFi Cas9). In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 or a variant thereof is from Streptococcus pyogenes. In some embodiments, the Cas9 or a variant thereof is an enhanced specificity Cas9 (eSpCas9). In some embodiments, the Cas9 or a variant thereof is a high fidelity Cas9 (HiFiCas9).

In some of any such embodiments, the recombinant nuclease is a TALEN. In some of any such embodiments, the recombinant nuclease is a ZFN.

In some of any such embodiments, the recombinant nuclease is introduced into the cell by introducing a nucleic acid encoding the recombinant nuclease into the cell. In some of any such embodiments, the TALEN is introduced into the cell by introducing a nucleic acid encoding the TALEN into the cell. In some of any such embodiments, the ZFN is introduced into the cell by introducing a nucleic acid encoding the ZFN into the cell. In some of any such embodiments, the recombinant nuclease is introduced into the cell as a protein. In some of any such embodiments, the TALEN is introduced into the cell as a protein. In some of any such embodiments, the ZFN is introduced into the cell as a protein. In some of any such embodiments, the Cas nuclease is introduced into the cell as a protein.

In some of any such embodiments, the cleavage site is at a position that is less than 200, 180, 160, 140, 120, 100, 90, 80, 70, 60, 50, 40, 30, or 20 nucleotides from the SNP. In some embodiments, the cleavage site is at a position that is less than 200 nucleotides from the SNP. In some embodiments, the cleavage site is at a position that is less than 180 nucleotides from the SNP. In some embodiments, the cleavage site is at a position that is less than 160 nucleotides from the SNP. In some embodiments, the cleavage site is at a position that is less than 140 nucleotides from the SNP. In some embodiments, the cleavage site is at a position that is less than 120 nucleotides from the SNP. In some embodiments, the cleavage site is at a position that is less than 100 nucleotides from the SNP. In some embodiments, the cleavage site is at a position that is less than 90 nucleotides from the SNP. In some embodiments, the cleavage site is at a position that is less than 80 nucleotides from the SNP. In some embodiments, the cleavage site is at a position that is less than 70 nucleotides from the SNP. In some embodiments, the cleavage site is at a position that is less than 60 nucleotides from the SNP. In some embodiments, the cleavage site is at a position that is less than 50 nucleotides from the SNP. In some embodiments, the cleavage site is at a position that is less than 40 nucleotides from the SNP. In some embodiments, the cleavage site is at a position that is less than 30 nucleotides from the SNP. In some embodiments, the cleavage site is at a position that is less than 20 nucleotides from the SNP.

In some of any such embodiments, the ssODN comprises a nucleic acid sequence that is substantially homologous to a targeting sequence in the target gene that includes the SNP. In some embodiments, the ssODN comprises a nucleic acid sequence that is substantially homologous to a targeting sequence in the target gene, wherein the targeting sequence comprises the SNP. In some of any such embodiments, the nucleic acid sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to the target gene. In some of any such embodiments, the nucleic acid sequence is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence. In some of any such embodiments, the nucleic acid sequence has at least 85% sequence identity to the target gene. In some of any such embodiments, the nucleic acid sequence is at least 85% homologous to the targeting sequence. In some of any such embodiments, the nucleic acid sequence has at least 90% sequence identity to the target gene. In some of any such embodiments, the nucleic acid sequence is at least 90% homologous to the targeting sequence. In some of any such embodiments, the nucleic acid sequence has at least 95% sequence identity to the target gene. In some of any such embodiments, the nucleic acid sequence is at least 95% homologous to the targeting sequence. In some of any such embodiments, the nucleic acid sequence is not homologous to the targeting sequence at the SNP. In some embodiments, the ssODN includes a nucleic acid sequence that is not homologous to the targeting sequence at the nucleotide of the SNP. In some of any such embodiments, the targeting sequence has a length that is between 50 and 500 nucleotides in length, optionally between 50 and 450, 50 and 400, 50 and 350, 50 and 300, 50 and 250, 50 and 200, 50 and 175, 50 and 150, 50 and 125, 50 and 100, 75 and 450, 75 and 400, 75 and 350, 75 and 300, 75 and 250, 75 and 200, 75 and 175, 75 and 150, 75 and 125, 75 and 100, 100 and 450, 100 and 400, 100 and 350, 100 and 300, 100 and 250, 100 and 200, 100 and 175, 100 and 150, or 100 and 125 nucleotides in length. In some of any such embodiments, the targeting sequence is between 50 and 500 nucleotides in length. In some of any such embodiments, the targeting sequence is between 75 and 250 nucleotides in length. In some of any such embodiments, the targeting sequence is between 150 and 200 nucleotides in length. In some of any such embodiments, the targeting sequence is between 75 and 150 nucleotides in length.

In some of any such embodiments, the targeting sequence includes the SNP and a protospacer adjacent motif (PAM) sequence. In some of any such embodiments, the targeting sequence includes a protospacer adjacent motif (PAM) sequence. In some of any such embodiments, the nucleic acid sequence comprises a PAM sequence that is homologous to the PAM sequence in the targeting sequence. In some of any such embodiments, the nucleic acid sequence comprises a PAM sequence that is not homologous to the PAM sequence in the targeting sequence at one or more positions that result in a silent mutation. In some embodiments, the ssODN includes a nucleic acid sequence that contains a PAM sequence that is not homologous to the PAM sequence in the targeting sequence at one or more nucleotide positions, wherein the integration of the ssODN into the targeting sequence results in a silent mutation in the PAM sequence. In some of any such embodiments, the nucleic acid sequence comprises one or more nucleotides that are not homologous to the targeting sequence, wherein the one or more nucleotides comprises one or more nucleotides that introduce a restriction site that is recognized by one or more restriction enzymes. In some embodiments, the ssODN includes a nucleic acid sequence that contains one or more nucleotides that are not homologous to the corresponding nucleotides of the targeting sequence, and wherein the one or more nucleotides includes one or more nucleotides that introduce a restriction site into the target gene that is recognized by one or more restriction enzymes.

In some of any such embodiments, after the integration of the ssODN into the target gene, the target gene comprises the corrected form of the SNP instead of the SNP. In some of any such embodiments, the corrected form of the SNP is not associated with PD. In some of any such embodiments, the corrected form of the SNP is a wildtype form of the SNP.

In some of any such embodiments, the target gene is human GBA1.

In some of any such embodiments, the SNP is rs76763715. In some of any such embodiments, the rs76763715 is a cytosine variant. In some of any such embodiments, the GBA1 comprising the SNP encodes a serine, rather than an asparagine, at amino acid position 370 (N370S). In some embodiments, the ssODN contains a 5′ ssODN arm and a 3′ ssODN arm, and wherein the 5′ ssODN arm contains the nucleic acid sequence set forth in any one of SEQ ID NOS: 1, 4, 25, 28, 31, 34, 37, 40, 43, 46, 49, 52, 55, 58, and 61; and/or the 3′ ssODN arm contains the nucleic acid sequence set forth in any one of SEQ ID NOS: 2, 26, 29, 32, 35, 38, 41, 44, 47, 50, 53, 56, 59, and 62.

In some of any such embodiments, the ssODN comprises a 5′ ssODN arm and a 3′ ssODN arm, and wherein the 5′ ssODN arm comprises the nucleic acid sequence of SEQ ID NO: 1, 4, or 31, and/or the 3′ ssODN arm comprises the nucleic acid sequence of SEQ ID NO: 2 or 32. In some of any such embodiments, the ssODN contains the nucleic acid sequence of SEQ ID NO: 3, 5, or 33. In some embodiments, the ssODN contains the nucleic acid sequence set forth in any one of SEQ ID NOS: 3, 5, 27, 30, 33, 36, 39, 42, 45, 48, 51, 54, 57, 60, and 63.

In some of any such embodiments, the ssODN comprises a 5′ ssODN arm and a 3′ ssODN arm, and wherein the 5′ ssODN arm comprises the nucleic acid sequence of SEQ ID NO: 1 or 4, and/or the 3′ ssODN arm comprises the nucleic acid sequence of SEQ ID NO: 2. In some of any such embodiments, the ssODN comprises the nucleic acid sequence of SEQ ID NO: 3 or 5. In some of any such embodiments, the ssODN comprises a 5′ ssODN arm and a 3′ ssODN arm, and wherein the 5′ ssODN arm comprises the nucleic acid sequence of SEQ ID NO: 31, and/or the 3′ ssODN arm comprises the nucleic acid sequence of SEQ ID NO: 32. In some of any such embodiments, the ssODN comprises the nucleic acid sequence of SEQ ID NO: 33. In some of any such embodiments, the ssODN comprises a 5′ ssODN arm and a 3′ ssODN arm, and wherein the 5′ ssODN arm comprises the nucleic acid sequence of SEQ ID NO: 25, and/or the 3′ ssODN arm comprises the nucleic acid sequence of SEQ ID NO: 26. In some of any such embodiments, the ssODN comprises the nucleic acid sequence of SEQ ID NO: 27. In some of any such embodiments, the ssODN comprises a 5′ ssODN arm and a 3′ ssODN arm, and wherein the 5′ ssODN arm comprises the nucleic acid sequence of SEQ ID NO: 28, and/or the 3′ ssODN arm comprises the nucleic acid sequence of SEQ ID NO: 29. In some of any such embodiments, the ssODN comprises the nucleic acid sequence of SEQ ID NO: 30. In some of any such embodiments, the ssODN comprises a 5′ ssODN arm and a 3′ ssODN arm, and wherein the 5′ ssODN arm comprises the nucleic acid sequence of SEQ ID NO: 34, and/or the 3′ ssODN arm comprises the nucleic acid sequence of SEQ ID NO: 35. In some of any such embodiments, the ssODN comprises the nucleic acid sequence of SEQ ID NO: 36. In some of any such embodiments, the ssODN comprises a 5′ ssODN arm and a 3′ ssODN arm, and wherein the 5′ ssODN arm comprises the nucleic acid sequence of SEQ ID NO: 37, and/or the 3′ ssODN arm comprises the nucleic acid sequence of SEQ ID NO: 38. In some of any such embodiments, the ssODN comprises the nucleic acid sequence of SEQ ID NO: 39. In some of any such embodiments, the ssODN comprises a 5′ ssODN arm and a 3′ ssODN arm, and wherein the 5′ ssODN arm comprises the nucleic acid sequence of SEQ ID NO: 40, and/or the 3′ ssODN arm comprises the nucleic acid sequence of SEQ ID NO: 41. In some of any such embodiments, the ssODN comprises the nucleic acid sequence of SEQ ID NO: 42. In some of any such embodiments, the ssODN comprises a 5′ ssODN arm and a 3′ ssODN arm, and wherein the 5′ ssODN arm comprises the nucleic acid sequence of SEQ ID NO: 43, and/or the 3′ ssODN arm comprises the nucleic acid sequence of SEQ ID NO: 44. In some of any such embodiments, the ssODN comprises the nucleic acid sequence of SEQ ID NO: 45. In some of any such embodiments, the ssODN comprises a 5′ ssODN arm and a 3′ ssODN arm, and wherein the 5′ ssODN arm comprises the nucleic acid sequence of SEQ ID NO: 46, and/or the 3′ ssODN arm comprises the nucleic acid sequence of SEQ ID NO: 47. In some of any such embodiments, the ssODN comprises the nucleic acid sequence of SEQ ID NO: 48. In some of any such embodiments, the ssODN comprises a 5′ ssODN arm and a 3′ ssODN arm, and wherein the 5′ ssODN arm comprises the nucleic acid sequence of SEQ ID NO: 49, and/or the 3′ ssODN arm comprises the nucleic acid sequence of SEQ ID NO: 50. In some of any such embodiments, the ssODN comprises the nucleic acid sequence of SEQ ID NO: 51. In some of any such embodiments, the ssODN comprises a 5′ ssODN arm and a 3′ ssODN arm, and wherein the 5′ ssODN arm comprises the nucleic acid sequence of SEQ ID NO: 52, and/or the 3′ ssODN arm comprises the nucleic acid sequence of SEQ ID NO: 53. In some of any such embodiments, the ssODN comprises the nucleic acid sequence of SEQ ID NO: 54. In some of any such embodiments, the ssODN comprises a 5′ ssODN arm and a 3′ ssODN arm, and wherein the 5′ ssODN arm comprises the nucleic acid sequence of SEQ ID NO: 55, and/or the 3′ ssODN arm comprises the nucleic acid sequence of SEQ ID NO: 56. In some of any such embodiments, the ssODN comprises the nucleic acid sequence of SEQ ID NO: 57. In some of any such embodiments, the ssODN comprises a 5′ ssODN arm and a 3′ ssODN arm, and wherein the 5′ ssODN arm comprises the nucleic acid sequence of SEQ ID NO: 58, and/or the 3′ ssODN arm comprises the nucleic acid sequence of SEQ ID NO: 59. In some of any such embodiments, the ssODN comprises the nucleic acid sequence of SEQ ID NO: 60. In some of any such embodiments, the ssODN comprises a 5′ ssODN arm and a 3′ ssODN arm, and wherein the 5′ ssODN arm comprises the nucleic acid sequence of SEQ ID NO: 61, and/or the 3′ ssODN arm comprises the nucleic acid sequence of SEQ ID NO: 62. In some of any such embodiments, the ssODN comprises the nucleic acid sequence of SEQ ID NO: 63.

In some of any such embodiments, the corrected form of the SNP is a thymine wildtype variant. In some of any such embodiments, after the integration of the ssODN into the GBA1, the GBA1 comprises the corrected form of the SNP and encodes an asparagine at amino acid position 370.

In some of any such embodiments, the SNP is rs421016. In some of any such embodiments, the rs421016 is a guanine variant. In some of any such embodiments, the GBA1 comprising the SNP encodes a proline, rather than a leucine, at amino acid position 444 (L444P). In some of any such embodiments, the corrected form of the SNP is an adenine wildtype variant. In some of any such embodiments, after the integration of the ssODN into the GBA1, the GBA1 comprises the corrected form of the SNP and encodes a leucine at amino acid position 444.

In some of any such embodiments, the SNP is rs2230288. In some of any such embodiments, the rs2230288 is a thymine variant. In some of any such embodiments, the GBA1 comprising the SNP encodes a lysine, rather than a glutamic acid, at position 326 (E326K). In some of any such embodiments, the corrected form of the SNP is a cytosine wildtype variant. In some of any such embodiments, after the integration of the ssODN into the GBA1, the GBA1 comprises the corrected form of the SNP and encodes a glutamic acid at position 326.

In some of any such embodiments, the sgRNA comprises a CRISPR targeting RNA (crRNA) sequence that is homologous to a sequence in the target gene that includes the cleavage site, optionally wherein the crRNA sequence has 100% sequence identity to the sequence in the target gene that includes the cleavage site. In some of any such embodiments, the sequence in the target gene that includes the cleavage site is immediately upstream of the PAM sequence. In some of any such embodiments, the crRNA sequence comprises the nucleic acid sequence set forth in any one of SEQ ID NOS: 8 and 13-24. In some of any such embodiments, the crRNA sequence comprises the nucleic acid sequence of SEQ ID NO: 8. In some of any such embodiments, the crRNA sequence comprises the nucleic acid sequence of SEQ ID NO: 13. In some of any such embodiments, the crRNA sequence comprises the nucleic acid sequence of SEQ ID NO: 14. In some of any such embodiments, the crRNA sequence comprises the nucleic acid sequence of SEQ ID NO: 15. In some of any such embodiments, the crRNA sequence comprises the nucleic acid sequence of SEQ ID NO: 16. In some of any such embodiments, the crRNA sequence comprises the nucleic acid sequence of SEQ ID NO: 17. In some of any such embodiments, the crRNA sequence comprises the nucleic acid sequence of SEQ ID NO: 18. In some of any such embodiments, the crRNA sequence comprises the nucleic acid sequence of SEQ ID NO: 19. In some of any such embodiments, the crRNA sequence comprises the nucleic acid sequence of SEQ ID NO: 20. In some of any such embodiments, the crRNA sequence comprises the nucleic acid sequence of SEQ ID NO: 21. In some of any such embodiments, the crRNA sequence comprises the nucleic acid sequence of SEQ ID NO: 22. In some of any such embodiments, the crRNA sequence comprises the nucleic acid sequence of SEQ ID NO: 23. In some of any such embodiments, the crRNA sequence comprises the nucleic acid sequence of SEQ ID NO: 24.

In some embodiments, the crRNA sequence and the ssODN sequence contain the nucleic acid sequences set forth in: SEQ ID NOS: 8 and 3, respectively; SEQ ID NOS: 8 and 5, respectively; SEQ ID NOS: 8 and 33, respectively; SEQ ID NOS: 13 and 27, respectively; SEQ ID NOS: 14 and 30, respectively; SEQ ID NOS: 15 and 36, respectively; SEQ ID NOS: 16 and 39, respectively; SEQ ID NOS: 17 and 42, respectively; SEQ ID NOS: 18 and 45, respectively; SEQ ID NOS: 19 and 48, respectively; SEQ ID NOS: 20 and 51, respectively; SEQ ID NOS: 21 and 54, respectively; SEQ ID NOS: 22 and 57, respectively; SEQ ID NOS: 23 and 60, respectively; or SEQ ID NOS: 24 and 63, respectively.

In some of any such embodiments, the endogenous target gene comprises a sense strand and an antisense strand, and the DNA break comprises a single strand break (SSB) at a cleavage site in the sense strand or the antisense strand. In some of any such embodiments, the endogenous target gene comprises a sense strand and an antisense strand, and the DNA break comprises a SSB at a cleavage site in the sense strand, and a SSB at a cleavage site in the antisense strand, thereby resulting in a DSB. In some of any such embodiments, the endogenous target gene comprises a sense strand and an antisense strand, and the DNA break comprises a single strand break (SSB) at a cleavage site within the endogenous target gene. In some of any such embodiments, the endogenous target gene comprises a sense strand and an antisense strand, and the DNA break comprises a SSB at a cleavage site in the sense strand, and a SSB at a cleavage site in the antisense strand, thereby resulting in a DSB.

In some of any such embodiments, the SSB is induced by one or more agents comprising a recombinant nuclease. In some of any such embodiments, the SSB in the sense strand and the SSB in the antisense strand are induced by one or more agents comprising a recombinant nuclease. In some of any such embodiments, the recombinant nuclease lacks the ability to induce a DSB by cleaving both strands of double stranded DNA. In some of any such embodiments, the one or more agents comprises a recombinant nuclease, a first sgRNA, and a second sgRNA.

In some of any such embodiments, the recombinant nuclease is selected from the group consisting of a Cas nuclease, a transcription activator-like effector nuclease (TALEN), and a zinc finger nuclease (ZFN). In some of any such embodiments, the recombinant nuclease is a Cas nuclease.

In some of any such embodiments, (i) the Cas nuclease and the first sgRNA are in a complex when they are introduced into the cell; and/or (ii) the Cas nuclease and the second sgRNA are in a complex when they are introduced into the cell. In some of any such embodiments, the Cas nuclease and the first sgRNA are in a complex when they are introduced into the cell. In some of any such embodiments, the Cas nuclease and the second sgRNA are in a complex when they are introduced into the cell. In some of any such embodiments, the Cas nuclease and the first sgRNA are in a complex when they are introduced into the cell and the Cas nuclease and the second sgRNA are in a complex when they are introduced into the cell. In some of any such embodiments, (i) the Cas nuclease and the first sgRNA are introduced into the cell as a ribonucleoprotein (RNP) complex; and/or (ii) the Cas nuclease and the second sgRNA are introduced into the cell as a RNP complex. In some of any such embodiments, the Cas nuclease and the first sgRNA are introduced into the cell as a ribonucleoprotein (RNP) complex. In some of any such embodiments, the Cas nuclease and the second sgRNA are introduced into the cell as a RNP complex. In some of any such embodiments, the Cas nuclease and the first sgRNA are introduced into the cell as a ribonucleoprotein (RNP) complex, and the Cas nuclease and the second sgRNA are introduced into the cell as a RNP complex.

In some of any such embodiments, the Cas nuclease is introduced into the cell by introducing a nucleic acid encoding the Cas nuclease into the cell. In some of any such embodiments, the nucleic acid encoding the Cas nuclease is DNA. In some of any such embodiments, the nucleic acid encoding the Cas nuclease is RNA.

In some of any such embodiments, the Cas nuclease comprises one or more mutations such that the Cas nuclease is converted into a nickase that lacks the ability to cleave both strands of a double stranded DNA molecule. In some of any such embodiments, the Cas nuclease comprises one or more mutations such that the Cas nuclease is converted into a nickase that is able to cleave only one strand of a double stranded DNA molecule. In some of any such embodiments, the Cas nuclease is selected from the group consisting of Cas3, Cas9, Cas10, Cas12, and Cas13. In some of any such embodiments, the Cas nuclease is Cas9. In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 comprises one or more mutations in the RuvC catalytic domain, optionally wherein the one or more mutations is in one or more of the RuvC I, RuvC II, or RuvC III motifs. In some of any such embodiments, the one or more mutations comprises a D10A mutation in the RuvC I motif. In some of any such embodiments, the Cas9 comprises one or more mutations in the HNH catalytic domain. In some of any such embodiments, the one or more mutations in the HNH catalytic domain is selected from the group consisting of H840A, H854A, and H863A. In some of any such embodiments, the one or more mutations in the HNH catalytic domain comprises a H840A mutation. In some of any such embodiments, the Cas9 comprises a mutation selected from the group consisting of D10A, H840A, H854A, and H863A.

In some of any such embodiments, the recombinant nuclease is a TALEN. In some of any such embodiments, the TALEN is introduced into the cell by introducing a nucleic acid encoding the TALEN into the cell. In some of any such embodiments, the TALEN is introduced into the cell as a protein. In some of any such embodiments, the TALEN comprises one or more mutations such that the TALEN is converted into a nickase that lacks the ability to cleave both strands of a double stranded DNA molecule. In some of any such embodiments, the TALEN comprises one or more mutations such that the TALEN is converted into a nickase that is able to cleave only one strand of a double stranded DNA molecule.

In some of any such embodiments, the recombinant nuclease is a ZFN. In some of any such embodiments, the ZFN is introduced into the cell by introducing a nucleic acid encoding the ZFN into the cell. In some of any such embodiments, the ZFN is introduced into the cell as a protein.

In some of any such embodiments, the cleavage site is at a position that is less than 200, 180, 160, 140, 120, 100, 90, 80, 70, 60, 50, 40, 30, or 20 nucleotides from the SNP. In some embodiments, the cleavage site is at a position that is less than 200 nucleotides from the SNP. In some embodiments, the cleavage site is at a position that is less than 180 nucleotides from the SNP. In some embodiments, the cleavage site is at a position that is less than 160 nucleotides from the SNP. In some embodiments, the cleavage site is at a position that is less than 140 nucleotides from the SNP. In some embodiments, the cleavage site is at a position that is less than 120 nucleotides from the SNP. In some embodiments, the cleavage site is at a position that is less than 100 nucleotides from the SNP. In some embodiments, the cleavage site is at a position that is less than 90 nucleotides from the SNP. In some embodiments, the cleavage site is at a position that is less than 80 nucleotides from the SNP. In some embodiments, the cleavage site is at a position that is less than 70 nucleotides from the SNP. In some embodiments, the cleavage site is at a position that is less than 60 nucleotides from the SNP. In some embodiments, the cleavage site is at a position that is less than 50 nucleotides from the SNP. In some embodiments, the cleavage site is at a position that is less than 40 nucleotides from the SNP. In some embodiments, the cleavage site is at a position that is less than 30 nucleotides from the SNP. In some embodiments, the cleavage site is at a position that is less than 20 nucleotides from the SNP. In some of any such embodiments, the cleavage site in the sense strand is at a position that is less than 200, 180, 160, 140, 120, 100, 90, 80, 70, 60, 50, 40, 30, or 20 nucleotides from the SNP; and/or the cleavage site in the antisense strand is at a position that is less than 200, 180, 160, 140, 120, 100, 90, 80, 70, 60, 50, 40, 30, or 20 nucleotides from the SNP.

In some of any such embodiments, the ssODN comprises a nucleic acid sequence that is substantially homologous to a targeting sequence in the target gene that includes the SNP. In some of any such embodiments, the nucleic acid sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to the target gene. In some of any such embodiments, the nucleic acid sequence is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence. In some of any such embodiments, the nucleic acid sequence has at least 85% sequence identity to the target gene. In some of any such embodiments, the nucleic acid sequence is at least 85% homologous to the targeting sequence. In some of any such embodiments, the nucleic acid sequence has at least 90% sequence identity to the target gene. In some of any such embodiments, the nucleic acid sequence is at least 90% homologous to the targeting sequence. In some of any such embodiments, the nucleic acid sequence has at least 95% sequence identity to the target gene. In some of any such embodiments, the nucleic acid sequence is at least 95% homologous to the targeting sequence. In some of any such embodiments, the nucleic acid sequence is not homologous to the targeting sequence at the SNP.

In some of any such embodiments, the targeting sequence has a length that is between 50 and 500 nucleotides in length, optionally between 50 and 450, 50 and 400, 50 and 350, 50 and 300, 50 and 250, 50 and 200, 50 and 175, 50 and 150, 50 and 125, 50 and 100, 75 and 450, 75 and 400, 75 and 350, 75 and 300, 75 and 250, 75 and 200, 75 and 175, 75 and 150, 75 and 125, 75 and 100, 100 and 450, 100 and 400, 100 and 350, 100 and 300, 100 and 250, 100 and 200, 100 and 175, 100 and 150, or 100 and 125 nucleotides in length. In some of any such embodiments, the targeting sequence has a length that is between 50 and 500 nucleotides in length. In some of any such embodiments, the targeting sequence has a length that is between 75 and 250 nucleotides in length. In some of any such embodiments, the targeting sequence has a length that is between 150 and 200 nucleotides in length. In some of any such embodiments, the targeting sequence has a length that is between 75 and 150 nucleotides in length.

In some of any such embodiments, the sense strand comprises the targeting sequence, and wherein the targeting sequence includes the SNP and a protospacer adjacent motif (PAM) sequence. In some of any such embodiments, the antisense strand comprises a sequence that is complementary to the targeting sequence and includes a PAM sequence. In some of any such embodiments, the antisense strand comprises the targeting sequence, and wherein the targeting sequence includes the SNP and a PAM sequence. In some of any such embodiments, the sense strand comprises a sequence that is complementary to the targeting sequence and includes a PAM sequence.

In some of any such embodiments, the nucleic acid sequence comprises a PAM sequence that is homologous to the PAM sequence in the targeting sequence. In some of any such embodiments, the nucleic acid sequence comprises a PAM sequence that is not homologous to the PAM sequence in the targeting sequence at one or more positions that result in a silent mutation. In some of any such embodiments, the nucleic acid sequence comprises one or more nucleotides that are not homologous to the targeting sequence, and wherein the one or more nucleotides comprises one or more nucleotides that introduce a restriction site that is recognized by one or more restriction enzymes.

In some of any such embodiments, after the integration of the ssODN into the target gene, the target gene comprises the corrected form of the SNP instead of the SNP. In some of any such embodiments, the corrected form of the SNP is not associated with PD. In some of any such embodiments, the corrected form of the SNP is a wildtype form of the SNP. In some of any such embodiments, the target gene is human GBA1.

In some of any such embodiments, the SNP is rs76763715. In some of any such embodiments, the rs76763715 is a cytosine variant. In some of any such embodiments, the GBA1 comprising the SNP encodes a serine, rather than an asparagine, at amino acid position 370 (N370S). In some of any such embodiments, the corrected form of the SNP is a thymine wildtype variant. In some of any such embodiments, after the integration of the ssODN into the GBA1, the GBA1 comprises the corrected form of the SNP and encodes an asparagine at amino acid position 370.

In some of any such embodiments, the SNP is rs421016. In some of any such embodiments, the rs421016 is a guanine variant. In some of any such embodiments, the GBA1 comprising the SNP encodes a proline, rather than a leucine, at amino acid position 444 (L444P). In some of any such embodiments, the corrected form of the SNP is an adenine wildtype variant. In some of any such embodiments, after the integration of the ssODN into the GBA1, the GBA1 comprises the corrected form of the SNP and encodes a leucine at amino acid position 444.

In some of any such embodiments, the SNP is rs2230288. In some of any such embodiments, the rs2230288 is a thymine variant. In some of any such embodiments, the GBA1 comprising the SNP encodes a lysine, rather than a glutamic acid, at position 326 (E326K). In some of any such embodiments, the corrected form of the SNP is a cytosine wildtype variant. In some of any such embodiments, after the integration of the ssODN into the GBA1, the GBA1 comprises the corrected form of the SNP and encodes a glutamic acid at position 326.

In some of any such embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some of any such embodiments, the crRNA sequence of the first sgRNA has 100% sequence identity to the sequence in the sense strand of the target gene that includes the cleavage site; and/or the crRNA sequence of the second sgRNA has 100% sequence identity to the sequence in the antisense strand of the target gene that includes the cleavage site. In some of any such embodiments, the crRNA sequence of the first sgRNA has 100% sequence identity to the sequence in the sense strand of the target gene that includes the cleavage site. In some of any such embodiments, the crRNA sequence of the second sgRNA has 100% sequence identity to the sequence in the antisense strand of the target gene that includes the cleavage site. In some of any such embodiments, the crRNA sequence of the first sgRNA has 100% sequence identity to the sequence in the sense strand of the target gene that includes the cleavage site, and the crRNA sequence of the second sgRNA has 100% sequence identity to the sequence in the antisense strand of the target gene that includes the cleavage site.

In some of any such embodiments, the sequence in the sense strand of the target gene that includes the cleavage site is immediately upstream of the PAM sequence; and/or the sequence in the antisense strand of the target gene that includes the cleavage site is immediately upstream of the PAM sequence. In some of any such embodiments, the sequence in the sense strand of the target gene that includes the cleavage site is immediately upstream of the PAM sequence. In some of any such embodiments, the sequence in the antisense strand of the target gene that includes the cleavage site is immediately upstream of the PAM sequence. In some of any such embodiments, the sequence in the sense strand of the target gene that includes the cleavage site is immediately upstream of the PAM sequence, and the sequence in the antisense strand of the target gene that includes the cleavage site is immediately upstream of the PAM sequence.

In some of any such embodiments, the cell is an induced pluripotent stem cell (iPSC). In some of any such embodiments, the iPSC is artificially derived from a non-pluripotent cell from a subject. In some of any such embodiments, the non-pluripotent cell is a fibroblast. In some of any such embodiments, the subject has Parkinson's Disease.

In some of any such embodiments, after the integration of the ssODN into the target gene, the method further comprises contacting DNA isolated from the cell with the one or more restriction enzymes. In some of any such embodiments, after the contacting, the method further comprises determining whether the DNA isolated from the cell has been cleaved at the restriction site. In some of any such embodiments, if the DNA has been cleaved, the cell is identified as a cell comprising an integrated ssODN.

In some of any such embodiments, after integration of the ssODN into the target gene, the method further comprises one or more of whole genome sequencing (WGS), targeted Sanger sequencing, and deep exome sequencing. In some embodiments, after integration of the ssODN into the target gene, the method further includes determining whether the cell comprises an integrated ssODN. In some embodiments, the determining is by one or more of of CIRCLE-seq, genomic qPCR, whole genome sequencing (WGS), targeted Sanger sequencing, and deep exome sequencing. In some of any such embodiments, after integration of the ssODN into the target gene, the method further comprises whole genome sequencing (WGS). In some of any such embodiments, after integration of the ssODN into the target gene, the method further comprises targeted Sanger sequencing. In some of any such embodiments, after integration of the ssODN into the target gene, the method further comprises deep exome sequencing.

Also provided herein is a complex for correcting a gene variant associated with Parkinson's Disease, comprising: a Cas nuclease; and a sgRNA comprising a CRISPR targeting RNA (crRNA) sequence that is homologous to a sequence in a target gene that includes a cleavage site, wherein the target gene is human GBA1 and includes a single nucleotide polymorphism (SNP) that is associated with Parkinson's Disease.

In some embodiments, the Cas nuclease is selected from the group consisting of Cas3, Cas9, Cas10, Cas12, and Cas13. In some of any such embodiments, the Cas nuclease is Cas9. In some of any such embodiments, the Cas nuclease is Cas9 or a variant thereof. In some embodiments, the Cas9 or a variant thereof is a Cas9 variant that exhibits reduced off-target effector activity. In some embodiments, the Cas9 or a variant thereof is an enhanced specificity Cas 9 (eSpCas9). In some embodiments, the Cas nuclease is a high fidelity Cas9 (HiFi Cas9). In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 or a variant thereof is from Streptococcus pyogenes. In some embodiments, the Cas9 or a variant thereof is a high fidelity Cas 9 (HiFiCas9). In some embodiments, the Cas nuclease is an enhanced specificity Cas9 (eSpCas9).

In some of any such embodiments, the sgRNA comprises a CRISPR targeting RNA (crRNA) sequence that is homologous to a sequence in the target gene that includes the cleavage site. In some of any such embodiments, the crRNA sequence has 100% sequence identity to the sequence in the target gene that includes the cleavage site. In some of any such embodiments, the crRNA sequence comprises the nucleic acid sequence set forth in any one of SEQ ID NOS: 8 and 13-24. In some of any such embodiments, the crRNA sequence comprises the nucleic acid sequence of SEQ ID NO: 8. In some of any such embodiments, the crRNA sequence comprises the nucleic acid sequence of SEQ ID NO: 13. In some of any such embodiments, the crRNA sequence comprises the nucleic acid sequence of SEQ ID NO: 14. In some of any such embodiments, the crRNA sequence comprises the nucleic acid sequence of SEQ ID NO: 15. In some of any such embodiments, the crRNA sequence comprises the nucleic acid sequence of SEQ ID NO: 16. In some of any such embodiments, the crRNA sequence comprises the nucleic acid sequence of SEQ ID NO: 17. In some of any such embodiments, the crRNA sequence comprises the nucleic acid sequence of SEQ ID NO: 18. In some of any such embodiments, the crRNA sequence comprises the nucleic acid sequence of SEQ ID NO: 19. In some of any such embodiments, the crRNA sequence comprises the nucleic acid sequence of SEQ ID NO: 20. In some of any such embodiments, the crRNA sequence comprises the nucleic acid sequence of SEQ ID NO: 21. In some of any such embodiments, the crRNA sequence comprises the nucleic acid sequence of SEQ ID NO: 22. In some of any such embodiments, the crRNA sequence comprises the nucleic acid sequence of SEQ ID NO: 23. In some of any such embodiments, the crRNA sequence comprises the nucleic acid sequence of SEQ ID NO: 24.

In some of any such embodiments, the Cas nuclease and the sgRNA form a ribonucleoprotein (RNP) complex.

Also provided herein is a combination for correcting a gene variant associated with Parkinson's disease, including: a Cas nuclease; a sgRNA containing a CRISPR targeting RNA (crRNA) sequence that is homologous to a sequence in a target gene that includes a cleavage site, wherein the target gene is human GBA1 and includes a single nucleotide polymorphism (SNP) that is associated with Parkinson's Disease; and a single-stranded DNA oligonucleotide (ssODN), wherein the ssODN is homologous to the target gene and contains a corrected form of the SNP.

In some embodiments, the Cas nuclease is selected from the group consisting of Cas3, Cas9, Cas10, Cas12, and Cas13. In some of any such embodiments, the Cas nuclease is Cas9. In some of any such embodiments, the Cas nuclease is Cas9 variant. In some embodiments, the Cas9 variant is a Cas9 variant that exhibits reduced off-target effector activity. In some embodiments, the Cas9 or a variant thereof is an enhanced specificity Cas 9 (eSpCas9). In some embodiments, the Cas nuclease is a high fidelity Cas9 (HiFi Cas9). In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 or a variant thereof is from Streptococcus pyogenes. In some embodiments, the Cas9 or a variant thereof is a high fidelity Cas 9 (HiFiCas9). In some embodiments, the Cas nuclease is an enhanced specificity Cas9 (eSpCas9).

In some embodiments, the crRNA sequence has 100% sequence identity to the sequence in the target gene that includes the cleavage site. In some embodiments, the crRNA sequence contains the nucleic acid sequence set forth in any one of SEQ ID NOS: 8 and 13-24. In some embodiments, the Cas nuclease and the sgRNA form a ribonucleoprotein (RNP) complex. In some embodiments, the ssODN sequence contains the nucleic acid sequence set forth in any one of SEQ ID NOS: 3, 5, 27, 30, 33, 36, 39, 42, 45, 48, 51, 54, 57, 60, and 63. In some embodiments, the crRNA sequence and the ssODN sequence contain the nucleic acid sequences set forth in: SEQ ID NOS: 8 and 3, respectively; SEQ ID NOS: 8 and 5, respectively; SEQ ID NOS: 8 and 33, respectively; SEQ ID NOS: 13 and 27, respectively; SEQ ID NOS: 14 and 30, respectively; SEQ ID NOS: 15 and 36, respectively; SEQ ID NOS: 16 and 39, respectively; SEQ ID NOS: 17 and 42, respectively; SEQ ID NOS: 18 and 45, respectively; SEQ ID NOS: 19 and 48, respectively; SEQ ID NOS: 20 and 51, respectively; SEQ ID NOS: 21 and 54, respectively; SEQ ID NOS: 22 and 57, respectively; SEQ ID NOS: 23 and 60, respectively; or EQ ID NOS: 24 and 63, respectively.

Also provided herein is a complex for correcting a gene variant associated with Parkinson's Disease, comprising: a Cas nuclease; and a first sgRNA comprising a CRISPR targeting RNA (crRNA) sequence that is homologous to a sequence in a target gene; wherein the target gene is human GBA1 and includes a single nucleotide polymorphism (SNP) that is associated with Parkinson's Disease.

In some embodiments, the target gene comprises a sense strand and an antisense strand, and the crRNA sequence is homologous to a sequence in the sense strand that includes a cleavage site. In some of any such embodiments, the target gene comprises a sense strand and an antisense strand, and the crRNA sequence is homologous to a sequence in the antisense strand that includes a cleavage site. In some of any such embodiments, the crRNA sequence has 100% sequence identity to the sequence in the sense strand that includes the cleavage site. In some of any such embodiments, the crRNA sequence has 100% sequence identity to the sequence in the antisense strand that includes the cleavage site.

In some of any such embodiments, the Cas nuclease comprises one or more mutations such that the Cas nuclease is converted into a nickase that lacks the ability to cleave both strands of a double stranded DNA molecule. In some of any such embodiments, the Cas nuclease comprises one or more mutations such that the Cas nuclease is converted into a nickase that is able to cleave only one strand of a double stranded DNA molecule. In some of any such embodiments, the Cas nuclease is selected from the group consisting of Cas3, Cas9, Cas10, Cas12, and Cas13. In some of any such embodiments, the Cas nuclease is Cas9. In some of any such embodiments, the Cas9 is from a bacteria selected from the group consisting of Streptococcus pyogenes, Staphylococcus aureus, Neisseria meningitides, Campylobacter jejuni, and Streptococcus thermophilis. In some of any such embodiments, the Cas9 is from Streptococcus pyogenes. In some of any such embodiments, the Cas9 comprises one or more mutations in the RuvC I, RuvC II, or RuvC III motifs. In some of any such embodiments, the one or more mutations comprises a D10A mutation in the RuvC I motif. In some of any such embodiments, the Cas9 comprises one or more mutations in the HNH catalytic domain. In some of any such embodiments, the one or more mutations in the HNH catalytic domain is selected from the group consisting of H840A, H854A, and H863A. In some of any such embodiments, the one or more mutations in the HNH catalytic domain comprises a H840A mutation. In some of any such embodiments, the Cas9 comprises a mutation selected from the group consisting of D10A, H840A, H854A, and H863A. In some of any such embodiments, the Cas nuclease and the first sgRNA form a ribonucleoprotein (RNP) complex.

Also provided herein is a pair of complexes for correcting a gene variant associated with Parkinson's Disease, comprising: (1) a first Cas nuclease; and a first sgRNA comprising a CRISPR targeting RNA (crRNA) sequence that is homologous to a sequence in a target gene; and (2) a second Cas nuclease; and a second sgRNA comprising a crRNA sequence that is homologous to a sequence in the target gene; wherein the target gene comprises a sense strand and an antisense strand; wherein the crRNA sequence of the first sgRNA is homologous to a sequence in the sense strand that includes a cleavage site, and the crRNA sequence of the second sgRNA is homologous to a sequence in the antisense strand that includes a cleavage site; and wherein the target gene is human GBA1 and includes a single nucleotide polymorphism (SNP) that is associated with Parkinson's Disease.

In some embodiments, the SNP is situated between the cleavage site of the sense strand and the cleavage site of the antisense strand. In some of any such embodiments, the first Cas nuclease and the second Cas nuclease comprise one or more mutations such that the first Cas nuclease and the second Cas nuclease are each converted into a nickase that lacks the ability to cleave both strands of a double stranded DNA molecule. In some of any such embodiments, the first Cas nuclease and the second Cas nuclease comprise one or more mutations such that the first Cas nuclease and the second Cas nuclease are each converted into a nickase that is able to cleave only one strand of a double stranded DNA molecule.

In some of any such embodiments, the first Cas nuclease and the second Cas nuclease is selected from the group consisting of Cas3, Cas9, Cas10, Cas12, and Cas13. In some of any such embodiments, the first Cas nuclease and the second Cas nuclease is Cas9. In some embodiments, the Cas nuclease is an enhanced specificity Cas9 (eSpCas9). In some embodiments, the Cas nuclease is a high fidelity Cas9 (HiFi Cas9). In some of any such embodiments, the first Cas nuclease and the second Cas nuclease is from a bacteria selected from the group consisting of Streptococcus pyogenes, Staphylococcus aureus, Neisseria meningitides, Campylobacter jejuni, and Streptococcus thermophilis. In some of any such embodiments, the first Cas nuclease and the second Cas nuclease is from Streptococcus pyogenes. In some of any such embodiments, the first Cas nuclease and the second Cas nuclease comprises one or more mutations in the RuvC I, RuvC II, or RuvC III motifs. In some of any such embodiments, the one or more mutations comprises a D10A mutation in the RuvC I motif. In some of any such embodiments, the first Cas nuclease and the second Cas nuclease comprises one or more mutations in the HNH catalytic domain. In some of any such embodiments, the one or more mutations in the HNH catalytic domain is selected from the group consisting of H840A, H854A, and H863A. In some of any such embodiments, the one or more mutations in the HNH catalytic domain comprises a H840A mutation. In some of any such embodiments, the first Cas nuclease and the second Cas nuclease comprises a mutation selected from the group consisting of D10A, H840A, H854A, and H863A.

In some of any such embodiments, the crRNA sequence of the first sgRNA has 100% sequence identity to the sequence in the sense strand that includes the cleavage site. In some of any such embodiments, the crRNA sequence of the second sgRNA has 100% sequence identity to the sequence in the antisense strand that includes the cleavage site. In some of any such embodiments, (i) the first Cas nuclease and the first sgRNA form a ribonucleoprotein (RNP) complex; and/or (ii) the second Cas nuclease and the second sgRNA form a RNP complex. In some of any such embodiments, the first Cas nuclease and the first sgRNA form a ribonucleoprotein (RNP) complex. In some of any such embodiments, the second Cas nuclease and the second sgRNA form a RNP complex. In some of any such embodiments, (i) the first Cas nuclease and the first sgRNA form a ribonucleoprotein (RNP) complex and (ii) the second Cas nuclease and the second sgRNA form a RNP complex.

Also provided herein is a nucleic acid, comprising the nucleic acid sequence of any of SEQ ID NOS: 8 and 13-24.

Also provided herein is a nucleic acid, comprising the nucleic acid sequence of SEQ ID NO: 8.

Also provided herein is a nucleic acid, comprising the nucleic acid sequence of SEQ ID NO: 13.

Also provided herein is a nucleic acid, comprising the nucleic acid sequence of SEQ ID NO: 14.

Also provided herein is a nucleic acid, comprising the nucleic acid sequence of SEQ ID NO: 16.

Also provided herein is a nucleic acid, comprising the nucleic acid sequence of SEQ ID NO: 17.

Also provided herein is a nucleic acid, comprising the nucleic acid sequence of SEQ ID NO: 18.

Also provided herein is a nucleic acid, comprising the nucleic acid sequence of any of SEQ ID NOS: 1, 4, 25, 28, 31, 34, 37, 40, 43, 46, 49, 52, 55, 58, and 61.

Also provided herein is a nucleic acid, comprising the nucleic acid sequence of SEQ ID NO: 1 or 4.

Also provided herein is a nucleic acid, comprising the nucleic acid sequence of SEQ ID NO: 26.

Also provided herein is a nucleic acid, comprising the nucleic acid sequence of SEQ ID NO: 29.

Also provided herein is a nucleic acid, comprising the nucleic acid sequence of SEQ ID NO: 38.

Also provided herein is a nucleic acid, comprising the nucleic acid sequence of SEQ ID NO: 41.

Also provided herein is a nucleic acid, comprising the nucleic acid sequence of SEQ ID NO: 44.

Also provided herein is a nucleic acid, comprising the nucleic acid sequence of any of SEQ ID NOS: 2, 26, 29, 32, 35, 38, 41, 44, 47, 50, 53, 56, 59, and 62.

Also provided herein is a nucleic acid, comprising the nucleic acid sequence of SEQ ID NO: 2.

Also provided herein is a nucleic acid, comprising the nucleic acid sequence of SEQ ID NO: 26.

Also provided herein is a nucleic acid, comprising the nucleic acid sequence of SEQ ID NO: 29.

Also provided herein is a nucleic acid, comprising the nucleic acid sequence of SEQ ID NO: 38.

Also provided herein is a nucleic acid, comprising the nucleic acid sequence of SEQ ID NO: 41.

Also provided herein is a nucleic acid, comprising the nucleic acid sequence of SEQ ID NO: 44.

Also provided herein is a nucleic acid, comprising the nucleic acid sequence of SEQ ID NO: 3, 5, 27, 30, 33, 36, 39, 42, 45, 48, 51, 54, 57, 60, and 63.

Also provided herein is a nucleic acid, comprising the nucleic acid sequence of SEQ ID NO: 3 or 5.

Also provided herein is a nucleic acid, comprising the nucleic acid sequence of SEQ ID NO: 27.

Also provided herein is a nucleic acid, comprising the nucleic acid sequence of SEQ ID NO: 30.

Also provided herein is a nucleic acid, comprising the nucleic acid sequence of SEQ ID NO: 39.

Also provided herein is a nucleic acid, comprising the nucleic acid sequence of SEQ ID NO: 42.

Also provided herein is a nucleic acid, comprising the nucleic acid sequence of SEQ ID NO: 45.

Also provided herein is a cell produced by the method of any one of the provided embodiments.

Also provided herein is a cell identified by the method of any one of the provided embodiments.

Also provided herein is a method for selecting for a cell comprising an integrated ssODN, comprising contacting DNA isolated from a cell derived from the cell of any one of the provided embodiments with the one or more restriction enzymes; and determining whether the DNA isolated from the cell has been cleaved at the restriction site, wherein, if the DNA has been cleaved, the cell is identified as a cell comprising an integrated ssODN.

Also provided herein is a method for selecting for a cell comprising a corrected SNP, comprising sequencing DNA isolated from a cell derived from the cell of any one of the provided embodiments; and determining whether the target gene comprises a corrected form of the SNP, wherein, if the target gene comprises a corrected form of the SNP, the cell is identified as a cell comprising a corrected SNP. In some embodiments, the sequencing comprises one or more of whole genome sequencing (WGS), targeted Sanger sequencing, and deep exome sequencing. In some embodiments, the sequencing comprises whole genome sequencing (WGS). In some embodiments, the sequencing comprises targeted Sanger sequencing. In some embodiments, the sequencing comprises deep exome sequencing.

Also provided herein is a population of the cell of any one of the provided embodiments. In some embodiments, the population is a population of pluripotent stem cells (PSCs). In some embodiments, the pluripotent stem cells are induced pluripotent stem cells (iPSCs).

Also provided is an induced pluripotent stem cell (iPSC) containing a single-strain DNA oligonucleotide (ssODN) integrated into a target gene, wherein: the target gene is human GBA1 and contains a corrected single nucleotide polymorphism (SNP), wherein the non-corrected SNP is associated with Parkinson's Disease; the integrated ssODN contains the corrected SNP instead of the non-corrected SNP; and (i) the ssODN comprises a protospacer adjacent motif (PAM) sequence that differs from a PAM sequence in the GBA1 target gene by at least one nucleotide position, wherein the integrated ssODN introduces a silent mutation in the PAM sequence of the target gene; and/or (ii) the ssODN comprises one or more nucleotides that are not homologous to the corresponding nucleotides of the GBA1 target gene, wherein the integrated ssODN introduces a restriction site in the target gene. In some embodiments, the ssODN contains the nucleic acid sequence set forth in any one of SEQ ID NOS:3, 5, 27, 30, 33, 36, 39, 42, 45, 48, 51, 54, 57, 60, and 63.

Also provided herein is a method of differentiating neural cells, the method comprising: (a) performing a first incubation comprising culturing the pluripotent stem cells of any one of the provided embodiments in a non-adherent culture vessel under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation comprising culturing cells of the spheroid in a substrate-coated culture vessel under conditions to neurally differentiate the cells.

In some embodiments, the cells are exposed to the inhibitor of TGF-β/activing-Nodal signaling up to a day at or before day 7. In some of any such embodiments, the cells are exposed to the inhibitor of TGF-β/activing-Nodal beginning at day 0 and through day 6, inclusive of each day. In some of any such embodiments, the cells are exposed to the at least one activator of SHH signaling up to a day at or before day 7. In some of any such embodiments, the cells are exposed to the at least one activator of SHH signaling beginning at day 0 and through day 6, inclusive of each day. In some of any such embodiments, the cells are exposed to the inhibitor of BMP signaling up to a day at or before day 11. In some of any such embodiments, the cells are exposed to the inhibitor of BMP signaling beginning at day 0 and through day 10, inclusive of each day. In some of any such embodiments, the cells are exposed to the inhibitor of GSK3β signaling up to a day at or before day 13. In some of any such embodiments, the cells are exposed to the inhibitor of GSK3b signaling beginning at day 0 and through day 12, inclusive of each day.

In some of any such embodiments, culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

In some of any such embodiments, the cells are exposed to BAGCT and the inhibitor of Notch signaling beginning on day 11. In some of any such embodiments, the cells are exposed to BAGCT and the inhibitor of Notch signaling beginning at day 11 and until harvest of the neurally differentiated cells, optionally until day 18, optionally until day 25.

In some of any such embodiments, the inhibitor of TGF-β/activing-Nodal signaling is SB431542. In some of any such embodiments, the at least one activator of SHH signaling is SHH or purmorphamine. In some of any such embodiments, the inhibitor of BMP signaling is LDN193189. In some of any such embodiments, the inhibitor of GSK3β signaling is CHIR99021.

Also provided herein is a method of differentiating neural cells, the method comprising: exposing the pluripotent stem cells of any one of the provided embodiments to: (a) an inhibitor of bone morphogenetic protein (BMP) signaling; (b) an inhibitor of TGF-β/activing-Nodal signaling; (c) at least one activator of Sonic Hedgehog (SHH) signaling; and (d) at least one inhibitor of GSK3β signaling.

In some embodiments, during the exposing, the pluripotent stem cells are attached to a substrate. In some embodiments, during the exposing, the pluripotent stem cells are in a non-adherent culture vessel under conditions to produce a cellular spheroid.

In some of any such embodiments, the inhibitor of TGF-β/activing-Nodal signaling is SB431542. In some of any such embodiments, the at least one activator of SHH signaling is SHH or purmorphamine. In some of any such embodiments, the inhibitor of BMP signaling is LDN193189. In some of any such embodiments, the at least one inhibitor of GSK3β signaling is CHIR99021.

In some of any such embodiments, the exposing results in a population of differentiated neural cells. In some of any such embodiments, the differentiated neural cells are floor plate midbrain progenitor cells, determined dopamine (DA) neuron progenitor cells, and/or dopamine (DA) neurons. In some of any such embodiments, the differentiated neural cells are determined dopamine (DA) neuron progenitor cells.

Also provided herein is a therapeutic composition of cells produced by the method of any one of the provided embodiments.

In some embodiments, at least 10%, at least 20%, at least 30%, at least 40%, or at least 50% of the cells of the composition comprise the corrected form of the SNP instead of the SNP. In some embodiments, at least 10% of the cells of the composition comprise the corrected form of the SNP instead of the SNP. In some embodiments, at least 20% of the cells of the composition comprise the corrected form of the SNP instead of the SNP. In some embodiments, at least 30% of the cells of the composition comprise the corrected form of the SNP instead of the SNP. In some embodiments, at least 40% of the cells of the composition comprise the corrected form of the SNP instead of the SNP. In some embodiments, at least 50% of the cells of the composition comprise the corrected form of the SNP instead of the SNP. In some embodiments, at least 60% of the cells of the composition comprise the corrected form of the SNP instead of the SNP. In some embodiments, at least 70% of the cells of the composition comprise the corrected form of the SNP instead of the SNP. In some embodiments, at least 80% of the cells of the composition comprise the corrected form of the SNP instead of the SNP. In some embodiments, at least 90% of the cells of the composition comprise the corrected form of the SNP instead of the SNP.

In some embodiments, cells of the therapeutic composition express EN1 and/or CORIN. In some embodiments, cells of the therapeutic composition express EN1. In some embodiments, cells of the therapeutic composition express CORIN. In some embodiments, cells of the composition express EN1 and CORIN and less than 10% of the total cells in the composition express TH. In some embodiments, less than 5% of the total cells in the composition express TH. In some embodiments, cells of the composition express EN1 and CORIN and less than 10% of the total cells in the composition express TH.

Also provided herein is a therapeutic composition of cells produced by the method of any one of the provided embodiments.

Also provided herein is a method of treatment, comprising administering to a subject a therapeutically effective amount of the therapeutic composition of any one of the provided embodiments.

In some embodiments, the cells of the therapeutic composition are autologous to the subject. In some of any such embodiments, the subject has Parkinson's disease.

In some of any such embodiments, the administering comprises delivering cells of a composition by stereotactic injection. In some of any such embodiments, the administering comprises delivering cells of a composition through a catheter. In some of any such embodiments, the cells are delivered to the striatum of the subject.

Also provided herein is use of the composition of any one of the provided embodiments, for the treatment of Parkinson's Disease.

### BRIEF DESCRIPTION OF THE DRAWINGS

FIGS. 1A and 1B show heatmaps (left panels) depicting the homozygous presence (dark grey), heterozygous presence (light grey), or absence (white), of particular SNPs associated with Parkinson's Disease across donor samples, along with a graph showing the effect size of the genetic risk score (GRS) for each SNP (right panels).

FIG. 2 shows quantitative PCR (qPCR) results from a Sendai clearance test for nine (9) iPSC clones tested prior to (pre-treatment) and after (post-treatment) the cells were exposed to treatment intended to kill the Sendai virus.

FIG. 3 shows results from a SNP genotyping analysis performed on nine (9) iPSC clones.

FIGS. 4A and 4B show an exemplary gene editing approach to correct the rs76763715 SNP within human GBA1 that is associated with PD. FIGS. 4A and 4B depict the GBA1 target gene (FIG. 4A) and the GBAP1 pseuodogene (FIG. 4B), and are annotated with the rs76763715 SNP that results in a N370S mutation, a donor template containing a 5′ ssODN arm and a 3′ ssODN arm, the crRNA portion of the sgRNA, the PAM sequence, the cleavage (“cut”) site within the sgRNA, and the introduction of a guanine-to-alanine substitution that introduces a restriction site recognized by the BstX1 restriction enzyme.

FIG. 5A shows 13 different sgRNAs used in an exemplary gene editing approach to correct the rs76763715 SNP within human GBA1 that is associated with PD.

FIG. 5B shows the cutting efficiency of 13 different crRNA sequences of sgRNAs in combination with the Cas9, eSpCas9, or HiFiCas9 nuclease in a cell-free experiment. For sgRNAs 1-13, the top bar represents the GBA1 gene and the bottom bar represents the GBAP1 gene. For the positive control, the top bar represents the RELA gene and the bottom bar represents the CDC42BPB gene.

FIG. 5C shows the cutting efficiency of crRNA sequences of sgRNAs 1, 2, 5, 6, and 7 in combination with Cas9 (single molecule), eSpCas9 (single molecule), HiFiCas9 (cr-tracrRNA), or HiFiCas9 (single molecule).

FIG. 5D shows the percentage of hybrid reads and the cutting and editing (HDR) efficiency of cRNA sequences of sgRNAs 1, 6, and 7 in combination with Cas9.

FIG. 5E shows the percentage of hybrid reads and the cutting and editing (HDR) efficiency of crRNA sequences of sgRNAs 1, 6, and 7 in combination with HiFiCas9.

FIG. 5F shows the percentage of hybrid reads and the cutting and editing (HDR) efficiency of crRNA sequences of sgRNAs 1, 6, and 7 in combination with eSpCas9.

FIG. 6 shows the activity of the Gcase protein encoded by the GBA1 gene in iPSCs that were edited to remove the N307S variant (edited), cells that incurred a ˜16 kb deletion through editing (˜16 kb deletion), unedited cells of the parent N370S clone (unedited), clones of the parent harboring the N370S variant (N370S), and healthy cells (control).

FIG. 7 shows an exemplary non-adherent protocol for the differentiation of pluripotent stem cells into determined dopaminergic (DA) neuron progenitor cells or DA neurons.

FIG. 8 shows an exemplary adherent protocol for the differentiation of pluripotent stem cells into determined dopaminergic (DA) neuron progenitor cells or DA neurons.

### DETAILED DESCRIPTION

The present disclosure relates to methods of correcting a genetic variation of a target gene, e.g., a single nucleotide polymorphism (SNP), associated with Parkinson's Disease (PD). In particular, the present disclosure relates to methods of correcting a genetic variation in GBA1. The provided methods include correcting the genetic variation, e.g. GBA1, in a cell from a subject with PD for use of such cells or descendants of such cells in replacement cell therapy for treating PD. In particular embodiments, the cell is a a pluripotent stem cell, and, in some embodiments, the present disclosure further includes methods of lineage specific differentiation of such pluripotent stem cells, containing a corrected gene variant. The corrected and/or differentiated cells made using the methods provided herein are further contemplated for various uses including, but not limited to, use as a therapeutic to reverse disease of, damage to, or a lack of, a certain cell type, such as dopaminergic (DA) neurons, microglia, astrocytes, or oligodendrocytes, in a patient.

Specifically described are methods of correcting a gene variant, e.g., a SNP, associated with PD, and methods for differentiating cells, e.g., pluripotent stem cells, into one or more neural cell types.

Parkinson's disease (PD) is the second most common neurodegenerative, estimated to affect 4-5 million patients worldwide. This number is predicted to more than double by 2030. PD is the second most common neurodegenerative disorder after Alzheimer's disease, affecting approximately 1 million patients in the US with 60,000 new patients diagnosed each year. Currently there is no cure for PD, which is characterized pathologically by a selective loss of midbrain DA neurons in the substantia nigra. A fundamental characteristic of PD is therefore progressive, severe and irreversible loss of midbrain dopamine (DA) neurons resulting in ultimately disabling motor dysfunction.

Mutations in certain genes can increase the risk of developing neurodegenerative diseases, such as PD or Parkinsonism. For instance, certain mutations in the GBA1 gene have been associated with the development of PD and Parkinsonism. It has been estimated that at least 7-10% of PD patients have a GBA1 mutation, that GBA1 mutations increase risk for developing PD by 20- to 30-fold, and that 30% of carriers of a GBA1 mutation will develop PD by 80 years of age. See Migdalska-Richards and Schapira, J. Neurochem. (2016); 139 (Suppl 1): 77-90.

The mutations that are associated with the development of PD and Parkinsonism include mutations in the GBA1 gene that result in an N370S amino acid change due to the presence of a serine, rather than an asparagine, at amino acid position 370 in the expressed Glucocerebrosidase (GCase) enzyme. Other mutations in the GBA1 gene that are associated with the development of PD and Parkinsonism include mutations that result in an L444P amino acid change due to the presence of a proline, rather than a leucine, at position 444 in the expressed GCase enzyme, and mutations that result in an E326K amino acid change due to the presence of a lysine, rather than a glutamic acid, at position 326 in the expressed GCase enzyme.

The provided embodiments address problems related to the use of iPSCs derived from a subject, such as a subject having PD, that contain a gene variant that increases the risk of developing PD. For instance, a strategy for the treatment of PD includes the differentiation of iPSCs derived from a patient with PD into certain cells, such as dopaminergic (DA) neurons, for autologous transplantation into the patient. However, if the patient's cells include a gene variant associated with the development of PD, which may have contributed to the patient's development of PD that led to the need for such cell transplantation, then the transplanted cells, e.g., DA neurons, would contribute to an increased risk of recurrence of PD in the patient by containing the gene variant associated with an increased risk of PD. Thus, correcting a gene variant associated with PD in iPSCs derived from a patient would allow for the benefits of autologous transplantation (e.g., avoiding ethical concerns, and avoiding risks of immune rejection) while reducing the risk of disease recurrence by changing a gene variant from one associated with an increased risk of PD into a wild type form that is not associated with an increased risk of PD, thereby reducing the risk that the patient, following transplantation, would re-develop PD.

Moreover, the human GBA1 gene has a pseudogene known as glucosylceramidase beta pseudogene 1 (GBAP1) that is approximately 96% homologous to GBA1. Horowitz et al., Genomics (1989), Vol. 4(1): 87-96. Strategies for correcting gene variants in the GBA1 gene through gene editing run the risk of adversely affecting the GBAP1 psuedogene by also targeting its gene sequence due to the homology between GBA1 and GBAP1. Off-target cleavage (e.g. DSBs) of one or more genes with high sequence homology with a target gene has been demonstrated. Cradick et al., Nucleic Acids Res (2013) 41(20):9584-92. Furthermore, it is observed herein that if DSBs are introduced in both the GBA1 and GBAP1 genes, a large (i.e. ˜16 kB) deletion can occur due to the proximity of the two genes and their high sequence homology. Thus, strategies are needed that correct the gene variant in the GBA1 gene without adversely affecting the GBAP1 psuedogene. The provided embodiments include such strategies.

The present disclosure also relates to methods of lineage specific differentiation of pluripotent stem cells (PSCs), such as embryonic stem (ES) cells or induced pluripotent stem cells (iPSCs) that have been edited to correct a gene variant associated with PD, such as a gene variant in the human GBA1 locus. Specifically described are methods of directing lineage specific differentiation of PSCs or iPSCs into floor plate midbrain progenitor cells, determined dopamine (DA) neuron progenitor cells (DDPCs), and/or dopamine (DA) neurons; or into glial cells, such as microglia, astrocytes, oligodendrocytes, or ependymocytes. The differentiated cells made using the methods provided herein are further contemplated for various uses including, but not limited to, use as a therapeutic to reverse disease of, or damage to, a lack of dopamine neurons in a patient.

Provided herein are methods for lineage specific differentiation of pluripotent stem cells (PSCs), such as embryonic stem (ES) cells or induced pluripotent stem cells (iPSCs) into floor plate midbrain progenitor cells, determined dopamine (DA) neuron progenitor cells, and/or dopamine (DA) neurons; or into glial cells, such as microglia, astrocytes, oligodendrocytes, or ependymocytes. In some aspects, PSCs are differentiated into floor plate midbrain progenitor cells. In some aspects, such floor plate midbrain progenitor cells are further differentiated into determined dopamine (DA) neuron progenitor cells. In some aspects, such determined dopamine (DA) neuron progenitor cells are further differentiated into dopamine (DA) neurons. In some aspects, PSCs are differentiated into floor plate midbrain progenitor cells, then into determined dopamine (DA) neuron progenitor cells, and finally, into dopamine (DA) neurons.

The provided embodiments address problems related to characteristics of Parkinson's disease (PD) including the selective degeneration of midbrain dopamine (mDA) neurons in patients' brains. Because PD symptoms are primarily due to the selective loss of DA neurons in the substantia nigra of the ventral midbrain, PD is considered suitable for cell replacement therapeutic strategies.

A challenge in developing a cell based therapy for PD has been the identification of an appropriate cell source for use in neuronal replacement. The search for an appropriate cell source is decades-long, and many potential sources for DA neuron replacement have been proposed. Kriks, Protocols for generating ES cell-derived dopamine neurons in Development and engineering of dopamine neurons (eds. Pasterkamp, R. J., Smidt, & Burbach) Landes Biosciences (2008); Fitzpatrick, et al., Antioxid. Redox. Signal. (2009) 11:2189-2208. Several of these sources progressed to early stage clinical trials including catecholaminergic cells from the adrenal medulla, carotid body transplants, or encapsulated retinal pigment epithelial cells. Madrazo, et al., N. Engl. J. Med. (1987) 316: 831-34; Arjona, et al., Neurosurgery (2003) 53: 321-28; Spheramine trial Bakay, et al., Front Biosci. (2004) 9:592-602. However, those trials largely failed to show clinical efficacy and resulted in poor long-term survival and low DA release from the grafted cells.

Another approach was the transplantation of fetal midbrain DA neurons, such as was performed in over 300 patients worldwide. Brundin, et al., Prog. Brain Res. (2010) 184:265-94; Lindvall, & Kokaia, J. Clin. Invest (2010) 120:29-40. Therapy using human fetal tissue in these patients demonstrated evidence of DA neuron survival and in vivo DA release up to 10 or 20 years after transplantation in some patients. In many patients, though, fetal tissue transplantation fails to replace DA neuronal function. Further, fetal tissue transplantation is plagued by challenges including low quantity and quality of donor tissue, ethical and practical issues surrounding tissue acquisition, and the poorly defined heterogeneous nature of transplanted cells, which are some of the factors contributing to the variable clinical outcomes. Mendez, et al. Nature Med. (2008); Kordower, et al. N. Engl. J. Med. (1995) 332:1118-24; and Piccini, et al. Nature Neuroscience (1999) 2:1137-40. Hypotheses as to the limited efficacy observed in the human fetal grafting trials include that fetal grafting may not provide a sufficient number of cells at the correct developmental stage and that fetal tissue is quite poorly defined by cell type and variable with regard to the stage and quality of each tissue sample. Bjorklund, et al. Lancet Neurol. (2003) 2:437-45. A further contributing factor may be inflammatory host response to the graft. Id.

Stem cell-derived cells, such as pluripotent stem cells (PSCs), are contemplated as a source of cells for applications in regenerative medicine. Pluripotent stem cells have the ability to undergo self-renewal and give rise to all cells of the tissues of the body. PSCs include two broad categories of cells: embryonic stem (ES) cells and induced pluripotent stem cells (iPSCs). ES cells are derived from the inner cell mass of preimplantation embryos and can be maintained indefinitely and expanded in their pluripotent state in vitro. Romito and Cobellis, Stem Cells Int. (2016) 2016:9451492. iPSCs can be obtained by reprogramming (“dedifferentiating”) adult somatic cells to become more ES cell-like, including having the ability to expand indefinitely and differentiate into all three germ layers. Id.

Pluripotent stem cells such as ES cells have been tested as sources for generating engraftable cells. Early studies in the 1990s using mouse ES cells demonstrated the feasibility of deriving specific lineages from pluripotent cells in vitro, including neurons. Okabe, et al., Mech. Dev. (1996) 59:89-102; Bain, et al., Dev. Biol. (1995) 168v342-357. Midbrain DA neurons were generated using a directed differentiation strategy based on developmental insights from early explants studies. Lee, et al., Nat. Biotechnol. (2000) 18v675-679; Ye, et al., Cell (1998) 93:755-66. However, these efforts did not result in cell populations containing high percentages of midbrain DA neurons or cells capable of restoring neuronal function in vivo. Additionally, the resulting populations contained a mixture of cell types in addition to midbrain DA neurons.

Existing strategies for using human PSCs (hPSCs) for cell therapy have not been entirely satisfactory. DA neurons derived from human PSCs generally have displayed poor in vivo performance, failing to compensate for the endogenous loss of neuronal function. Tabar, et al. Nature Med. (2008) 14:379-81; Lindvall and Kokaia, J. Clin. Invest (2010) 120: 29-40.

More recently, preclinical studies in which human ES cells were first differentiated into midbrain floor intermediates, and then further into DA neurons, exhibited in vivo survival and led to motor deficit recovery in animal models. Krik et al., Nature (2011) 480:547-51; Kirkeby et al., Cell Rep. (2012) 1:703-14. Despite these advances, the use of embryonic stem cells is plagued by ethical concerns, as well as the possibility that such cells may form tumors in patients. Finally, ES cell-derived transplants may cause immune reactions in patients in the context of allogeneic stem cell transplant.

The use of induced pluripotent stem cells (iPSCs), rather than ES-derived cells, has the advantages of avoiding ethical concerns. Further, derivation of iPSCs from a patient to be treated (i.e. the patient receives an autologous cell transplant) avoids risks of immune rejection inherent in the use of embryonic stem cells. As previous studies revealed that poor standardization of transplanted cell material contributes to high variability, new methods of producing substantial numbers of standardized cells, such as for autologous stem cell transplant, are needed. Lindvall and Kokaia, J. Clin. Invest (2010) 120: 29-40.

A study is currently underway in which human iPSCs were differentiated into DA neuron precursors and transplanted into the striatum of a human. However, the ability of these cells to survive, engraft, and innervate other cells in vivo has not yet been reported. Takahashi, Brain Res. (2017) 230:213-26 (2017); Cyranoski, D., Nature (2018) available at doi.org/10.1038/d41586-018-07407-9.

Thus, existing strategies have not yet proved to be successful in producing a population of differentiated cells for use in engraftment procedures for restoring neuronal function in vivo. Provided herein are methods of differentiating PSCs into determined dopaminergic neuron progenitor cells (DDPCs) and/or DA neurons cells. In particular, the provided methods are based on findings that initiating a culture of PSCs as non-adherent cells in the presence of SB, LDN, SHH, PUR, and CHIR to generate spheroid(s), followed by a further incubation of cells of the spheroid on a substrate-coated plate produces differentiated cells with superior properties. For example, cells produced by the methods described herein exhibit expression of A9 specific markers, evidencing their fate as A9 dopamine neurons and suitability for transplant, engraftment, and innervation of other cells in vivo.

Further, unlike previously reported methods, the differentiated cells produced by the methods described herein demonstrate physiological consistency. Importantly, this physiological consistency is maintained across cells differentiated from different subjects. This method therefore reduces variability both within and among subjects, and allows for better predictability of cell behavior in vivo. These benefits are associated with a successful therapeutic strategy, especially in the setting of autologous stem cell transplant, where cells are generated separately for each patient. Such reproducibility benefits among different subjects may also enable scaling in manufacturing and production processes.

Collectively, the methods described herein, including those for correcting gene variants and those for differentiating cells containing the corrected gene variants, can be used in combination to provide the benefits described above.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a small guide RNA), or micro RNA.

The term “gene variant associated with Parkinson's Disease,” or “gene variant associated with PD,” or the like, refers to a variant of a gene, such as a single nucleotide polymorphism (SNP) or a mutation, where the presence of that variant in subjects, in either heterozygous or homozygous form, has been associated with an increased risk of developing Parkinson's Disease for those subjects, as compared to the risk of developing Parkinson's Disease for the general population. The term “SNP associated with Parkinson's Disease,” or “SNP associated with PD,” or “SNP that is associated with PD,” or the like, refers to a single nucleotide polymorphism (SNP), where the presence of that particular SNP in subjects, in either heterozygous or homozygous form, has been associated with an increased risk of developing Parkinson's Disease for those subjects, as compared to the risk of developing Parkinson's Disease for the general population. The increased risk of developing Parkinson's Disease can be an increased risk of developing Parkinson's Disease over the course of a lifetime or by a certain age, such as by, e.g., 40 years of age, 45 years of age, 50 years of age, 55 years of age, 60 years of age, 65 years of age, 70 years of age, 75 years of age, or 80 years of age. The general population can either be the general population worldwide, or the general population in one or more countries, continents, or regions, such as the United States. The extent of the increased risk is not particularly limited and can be, e.g., a risk that is or is at least 0.5-fold, 1-fold, 2-fold, 3-fold, 4-fold, 5-fold, 10-fold, 15-fold, 20-fold, 25-fold, or 30-fold higher than the risk for the general population.

As used herein, the term “stem cell” refers to a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

As used herein, the term “adult stem cell” refers to an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers.

As used herein, the terms “induced pluripotent stem cell,” “iPS” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

As used herein, the term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

As used herein, the term “pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

As used herein, the term “reprogramming” refers to the process of dedifferentiating a non-pluripotent cell into a cell exhibiting pluripotent stem cell characteristics.

As used herein, the term “adherent culture vessel” refers to a culture vessel to which a cell may attach via extracellular matrix molecules and the like, and requires the use of an enzyme (e.g., trypsin, dispase, etc.) for detaching cells from the culture vessel. An “adherent culture vessel” is opposed to a culture vessel to which cell attachment is reduced and does not require the use of an enzyme for removing cells from the culture vessel.

As used herein, the term “non-adherent culture vessel” refers to a culture vessel to which cell attachment is reduced or limited, such as for a period of time. A non-adherent culture vessel may contain a low attachment or ultra-low attachment surface, such as may be accomplished by treating the surface with a substance to prevent cell attachment, such as a hydrogel (e.g. a neutrally charged and/or hydrophilic hydrogel) and/or a surfactant (e.g. pluronic acid). A non-adherent culture vessel may contain rounded or concave wells, and/or microwells (e.g. Aggrewells™). In some embodiments, a non-adherent culture vessel is an Aggrewell™ plate. For non-adherent culture vessels, use of an enzyme to remove cells from the culture vessel may not be required.

As used herein, the term “cell culture” may refer to an in vitro population of cells residing outside of an organism. The cell culture can be established from primary cells isolated from a cell bank or animal, or secondary cells that are derived from one of these sources and immortalized for long-term in vitro cultures.

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use, such as in a mammalian subject (e.g., a human). A pharmaceutical composition typically comprises an effective amount of an active agent (e.g., cells) and a carrier, excipient, or diluent. The carrier, excipient, or diluent is typically a pharmaceutically acceptable carrier, excipient or diluent, respectively.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, a “subject” is a mammal, such as a human or other animal, and typically is human.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The “CRISPR/Cas” system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, Cas9 in complex with guide and activating RNA to recognize and cleave foreign nucleic acid. Guide RNAs having the activity of both a guide RNA and an activating RNA are also known in the art. In some cases, such dual activity guide RNAs are referred to as a small guide RNA (sgRNA).

The term term “Cas9” refers to an RNA-mediated nuclease (e.g., of bacterial or archeal orgin, or derived therefrom). Exemplary RNA-mediated nuclases include the foregoing Cas9 proteins and homologs thereof, and include but are not limited to, CPF1 (See, e.g., Zetsche et al., Cell, Volume 163, Issue 3, p 759-771, 22 Oct. 2015). Similarly, as used herein, the term “Cas9 ribonucleoprotein” complex and the like refers to a complex between the Cas9 protein, and a crRNA (e.g., guide RNA or small guide RNA), the Cas9 protein and a trans-activating crRNA (tracrRNA), the Cas9 protein and a small guide RNA, or a combination thereof (e.g., a complex containing the Cas9 protein, a tracrRNA, and a crRNA guide RNA).

The phrase “editing” in the context of editing of a genome of a cell refers to inducing a structural change in the sequence of the genome at a target genomic region. For example, the editing can take the form of inducing an insertion deletion (indel) mutation into a sequence of the genome at a target genomic region. Such editing can be performed by inducing a double stranded break within a target genomic region, or a pair of single stranded nicks on opposite strands and flanking the target genomic region. Methods for inducing single or double stranded breaks at or within a target genomic region include the use of a Cas (e.g. Cas9) nuclease domain, or a derivative thereof, and a guide RNA, or pair of guide RNAs, directed to the target genomic region.

As used herein, the phrase “introducing” or “delivering” in the context of introducing or delivering a Cas (e.g. Cas9) ribonucleoprotein complex or introducing a Cas (e.g. Cas9) nuclease domain refers to the translocation of the Cas (e.g. Cas9) protein or Cas (e.g. Cas9) ribonucleoprotein complex from outside a cell to inside the cell. In some cases, introducing or delivering refers to translocation of the Cas (e.g. Cas9) or Cas (e.g. Cas9) ribonucleoprotein from outside the cell to inside the nucleus of the cell. Various methods of such translocation are contemplated, including but not limited to, electroporation, contact with nanowires or nanotubes, receptor mediated internalization, translocation via cell penetrating peptides, liposome mediated translocation, and the like.

“Homology-directed repair” or “HDR” refers to the process of repairing DNA damage in cells using a homologous nucleic acid (e.g., an endogenous homologous sequence, e.g., a sister chromatid, or an exogenous nucleic acid, e.g., a template nucleic acid). In a normal cell, HDR typically involves a series of steps such as recognition of the break, stabilization of the break, resection, stabilization of single stranded DNA, formation of a DNA crossover intermediate, resolution of the crossover intermediate, and ligation.

“Single-stranded DNA oligonucleotide” or “ssODN” refers to a DNA oligonucleotide that can be utilized by a cell as a template for HDR. Generally, the ssODN has at least one region of homology to a target site. In some cases, the ssODN has two homologous regions flanking a region that contains a mutation or a heterologous sequence to be inserted at a target cut site.

### II. METHODS OF CORRECTING GENE VARIANTS

Provided herein are methods of correcting a gene variant, e.g., a single nucleotide polymorphism (SNP), associated with Parkinson's Disease (PD), in a target gene, e.g., GBA1.

Provided here are methods of correcting a gene variant associated with Parkinson's Disease, the method comprising: introducing, into a cell, one or more agents capable of inducing a DNA break within an endogenous target gene in the cell, wherein the target gene is human GBA1 and includes a gene variant that is associated with Parkinson's Disease; and introducing, into the cell, a donor template, wherein the donor template is homologous to the target gene and comprises a corrected form of the gene variant, wherein the introducing of the one or more agents and the donor template results in homology-directed repair (HDR) and integration of the donor template into the target gene.

Also provided here are methods of correcting a gene variant associated with Parkinson's Disease, the method comprising: introducing, into a cell, one or more agents comprising a recombinant nuclease for inducing a DNA break within an endogenous target gene in the cell, wherein the target gene is human GBA1 and includes a single nucleotide polymorphism (SNP) that is associated with Parkinson's Disease; and introducing, into the cell, a single-stranded DNA oligonucleotide (ssODN), wherein the ssODN is homologous to the target gene and comprises a corrected form of the SNP, wherein the introducing of the one or more agents and the ssODN results in homology-directed repair (HDR) and integration of the ssODN into the target gene.

Also provided here are methods of correcting a gene variant associated with Parkinson's Disease, the method comprising: introducing, into a cell, a donor template; wherein the cell comprises a DNA break within an endogenous target gene in the cell, wherein the target gene is human GBA1 and includes a gene variant that is associated with Parkinson's Disease, wherein the donor template is homologous to the target gene and comprises a corrected form of the gene variant, and wherein the introducing results in HDR and integration of the donor template into the target gene.

Also provided here are methods of correcting a gene variant associated with Parkinson's Disease, the method comprising: introducing, into a cell, a single-stranded DNA oligonucleotide (ssODN); wherein the cell comprises a DNA break within an endogenous target gene in the cell, wherein the target gene is human GBA1 and includes a single nucleotide polymorphism (SNP) that is associated with Parkinson's Disease, wherein the ssODN is homologous to the target gene and comprises a corrected form of the SNP, and wherein the introducing results in HDR and integration of the ssODN into the target gene.

The provided methods, in some embodiments, result in correction of a gene variant, e.g., SNP, associated with Parkinson's Disease by integrating the donor template, e.g., ssODN, that comprises a corrected form of the gene variant, e.g., SNP, into the target gene, thereby resulting in a corrected target gene that no longer includes the gene variant, e.g., a SNP associated with Parkinson's Disease.

The provided methods, in some embodiments, include a recombinant nuclease that is capable of inducing cleavage of both strands of a double stranded DNA molecule. The provided methods, in some embodiments, include a recombinant nuclease that is not capable of inducing cleavage of both strands of a double stranded DNA molecule, e.g., the recombinant nuclease is a nickase that is capable of only cleaving one strand of a double stranded DNA molecule.

### A. Samples, Cells, and Cell Preparations

In embodiments of the provided methods, cells are engineered to correct a gene variant in a target gene associated with PD, e.g., by introducing one or more components as described herein. In some embodiments, the cell is a pluripotent stem cell. Various sources of pluripotent stem cells can be used in the method, including embryonic stem (ES) cells and induced pluripotent stem cells (iPSCs). In some embodiments, the cell is an iPSC. In some embodiments, the pluripotent stem cell is an iPSC. In some embodiments, the pluripotent stem cell is an iPSC, artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka, Cell (2006) 126: 663-76.

In some embodiments, the cell is a pluripotent stem cell. In some embodiments, the cell is a pluripotent stem cell that was artificially derived from a non-pluripotent cell of a subject. In some embodiments, the non-pluripotent cell is a fibroblast. In some embodiments, the subject is a human. In some embodiments, the subject is a human with Parkinson's Disease. In some embodiments, the pluripotent stem cell is an iPSC.

In some aspects, pluripotency refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphology associated with pluripotent stem cells.

Methods for generating iPSCs are known. For example, mouse iPSCs were reported in 2006 (Takahashi and Yamanaka), and human iPSCs were reported in late 2007 (Takahashi et al. and Yu et al.). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, the PSCs (e.g. iPSCs) are from a subject having a gene variant, e.g., SNP, in GBA1 that is associated with PD. The gene variant in GBA1 that is associated with PD is not limited and can be any gene variant, e.g., SNP, in GBA1 that is associated with PD, e.g., is associated with an increased risk of developing PD. In some embodiments, the gene variant is a mutation in the GBA1 gene that results in an N370S amino acid change due to the presence of a serine, rather than an asparagine, at amino acid position 370 in the expressed GCase enzyme; or is a mutation in the GBA1 gene that results in an L444P amino acid change due to the presence of a proline, rather than a leucine, at position 444 in the expressed GCase enzyme; or is a mutation that results in an E326K amino acid change due to the presence of a lysine, rather than a glutamic acid, at position 326 in the expressed GCase enzyme. In some embodiments, the gene variant is a SNP in the GBA1 gene selected from the group consisting of rs76763715, rs421016, and rs2230288.

In some embodiments, the PSCs (e.g. iPSCs) are autologous to the subject to be treated, i.e. the PSCs are derived from the same subject to whom the differentiated cells that were previously corrected for one or more gene variant(s), e.g., SNP(s), associated with PD, are administered.

In some embodiments, non-pluripotent cells (e.g., fibroblasts) derived from patients having Parkinson's disease (PD) are reprogrammed to become iPSCs before correction of one or more gene variant(s) and/or differentiation into neural and/or neuronal cells. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients having PD are reprogrammed to become iPSCs before correction of one or more gene variant(s) and/or differentiation into determined DA neuron progenitors cells and/or DA neurons, such as by use of the non-integrating Sendai virus to reprogram the cells (e.g., use of CTS™ CytoTune™-iPS 2.1 Sendai Reprogramming Kit). In some embodiments, the resulting corrected and differentiated cells are then administered to the patient from whom they are derived in an autologous stem cell transplant. In some embodiments, the PSCs (e.g., iPSCs) are allogeneic to the subject to be treated, i.e. the PSCs are derived from a different individual than the subject to whom the corrected and differentiated cells will be administered. In some embodiments, non-pluripotent cells (e.g., fibroblasts) derived from another individual (e.g. an individual not having a neurodegenerative disorder, such as Parkinson's disease) are reprogrammed to become iPSCs before correction of one or more gene variant(s) and/or differentiation into determined DA neuron progenitor cells and/or DA neurons. In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting corrected and differentiated cells are then administered to an individual who is not the same individual from whom the corrected and differentiated cells are derived (e.g. allogeneic cell therapy or allogeneic cell transplantation).

In any of the provided embodiments, the PSCs described herein (e.g. allogeneic cells) may be genetically engineered to be hypoimmunogenic. Methods for reducing the immunogenicity are known, and include ablating polymorphic HLA-A/-B/-C and HLA class II molecule expression and introducing the immunomodulatory factors PD-L1, HLA-G, and CD47 into the AAVS1 safe harbor locus in differentiated cells. Han et al., PNAS (2019) 116(21):10441-46. Thus, in some embodiments, the PSCs described herein are engineered to delete highly polymorphic HLA-A/-B/-C genes and to introduce immunomodulatory factors, such as PD-L1, HLA-G, and/or CD47, into the AAVS1 safe harbor locus.

In some embodiments, following correction of one or more gene variant(s), PSCs (e.g., iPSCs) are cultured in the absence of feeder cells, until they reach 80-90% confluency, at which point they are harvested and further cultured for differentiation (day 0). In one aspect of the method described herein, once iPSCs reach 80-90% confluence, they are washed in phosphate buffered saline (PBS) and subjected to enzymatic dissociation, such as with Accutase™, until the cells are easily dislodged from the surface of a culture vessel. The dissociated iPSCs are then re-suspended in media for downstream differentiation into the desired cell type(s), such as determined DA neuron progenitor cells and/or DA neurons. Section III, below, provides exemplary methods for differentiation of PSCs, e.g., iPSCs, that have been corrected by the provided methods.

In some embodiments, following correction of one or more gene variant(s), the PSCs are resuspended in a basal induction media. In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with serum replacement, a Rho-associated protein kinase (ROCK) inhibitor, and various small molecules, for differentiation. In some embodiments, the PSCs are resuspended in the same media they will be cultured in for at least a portion of the first incubation.

### B. Cleavage Sites, Endogenous Target Genes, and Gene Variants

The provided methods involve, in some embodiments, inducing a DNA break within an endogenous target gene in a cell, e.g., a cell as described in Section II.A., such as a PSC, e.g., iPSC, derived from a subject having a gene variant in the human GBA1 locus associated with PD. Also provided are methods that involve, in some embodiments, a cell that comprises a DNA break within an endogenous target gene in the cell, e.g., a cell as described in Section II.A., such as a PSC, e.g., iPSC, derived from a subject having a gene variant in the human GBA1 locus associated with PD.

In some embodiments, the DNA break is a double strand break (DSB) at a cleavage site within the endogenous target gene. In some embodiments, a double strand break (DSB) is induced in an endogenous target gene, e.g., GBA1, that comprises a gene variant associated with Parkinson's Disease (PD). In some embodiments, the DSB is induced by a recombinant nuclease that is capable of inducing a DSB by cleaving both strands of double stranded DNA at a cleavage site. An example of a recombinant nuclease that is capable of inducing a DSB by cleaving both strands of double stranded DNA at a cleavage site is Cas9, e.g., wildtype Cas9 or a Cas9 that does not include one or more mutations that disrupt cleavage activity.

In some embodiments, the DNA break comprises a single strand break (SSB) at a cleavage site in the sense strand or the antisense strand of the target gene. In some embodiments, the DNA break comprises a SSB at a cleavage site in the sense strand, and a SSB at a cleavage site in the antisense strand, thereby resulting in a DSB. In some embodiments, the DSB is induced by a pair of recombinant nucleases, e.g., nickases, that are each capable of inducing a single strand break (SSB) in opposite DNA strands at different cleavage sites, e.g., at a cleavage site upstream of the gene variant in one strand and at a cleavage site downstream of the gene variant in the other strand of the target gene. In some embodiments, a first of the pair of nickases forms a complex with a first guide RNA, e.g., a first sgRNA, for targeting cleavage to one strand, e.g., the sense strand, and the second of the pair of nickases forms a cmplex with a second guide RNA, e.g., a second sgRNA, for targeting cleavage to the other strand, e.g., the antisense strand.

In some embodiments, a double strand break (DSB) is induced at a cleavage site in an endogenous target gene that comprises a gene variant associated with Parkinson's Disease (PD).

In some embodiments, a DSB is induced through a SSB on each of the opposite strands, i.e., the sense strand and the antisense strand, of an endogenous target gene that comprises a gene variant associated with PD.

In general, genes are located in double stranded DNA that includes a sense strand and an antisense strand, which are complementary to one another. The sense strand is also referred to as the coding strand because its sequence is the DNA version of the RNA sequence that is transcribed. The antisense strand is also referred to as the template strand because its sequence is complementary to the RNA sequence that is transcribed. Thus, in some embodiments, the target gene, e.g., GBA1, includes a sense strand and an antisense strand. In some embodiments, the target gene, e.g., GBA1, comprises a targeting sequence that includes the gene variant associated with PD. In some embodiments, the gene variant associated with PD is a single nucleotide polymorphism (SNP). Accordingly, in some embodiments, a double strand break (DSB) is induced at a cleavage site in an endogenous target gene, e.g., GBA1, that comprises a SNP that is associated with PD.

In some embodiments, a double strand break (DSB) is induced at a cleavage site in the endogenous locus that encodes the beta-glucocerebrosidase (GCase) enzyme, also known as lysosomal acid glucosylceramidase. In humans, GCase is encoded by the beta-glucocerebrosidase (GBA1) gene. In some embodiments, the cleavage site is in an exon in the GBA1 locus. In some embodiments, the cleavage site is in an intron in the GBA1 locus. In some embodiments, the GBA1 locus includes a gene variant, e.g., a single nucleotide polymorphism (SNP), associated with Parkinson's Disease (PD).

In some embodiments, the target gene is human GBA1. In some embodiments, the human GBA1 encodes the amino acid sequence of SEQ ID NO: 7. In some embodiments, the human GBA1 encodes the amino acid sequence of SEQ ID NO: 9. In some embodiments, the human GBA1 encodes the amino acid sequence of SEQ ID NO: 10.

In some embodiments, the target gene is human GBA1 and comprises a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the nucleotide sequence as set forth in SEQ ID NO: 12. In some embodiments, the target gene is human GBA1 and comprises a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the nucleotide sequence as set forth in SEQ ID NO: 12, wherein the human GBA1 encodes the amino acid sequence of SEQ ID NO: 7. In some embodiments, the target gene is human GBA1 and comprises a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to a portion of the nucleotide sequence as set forth in SEQ ID NO: 12, e.g., a sequence of 50 nucleotides, 75 nucleotides, 100 nucleotides, 125 nucleotides, 150 nucleotides, 175 nucleotides, 200 nucleotides, 225 nucleotides, 250 nucleotides, 275 nucleotides, 300 nucleotides, 325 nucleotides, 350 nucleotides, 375 nucleotides, 400 nucleotides, 425 nucleotides, 450 nucleotides, 475 nucleotides, or 500 nucleotides, comprised within the nucleotide sequence of SEQ ID NO: 12. In some embodiments, the target gene is human GBA1 and comprises a nucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to a portion of the nucleotide sequence as set forth in SEQ ID NO: 12, e.g., a sequence of 50 nucleotides, 75 nucleotides, 100 nucleotides, 125 nucleotides, 150 nucleotides, 175 nucleotides, 200 nucleotides, 225 nucleotides, 250 nucleotides, 275 nucleotides, 300 nucleotides, 325 nucleotides, 350 nucleotides, 375 nucleotides, 400 nucleotides, 425 nucleotides, 450 nucleotides, 475 nucleotides, or 500 nucleotides, comprised within the nucleotide sequence of SEQ ID NO: 12, wherein the human GBA1 encodes the amino acid sequence of SEQ ID NO: 7.

In some embodiments, the gene variant associated with PD is any gene variant in the human GBA1 locus that is associated with PD. In some of any such embodiments, the gene variant is a single nucleotide polymorphism (SNP). Accordingly, in some embodiments, the target gene, e.g., GBA1, includes a SNP that is associated with PD.

In some embodiments, the gene variant associated with PD is a gene variant in the GBA1 locus that encodes a variant of GCase that includes serine, rather than asparagine, at position 370 (N370S) in GCase. In some embodiments, the gene variant associated with PD is a rs76763715 SNP. In some embodiments, the gene variant associated with PD is a gene variant at rs76763715 that causes an amino acid substitution of asparagine to serine at position 370 (N370S) in GCase, compared to wildtype GCase. In some embodiments, the gene variant associated with PD is caused by the presence of a cytosine in place of a thymine (T>C) at the rs76763715 SNP, which causes an amino acid substitution of asparagine to serine at position 370 (N370S) in GCase, compared to wildtype GCase. In some of any such embodiments, the gene variant is a SNP. In some embodiments, the target gene is human GBA1 and encodes an amino acid sequence comprising the amino acid sequence of SEQ ID NO: 7, wherein the GBA1 includes a gene variant that encodes a serine, rather than an asparagine, at position 370 (N370S).

In some embodiments, the gene variant associated with PD is a gene variant in the human GBA1 locus that encodes a variant of GCase that includes proline, rather than leucine, at position 444 (L444P) in GCase. In some embodiments, the gene variant associated with PD is a rs421016 SNP. In some embodiments, the gene variant associated with PD is a gene variant at rs421016 that causes an amino acid substitution of leucine to proline at position 444 (L444P) in GCase, compared to wildtype GCase. In some embodiments, the gene variant associated with PD is caused by the presence of a guanine in place of an adenine (A>G) at the rs421016 SNP, which causes an amino acid substitution of leucine to proline at position 444 (L444P) in GCase, compared to wildtype GCase. In some of any such embodiments, the gene variant is a SNP. In some embodiments, the target gene is human GBA1 and encodes an amino acid sequence comprising the amino acid sequence of SEQ ID NO: 9, wherein the GBA1 includes a gene variant that encodes a proline, rather than a leucine, at position 444 (L444P).

In some embodiments, the gene variant associated with PD is a gene variant in the GBA1 locus that includes lysine, rather than glutamic acid, at position 326 (E326K) in GCase. In some embodiments, the gene variant associated with PD is a rs2230288 SNP. In some embodiments, the gene variant associated with PD is a gene variant at rs2230288 that causes an amino acid substitution of glutamic acid to lysine at position 326 (E326K) in GCase, compared to wildtype GCase. In some embodiments, the gene variant associated with PD is caused by the presence of a thymine in place of an cytosine (C>T) at the rs2230288 SNP, which causes an amino acid substitution of glutamic acid to lysine at position 326 (E326K) in GCase, compared to wildtype GCase. In some of any such embodiments, the gene variant is a SNP. In some embodiments, the target gene is human GBA1 and encodes an amino acid sequence comprising the amino acid sequence of SEQ ID NO: 10, wherein the GBA1 includes a gene variant that encodes lysine, rather than glutamic acid, at position 326 (E326K).

In some embodiments, the cleavage site, e.g., the cleavage site on the sense strand and/or the cleavage site on the antisense strand, is located near the gene variant, e.g., SNP. In some embodiments, the cleavage site is located near the gene variant, such as at a positon that is less than 200, 190, 180, 170, 160, 150, 140, 130, 120, 110, 110, 90, 80, 70, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, or 5 nucleotides from the position of the nucleotide(s) causing the gene variant. In some embodiments, the cleavage site is located at a position that is less than 50 nucleotides from the position of the nucleotide(s) causing the gene variant. In some embodiments, the cleavage site is located at a position that is less than 40 nucleotides from the position of the nucleotide(s) causing the gene variant. In some embodiments, the cleavage site is located at a position that is less than 30 nucleotides from the position of the nucleotide(s) causing the gene variant. In some embodiments, the cleavage site is located at a position that is between 5 and 50 nucleotides from the position of the nucleotide(s) causing the gene variant, such as between 10 and 50, 10 and 40, 15 and 50, 15 and 40, 20 and 40, 10 and 35, 15 and 35, 15 and 30, or 20 and 30 nucleotides from the position of the nucleotide(s) causing the gene variant. In some embodiments, the cleavage site is at a position that is less than 200, 180, 160, 140, 120, 100, 90, 80, 70, 60, 50, 40, 30, or 20 nucleotides from the gene variant.

In some embodiments, the cleavage site is located near the SNP, such as at a positon that is less than 200, 190, 180, 170, 160, 150, 140, 130, 120, 110, 110, 90, 80, 70, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, or 5 nucleotides from the SNP. In some embodiments, the cleavage site is located at a position that is less than 50 nucleotides from the SNP. In some embodiments, the cleavage site is located at a position that is less than 40 nucleotides from the SNP. In some embodiments, the cleavage site is located at a position that is less than 30 nucleotides from the SNP. In some embodiments, the cleavage site is located at a position that is less than 20 nucleotides from the SNP. In some embodiments, the cleavage site is located at a position that is less than 10 nucleotides from the SNP. In some embodiments, the cleavage site is located at a position that is between 5 and 50 nucleotides from the SNP, such as between 10 and 50, 10 and 40, 15 and 50, 15 and 40, 20 and 40, 10 and 35, 15 and 35, 15 and 30, or 20 and 30 nucleotides from the SNP. In some embodiments, the cleavage site is at a position that is less than 200, 180, 160, 140, 120, 100, 90, 80, 70, 60, 50, 40, 30, or 20 nucleotides from the SNP.

In some embodiments, the cleavage site in the sense strand is at a position that is less than 200, 180, 160, 140, 120, 100, 90, 80, 70, 60, 50, 40, 30, or 20 nucleotides from the SNP; and/or the cleavage site in the antisense strand is at a position that is less than 200, 180, 160, 140, 120, 100, 90, 80, 70, 60, 50, 40, 30, or 20 nucleotides from the SNP.

In some embodiments, at the cleavage site where a DSB has occurred, the action of cellular DNA repair mechanisms can, in the presence of a donor template comprising a corrected form of the gene variant, e.g., a donor template comprising a corrected form of the SNP, alter the DNA sequence based on the donor template, such as by integration of the nucleic acid sequences contained in the donor template through homology-directed repair (HDR).

In some embodiments, at the cleavage sites where a DSB has occurred through a SSB on the sense strand and a SSB on the antisense strand, the action of cellular DNA repair mechanisms can, in the presence of a donor template comprising a corrected form of the gene variant, e.g., a donor template comprising a corrected form of the SNP, alter the DNA sequence based on the donor template, such as by integration of the nucleic acid sequences contained in the donor template through homology-directed repair (HDR).

### C. Agents Capable of Inducing a Double Strand Break (DSB)

In some embodiments, the methods of correcting gene variants involve introducing a DNA break, e.g., a single strand break (SSB) or a double strand break (DSB) at one or more cleavage sites, e.g., one or more sites in the GBA1 locus. Methods for inducing a DNA break, e.g., a SSB or a DSB, including those described herein, can involve use of one or more agent(s) capable of inducing a DNA break, e.g., a SSB or a DSB at one or more cleavage site(s) in the endogenous target gene, e.g., GBA1, such that repair of the DNA break, e.g., DSB, or of the DSB caused by a SSB on each strand, by HDR using a donor template comprising a corrected form of the gene variant, e.g., SNP, can result in the insertion of a sequence of interest, e.g., a sequence that includes a wildtype variant of a gene variant associated with PD, at or near the cleavage site. Also provided are one or more agent(s) capable of inducing a DNA break, e.g., a SSB or a DSB, for use in the methods provided herein. In some embodiments, the one or more agent(s) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) can be used in combination with a donor template, e.g., an ssODN, for HDR-mediated integration of the donor template into the target gene, e.g., GBA1, such as at the targeting sequence. In some embodiments, the one or more agent(s) can be used in combination with a donor template, e.g., an ssODN, and a guide RNA, e.g., a sgRNA, for HDR-mediated integration of the donor template into the target gene, e.g., GBA1, such as at the targeting sequence. In some embodiments, the one or more agent(s) can be used in combination with a donor template, e.g., an ssODN, and a first guide RNA, e.g., a first sgRNA, and a second guide RNA, e.g., a second sgRNA, for HDR-mediated integration of the donor template into the target gene, e.g., GBA1, such as at the targeting sequence.

In some embodiments, the method involves introducing, into a cell, one or more agent(s) capable of inducing a DNA break within an endogenous target gene, e.g., GBA1, in the cell. In some embodiments, the DNA break is a DSB at a cleavage site within the endogenous target gene, e.g., GBA1, In some embodiments, the DNA break comprises a SSB at a cleavage site in the sense strand or the antisense strand. In some embodiments, the DNA break comprises a SSB at a cleavage site in the sense strand, and a SSB at a cleavage site in the antisense strand, thereby resulting in a DSB.

In some embodiments, the method involves introducing, into a cell, one or more agent(s) capable of inducing a DSB at a cleavage site within an endogenous target gene, e.g., GBA1, in the cell. In some embodiments, the one or more agent(s) capable of inducing a DSB comprise a recombinant nuclease. Accordingly, in some embodiments, the method involves introducing, into a cell, one or more agent(s) comprising a recombinant nuclease for inducing a DSB at a cleavage site within an endogenous target gene, e.g., GBA1, in the cell. In some embodiments, the recombinant nuclease is a Cas nuclease, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene, e.g., GBA1, that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, e.g., GBA1, that includes a cleavage site, such as the targeting sequence.

In some embodiments, the one or more agent(s) capable of inducing a DSB involve use of the CRISPR/Cas gene editing system. In some embodiments, the one or more agent(s) comprise a recombinant nuclease. In some embodiments, the one or more agent(s) capable of inducing a DSB comprise a recombinant nuclease and a guide RNA, e.g., a sgRNA. In some embodiments, the recombinant nuclease is a Cas nuclease. In some embodiments, the Cas nuclease is selected from the group consisting of Cas3, Cas9, Cas10, Cas12, and Cas13. In some embodiments, the Cas nuclease is Cas9. In some embodiments, the one or more agent(s) capable of inducing a DSB comprise Cas9 or a functional fragment thereof, and a guide RNA, e.g., sgRNA. The guide RNA, in some embodiments, binds to the recombinant nuclease and targets the recombinant nuclease to a specific location within the target gene, e.g., GBA1, such as at a location within the target gene that is or includes the cleavage site. In some embodiments, the recombinant nuclease is a Cas nuclease from any bacterial species, or is a functional fragment thereof. In some embodiments, the recombinant nuclease is Cas9 nuclease. The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9).

In some embodiments, the Cas nuclease is an “enhanced specificity” Cas9. In some embodiments, the enhanced specificity Cas9 nuclease is enhanced specificity SpCas9 (eSpCas9). Slaymaker et al., Science (2016) 351(6268):84-8. In some embodiments, the Cas nuclease is a “high fidelity” Cas9. In some embodiments, the eSpCas9 is a wildtype spCas9 nuclease comprising each of K848A, K1003A, and R1060A variants. Thus, in some embodiments, the recombinant nuclease is SpCas9 (K848A/K1003A/R1060A), also known as eSpCas9.

In some embodiments, the high fidelity Cas9 nuclease is a high fidelity Cas9 (HiFi Cas9). Kleinstiver et al., Nature (2016) 529(7587):490-5. In some embodiments, the HiFiCas9 is a wildtype spCas9 nuclease comprising each of N497A, R661A, Q695A, and Q926A variants. Thus, in some embodiments, the recombinant nuclease is spCas9 (N497A/R661A/Q695A/Q926A), also known as HiFiCas9.

In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the recombinant nuclease, e.g., Cas9, is targeted to the cleavage site by interacting with a guide RNA, e.g., sgRNA, that hybridizes to a DNA sequence that immediately precedes a Protospacer Adjacent Motif (PAM) sequence. In some embodiments, the guide RNA, e.g., sgNA, that is specific to a target gene of interest, e.g., human GBA1 locus, is used to target the recombinant nuclease, e.g., Cas9, to induce a DSB at a cleavage site within the target gene. In general, a guide RNA, e.g., sgRNA, is any nucleotide sequence comprising a sequence, e.g., a crRNA sequence, that has sufficient complementarity with a target gene sequence, such as the human GBA1 locus, to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene, e.g., GBA1, that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the method involves introducing, into a cell, one or more agent(s) capable of inducing a SSB at a cleavage site within the sense strand and a SSB at a cleavage site within the antisense strand of an endogenous target gene, e.g., GBA1, in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) comprising a recombinant nuclease for inducing a SSB at a cleavage site in the sense strand and a SSB at a cleavage site in the antisense strand within an endogenous target gene, e.g., GBA1, in the cell. Although, in some embodiments, it is described that “a” or “the” recombinant nuclease induces a SSB in the antisense strand a SSB in the sense strand, it is to be understood that this includes situations where two of the same recombinant nuclease is used, such that one of the recombinant nucleases induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the recombinant nuclease is a Cas nuclease, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene, e.g., GBA1, that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, e.g., GBA1, that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the recombinant nuclease is a Cas nuclease. In some embodiments, the Cas nuclease comprises one or more mutations such that the Cas nuclease is converted into a nickase that lacks the ability to cleave both strands of a double stranded DNA molecule. In some embodiments, the Cas nuclease comprises one or more mutations such that the Cas nuclease is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas nuclease comprises one or more mutations in the RuvC catalytic domain or the HNH catalytic domain. In some embodiments, the recombinant nuclease is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the Cas nuclease is selected from the group consisting of Cas3, Cas9, Cas10, Cas12, and Cas13. In some embodiments, the Cas nuclease is Cas9. In some embodiments, the Cas nuclease is a variant of Cas9 (e.g. SpCas9) that exhibits reduced off-target effector activity. In some embodiments, the Cas9 (e.g. SpCas9) variant exhibits off-target effector activity (i.e. off-target cleavage) that is reduced by between about 50% and 100% compared to a wildtype Cas9 (e.g. SpCas9). In some embodiments, off-target effector activity of a Cas9 (e.g. SpCas9) variant is reduced by at least about 50%, at least about 60%, at least about 70%, at least about 80%, at least about 90%, or at least about 95% compared to a wildtype Cas9 (e.g. SpCas9). In some embodiments, the Cas9 variant is enhanced specificity Cas9 (eSpCas9). In some embodiments, the Cas9 variant is high fidelity Cas9 (HiFi Cas9). In some embodiments, the one or more agent(s) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene, e.g., GBA1, such as at a location within the sense strand or the antisense strand of the target gene that is or includes the cleavage site. In some embodiments, the recombinant nuclease is a Cas nuclease from any bacterial species, or is a functional fragment thereof. In some embodiments, the recombinant nuclease is Cas9 nuclease. The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation.

In some embodiments, the recombinant nuclease, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the recombinant nuclease, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the recombinant nuclease, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest, e.g., human GBA1 locus, is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest, e.g., human GBA1 locus, is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest, e.g., human GBA1 locus, is used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest, e.g., human GBA1 locus, is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest, e.g., human GBA1 locus, is used to target the recombinant nuclease, e.g., Cas9, to induce a SSB at a cleavage site within the sense strand of the target gene; and the second guide RNA, e.g., second sgNA, that is specific to the antisense strand of a target gene of interest, e.g., human GBA1 locus, is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest, e.g., human GBA1 locus, is used to target the recombinant nuclease, e.g., Cas9, to induce a SSB at a cleavage site within the antisense strand of the target gene; and the second guide RNA, e.g., second sgNA, that is specific to the sense strand of a target gene of interest, e.g., human GBA1 locus, is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence, such as the human GBA1 locus, to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene, e.g., GBA1, that is homologous to a sequence comprised within the guide RNA, e.g., sgRNA. In some embodiments, the cleavage site of the sense strand is situated at a site within the sense strand of the target gene, e.g., GBA1, that is homologous to a sequence comprised within the first guide RNA, e.g., the first sgRNA. In some embodiments, the cleavage site of the antisense strand is situated at a site within the antisense strand of the target gene, e.g., GBA1, that is homologous to a sequence comprised within the first guide RNA, e.g., the first sgRNA. In some embodiments, the cleavage site of the sense strand is situated at a site within the sense strand of the target gene, e.g., GBA1, that is homologous to a sequence comprised within the second guide RNA, e.g., the second sgRNA. In some embodiments, the cleavage site of the antisense strand is situated at a site within the antisense strand of the target gene, e.g., GBA1, that is homologous to a sequence comprised within the second guide RNA, e.g., the second sgRNA. In some embodiments, the cleavage site of the sense strand is situated at a site within the sense strand of the target gene, e.g., GBA1, that is homologous to a sequence comprised within the first guide RNA, e.g., the first sgRNA; and the cleavage site of the antisense strand is situated at a site within the antisense strand of the target gene, e.g., GBA1, that is homologous to a sequence comprised within the second guide RNA, e.g., the second sgRNA. In some embodiments, the cleavage site of the antisense strand is situated at a site within the antisense strand of the target gene, e.g., GBA1, that is homologous to a sequence comprised within the first guide RNA, e.g., the first sgRNA; and the cleavage site of the sense strand is situated at a site within the sense strand of the target gene, e.g., GBA1, that is homologous to a sequence comprised within the second guide RNA, e.g., the second sgRNA. In some embodiments, the cleavage site of the antisense strand is situated at a site within the antisense strand of the target gene, e.g., GBA1, that is homologous to a sequence comprised within the second guide RNA, e.g., the second sgRNA; and the cleavage site of the sense strand is situated at a site within the sense strand of the target gene, e.g., GBA1, that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the PAM sequence on the sense strand and the PAM sequence on the antisense strand comprise the same nucleic acid sequence, which can be any PAM sequence disclosed herein. In some embodiments, the PAM sequence on the sense strand and the PAM sequence on the antisense strand each comprise a different nucleic acid sequence, each of which can be any of the PAM sequences disclosed herein.

In some embodiments, the PAM sequence that is recognized by a recombinant nuclease, e.g., Cas9, differs depending on the particular recombinant nuclease and the bacterial species it is from. In some embodiments, the PAM sequence recognized by SpCas9 is the nucleotide sequence 5′-NGG-3′ (SEQ ID NO: 64), where “N” is any nucleotide. In some embodiments, a PAM sequence recognized by SaCas9 is the nucleotide sequence 5′-NGRRT-3′ (SEQ ID NO: 65) or the nucleotide sequence 5′-NGRRN-3′ (SEQ ID NO: 66), where “N” is any nucleotide and “R” is a purine (e.g., guanine or adenine). In some embodiments, a PAM sequence recognized by NmeCas9 is the nucleotide sequence 5′-NNNNGATT-3′ (SEQ ID NO: 67), where “N” is any nucleotide. In some embodiments, a PAM sequence recognized by CjCas9 is the nucleotide sequence 5′-NNNNRYAC-3′ (SEQ ID NO: 68), where “N” is any nucleotide, “R” is a purine (e.g., guanine or adenine), and “Y” is a pyrimidine (e.g., cytosine or thymine). In some embodiments, a PAM sequence recognized by StCas9 is the nucleotide sequence 5′-NNAGAAW-3′ (SEQ ID NO: 69), where “N” is any nucleotide and “W” is adenine or thymine.

In some embodiments, the recombinant nuclease is Cas9 and the PAM sequence is the nucleotide sequence: (a) 5′-NGG-3′; (b) 5′-NGRRT-3′ or 5′-NGRRN-3′; (c) 5′-NNNNGATT-3′; (d) 5′-NNNNRYAC-3′; or (e) 5′-NNAGAAW-3′; where “N” is any nucleotide, “R” is a purine (e.g., guanine or adenine), “Y” is a pyrimidine (e.g., cytosine or thymine), and “W” is adenine or thymine. In some embodiments, the recombinant nuclease is Cas9, e.g., SpCas9, and the PAM sequence is 5′-NGG-3′, where “N” is any nucleotide. In some embodiments, the recombinant nuclease is Cas9, e.g., SaCas9, and the PAM sequence is 5′-NGRRT-3′ or 5′-NGRRN-3′, where “N” is any nucleotide and “R” is a purine, such as guanine or adenine. In some embodiments, the recombinant nuclease is Cas9, e.g., NmeCas9, and the PAM sequence is 5′-NNNNGATT-3′, where “N” is any nucleotide. In some embodiments, the recombinant nuclease is Cas9, e.g., CjCas9, and the PAM sequence is 5′-NNNNRYAC-3′, where “N” is any nucleotide, “R” is a purine, such as guanine or adenine, and “Y” is a pyrimidine, such as cytosine or thymine. In some embodiments, the recombinant nuclease is Cas9, e.g., StCas9, and the PAM sequence is 5′-NNAGAAW-3′, where “N” is any nucleotide and “W” is adenine or thymine.

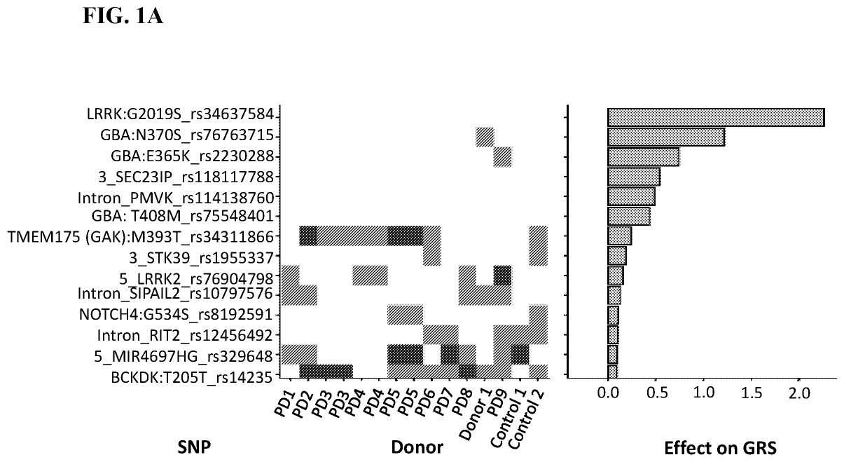

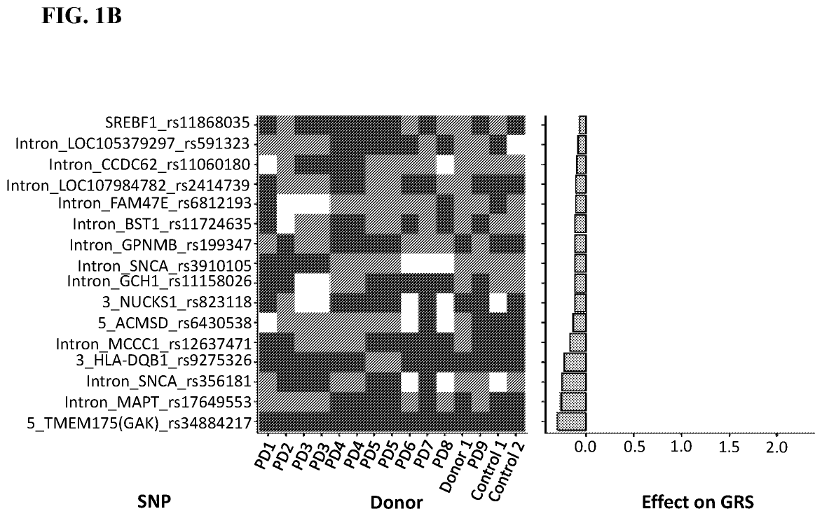

Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene, e.g., GBA1, that includes the cleavage site. In some embodiments, the crRNA comprises a nucleotide sequence that is 100% homologous to a portion of the target gene, e.g., GBA1, that includes the cleavage site. In some embodiments, the portion of the target gene, e.g., GBA1, that includes the cleavage site is a portion of the sense strand of the target gene that includes the cleavage site. In some embodiments, the portion of the target gene, e.g., GBA1, that includes the cleavage site is a portion of the antisense strand of the target gene that includes the cleavage site.

In some embodiments, the sgRNA comprises a crRNA sequence that is homologous to a sequence in the target gene, e.g., GBA1, that includes the cleavage site. In some embodiments, the first sgRNA comprises a crRNA sequence that is homologous to a sequence in the sense strand of the target gene, e.g., GBA1, that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene, e.g., GBA1, that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene, e.g., GBA1, that includes the cleavage site. In some embodiments, the crRNA sequence of the first sgRNA has 100% sequence identity to a sequence in the sense strand of the target gene, e.g., GBA1, that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene, e.g., GBA1, that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/nature13011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of the target gene, e.g., human GBA1, that includes the cleavage site. In some embodiments, the crRNA is homologous to a portion of the sense strand of the target gene, e.g., human GBA1, that includes the cleavage site. In some embodiments, the crRNA is homologous to a portion of the antisense strand of the target gene, e.g., human GBA1, that includes the cleavage site. In some embodiments, the crRNA of the first sgRNA is homologous to a portion of the sense strand of the target gene, e.g., human GBA1, that includes the cleavage site; and the crRNA of the second sgRNA is homologous to a portion of the antisense strand of the target gene, e.g., human GBA1, that includes the cleavage site.

In some embodiments, the crRNA is homologous to a portion of the antisense strand of the target gene, e.g., human GBA1, that includes the cleavage site. In some embodiments, the crRNA is homologous to a portion of the sense strand of the target gene, e.g., human GBA1 that includes the cleavage site. In some embodiments, the crRNA of the first sgRNA is homologous to a portion of the antisense strand of the target gene, e.g., human GBA1, that includes the cleavage site; and the crRNA of the second sgRNA is homologous to a portion of the sense strand of the target gene, e.g., human GBA1, that includes the cleavage site.

In some embodiments, the crRNA is homologous to a portion of the target gene, e.g., human GBA1, that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene, e.g., GBA1, that includes the cleavage site is on the sense strand. In some embodiments, the portion of the target gene, e.g., GBA1, that includes the cleavage site is on the antisense strand.

In some embodiments, the crRNA is homologous to a portion of the target gene, e.g., human GBA1, that includes the cleavage site, and there is no more than 40 nucleotides between the position of the nucleotide(s) causing the gene variant, e.g., SNP, and the portion of the target gene that is homologous to the crRNA, such as between 1 and 15, 1 and 20, 1 and 25, 1 and 30, 1 and 35, 1 and 40, 5 and 10, 5 and 15, 5 and 20, 5 and 25, 5 and 30, 5 and 35, 5 and 40, 10 and 20, 10 and 25, 10 and 30, 10 and 35, 10 and 40, 15 and 25, 15 and 30, 15 and 35, 15 and 40, 20 and 30, 20 and 35, or 20 and 40 nucleotides between the position of the nucleotide(s) causing the gene variant, e.g., SNP, and the portion of the target gene that is homologous to the crRNA. In some embodiments, the portion of the target gene, e.g., human GBA1, that includes the cleavage site is on the sense strand. In some embodiments, the portion of the target gene, e.g., human GBA1 that includes the cleavage site is on the antisense strand.

In some embodiments, the crRNA is homologous to a portion of the target gene, e.g., human GBA1, that includes the cleavage site, and there is no more than 40 nucleotides between the SNP and the portion of the target gene that is homologous to the crRNA, such as between 1 and 15, 1 and 20, 1 and 25, 1 and 30, 1 and 35, 1 and 40, 5 and 10, 5 and 15, 5 and 20, 5 and 25, 5 and 30, 5 and 35, 5 and 40, 10 and 20, 10 and 25, 10 and 30, 10 and 35, 10 and 40, 15 and 25, 15 and 30, 15 and 35, 15 and 40, 20 and 30, 20 and 35, or 20 and 40 nucleotides between the SNP and the portion of the target gene that is homologous to the crRNA.

In some embodiments, the crRNA is homologous to a portion, i.e., sequence, in the sense strand or the antisense strand of the target gene, e.g., GBA1, that includes the cleavage site and is immediately upstream of the PAM sequence.

In some embodiments, the crRNA comprises a nucleotide sequence set forth in any of SEQ ID NOS: 8 or 13-24, or a nucleotide sequence having at least 80%, 85%, 90%, or 95% sequence identity to a nucleotide sequence set forth in any of SEQ ID NOS: 8 or 13-24. In some embodiments, the crRNA comprises the nucleotide sequence GGACAAAGGCAAAGAGACAA (SEQ ID NO: 8). In some embodiments, the crRNA comprises a nucleotide sequence having at least 80%, 85%, 90%, or 95% sequence identity to the nucleotide sequence GGACAAAGGCAAAGAGACAA (SEQ ID NO: 8). In some embodiments, the crRNA comprises the nucleotide sequence AGCCGACCACATGGTACAGG (SEQ ID NO: 13). In some embodiments, the crRNA comprises a nucleotide sequence having at least 80%, 85%, 90%, or 95% sequence identity to the nucleotide sequence AGCCGACCACATGGTACAGG (SEQ ID NO: 13). In some embodiments, the crRNA comprises the nucleotide sequence GTACAGGAGGCTCTAGGGTA (SEQ ID NO: 14). In some embodiments, the crRNA comprises a nucleotide sequence having at least 80%, 85%, 90%, or 95% sequence identity to the nucleotide sequence GTACAGGAGGCTCTAGGGTA (SEQ ID NO: 14). In some embodiments, the crRNA comprises the nucleotide sequence GAGACAAAGGCTCAACACTG (SEQ ID NO: 15). In some embodiments, the crRNA comprises a nucleotide sequence having at least 80%, 85%, 90%, or 95% sequence identity to the nucleotide sequence GAGACAAAGGCTCAACACTG (SEQ ID NO: 15). In some embodiments, the crRNA comprises the nucleotide sequence AAGAGACAAAGGCTCAACAC (SEQ ID NO: 16). In some embodiments, the crRNA comprises a nucleotide sequence having at least 80%, 85%, 90%, or 95% sequence identity to the nucleotide sequence AAGAGACAAAGGCTCAACAC (SEQ ID NO: 16). In some embodiments, the crRNA comprises the nucleotide sequence ACATGGTACAGGAGGCTCTA (SEQ ID NO: 17). In some embodiments, the crRNA comprises a nucleotide sequence having at least 80%, 85%, 90%, or 95% sequence identity to the nucleotide sequence ACATGGTACAGGAGGCTCTA (SEQ ID NO: 17). In some embodiments, the crRNA comprises the nucleotide sequence AGGCTCTAGGGTAAGGACAA (SEQ ID NO: 18). In some embodiments, the crRNA comprises a nucleotide sequence having at least 80%, 85%, 90%, or 95% sequence identity to the nucleotide sequence AGGCTCTAGGGTAAGGACAA (SEQ ID NO: 18). In some embodiments, the crRNA comprises the nucleotide sequence CTAGAGCCTCCTGTACCATG (SEQ ID NO: 19). In some embodiments, the crRNA comprises a nucleotide sequence having at least 80%, 85%, 90%, or 95% sequence identity to the nucleotide sequence CTAGAGCCTCCTGTACCATG (SEQ ID NO: 19). In some embodiments, the crRNA comprises the nucleotide sequence CACATGGTACAGGAGGCTCT (SEQ ID NO: 20). In some embodiments, the crRNA comprises a nucleotide sequence having at least 80%, 85%, 90%, or 95% sequence identity to the nucleotide sequence CACATGGTACAGGAGGCTCT (SEQ ID NO: 20). In some embodiments, the crRNA comprises the nucleotide sequence AGCCTCCTGTACCATGTGGT (SEQ ID NO: 21). In some embodiments, the crRNA comprises a nucleotide sequence having at least 80%, 85%, 90%, or 95% sequence identity to the nucleotide sequence AGCCTCCTGTACCATGTGGT (SEQ ID NO: 21). In some embodiments, the crRNA comprises the nucleotide sequence AGTCGGTCCAGCCGACCACA (SEQ ID NO: 22). In some embodiments, the crRNA comprises a nucleotide sequence having at least 80%, 85%, 90%, or 95% sequence identity to the nucleotide sequence AGTCGGTCCAGCCGACCACA (SEQ ID NO: 22). In some embodiments, the crRNA comprises the nucleotide sequence ATGTGGTCGGCTGGACCGAC (SEQ ID NO: 23). In some embodiments, the crRNA comprises a nucleotide sequence having at least 80%, 85%, 90%, or 95% sequence identity to the nucleotide sequence ATGTGGTCGGCTGGACCGAC (SEQ ID NO: 23). In some embodiments, the crRNA comprises the nucleotide sequence TCCAGCCGACCACATGGTAC (SEQ ID NO: 24). In some embodiments, the crRNA comprises a nucleotide sequence having at least 80%, 85%, 90%, or 95% sequence identity to the nucleotide sequence TCCAGCCGACCACATGGTAC (SEQ ID NO: 24).

In some embodiments, the crRNA does not hybridize to a portion of the target gene, e.g., GBA1, that inclues the gene variant, e.g., SNP, associated with PD. In some embodiments, the crRNA does hybridize to a portion of the target gene, e.g., GBA1, that inclues the gene variant, e.g., SNP, associated with PD. In some embodiments, the crRNA of the first sgRNA hybridizes to a portion of the target gene, e.g., GBA1, that inclues the gene variant, e.g., SNP, associated with PD, but the crRNA of the second sgRNA does not hybridizes to a portion of the target gene that inclues the gene variant, e.g., SNP.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

Also provided herein is a complex, e.g., RNA complex, comprising one or more agent(s) capable of inducing a DSB comprises a recombinant nuclease, e.g., Cas9, and a guide RNA, e.g., sgRNA. In some embodiments, the recombinant nuclease is capable of inducing a DSB at a cleavage site within an endogenous target gene, e.g., GBA1, in a cell. In some embodiments, the target gene is human GBA1. In some embodiments, the human GBA1 includes a gene variant associated with PD. In some embodiments, the recombinant nuclease is any recombinant nuclease as described herein, e.g., in Section II.C. In some embodiments, the guide RNA is any guide RNA as described herein, e.g., in Section II.C. In some embodiments, the recombinant nuclease is a Cas nuclease. In some embodiments, the Cas nuclease is selected from the group consisting of Cas3, Cas9, Cas10, Cas12, and Cas13. In some embodiments, the Cas nuclease is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the guide RNA is an sgRNA and comprises a CRISPR targeting RNA (crRNA) sequence that is homologous to a sequence in the target gene, e.g., GBA1, that includes the cleavage site. In some embodiments, the crRNA sequence has 100% sequence identity to the sequence in the target gene, e.g., GBA1, that includes the cleavage site. In some embodiments, the crRNA sequence comprises a nucleic acid sequence set forth in any one of SEQ ID NO: 8 and 13-24. In some embodiments, the crRNA sequence comprises the nucleic acid sequence of SEQ ID NO: 8. In some embodiments, the crRNA sequence comprises the nucleic acid sequence of SEQ ID NO: 13. In some embodiments, the crRNA sequence comprises the nucleic acid sequence of SEQ ID NO: 14. In some embodiments, the crRNA sequence comprises the nucleic acid sequence of SEQ ID NO: 15. In some embodiments, the crRNA sequence comprises the nucleic acid sequence of SEQ ID NO: 16. In some embodiments, the crRNA sequence comprises the nucleic acid sequence of SEQ ID NO: 17. In some embodiments, the crRNA sequence comprises the nucleic acid sequence of SEQ ID NO: 18. In some embodiments, the crRNA sequence comprises the nucleic acid sequence of SEQ ID NO: 19. In some embodiments, the crRNA sequence comprises the nucleic acid sequence of SEQ ID NO: 20. In some embodiments, the crRNA sequence comprises the nucleic acid sequence of SEQ ID NO: 21. In some embodiments, the crRNA sequence comprises the nucleic acid sequence of SEQ ID NO: 22. In some embodiments, the crRNA sequence comprises the nucleic acid sequence of SEQ ID NO: 23. In some embodiments, the crRNA sequence comprises the nucleic acid sequence of SEQ ID NO: 24. In some embodiments, the Cas nuclease and the sgRNA form a ribonucleoprotein (RNP) complex.

Also provided herein is a complex, e.g., RNA complex, comprising one or more agent(s) capable of inducing a DSB comprises a recombinant nuclease, e.g., Cas9; and a first guide RNA, e.g., a first sgRNA; or a second guide RNA, e.g., a second sgRNA. In some embodiments, the recombinant nuclease is any recombinant nuclease as described herein, e.g., in Section II.C. In some embodiments, the first guide RNA is any guide RNA or first guide RNA, e.g., first sgRNA, as described herein, e.g., in Section II.C; and the second guide RNA is any guide RNA or second guide RNA, e.g., second sgRNA, as described herein, e.g., in Section II.C. In some embodiments, the recombinant nuclease is a Cas nuclease. In some embodiments, the recombinant nuclease is a Cas nuclease; the first guide RNA is a first sgRNA comprising a CRISPR targeting RNA (crRNA) sequence that is homologous to a sequence in a target gene, e.g., GBA1; or the second guide RNA is a second sgRNA comprising a crRNA sequence that is homologous to a sequence in the target gene, wheren the target gene comprises a sense strand and an antisense strand; wherein the crRNA sequence of the first sgRNA or the second sgRNA is homologous to a sequence in the sense strand that includes a cleavage site, or the crRNA sequence of the first sgRNA or the second sgRNA is homologous to a sequence in the antisense strand that includes a cleavage site; and wherein the target gene is human GBA1 and includes a single nucleotide polymorphism (SNP) that is associated with Parkinson's Disease. In some embodiments, the Cas nuclease comprises one or more mutations such that the Cas nuclease is converted into a nickase that lacks the ability to cleave both strands of a double stranded DNA molecule. In some embodiments, the Cas nuclease comprises one or more mutations such that the Cas nuclease is converted into a nickase that is able to cleave only one strand of a double stranded DNA molecule. In some embodiments, the Cas nuclease is selected from the group consisting of Cas3, Cas9, Cas10, Cas12, and Cas13. In some embodiments, the Cas nuclease is Cas9. In some embodiments, the Cas nuclease is an enhanced specificity Cas9 (eSpCas9). In some embodiments, the Cas nuclease is a high fidelity Cas9 (HiFi Cas9). In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of D10A, H840A, H854A, and H863A. In some embodiments, the crRNA sequence of the first sgRNA has 100% sequence identity to the sequence in the sense strand that includes the cleavage site. In some embodiments, the crRNA sequence of the second sgRNA has 100% sequence identity to the sequence in the antisense strand that includes the cleavage site. In some embodiments, (i) the Cas nuclease and the first sgRNA form a ribonucleoprotein (RNP) complex; or (ii) the Cas nuclease and the second sgRNA form a RNP complex.

Also provided herein is a pair of complexes, e.g., for correcting a gene variant associated with Parkinson's Disease, comprising: (1) a first Cas nuclease; and a first sgRNA comprising a CRISPR targeting RNA (crRNA) sequence that is homologous to a sequence in a target gene, e.g., GBA1; and (2) a second Cas nuclease; and a second sgRNA comprising a crRNA sequence that is homologous to a sequence in the target gene; wherein the target gene comprises a sense strand and an antisense strand; wherein the crRNA sequence of the first sgRNA is homologous to a sequence in the sense strand that includes a cleavage site, and the crRNA sequence of the second sgRNA is homologous to a sequence in the antisense strand that includes a cleavage site; and wherein the target gene is human GBA1 and includes a single nucleotide polymorphism (SNP) that is associated with Parkinson's Disease. In some embodiments, the SNP is situated between the cleavage site of the sense strand and the cleavage site of the antisense strand. In some embodiments, the first Cas nuclease is any Cas nuclease as described herein, e.g., in Section II.C. In some embodiments, the first guide RNA is any guide RNA or first guide RNA, e.g., first sgRNA, as described herein, e.g., in Section II.C; and the second guide RNA is any guide RNA or second guide RNA, e.g., second sgRNA, as described herein, e.g., in Section II.C. In some embodiments, the first Cas nuclease and the second Cas nuclease comprise one or more mutations such that the first Cas nuclease and the second Cas nuclease are each converted into a nickase that lacks the ability to cleave both strands of a double stranded DNA molecule. In some embodiments, the first Cas nuclease and the second Cas nuclease comprise one or more mutations such that the first Cas nuclease and the second Cas nuclease are each converted into a nickase that is able to cleave only one strand of a double stranded DNA molecule. In some embodiments, the first Cas nuclease and the second Cas nuclease is selected from the group consisting of Cas3, Cas9, Cas10, Cas12, and Cas13. In some embodiments, the first Cas nuclease and the second Cas nuclease is Cas9. In some embodiments, the Cas nuclease is an enhanced specificity Cas9 (eSpCas9). In some embodiments, the Cas nuclease is a high fidelity Cas9 (HiFi Cas9). In some embodiments, the first Cas nuclease and the second Cas nuclease is from a bacteria selected from the group consisting of Streptococcus pyogenes, Staphylococcus aureus, Neisseria meningitides, Campylobacter jejuni, and Streptococcus thermophilis. In some embodiments, the first Cas nuclease and the second Cas nuclease is from Streptococcus pyogenes. In some embodiments, the first Cas nuclease and the second Cas nuclease comprises one or more mutations in the RuvC I, RuvC II, or RuvC III motifs. In some embodiments, the one or more mutations comprises a D10A mutation in the RuvC I motif. In some embodiments, the first Cas nuclease and the second Cas nuclease comprises one or more mutations in the HNH catalytic domain. In some embodiments, the one or more mutations in the HNH catalytic domain is selected from the group consisting of H840A, H854A, and H863A. In some embodiments, the one or more mutations in the HNH catalytic domain comprises a H840A mutation. In some embodiments, the first Cas nuclease and the second Cas nuclease comprises a mutation selected from the group consisting of D10A, H840A, H854A, and H863A. In some embodiments, the crRNA sequence of the first sgRNA has 100% sequence identity to the sequence in the sense strand that includes the cleavage site. In some embodiments, the crRNA sequence of the second sgRNA has 100% sequence identity to the sequence in the antisense strand that includes the cleavage site. In some embodiments, (i) the first Cas nuclease and the first sgRNA form a ribonucleoprotein (RNP) complex; and/or (ii) the second Cas nuclease and the second sgRNA form a RNP complex.

Also provided herein is an isolated nucleic acid, e.g., an isolated nucleic acid for use in a method of correcting a gene variant associated with PD, comprising the nucleic acid sequence of any of the guide RNAs, e.g., sgRNAs, or crRNAs, described herein. In some embodiments, the crRNA comprises a nucleotide sequence as set forth in any one of SEQ ID NOS: 8 and 13-24, or a nucleotide sequence having at least 80%, 85%, 90%, or 95% sequence identity to a nucleotide sequence as set forth in any one of SEQ ID NOS: 8 and 13-24. In some embodiments, the crRNA comprises the nucleic acid sequence as set forth in SEQ ID NO: 8. In some embodiments, the crRNA comprises a nucleotide sequence having at least 80%, 85%, 90%, or 95% sequence identity to the nucleotide sequence as set forth in SEQ ID NO: 8. In some embodiments, the crRNA comprises the nucleic acid sequence as set forth in SEQ ID NO: 13. In some embodiments, the crRNA comprises a nucleotide sequence having at least 80%, 85%, 90%, or 95% sequence identity to the nucleotide sequence as set forth in SEQ ID NO: 13. In some embodiments, the crRNA comprises the nucleic acid sequence as set forth in SEQ ID NO: 14. In some embodiments, the crRNA comprises a nucleotide sequence having at least 80%, 85%, 90%, or 95% sequence identity to the nucleotide sequence as set forth in SEQ ID NO: 14. In some embodiments, the crRNA comprises the nucleic acid sequence as set forth in SEQ ID NO: 15. In some embodiments, the crRNA comprises a nucleotide sequence having at least 80%, 85%, 90%, or 95% sequence identity to the nucleotide sequence as set forth in SEQ ID NO: 15. In some embodiments, the crRNA comprises the nucleic acid sequence as set forth in SEQ ID NO: 16. In some embodiments, the crRNA comprises a nucleotide sequence having at least 80%, 85%, 90%, or 95% sequence identity to the nucleotide sequence as set forth in SEQ ID NO: 16. In some embodiments, the crRNA comprises the nucleic acid sequence as set forth in SEQ ID NO: 17. In some embodiments, the crRNA comprises a nucleotide sequence having at least 80%, 85%, 90%, or 95% sequence identity to the nucleotide sequence as set forth in SEQ ID NO: 17. In some embodiments, the crRNA comprises the nucleic acid sequence as set forth in SEQ ID NO: 18. In some embodiments, the crRNA comprises a nucleotide sequence having at least 80%, 85%, 90%, or 95% sequence identity to the nucleotide sequence as set forth in SEQ ID NO: 18. In some embodiments, the crRNA comprises the nucleic acid sequence as set forth in SEQ ID NO: 19. In some embodiments, the crRNA comprises a nucleotide sequence having at least 80%, 85%, 90%, or 95% sequence identity to the nucleotide sequence as set forth in SEQ ID NO: 19. In some embodiments, the crRNA comprises the nucleic acid sequence as set forth in SEQ ID NO: 20. In some embodiments, the crRNA comprises a nucleotide sequence having at least 80%, 85%, 90%, or 95% sequence identity to the nucleotide sequence as set forth in SEQ ID NO: 20. In some embodiments, the crRNA comprises the nucleic acid sequence as set forth in SEQ ID NO: 21. In some embodiments, the crRNA comprises a nucleotide sequence having at least 80%, 85%, 90%, or 95% sequence identity to the nucleotide sequence as set forth in SEQ ID NO: 21. In some embodiments, the crRNA comprises the nucleic acid sequence as set forth in SEQ ID NO: 22. In some embodiments, the crRNA comprises a nucleotide sequence having at least 80%, 85%, 90%, or 95% sequence identity to the nucleotide sequence as set forth in SEQ ID NO: 22. In some embodiments, the crRNA comprises the nucleic acid sequence as set forth in SEQ ID NO: 23. In some embodiments, the crRNA comprises a nucleotide sequence having at least 80%, 85%, 90%, or 95% sequence identity to the nucleotide sequence as set forth in SEQ ID NO: 23. In some embodiments, the crRNA comprises the nucleic acid sequence as set forth in SEQ ID NO: 24. In some embodiments, the crRNA comprises a nucleotide sequence having at least 80%, 85%, 90%, or 95% sequence identity to the nucleotide sequence as set forth in SEQ ID NO: 24.

### D. Homology-Directed Repair (HDR)

In some aspects, the provided embodiments involve targeted integration of a specific part of a nucleic acid sequence, such as a donor template, at a particular location, e.g., at a gene variant associated with PD, such as at a gene variant in GBA1 that is associated with PD.

In some embodiments, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break.

In some embodiments, HDR is utilized for targeted integration or insertion of a nucleic acid sequence(s), e.g., a donor template, at one or more gene variant site(s) in one or more target gene(s), e.g., GBA1. In some embodiments, HDR can be used to alter a gene variant, e.g., to alter a gene variant associated with PD into a wildtype form of the gene variant, or to integrate a donor template comprising a corrected form of the gene variant, e.g., SNP, into a target gene, e.g., GBA1, at a particular location, and/or to edit or correct a gene variant, e.g., mutation or single nucleotide polymorphism (SNP), in a particular target gene.

Agents capable of inducing a DSB, such as Cas nucleases (e.g. Cas9), TALENs, and ZFNs, promote genomic editing by inducing a DSB at a cleavage site within a target gene, e.g., GBA1, as discussed, e.g., in Section II.C.

Agents capable of inducing a SSB, also sometimes referred to as a nick, include recombinant nucleases, e.g., Cas9, having nickase activity, such as, e.g., those described in Section II.C. Examples of agents having nickase activity includes, e.g., a Cas9 from Streptococcus pyogenes that comprises a mutation selected from the group consisting of of D10A, H840A, H854A, and H863A.

Upon cleavage by one of these agents, the target gene, e.g., GBA1, with the SSBs or the DSB undergoes one of two major pathways for DNA damage repair: (1) the error-prone non-homologous end joining (NHEJ), or (2) the high-fidelity homology-directed repair (HDR) pathway.

In some embodiments, cells in which SSBs or a DSB was previously induced by one or more agent(s) comprising a recombinant nuclease, are obtained, and a donor template, e.g., ssODN, is introduced to result in HDR and integration of the donor template into the target gene, e.g., GBA1.

In general, in the absence of a repair template, e.g., a donor template, such as a ssODN, the NHEJ process re-ligates the ends of the cleaved DNA strands, which frequently results in nucleotide deletions and insertions at the cleavage site.

Alteration of nucleic acid sequences at a gene variant site, such as a gene variant in human GBA1 that is associated with PD, can occur by HDR by integrating an exogenously provided donor template that includes one or more nucleotide changes that reflects a form of the gene variant that is not associated with PD, such as a wildtype form of the particular gene variant, e.g., a donor template comprising a corrected form of the gene variant, e.g., SNP. The HDR pathway can occur by way of the canonical HDR pathway or the alternative HDR pathway. Unless otherwise indicated, the term “HDR” or “homology-directed repair” as used herein encompasses both canonical HDR and alternative HDR.

Canonical HDR or “canonical homology-directed repair” or cHDR,” are used interchangeably, and refers to the process of repairing DNA damage using a homologous nucleic acid (e.g., an endogenous homologous sequence, such as a sister chromatid; or an exogenous nucleic acid, such as a donor template). Canonical HDR typically acts when there has been a significant resection at the DSB, forming at least one single-stranded portion of DNA. In a normal cell, canonical HDR typically involves a series of steps such as recognition of the break, stabilization of the break, resection, stabilization of single-stranded DNA, formation of a DNA crossover intermediate, resolution of the crossover intermediate, and ligation. The canonical HDR process requires RAD51 and BRCA2, and the homologous nucleic acid, e.g., donor templae, is typically double-stranded. In canonical HDR, a double-stranded polynucleotide, e.g., a double stranded donor template, is introduced, which comprises a sequence that is homologous to the targeting sequence that comprises the gene variant associated with PD, and which will either be directly integrated into the targeting sequence or will be used as a template to insert the sequence, or a portion the sequence, of the donor template into the target gene, e.g., GBA1. After resection at the break, repair can progress by different pathways, e.g., by the double Holliday junction model (also referred to as the double strand break repair, or DSBR, pathway), or by the synthesis-dependent strand annealing (SDSA) pathway.

In the double Holliday junction model, strand invasion occurs by the two single stranded overhangs of the targeting sequence to the homologous sequences in the double-stranded polynucleotde, e.g., double stranded donor template, which results in the formation of an intermediate with two Holliday junctions. The junctions migrate as new DNA is synthesized from the ends of the invading strand to fill the gap resulting from the resection. The end of the newly synthesized DNA is ligated to the resected end, and the junctions are resolved, resulting in the insertion at the targeting sequence, or a portion of the targeting sequence that includes the gene variant. Crossover with the polynucleotide, e.g., donor template, may occur upon resolution of the junctions.

In the SDSA pathway, only one single stranded overhang invades the polynucleotide, e.g., donor template, and new DNA is synthesized from the end of the invading strand to fill the gap resulting from resection. The newly synthesized DNA then anneals to the remaining single stranded overhang, new DNA is synthesized to fill in the gap, and the strands are ligated to produce the modified DNA duplex.

Alternative HDR, or “alternative homology-directed repair,” or “alternative HDR,” are used interchangeably, and refers, in some embodiments, to the process of repairing DNA damage using a homologous nucleic acid (e.g., an endogenous homologous sequence, such as a sister chromatid; or an exogenous nucleic acid, such as a donor template). Alternative HDR is distinct from canonical HDR in that the process utilizes different pathways from canonical HDR, and can be inhibited by the canonical HDR mediators, RAD51 and BRCA2. Moreover, alternative HDR is also distinguished by the involvement of a single-stranded or nicked homologous nucleic acid template, e.g., donor template, whereas canonical HDR generally involves a double-stranded homologous template. In the alternative HDR pathway, a single strand template polynucleotide, e.g., donor template, is introduced. A nick, single strand break, or DSB at the cleavage site, for altering a desired target site, e.g., a gene variant in a target gene, e.g., GBA1, is mediated by a nuclease molecule, e.g., any of the nucleases as described, for instance, in Section II.C, and resection at the break occurs to reveal single stranded overhangs. Incorporation of the sequence of the template polynucleotide, e.g., donor template, to correct or alter the target site of the DNA typically occurs by the SDSA pathway, as described herein.

In some embodiments, HDR is carried out by introducing, into a cell, one or more agent(s) capable of inducing a DSB, such as any of those as described in Section II.C, and a donor template, e.g., ssODN, such as any of those described in Section II.E. The introducing can be carried out by any suitable delivery means, such as any of those as described in Section II.F. The conditions under which HDR is allowed to occur can be any conditions suitable for carrying out HDR in a cell.

In some embodiments, HDR is carried out by introducing, into a cell, one or more agent(s) capable of inducing a SSB in each stand, such as any of those as described in Section II.C, and a donor template, e.g., ssODN, such as any of those described in Section II.E. The introducing can be carried out by any suitable delivery means, such as any of those as described in Section II.F. The conditions under which HDR is allowed to occur can be any conditions suitable for carrying out HDR in a cell.

### E. Donor Templates

In some embodiments, the provided methods include the use of a donor template, e.g., a donor template comprising a corrected form of the gene variant, e.g., SNP, that is homologous to a portion(s) of the targeting sequence in the target gene, e.g., GBA1. In some embodiments, the targeting sequence is comprised within the sense strand. In some embodiments, the targeting sequence is comprised within the antisense strand. Also provided, in some embodiments, are donor templates for use in the methods provided herein, e.g., as templates for HDR-mediated integration of a corrected form of the gene variant, e.g., SNP. After integration of the “corrected form” of the gene variant, e.g., SNP, into the target gene, e.g., GBA1, the target gene no longer includes the gene variant associated with PD due to one or more nucleotide changes that was/were introduced by the donor template.

In some embodiments, after integration of the donor template, e.g., ssODN, comprising a corrected form of the gene variant, e.g., SNP, into the target gene, e.g., GBA1, the target gene comprises the corrected form of the gene variant, e.g., SNP, instead of the gene variant, e.g., SNP, that is associated with PD. In some embodiments, after integration of the donor template, e.g., ssODN, comprising a corrected form of the SNP into the target gene, e.g., GBA1, the target gene comprises the corrected form of the SNP instead of the SNP that is associated with PD. In some embodiments, the corrected form of the SNP is not associated with PD. In some embodiments, the corrected form of the SNP is a wildtype form of the SNP. In some embodiments, the corrected form of the SNP is the major allele of the SNP.

In some embodiments, the donor template comprises a nucleic acid sequence that is homologous to the nucleic acid sequence of the targeting sequence, except for one or more nucleotide(s). In some embodiments, the donor template is homologous to the nucleic acid sequence of the targeting sequence except for one or more nucleotide(s) of the gene variant, e.g., SNP, that results in the gene variant being associated with PD. In some embodiments, the donor template comprises a nucleic acid sequence that is not homologous to the targeting sequence at the SNP. In some embodiments, the donor template contains one or more homology sequences, e.g., homology arms, linked to or flanking the one or more nucleotide(s) of the corrected form of the gene variant, e.g., SNP, that differ from the homologous sequence in the gene variant associated with PD. In general, the homologous sequence(s) are used to target the donor template for HDR-mediated integration into the sequence of the targeting sequence within the target gene, e.g., GBA1, thereby resulting in integration of the corrected form of the gene variant, e.g., SNP.

In some embodiments, the donor template comprises the nucleic acid sequence of the targeting sequence except for differing by including: (a) one or more nucleotide(s) of the corrected form of the gene variant, e.g., SNP; and/or (b) one or more nucleotide(s) that introduce a restriction site that is recognized by one or more restriction enzymes; and/or (c) one or more nucleotide(s) that introduce one or more silent mutations. In some embodiments, the corrected form of the gene variant, e.g., SNP, is the wildtype form of the gene variant, e.g., SNP.

In some embodiments, the donor template comprises a nucleic acid sequence comprising one or more nucleotides that are not homologous to the targeting sequence, wherein the one or more nucleotides comprises one or more nucleotides that introduce a restriction site that is recognized by one or more restriction enzymes. In some embodiments, the donor template comprises a nucleic acid sequence comprising one or more nucleotides that are not homologous to the targeting sequence, wherein the one or more nucleotides comprises (i) one or more nucleotides of the corrected form of the gene variant, e.g., SNP, and (ii) one or more nucleotides that introduce a restriction site that is recognized by one or more restriction enzymes. In some embodiments, the one or more nucleotides that introduce a restriction site that is recognized by one or more restriction enzymes result in a silent mutation(s). In some embodiments, the donor template comprises a nucleic acid sequence comprising one or more nucleotides that are not homologous to the targeting sequence, wherein the one or more nucleotides comprises (i) one or more nucleotides of the corrected form of the gene variant, e.g., SNP, and (ii) one or more nucleotides that introduce a restriction site that is recognized by one or more restriction enzymes; and (iii) one or more nucleotides that introduce one or more silent mutations.

In some embodiments, the donor template is used in conjunction with the one or more agent(s) capable of inducing a DNA break, e.g., a SSB or a DSB, to replace the sequence of the gene variant associated with PD with the sequence of the corrected form of the gene variant, e.g., SNP. In some embodiments, the donor template is used in conjunction with the one or more agent(s) capable of inducing a DSB and the guide RNA, e.g., sgRNA, to replace the sequence of the gene variant associated with PD with the sequence of the corrected form of the gene variant, e.g., SNP. In some embodiments, the donor template is used in conjunction with the one or more agent(s) capable of inducing a SSB; the first guide RNA, e.g., the first sgRNA; and the second guide RNA, e.g., the second sgRNA, to replace the sequence of the gene variant associated with PD with the sequence of the corrected form of the gene variant, e.g., SNP.

In some embodiments, the donor template comprises a nucleic acid sequence that is homologous to the cleavage site in the target gene, e.g., GBA1. In some embodiments, the donor template comprises a nucleic acid sequence that is homologous to the cleavage site in the sense strand of the target gene, e.g., GBA1. In some embodiments, the donor template comprises a nucleic acid sequence that is homologous to the cleavage site in the antisense strand of the target gene, e.g., GBA1.

In some embodiments, the donor template has a length that is between 50 and 500 nucleotides in length. In some embodiments, the donor template has a length that is between 50 and 450, 50 and 400, 50 and 350, 50 and 300, 50 and 250, 50 and 200, 50 and 175, 50 and 150, 50 and 125, or 50 and 100 nucleotides in length. In some embodiments, the donor template has a length that is between 75 and 450, 75 and 400, 75 and 350, 75 and 300, 75 and 250, 75 and 200, 75 and 175, 75 and 150, 75 and 125, or 75 and 100 nucleotides in length. In some embodiments, the donor template has a length that is between 100 and 450, 100 and 400, 100 and 350, 100 and 300, 100 and 250, 100 and 200, 100 and 175, 100 and 150, or 100 and 125 nucleotides in length. In some embodiments, the donor template has a length that is between 80 and 500, 80 and 450, 80 and 400, 80 and 350, 80 and 300, 80 and 250, 80 and 200, 80 and 175, 80 and 150, 80 and 125, or 80 and 100 nucleotides in length. In some embodiments, the donor template has a length that is, is about, is at least, or is at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 205, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides in length.

In some embodiments, the target gene, e.g., GBA1, includes a sense strand and an antisense strand, and the sense strand comprises the targeting sequence. In some embodiments, the target gene, e.g., GBA1, includes a sense strand and an antisense strand, and the antisense strand comprises the targeting sequence.

In some embodiments, the donor template comprises a nucleic acid sequence that is substantially homologous to a targeting sequence in the target gene, e.g., GBA1, that includes the gene variant, e.g., SNP. In some embodiments, the targeting sequence is comprised within the sense strand. In some embodiments, the targeting sequence is comprised within the antisense strand. When used in reference to the nucleic acid sequence of a donor template, such as a ssODN, the term “substantially homologous” refers to a nucleic acid sequence having a degree of identity to a DNA sequence within a target gene, e.g., GBA1, of at least 80%, preferably at least 90%, more preferably at least 95%. In some embodiments, the nucleic acid sequence is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence. In some embodiments, the donor template comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to a portion of the human GBA1 gene that comprises the gene variant, e.g., SNP. In some embodiments, the donor template comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to a sequence in human GBA1 that comprises the gene variant, e.g., SNP, and is, is about, or is at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 205, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides in length.

In some embodiments, the targeting sequence comprises the gene variant, e.g., SNP, and a protospacer adjacent motif (PAM) sequence.

In some embodiments, the sense strand comprises the targeting sequence, and the targeting sequence includes the SNP and a protospacer adjacent motif (PAM) sequence. In some embodiments, the antisense strand comprises a sequence that is complementary to the targeting sequence and includes a PAM sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence.

In some embodiments, the antisense strand comprises the targeting sequence, and the targeting sequence includes the SNP and a protospacer adjacent motif (PAM) sequence. In some embodiments, the sense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the donor template, e.g., ssODN, comprises a nucleic acid sequence comprising a PAM sequence that is homologous to the PAM sequence in the targeting sequence. In some embodiments, the donor template, e.g., ssODN, comprises a nucleic acid sequence comprising a PAM sequence that is not homologous to the PAM sequence in the targeting sequence at one or more positions that result in a silent mutation. In some embodiments, the one or more positions that result in a silent mutation in the PAM sequence such that the mutated PAM sequence is not recognized by the recombinant nuclease.

The introduction of one or more nucleotide changes by the donor template that results in a silent mutation in the PAM sequence can be beneficial because it would prevent, or diminish the likelihood of, the re-cutting of corrected gene variants because the donor template introduced a mutated PAM sequence that is not recognized by the recombinant nuclease.

In some embodiments, the targeting sequence has a length that is between 50 and 500 nucleotides in length. In some embodiments, the targeting sequence has a length that is between 50 and 450, 50 and 400, 50 and 350, 50 and 300, 50 and 250, 50 and 200, 50 and 175, 50 and 150, 50 and 125, or 50 and 100 nucleotides in length. In some embodiments, the targeting sequence has a length that is between 75 and 450, 75 and 400, 75 and 350, 75 and 300, 75 and 250, 75 and 200, 75 and 175, 75 and 150, 75 and 125, or 75 and 100 nucleotides in length. In some embodiments, the targeting sequence has a length that is between 100 and 450, 100 and 400, 100 and 350, 100 and 300, 100 and 250, 100 and 200, 100 and 175, 100 and 150, or 100 and 125 nucleotides in length. In some embodiments, the targeting sequence has a length that is between 80 and 500, 80 and 450, 80 and 400, 80 and 350, 80 and 300, 80 and 250, 80 and 200, 80 and 175, 80 and 150, 80 and 125, or 80 and 100 nucleotides in length. In some embodiments, the targeting sequence has a length that is, is about, is at least, or is at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 205, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, or 500 nucleotides in length.

In some embodiments, the donor template comprises a nucleic acid sequence that is not homologous to the targeting sequence at the SNP position.

In some embodiments, the donor template, e.g., ssODN, comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence, and is not homologous to the targeting sequence at the SNP position.

In some embodiments, the donor template, e.g., ssODN, comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence, and is not homologous to the targeting sequence at the SNP position and at one or more nucleotide(s) of the PAM sequence.

In some embodiments, the donor template, e.g., ssODN, comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence, and is not homologous to the targeting sequence at (i) the SNP position, (ii) one or more nucleotide(s) of the PAM sequence, and (iii) one or more nucleotide(s) that introduce a restriction site that is recognized by one or more restriction enzymes.

In some embodiments, the donor template, e.g., ssODN, comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence, and is not homologous to the targeting sequence at (i) the SNP position, and/or (ii) one or more nucleotide(s) of the PAM sequence, and/or (iii) one or more nucleotide(s) that introduce a restriction site that is recognized by one or more restriction enzymes.

The introduction of a restriction site, particularly those that result in a silent mutation, can be beneficial because it would allow for screening cells to identify those that incorporated the donor template since the donor template includes the restriction site at that specific position but the native sequence of the target gene, e.g., GBA1, does not. Screening can be carried out, for instance, by exposing isolated DNA from a clone of the cell to a restriction enzyme that recognizes that particular restriction site under conditions suitable to promote cleavage, thereby allowing for cleavage of the DNA at that particular site, which can be detected using conventional techniques. In some embodiments, the restriction site is recognized by a BstX1 restriction enzyme. In some embodiments, the restriction site introduced by the ssODN results from the introduction of a guanine (G) to alanine (A) substitution. In some embodiments, the restriction site is rs755952419 G>A. In some embodiments, the restriction site introduced by the ssODN results from the introduction of a guanine (G) to cytosine (C) substitutution. In some embodiments, the restriction site is rs753067275 G>C.

In some embodiments, the one or more nucleotide(s) of the donor template that are not homologous to the PAM sequence of the targeting sequence result in a silent mutation after integration of the donor template into the target gene, e.g., GBA1. In some embodiments, the nucleic acid sequence of the donor template comprises a PAM sequence that is not homologous to the PAM sequence in the targeting sequence at one or more positions that result in a silent mutation.

In some embodiments, the donor template is single-stranded. In some embodiments, the donor template is a single-stranded DNA oligonucleotide (ssODN). In some embodiments, the donor template is double-stranded.

In some embodiments, the ssODN comprises a 5′ ssODN arm and a 3′ ssODN arm. In some embodiments, the 5′ ssODN arm is directly linked to the 3′ ssODN arm. In some embodiments, the 5′ ssODN arm is homologous to the sequence of the target gene, e.g., GBA1, that is immediately upstream of the cleavage site, and the 3′ ssODN arm is homologous to the sequence of the target gene that is immediately downstream of the cleavage site.

In some embodiments, the 5′ ssODN arm and/or the 3′ ssODN arm has a length that is between 20 and 300, 20 and 250, 20 and 150, 20 and 100, 20 and 80, 20 and 60, or 20 and 40 nucleotides in length. In some embodiments, the 5′ ssODN arm and/or the 3′ ssODN arm has a length that is between 30 and 300, 30 and 250, 30 and 150, 30 and 100, 30 and 80, 30 and 60, or 30 and 40 nucleotides in length. In some embodiments, the 5′ ssODN arm and/or the 3′ ssODN arm has a length that is between 40 and 300, 40 and 250, 40 and 150, 40 and 100, 40 and 80, or 40 and 60 nucleotides in length. In some embodiments, the 5′ ssODN arm and/or the 3′ ssODN arm has a length that is between 50 and 300, 50 and 250, 50 and 150, 50 and 100, 50 and 80, or 50 and 60 nucleotides in length. In some embodiments, the 5′ ssODN arm and/or the 3′ ssODN arm has a length that is, is about, is at least, or is at least about 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 nucleotides in length.

In some embodiments, the donor template, e.g., ssODN, is homologous to the target gene and comprises a corrected form of the gene variant, e.g., SNP.

In some embodiments, the 5′ ssODN arm comprises the nucleic acid sequence set forth in any one of SEQ ID NOS: 1, 4, 25, 28, 31, 34, 37, 40, 43, 46, 49, 52, 55, 58, and 61.

In some embodiments, the 3′ ssODN arm comprises the nucleic acid sequence set forth in any one of SEQ ID NOS: 2, 26, 29, 32, 35, 38, 41, 44, 47, 50, 53, 56, 59, and 62.

In some embodiments, the ssODN comprises the nucleic acid sequence set forth in any of SEQ ID NOS: 3, 5, 27, 30, 33, 36, 39, 42, 45, 48, 51, 54, 57, 60, and 63.

In some embodiments, the 5′ ssODN arm comprises the nucleic acid sequence as follows: TTCCAGTCGGTCCAGCCGACCACATGGTACAGGAGGTTCTAGGGTAAGGACAAAGGCAAAG AGA (SEQ ID NO: 4). In some embodiments, the 3′ ssODN arm comprises the nucleic acid sequence as follows: CAAAGGCTCAACACTGGGGGTCCCCAGAGAGTGTAG (SEQ ID NO: 2). In some embodiments, the 5′ ssODN arm comprises the nucleic acid sequence as set forth in SEQ ID NO: 4), and the 3′ ssODN arm comprises the nucleic acid sequence as set forth in SEQ ID NO: 2.

In some embodiments, the ssODN comprises the following nucleic acid sequence: TTCCAGTCGGTCCAGCCGACCACATGGTACAGGAGGTTCTAGGGTAAGGACAAAGGCAAAG AGACAAAGGCTCAACACTGGGGGTCCCCAGAGAGTGTAG (SEQ ID NO: 5).

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 4. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 2. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 5.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 5. In some embodiments, the 5′ ssODN arm comprises the nucleic acid sequence as set forth in SEQ ID NO: 4. In some embodiments, the 3′ ssODN arm comprises the nucleic acid sequence as set forth in SEQ ID NO: 2. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 4. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 2. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 5.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence as follows: TTCCAGTCGGTCCAGCCAACCACATGGTACAGGAGGTTCTAGGGTAAGGACAAAGGCAAAG AGA (SEQ ID NO: 1). In some embodiments, the 3′ ssODN arm comprises the nucleic acid sequence as follows: CAAAGGCTCAACACTGGGGGTCCCCAGAGAGTGTAG (SEQ ID NO: 2). In some embodiments, the 5′ ssODN arm comprises the nucleic acid sequence as set forth in SEQ ID NO: 1), and the 3′ ssODN arm comprises the nucleic acid sequence as set forth in SEQ ID NO: 2.

In some embodiments, the ssODN comprises the following nucleic acid sequence: TTCCAGTCGGTCCAGCCAACCACATGGTACAGGAGGTTCTAGGGTAAGGACAAAGGCAAAG AGACAAAGGCTCAACACTGGGGGTCCCCAGAGAGTGTAG (SEQ ID NO: 3).

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 1. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 2. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 3.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 3. In some embodiments, the 5′ ssODN arm comprises the nucleic acid sequence as set forth in SEQ ID NO: 1. In some embodiments, the 3′ ssODN arm comprises the nucleic acid sequence as set forth in SEQ ID NO: 2. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 1. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 2. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 3.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 25. In some embodiments, the 3′ ssODN arm comprises the nucleic acid sequence set forth in SEQ ID NO: 26. In some embodiments, the 5′ ssODN arm comprises the nucleic acid sequence as set forth in SEQ ID NO: 25, and the 3′ ssODN arm comprises the nucleic acid sequence as set forth in SEQ ID NO: 26. In some embodiments, the ssODN comprises the nucleic acid sequence set forth in SEQ ID NO: 27.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 25. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 26. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 27.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 27. In some embodiments, the 5′ ssODN arm comprises the nucleic acid sequence as set forth in SEQ ID NO: 25. In some embodiments, the 3′ ssODN arm comprises the nucleic acid sequence as set forth in SEQ ID NO: 26. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 25. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 26. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 27.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 28. In some embodiments, the 3′ ssODN arm comprises the nucleic acid sequence set forth in SEQ ID NO: 29. In some embodiments, the 5′ ssODN arm comprises the nucleic acid sequence as set forth in SEQ ID NO: 28, and the 3′ ssODN arm comprises the nucleic acid sequence as set forth in SEQ ID NO: 29. In some embodiments, the ssODN comprises the nucleic acid sequence set forth in SEQ ID NO: 30.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 28. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 29. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 30.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 30. In some embodiments, the 5′ ssODN arm comprises the nucleic acid sequence as set forth in SEQ ID NO: 28. In some embodiments, the 3′ ssODN arm comprises the nucleic acid sequence as set forth in SEQ ID NO: 29. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 28. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 29. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 30.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 31. In some embodiments, the 3′ ssODN arm comprises the nucleic acid sequence set forth in SEQ ID NO: 32. In some embodiments, the 5′ ssODN arm comprises the nucleic acid sequence as set forth in SEQ ID NO: 31, and the 3′ ssODN arm comprises the nucleic acid sequence as set forth in SEQ ID NO: 32. In some embodiments, the ssODN comprises the nucleic acid sequence set forth in SEQ ID NO: 33.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 31. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 32. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 33.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 33. In some embodiments, the 5′ ssODN arm comprises the nucleic acid sequence as set forth in SEQ ID NO: 31. In some embodiments, the 3′ ssODN arm comprises the nucleic acid sequence as set forth in SEQ ID NO: 32. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 31. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 32. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 33.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 34. In some embodiments, the 3′ ssODN arm comprises the nucleic acid sequence set forth in SEQ ID NO: 35. In some embodiments, the 5′ ssODN arm comprises the nucleic acid sequence as set forth in SEQ ID NO: 34, and the 3′ ssODN arm comprises the nucleic acid sequence as set forth in SEQ ID NO: 35. In some embodiments, the ssODN comprises the nucleic acid sequence set forth in SEQ ID NO: 36.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 34. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 35. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 36.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 36. In some embodiments, the 5′ ssODN arm comprises the nucleic acid sequence as set forth in SEQ ID NO: 34. In some embodiments, the 3′ ssODN arm comprises the nucleic acid sequence as set forth in SEQ ID NO: 35. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 34. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 35. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 36.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 37. In some embodiments, the 3′ ssODN arm comprises the nucleic acid sequence set forth in SEQ ID NO: 37. In some embodiments, the 5′ ssODN arm comprises the nucleic acid sequence as set forth in SEQ ID NO: 37, and the 3′ ssODN arm comprises the nucleic acid sequence as set forth in SEQ ID NO: 38. In some embodiments, the ssODN comprises the nucleic acid sequence set forth in SEQ ID NO: 39.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 37. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 38. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 39.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 39. In some embodiments, the 5′ ssODN arm comprises the nucleic acid sequence as set forth in SEQ ID NO: 37. In some embodiments, the 3′ ssODN arm comprises the nucleic acid sequence as set forth in SEQ ID NO: 38. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 37. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 38. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 39.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 40. In some embodiments, the 3′ ssODN arm comprises the nucleic acid sequence set forth in SEQ ID NO: 41. In some embodiments, the 5′ ssODN arm comprises the nucleic acid sequence as set forth in SEQ ID NO: 40, and the 3′ ssODN arm comprises the nucleic acid sequence as set forth in SEQ ID NO: 41. In some embodiments, the ssODN comprises the nucleic acid sequence set forth in SEQ ID NO: 42.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 40. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 41. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 42.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 42. In some embodiments, the 5′ ssODN arm comprises the nucleic acid sequence as set forth in SEQ ID NO: 40. In some embodiments, the 3′ ssODN arm comprises the nucleic acid sequence as set forth in SEQ ID NO: 41. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 40. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 41. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 42.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 43. In some embodiments, the 3′ ssODN arm comprises the nucleic acid sequence set forth in SEQ ID NO: 44. In some embodiments, the 5′ ssODN arm comprises the nucleic acid sequence as set forth in SEQ ID NO: 43, and the 3′ ssODN arm comprises the nucleic acid sequence as set forth in SEQ ID NO: 44. In some embodiments, the ssODN comprises the nucleic acid sequence set forth in SEQ ID NO: 45.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 43. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 44. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 45.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 45. In some embodiments, the 5′ ssODN arm comprises the nucleic acid sequence as set forth in SEQ ID NO: 43. In some embodiments, the 3′ ssODN arm comprises the nucleic acid sequence as set forth in SEQ ID NO: 44. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 43. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 44. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 45.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 46. In some embodiments, the 3′ ssODN arm comprises the nucleic acid sequence set forth in SEQ ID NO: 47. In some embodiments, the 5′ ssODN arm comprises the nucleic acid sequence as set forth in SEQ ID NO: 46, and the 3′ ssODN arm comprises the nucleic acid sequence as set forth in SEQ ID NO: 47. In some embodiments, the ssODN comprises the nucleic acid sequence set forth in SEQ ID NO: 48.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 46. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 47. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 48.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 48. In some embodiments, the 5′ ssODN arm comprises the nucleic acid sequence as set forth in SEQ ID NO: 46. In some embodiments, the 3′ ssODN arm comprises the nucleic acid sequence as set forth in SEQ ID NO: 47. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 46. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 47. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 48.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 49. In some embodiments, the 3′ ssODN arm comprises the nucleic acid sequence set forth in SEQ ID NO: 50. In some embodiments, the 5′ ssODN arm comprises the nucleic acid sequence as set forth in SEQ ID NO: 49, and the 3′ ssODN arm comprises the nucleic acid sequence as set forth in SEQ ID NO: 50. In some embodiments, the ssODN comprises the nucleic acid sequence set forth in SEQ ID NO: 51.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 49. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 50. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 51.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 51. In some embodiments, the 5′ ssODN arm comprises the nucleic acid sequence as set forth in SEQ ID NO: 49. In some embodiments, the 3′ ssODN arm comprises the nucleic acid sequence as set forth in SEQ ID NO: 50. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 49. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 50. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 51.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 52. In some embodiments, the 3′ ssODN arm comprises the nucleic acid sequence set forth in SEQ ID NO: 53. In some embodiments, the 5′ ssODN arm comprises the nucleic acid sequence as set forth in SEQ ID NO: 52, and the 3′ ssODN arm comprises the nucleic acid sequence as set forth in SEQ ID NO: 53. In some embodiments, the ssODN comprises the nucleic acid sequence set forth in SEQ ID NO: 54.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 52. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 53. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 54.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 54. In some embodiments, the 5′ ssODN arm comprises the nucleic acid sequence as set forth in SEQ ID NO: 52. In some embodiments, the 3′ ssODN arm comprises the nucleic acid sequence as set forth in SEQ ID NO: 53. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 52. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 53. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 54.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 55. In some embodiments, the 3′ ssODN arm comprises the nucleic acid sequence set forth in SEQ ID NO: 56. In some embodiments, the 5′ ssODN arm comprises the nucleic acid sequence as set forth in SEQ ID NO: 55, and the 3′ ssODN arm comprises the nucleic acid sequence as set forth in SEQ ID NO: 56. In some embodiments, the ssODN comprises the nucleic acid sequence set forth in SEQ ID NO: 57.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 55. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 56. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 57.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 57. In some embodiments, the 5′ ssODN arm comprises the nucleic acid sequence as set forth in SEQ ID NO: 55. In some embodiments, the 3′ ssODN arm comprises the nucleic acid sequence as set forth in SEQ ID NO: 56. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 55. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 56. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO:57.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 58. In some embodiments, the 3′ ssODN arm comprises the nucleic acid sequence set forth in SEQ ID NO: 59. In some embodiments, the 5′ ssODN arm comprises the nucleic acid sequence as set forth in SEQ ID NO: 58, and the 3′ ssODN arm comprises the nucleic acid sequence as set forth in SEQ ID NO: 59. In some embodiments, the ssODN comprises the nucleic acid sequence set forth in SEQ ID NO: 60.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 58. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 59. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 60.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 60. In some embodiments, the 5′ ssODN arm comprises the nucleic acid sequence as set forth in SEQ ID NO: 58. In some embodiments, the 3′ ssODN arm comprises the nucleic acid sequence as set forth in SEQ ID NO: 59. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 58. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 59. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 60.

In some embodiments, the donor template, e.g., ssODN, is homologous to the target gene and comprises a corrected form of the gene variant, e.g., SNP, and comprises one or more nucleotide(s) that introduce a restriction site that is recognized by one or more restriction enzymes. In some embodiments, the 5′ ssODN arm comprises the nucleic acid sequence set forth in SEQ ID NO: 61. In some embodiments, the 3′ ssODN arm comprises the nucleic acid sequence set forth in SEQ ID NO: 62. In some embodiments, the 5′ ssODN arm comprises the nucleic acid sequence as set forth in SEQ ID NO: 61, and the 3′ ssODN arm comprises the nucleic acid sequence as set forth in SEQ ID NO: 62. In some embodiments, the ssODN comprises the nucleic acid sequence set forth in SEQ ID NO: 63.

In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 61. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 62. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 63.

Also provided herein is an isolated nucleic acid, e.g., an isolated nucleic acid for use in a method of correcting a gene variant associated with PD, comprising the nucleic acid sequence of any of the donor templates, e.g., ssODNs, or portions thereof, e.g., or 5′ ssODN arms, or 3′ ssODN arms, described herein. In some embodiments, the ssODN comprises the nucleic acid sequence as set forth in SEQ ID NO: 63. In some embodiments, the 5′ ssODN arm comprises the nucleic acid sequence as set forth in SEQ ID NO: 61. In some embodiments, the 3′ ssODN arm comprises the nucleic acid sequence as set forth in SEQ ID NO: 62. In some embodiments, the 5′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 61. In some embodiments, the 3′ ssODN arm comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 62. In some embodiments, the ssODN comprises a nucleic acid sequence having at least 80%, at least 85%, at least 90%, at least 91%, at least 92%, at least 93%, at least 94%, at least 95%, at least 96%, at least 97%, at least 98%, at least 99%, or 100% sequence identity to the nucleic acid sequence as set forth in SEQ ID NO: 63.

In some embodiments, the crRNA comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in any one of SEQ ID NOS: 8 and 13-24, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in any one of SEQ ID NOS: 3, 5, and 27, 30, 33, 36, 39, 42, 45, 48, 51, 54, 57, 60, and 63. In some embodiments, the crRNA comprises the nucleic acid sequence set forth in any one of SEQ ID NOS: 8 and 13-24, and the ssODN comprises the nucleic acid sequence set forth in any one of SEQ ID NOS: 3, 5, and 27, 30, 33, 36, 39, 42, 45, 48, 51, 54, 57, 60, and 63.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 8, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 3. In some embodiments, the crRNA comprises a nucleic acid sequence set forth in SEQ ID NO: 8, and the ssODN comprises the nucleic acid sequence set forth in SEQ ID NO: 3. In some embodiments, the crRNA comprises the nucleic acid sequence set forth in SEQ ID NO: 8, and the ssODN comprises the nucleic acid sequence set forth in SEQ ID NO: 3.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 8, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 5. In some embodiments, the crRNA comprises the nucleic acid sequence set forth in SEQ ID NO: 8, and the ssODN comprises the nucleic acid sequence set forth in SEQ ID NO: 5.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 13, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 27. In some embodiments, the crRNA comprises the nucleic acid sequence set forth in SEQ ID NO: 13, and the ssODN comprises the nucleic acid sequence set forth in SEQ ID NO: 27.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 14, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 30. In some embodiments, the crRNA comprises the nucleic acid sequence set forth in SEQ ID NO: 14, and the ssODN comprises the nucleic acid sequence set forth in SEQ ID NO: 30.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 8, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 33. In some embodiments, the crRNA comprises the nucleic acid sequence set forth in SEQ ID NO: 8, and the ssODN comprises the nucleic acid sequence set forth in SEQ ID NO: 33.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 15, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 36. In some embodiments, the crRNA comprises the nucleic acid sequence set forth in SEQ ID NO: 15, and the ssODN comprises the nucleic acid sequence set forth in SEQ ID NO: 36.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 16, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 39. In some embodiments, the crRNA comprises the nucleic acid sequence set forth in SEQ ID NO: 16, and the ssODN comprises the nucleic acid sequence set forth in SEQ ID NO: 39.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 17, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 42. In some embodiments, the crRNA comprises the nucleic acid sequence set forth in SEQ ID NO: 17, and the ssODN comprises the nucleic acid sequence set forth in SEQ ID NO: 42.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 18, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 45. In some embodiments, the crRNA comprises the nucleic acid sequence set forth in SEQ ID NO: 18, and the ssODN comprises the nucleic acid sequence set forth in SEQ ID NO: 45.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 19, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 48. In some embodiments, the crRNA comprises the nucleic acid sequence set forth in SEQ ID NO: 19, and the ssODN comprises the nucleic acid sequence set forth in SEQ ID NO: 48.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 20, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 51. In some embodiments, the crRNA comprises the nucleic acid sequence set forth in SEQ ID NO: 20, and the ssODN comprises the nucleic acid sequence set forth in SEQ ID NO: 51.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 21, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 54. In some embodiments, the crRNA comprises the nucleic acid sequence set forth in SEQ ID NO: 21, and the ssODN comprises the nucleic acid sequence set forth in SEQ ID NO:54.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 22, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 57. In some embodiments, the crRNA comprises the nucleic acid sequence set forth in SEQ ID NO: 22, and the ssODN comprises the nucleic acid sequence set forth in SEQ ID NO: 57.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 23, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 60. In some embodiments, the crRNA comprises the nucleic acid sequence set forth in SEQ ID NO: 23, and the ssODN comprises the nucleic acid sequence set forth in SEQ ID NO: 60.

In some embodiments, the crRNA comprises the nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 24, and the ssODN comprises a nucleic acid sequence having at least 90%, at least 91%, at least 92%, at least 93%, at least 94%, at least 95%, at least 96%, at least 97%, at least 98%, at least 99%, or 100% sequence identity to the nucleic acid sequence set forth in SEQ ID NO: 63. In some embodiments, the crRNA comprises the nucleic acid sequence set forth in SEQ ID NO: 24, and the ssODN comprises the nucleic acid sequence set forth in SEQ ID NO: 63.

In some embodiments, the donor template, e.g., ssODN, comprises a corrected form of the SNP. In some embodiments, the target gene is human GBA1, and, after the integration of the ssODN into the GBA1, the GBA1 encodes an amino acid sequence comprising the amino acid sequence of SEQ ID NO: 6.

In some embodiments, the SNP is rs76763715 and the corrected form of the SNP is a thymine wildtype variant. In some embodiments, the SNP is rs76763715, and, after the integration of the ssODN into the GBA1, the GBA1 comprises the corrected form of the SNP and encodes an asparagine at amino acid position 370. In some embodiments, the SNP is rs76763715, and, after the integration of the ssODN into the GBA1, the GBA1 encodes the amino acid sequence of SEQ ID NO: 6. In some embodiments, the SNP is rs76763715, the ssODN comprises a 5′ ssODN arm and a 3′ ssODN arm, and the 5′ ssODN arm comprises the nucleic acid sequence of SEQ ID NO: 4, and/or the 3′ ssODN arm comprises the nucleic acid sequence of SEQ ID NO: 2, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 5, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 1, and/or the 3′ ssODN arm comprises the nucleic acid sequence of SEQ ID NO: 2, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 3, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 25, and/or the 3′ ssODN arm comprises the nucleic acid sequence of SEQ ID NO: 26, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 27, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 28, and/or the 3′ ssODN arm comprises the nucleic acid sequence of SEQ ID NO: 29, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 30, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 31, and/or the 3′ ssODN arm comprises the nucleic acid sequence of SEQ ID NO: 32, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 33, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 34, and/or the 3′ ssODN arm comprises the nucleic acid sequence of SEQ ID NO: 35, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 36, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 37, and/or the 3′ ssODN arm comprises the nucleic acid sequence of SEQ ID NO: 38, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 39, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 40, and/or the 3′ ssODN arm comprises the nucleic acid sequence of SEQ ID NO: 41, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 42, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 43, and/or the 3′ ssODN arm comprises the nucleic acid sequence of SEQ ID NO: 44, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 45, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 46, and/or the 3′ ssODN arm comprises the nucleic acid sequence of SEQ ID NO: 47, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 48, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 49, and/or the 3′ ssODN arm comprises the nucleic acid sequence of SEQ ID NO: 50, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 51, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 52, and/or the 3′ ssODN arm comprises the nucleic acid sequence of SEQ ID NO: 53, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 54, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 55, and/or the 3′ ssODN arm comprises the nucleic acid sequence of SEQ ID NO: 56, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 57, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 58, and/or the 3′ ssODN arm comprises the nucleic acid sequence of SEQ ID NO: 59, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 60, and, after the integration of the ssODN into the GBA1, the GBA1 encodes the amino acid sequence of SEQ ID NO: 6.

In some embodiments, the SNP is rs76763715, the ssODN comprises a 5′ ssODN arm and a 3′ ssODN arm, and the 5′ ssODN arm comprises the nucleic acid sequence of SEQ ID NO: 61, and/or the 3′ ssODN arm comprises the nucleic acid sequence of SEQ ID NO: 62, and, after the integration of the ssODN into the GBA1, the GBA1 encodes the amino acid sequence of SEQ ID NO: 6. In some embodiments, the SNP is rs76763715, and the ssODN comprises the nucleic acid sequence of SEQ ID NO: 63, and, after the integration of the ssODN into the GBA1, the GBA1 encodes the amino acid sequence of SEQ ID NO: 6.

In some embodiments, the one or more agent(s) capable of inducing a DNA break comprises a recombinant nuclease, e.g., Cas9, and a guide RNA, e.g., sgRNA, wherein the recombinant nuclease, e.g., the Cas9, and the guide RNA, e.g., the sgRNA, are introduced into the cell as a RNP complex. In some embodiments, the RNP complex and the homology-directed repair (HDR) template (i.e. the ssODN) are introduced into the cell at a particular ratio of picomoles (pmol) RNP to pmol ssODN. In some embodiments, the recombinant nuclease is Cas9 or HiFiCas9. In some embodiments, the ratio of RNP:ssODN is between about 5:2 and 5:25 pmol. In some embodiments, the ratio of RNP:ssODN is between about 5:4 and 5:20 pmol. In some embodiments, the ratio of RNP:ssODN is about 120:96 pmol. In some embodiments, the ratio of RNP:ssODN is about 120:480 pmol. In some embodiments, the ratio of RNP:ssODN is about 240:192 pmol. In some embodiments, the ratio of RNP:ssODN is about 240:960 pmol. In some embodiments, the recombinant nuclease is eSpCas9. In some embodiments, the ratio of RNP:ssODN is between about 5:2 and 5:25 pmol. In some embodiments, the ratio of RNP:ssODN is between about 5:4 and 5:20 pmol. In some embodiments, the ratio of RNP:ssODN is between about 60:48 pmol and about 60:240 pmol. In some embodiments, the ratio of RNP:ssODN is about 60:48 pmol. In some embodiments, the ratio of RNP:ssODN is about 60:240 pmol.

In some embodiments, the SNP is rs421016 and the corrected form of the SNP is an adenine wildtype variant. In some embodiments, the SNP is rs421016, and, after the integration of the ssODN into the GBA1, the GBA1 comprises the corrected form of the SNP and encodes a leucine at amino acid position 444. In some embodiments, the SNP is rs421016, and, after the integration of the ssODN into the GBA1, the GBA1 encodes the amino acid sequence of SEQ ID NO: 6.

In some embodiments, the SNP is rs2230288 and the corrected form of the SNP is a cytosine wildtype variant. In some embodiments, the SNP is rs2230288, and, after the integration of the ssODN into the GBA1, the GBA1 comprises the corrected form of the SNP and encodes a glutamic acid at position 326. In some embodiments, the SNP is rs2230288, and, after the integration of the ssODN into the GBA1, the GBA1 encodes the amino acid sequence of SEQ ID NO: 6.

In some of any such embodiments, after integration of the ssODN into the GBA1, the GBA1 encodes the amino acid sequence of SEQ ID NO: 6, and the GBA1 comprises the nucleic acid sequence of SEQ ID NO: 11 or comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the nucleotide sequence as set forth in SEQ ID NO: 11. In some embodiments, after integration of the ssODN into the GBA1, the GBA1 encodes an asparagine at amino acid position 370, and the GBA1 comprises the nucleic acid sequence of SEQ ID NO: 11 or comprises a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to the nucleotide sequence as set forth in SEQ ID NO: 11.

### F. Delivery of Agents and Donor Templates

In some embodiments, the methods described herein involve introducing or delivering (i) one or more agent(s) capable of inducing a DNA break, such as a DSB or a SSB, such as any such agent(s) described in Section II.C, e.g., Cas9 and a sgRNA, or Cas9 and a first sgRNA and a second sgRNA; and (ii) a donor template, such as any such donor template described in Section II.E., to a cell, such as any such cell described in Section II.A, e.g., PSC or iPSC, using any of a number of known delivery methods and/or vehicles for introduction or transfer to cells, such as by using viral, e.g., lentiviral, vectors, delivery vectors, or any of the known methods and/or vehicles for delivering such agent(s), e.g., Cas9 proteins and sgRNAs, and donor templates, e.g., ssODN, to cells. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505. In some embodiments, the one or more agent(s) capable of inducing a DNA break are one or more agent(s) comprising a recombinant nuclease for inducing a DNA break. Accordingly, in some embodiments, the methods involve introducing or delivering (i) one or more agent(s) comprising a recombinant nuclease for inducing a DNA break, e.g., a DSB or a SSB; and (ii) a donor template, using any of such delivery methods and/or vehicles.

In some embodiments, one or more nucleic acid sequences encoding one or more components of the one or more agent(s) comprising a recombinant nuclease for inducing a DNA break is introduced into the cells, such as by any method that is described herein or is known for introducing nucleic acid sequences into a cell. In some embodiments, one or more vector(s) encoding one or more components of the one or more agent(s) comprising a recombinant nuclease for inducing a DNA break, such as any such agent(s) described in Section II.C, including, e.g., a Cas9 protein and a sgRNA, or a Cas9 protein and a first sgRNA and a second sgRNA, is introduced or delivered to the cell. In some embodiments, a vector encoding a Cas9 protein is introduced or delivered to the cell. In some embodiments, a vector encoding a sgRNA is introduced or delivered to the cell. In some embodiments, a vector encoding a first sgRNA is introduced or delivered to the cell. In some embodiments, a vector encoding a second sgRNA is introduced or delivered to the cell. In some embodiments, a vector encoding a first sgRNA and a second sgRNA is introduced or delivered to the cell. In some embodiments, a vector encoding a Cas9 protein and a vector encoding a sgRNA are introduced or delivered to the cell. In some embodiments, a vector encoding a Cas9 protein and (i) a vector encoding a first sgRNA; and/or (ii) a vector encoding a second sgRNA, are introduced or delivered to the cell. In some embodiments, a vector encoding a Cas9 protein and a vector encoding a first sgRNA and a second sgRNA, are introduced or delivered to the cell. In some embodiments, a vector encoding a Cas9 protein and a sgRNA is introduced or delivered to the cell. In some embodiments, a vector encoding a Cas9 protein, a first sgRNA, and a second sgRNA is introduced or delivered to the cell. In some embodiments, the one or more vector(s) is introduced by any available method, such as electroporation, particle gun, or calcium phosphate transfection, among other methods.

In some embodiments, introduction or delivery by electroporation comprises mixing the cells with the one or more vector(s) encoding one or more components of the one or more agent(s) comprising a recombinant nuclease for inducing a DNA break (e.g., a vector encoding Cas9 and a sgRNA, or a vector encoding Cas9 and a vector encoding sgRNA, or one or more vectors encoding a Cas9, a first sgRNA, and a second sgRNA), in a cartridge, chamber, or cuvette, and applying one or more electrical impulses of defined duration and amplitude. In some embodiments, introduction or delivery by electroporation is performed using a system in which cells are mixed with the one or more vector(s) encoding one or more components of the one or more agent(s) comprising a recombinant nuclease for inducing a DNA break (e.g., a vector encoding Cas9 and a sgRNA, or a vector encoding Cas9 and a vector encoding sgRNA, or one or more vectors encoding a Cas9, a first sgRNA, and a second sgRNA), in a vessel connected to a device, e.g., a pump, that feeds the mixture into a cartridge, chamber, or cuvette, wherein one or more electrical impulses of defined duration and amplitude are applied, afer which the cells are introduced or delivered to a second vessel.

In some embodiments, the one or more agent(s) comprising a recombinant nuclease for inducing a DNA break is introduced into the cell as one or more protein(s). Accordingly, in some embodiments, one or more of the one or more agent(s) is introduced into the cell as a protein. In some embodiments, the one or more agent(s) capable of inducing a DNA break comprises a recombinant nuclease, e.g., Cas9. In some embodiments, the Cas9 is capable of inducing a DSB. In some embodiments, the Cas9 is capable of inducing a SSB, such as by comprising one or more mutations that inactivate the RuvC catalytic domain or the HNH catalytic domain. In some embodiments, the one or more agent(s) comprising a recombinant nuclease for inducing a DNA break comprises a fusion protein comprising a DNA binding domain and a DNA cleavage domain. In some embodiments, the fusion protein is a TALEN or a ZFN. In some embodiments, the one or more protein(s) are introduced or delivered into the cell using electroporation or other physical delivery method, such as microinjection, particle gun, calcium phosphate transfection, or cell compression or squeezing (e.g., as described in Lee, et al, 2012, Nano Lett 12: 6322-27).

In some embodiments, introduction or delivery by electroporation comprises mixing the cells with the one or more protein(s) that are the one or more agent(s) comprising a recombinant nuclease for inducing a DNA break, in a cartridge, chamber, or cuvette, and applying one or more electrical impulses of defined duration and amplitude. In some embodiments, introduction or delivery by electroporation is performed using a system in which cells are mixed with the one or more proteins, in a vessel connected to a device, e.g., a pump, that feeds the mixture into a cartridge, chamber, or cuvette, wherein one or more electrical impulses of defined duration and amplitude are applied, afer which the cells are introduced or delivered to a second vessel.

In some embodiments, the one or more agent(s) comprising a recombinant nuclease for inducing a DNA break is introduced into the cell as a ribonucleoprotein (RNP) complex. In some embodiments, the one or more agent(s) capable of inducing a DNA break comprises a recombinant nuclease, e.g., Cas9, and a guide RNA, e.g., sgRNA, and the recombinant nuclease, e.g., the Cas9, and the guide RNA, e.g., the sgRNA, are introduced into the cell as a RNP complex. RNP complexes comprise a protein, such as a recombinant nuclease or a protein comprising a recombinant nuclease, and a ribonucleotide, such as RNA or guide RNA, e.g., sgRNA. In some embodiments, the recombinant nuclease is provided as a protein, and the guide RNA is provided as a transcribed or synthesized RNA. In some embodiments, the guide RNA, e.g., sgRNA, forms a RNP complex with the recombinant nuclease protein, e.g., Cas9, under suitable conditions prior to delivery to the cells. In some embodiments, the RNP complex comprises a Cas9 protein in complex with a sgRNA that targets the cleavage site within the target gene, e.g., GBA1. In some embodiments, the RNP complex is introduced or delivered into the cell using electroporation or other physical delivery method, such as particle gun, calcium phosphate transfection, or cell compression or squeezing (e.g., as described in Lee, et al, 2012, Nano Lett 12: 6322-27).

In some embodiments, the one or more agent(s) capable of inducing a DNA break comprises a recombinant nuclease, e.g., Cas9, a first guide RNA, e.g., first sgRNA, and a second guide RNA, e.g., second sgRNA; and the recombinant nuclease, the first guide RNA, and the second guide RNA, are introduced into the cell as a RNP complex. RNP complexes comprise a protein, such as a recombinant nuclease or a protein comprising a recombinant nuclease, and a ribonucleotide, such as RNA or guide RNA, e.g., sgRNA. In some embodiments, the RNP complex comprises the recombinant nuclease and a first guide RNA, e.g., a first sgRNA. In some embodiments, the RNP complex comprises the recombinant nuclease and a second guide RNA, e.g., a second sgRNA. In some embodiments, there is an RNP complex comprising the recombinant nuclease and the first guide RNA, e.g., the first sgRNA, and there is an RNP complex comprising the recombinant nuclease and the second guide RNA, e.g., the second sgRNA. In some embodiments, the recombinant nuclease is provided as a protein, and the guide RNA, e.g., the first guide RNA and/or the second guide RNA, is provided as a transcribed or synthesized RNA. In some embodiments, the guide RNA, such as the first guide RNA or the second guide RNA forms a RNP complex with the recombinant nuclease protein, e.g., Cas9, under suitable conditions prior to delivery to the cells. In some embodiments, the RNP complex comprises a Cas9 protein in complex with a sgRNA that targets the cleavage site within the target gene, e.g., GBA1. In some embodiments, the RNP complex comprises a Cas9 protein in complex with a sgRNA that targets the cleavage site within the sense strand of the target gene, e.g., GBA1. In some embodiments, the RNP complex comprises a Cas9 protein in complex with a sgRNA that targets the cleavage site within the antisense strand of the target gene, e.g., GBA1. In some embodiments, the RNP complex is introduced or delivered into the cell using electroporation or other physical delivery method, such as particle gun, calcium phosphate transfection, or cell compression or squeezing (e.g., as described in Lee, et al, 2012, Nano Lett 12: 6322-27).

In some embodiments, introduction or delivery by electroporation comprises mixing the cells with the RNP complex in a cartridge, chamber, or cuvette, and applying one or more electrical impulses of defined duration and amplitude. In some embodiments, introduction or delivery by electroporation is performed using a system in which cells are mixed with the RNP complex in a vessel connected to a device, e.g., a pump, that feeds the mixture into a cartridge, chamber, or cuvette, wherein one or more electrical impulses of defined duration and amplitude are applied, afer which the cells are introduced or delivered to a second vessel.

In some embodiments, the one or more nucleic acid sequences encoding one or more components of the one or more agent(s) comprising a recombinant nuclease for inducing a DNA break is introduced or delivered into the cells by a combination of a vector and a non-vector-based method. For instance, virosomes comprise liposomes combined with an inactivated virus (e.g., an HIV or influenza virus), which can result in a more efficient gene transfer.

In some embodiments, the one or more agent(s) comprising a recombinant nuclease for inducing a DNA break is introduced into the cells as RNA. In some embodiments, RNA encoding one or more agent(s) comprising a recombinant nuclease for inducing a DNA break, such as Cas9, is introduced into the cells; and/or a guide RNA, such as sgRNA, is introduced into the cells. In some embodiments, RNA encoding one or more agent(s) comprising a recombinant nuclease for inducing a DNA break, such as Cas9, is introduced into the cells; and/or a first guide RNA, such as a first sgRNA, and a second guide RNA, such as a second sgRNA, is introduced into the cells. In some embodiments, RNA encoding a recombinant nuclease, or a fusion protein comprising a DNA binding domain and a DNA cleavage domain; and/or (i) a guide RNA, or (ii) a first guide RNA and a secnd guide RNA, is delivered to the cells by any available or known method, such as by microinjection, electroporation, calcium phosphate transfection, or cell compression or squeezing (e.g., as described in Lee, et al, 2012, Nano Lett 12: 6322-27).

In some embodiments, introduction or delivery by electroporation comprises mixing the cells with the RNA encoding one or more agent(s) comprising a recombinant nuclease for inducing a DNA break, such as Cas9; and/or a guide RNA, such as sgRNA, in a cartridge, chamber, or cuvette, and applying one or more electrical impulses of defined duration and amplitude. In some embodiments, introduction or delivery by electroporation comprises mixing the cells with the RNA encoding one or more agent(s) comprising a recombinant nuclease for inducing a DNA break, such as Cas9; and/or a first guide RNA, such as a first sgRNA, and a second guide RNA, such as a second sgRNA, in a cartridge, chamber, or cuvette, and applying one or more electrical impulses of defined duration and amplitude. In some embodiments, introduction or delivery by electroporation is performed using a system in which cells are mixed with the one or more RNAs, e.g., RNA encoding one or more agent(s) comprising a recombinant nuclease for inducing a DNA break, and/or a guide RNA, or a first guide RNA and a second guide RNA, in a vessel connected to a device, e.g., a pump, that feeds the mixture into a cartridge, chamber, or cuvette, wherein one or more electrical impulses of defined duration and amplitude are applied, after which the cells are introduced or delivered to a second vessel. In some embodiments, the one or more RNAs can be conjugated to molecules to promote uptake by the cells.

In some embodiments, the donor template, including those as described in Section II.E, e.g., ssODN, is introduced into the cells in a nucleic acid form. In some embodiments, the donor template is introduced into the cells as an isolated nucleic acid sequence. In some embodiments, the donor template, e.g., ssODN, is introduced by any available method, such as electroporation, particle gun, or calcium phosphate transfection, among other methods.

In some embodiments, introduction or delivery by electroporation comprises mixing the cells with the donor template in a cartridge, chamber, or cuvette, and applying one or more electrical impulses of defined duration and amplitude. In some embodiments, introduction or delivery by electroporation is performed using a system in which cells are mixed with the donor template in a vessel connected to a device, e.g., a pump, that feeds the mixture into a cartridge, chamber, or cuvette, wherein one or more electrical impulses of defined duration and amplitude are applied, after which the cells are introduced or delivered to a second vessel.

In some embodiments, the methods provided herein include introducing or delivering the one or more agent(s) comprising a recombinant nuclease for inducing a DNA break, and introducing or delivering the donor template, in combination with one another, using one or more of any of the methods for introduction or delivery of the one or more agents and the donor template described herein, in combination with one another. In some embodiments, the one or more agents and the donor template are introduced or delivered simultaneously. In some embodiments, the one or more agents and the donor template are introduced or delivered sequentially, e.g., the one or more agents is introduced or delivered prior to the introduction or delivery of the donor template. In some embodiments, the one or more agent(s) comprising a recombinant nuclease for inducing a DNA break are introduced or delivered by a different method or means than the donor template. In some embodiments, the one or more agent(s) comprising a recombinant nuclease for inducing a DNA break are introduced or delivered by any method or means described herein, and the donor template is introduced or delivered by any method or means described herein.

In some embodiments, the one or more vector(s) encoding one or more components of the one or more agent(s) comprising a recombinant nuclease for inducing a DNA break, including, e.g., a Cas9 protein and a sgRNA, is introduced or delivered to the cell; and the donor template, e.g., ssODN, is introduced or delivered to the cell as a nucleic acid. In some embodiments, the one or more vector(s) encoding one or more components of the one or more agent(s) comprising a recombinant nuclease for inducing a DNA break, including, e.g., a Cas9 protein and a first sgRNA and a second sgRNA, is introduced or delivered to the cell; and the donor template, e.g., ssODN, is introduced or delivered to the cell as a nucleic acid. In some embodiments, the nucleic acid is DNA. In some embodiments, the one or more vector(s) and the donor template are introduced or delivered simultaneously. In some embodiments, the one or more vector(s) and the donor template are introduced or delivered sequentially e.g., the one or more agents is introduced or delivered prior to the introduction or delivery of the donor template. In some embodiments, the RNA complex comprising a recombinant nuclease, e.g., Cas9, and a guide RNA, e.g., sgRNA, is introduced or delivered to the cell; and the donor template, e.g., ssODN, is introduced into the cell. In some embodiments, the RNA complex comprising a recombinant nuclease, e.g., Cas9, and a first guide RNA, e.g., a first sgRNA, and a second guide RNA, e.g., a second sgRNA, is introduced or delivered to the cell; and the donor template, e.g., ssODN, is introduced into the cell. In some embodiments, the RNA complex and the donor template are introduced or delivered simultaneously. In some embodiments, the RNA complex and the donor template are introduced or delivered sequentially e.g., the RNA complex is introduced or delivered prior to the introduction or delivery of the donor template. In some embodiments, the RNA encoding one or more agent(s) comprising a recombinant nuclease for inducing a DNA break are introduced or delivered to the cell; and the donor template, e.g., ssODN, is introduced or delivered to the cell. In some embodiments, the RNA encoding one or more agent(s) and the donor template are introduced or delivered simultaneously. In some embodiments, the RNA encoding one or more agent(s) and the donor template are introduced or delivered sequentially e.g., the RNA encoding one or more agent(s) is introduced or delivered prior to the introduction or delivery of the donor template.

In some embodiments, the donor template is introduced or delivered at the same time as the one or more agent(s) comprising a recombinant nuclease for inducing a DNA break, e.g., Cas9 and sgRNA, or Cas9 and first sgRNA and second sgRNA, are delivered. In some embodiments, the donor template is introduced or delivered before or after the one or more agent(s) comprising a recombinant nuclease for inducing a DNA break are delivered. In some embodiments, the donor template is introduced or delivered less than or less than about 1 minute, 5 minutes, 10 minutes, 15 minutes, 30 minutes, 1 hour, 2 hours, 3 hours, 6 hours, 9 hours, 12 hours, 1 day, 2 days, 3 days, 4 days, 1 week, 2 weeks, or 4 weeks before or after the one or more agent(s) comprising a recombinant nuclease for inducing a DNA break are delivered. In some embodiments, the donor template is introduced or delivered less than or less than about 1 minute, 5 minutes, 10 minutes, 15 minutes, 30 minutes, 1 hour, 2 hours, 3 hours, 6 hours, 9 hours, 12 hours, 1 day, 2 days, 3 days, 4 days, 1 week, 2 weeks, or 4 weeks after the one or more agent(s) comprising a recombinant nuclease for inducing a DNA break are delivered. In some embodiments, the donor template is introduced or delivered less than or less than about 30 seconds, 1 minute, 2 minutes, 3 minutes, 4 minutes, 5 minutes, 6 minutes, 7 minutes, 8 minutes, 9 minutes, 10 minutes, 15 minutes, 20 minutes, 30 minutes, 40 minutes, 50 minutes, 1 hour, 1.5 hours, 2 hours, 3 hours, 4 hours, 5 hours, or 6 hours before the one or more agent(s) comprising a recombinant nuclease for inducing a DNA break are delivered. In some embodiments, the donor template is introduced or delivered more than 6 hours after delivery of the agents, e.g., less than 9 hours, 12 hours, 1 day, 2 days, 3 days, 4 days, 1 week, 2 weeks, 3 weeks, 4 weeks, 5 weeks, 6 weeks, 2 months, 3 months, 4 months, 5 months, 6 months, 9 months, or 12 months, after the one or more agent(s) comprising a recombinant nuclease for inducing a DNA break are delivered. In some embodiments, the donor template is introduced or delivered more than 1 year after the one or more agent(s) comprising a recombinant nuclease for inducing a DNA break are delivered.

In some embodiments, the introducing or delivery of the one or more agent(s) comprising a recombinant nuclease for inducing a DNA break, e.g., Cas9 and sgRNA, or Cas9 and a first sgRNA and a secnd sgRNA; and/or the introducing or delivery of the donor template, e.g., ssODN, is carried out under conditions that allow HDR and integration of the donor template, e.g., ssODN, into the target gene, e.g., GBA1. In some embodiments, the introducing or delivery of the one or more agent(s) comprising a recombinant nuclease for inducing a DNA break; and the introducing or delivery of the donor template, e.g., ssODN, results in HDR and integration of the donor template, e.g., ssODN, into the target gene, e.g., GBA1.

### G. Selection of Corrected Cells

In some embodiments, the cells that underwent correction for one or more gene variants associated with PD in accordance with the methods herein, e.g., as described in Section II.B-F, are screened and/or selected for cells, e.g., clones, where the donor template was integrated into the target gene, e.g., GBA1. In some embodiments, the donor template, e.g., ssODN, introduces a restriction site that is recognizable by one or more restriction enzymes, and the cells are screened for the presence of that introduced restriction site. In some embodiments, the donor template, e.g., ssODN, introduces a silent mutation in the PAM sequence. In some embodiments, the donor template, e.g., ssODN, introduces a restriction site that is recognizable by one or more restriction enzymes and introduces a silent mutation in the PAM sequence, and the cells are screened for the presence of that introduced restriction site. The introduction of a restriction site allows, in some embodiments, for the screening and/or identifying of cells that have incorporated the donor template having such a restriction site that is not present in the corresponding site in the endogenous target gene, e.g., GBA1. In some embodiments, this screening and/or identifying is performed on a cell of a population of cells derived from a parental cell that was corrected in accordance with the methods described herein, e.g., in Section II.A-F.

In some embodiments, the cells are assessed to identify changes attributable to the methods described herein, e.g, as described in Section II.B-F, such as CRISPR/Cas9 gene editing. In some embodiments, the assessment includes nucleic acid, e.g., DNA and/or RNA, sequencing. In some embodiments, the assessment includes one or more of whole genome sequencing (WGS), targeted Sanger sequencing, and deep exome sequencing. In some embodiments, the assessment includes one or more of CIRCLE-seq (Tsai, Nature Methods (2017) 14:607-14), genomic qPCR, whole genome sequencing (WGS), targeted Sanger sequencing, and deep exome sequencing.

In some embodiments, the cells are assessed by a method for selecting for a cell comprising a corrected SNP, comprising sequencing DNA isolated from a cell derived from the cell of any one of the embodiments of the methods described herein; and determining whether the target gene, e.g., GBA1, comprises a corrected form of the SNP, wherein, if the target gene comprises a corrected form of the SNP, the cell is identified as a cell comprising a corrected SNP. In some embodiments, the sequencing includes one or more of whole genome sequencing (WGS), targeted Sanger sequencing, and deep exome sequencing. In some embodiments, the sequencing includes one or more of CIRCLE-seq, genomic qPCR, whole genome sequencing (WGS), targeted Sanger sequencing, and deep exome sequencing.

In some embodiments, off-target cleavage (e.g. DSBs) of the GBAP1 gene may occur, such as due to its high sequence homology with GBA1. In some embodiments, DSBs are introduced in both the GBA1 and GBAP1 genes, resulting in a large (i.e. ˜16 kB) deletion, such as between the GBA1 and GBAP1 loci. In some embodiments, if the large deletion does not occur, both GBA1 and GBAP1 amplicons are sequenced by any of the sequencing methods described herein. In some embodiments, if the large deletion occurs, only a hybrid GBA1-GBAP1 amplicon is sequenced by any of the sequencing methods described herein. In some embodiments, the percentage of deletion events is calculated as:

hybrid reads/((GBA1 reads+GBAP1 reads)/2+hybrid reads)*100% Deletion %:

In some embodiments, the cells are assessed to identify changes attributable to the methods described herein, e.g, as described in Section II.B-F, such as CRISPR/Cas9 gene editing. In some embodiments, the assessment includes assessment of enzyme activity. In some embodiments, the enzyme is the beta-glucocerebrosidase enzyme encoded by GBA1.

In some embodiments, the cells are assessed by a method for selecting for a cell comprising a corrected SNP, comprising assessing beta-glucocerebrosidase activity in a cell derived from the cell of any one of the embodiments of the methods described herein; and determining whether the target gene, e.g., GBA1, comprises a corrected form of the SNP, wherein, if the activity of beta-glucocerebrosidase is increased compared to a non-edited cell comprising the SNP, the cell is identified as a cell comprising a corrected SNP.

In some embodiments, a population of the cell produced by any of the methods of correcting gene variants, e.g., SNPs, described herein, e.g., as described in Section II.B-F, are subjected to differentiation into, e.g., floor plate midbrain progenitor cells, determined dopaminergic (DA) neuron progenitor cells, and/or dopaminergic (DA) neurons, or into glial cells, e.g., microglia, astrocytes, oligodendrocytes, or ependymocytes, using any of the differentiation methods described herein, e.g., as described in Section III.

In some embodiments, cells derived from a clone that integrated the donor template, e.g., ssODN, into the target gene, e.g., GBA1, are subjected to differentiation into, e.g., floor plate midbrain progenitor cells, determined dopaminergic (DA) neuron progenitor cells, and/or dopaminergic (DA) neurons, or into glial cells, e.g., microglia, astrocytes, oligodendrocytes, or ependymocytes, using any of the differentiation methods described herein, e.g., as described in Section III. In some embodiments, cells derived from a clone that integrated the donor template, e.g., ssODN, into the target gene, e.g., GBA1, and does not include additional changes attributable to the methods described herein for gene correction, e.g., as described in Section II.B-F, e.g., CRISPR/Cas9 gene editing, are subjected to differentiation into, e.g., floor plate midbrain progenitor cells, determined dopaminergic (DA) neuron progenitor cells, and/or dopaminergic (DA) neurons, or into glial cells, e.g., microglia, astrocytes, oligodendrocytes, or ependymocytes, using any of the differentiation methods described herein, e.g., as described in Section III. In some embodiments, the “additional changes” are limited to those changes to the nucleic acid sequence of the target gene, e.g., GBA1, other than those derived from the donor template, that results in a change to the amino acid sequence of the protein encoded by the target gene, i.e., it does not include silent mutations. In some embodiments, the “additional changes” includes the large (i.e. ˜16 kB) deletion between the GBA1 and GBAP1 loci.

Also provided herein are methods for selecting a cell comprising an integrated ssODN, comprising contacting DNA isolated from a cell derived from the cell produced by any of the methods described herein, e.g., as described in Section II, with the one or more restriction enzymes; and determining whether the DNA isolated from the cell has been cleaved at the restriction site, wherein, if the DNA has been cleaved, the cell is identified as a cell comprising an integrated ssODN.

Also provided herein are methods for selecting for a cell comprising a corrected SNP, comprising sequencing DNA isolated from a cell derived from the cell produced by any of the methods described herein, e.g., as described in Section II; and determining whether the target gene, e.g., GBA1, comprises a corrected form of the SNP, wherein, if the target gene comprises a corrected form of the SNP, the cell is identified as a cell comprising a corrected SNP. In some embodiments, the sequencing comprises one or more of whole genome sequencing (WGS), targeted Sanger sequencing, and deep exome sequencing. In some embodiments, the sequencing comprises one or more of CIRCLE-seq, genomic qPCR, whole genome sequencing (WGS), targeted Sanger sequencing, and deep exome sequencing.

### III. METHOD FOR DIFFERENTIATING CELLS

Provided herein are methods of differentiating neural cells, such as by subjecting the cells, e.g., the iPSCs, that underwent correction of one or more gene variants associated with PD, e.g., as described herein in Section II. Unless otherwise indicated, the methods of differentiation provided herein involve the cells, e.g., the pluripotent stem cells, such as iPSCs, that underwent correction of one or more gene variants, e.g., SNPs, associated with PD, such as a gene variant in human GBA1, e.g., using any of the methods as described herein in Section II.

In some embodiments, the methods of differentiating neural cells can be methods that differentiate neural cells, e.g., the iPSCs, that underwent correction of one or more gene variants associated with PD, e.g., as described herein in Section II, into any neural cell type using any available or known method for inducing the differentiation of cells, e.g., pluripotent stem cells. In some embodiments, the method induces differentiation of the cells, e.g., pluripotent stem cells, into floor plate midbrain progenitor cells, determined dopaminergic (DA) neuron progenitor cells, and/or dopaminergic (DA) neurons. Any available and known method for inducing differentiation of the cells, e.g., pluripotent stem cells, into floor plate midbrain progenitor cells, determined dopaminergic (DA) neuron progenitor cells, and/or dopaminergic (DA) neurons can be used, including any of those described, e.g., in Section III.A.

In some embodiments, the method induces differentiation of the cells, e.g., pluripotent stem cells, into glial cells. In some embodiments, the glial cells are selected from the group consisting of microglia, astrocytes, oligodendrocytes, and ependymocytes.

In some embodiments, the method induces differentiation of the cells, e.g., pluripotent stem cells, into microglia or microglial-like cells. Any available and known method for inducing differentiation of the cells, e.g., pluripotent stem cells, into microglia or microglial-like cells can be used. Exemplary methods of inducing differentiation of pluripotent stem cells into microglia or microglial-like cells can be found in, e.g., Abud et al., Neuron (2017), Vol. 94: 278-293; Douvaras et al., Stem Cell Reports (2017), Vol. 8: 1516-1524; Muffat et al., Nature Medicine (2016), Vol. 22(11): 1358-1367; and Pandya et al., Nature Neuroscience (2017), Vol. 20(5): 753-759, the contents of which are hereby incorporated by reference in their entirety.

In some embodiments, the method induces differentiation of the cells, e.g., pluripotent stem cells, into astrocytes. Any available and known method for inducing differentiation of the cells, e.g., pluripotent stem cells, into astrocytes can be used. Exemplary methods of inducing differentiation of the cells, e.g. pluripotent stem cells, into astrocytes can be found in, e.g., TCW et al., Stem Cell Reports (2017), Vol. 9: 600-614, including the methods described in the references cited therein, e.g., in Table 1, the contents of which are hereby incorporated by reference in their entirety. Exemplary methods of inducing differentiation of pluripotent stem cells into astrocytes can include, in some embodiments, the use of commercially available kits, and provided methods for use of such kits, including, e.g., Astrocyte Medium, Catalog #1801 (ScienCell Research Laboratories, Carlsbad, CA); Astrocyte Medium, Catalog #A1261301 (ThermoFisher Scientific Inc, Waltham, MA); and AGM Astrocyte Growth Medium BulletKit, Catalog #CC-3186 (Lonza, Basel, Switzerland), the contents of which are hereby incorporated by reference in their entirety.

In some embodiments, the method induces differentiation of the cells, e.g., pluripotent stem cells, into oligodendrocytes. Any available and known method for inducing differentiation of the cells, e.g., pluripotent stem cells, into oligodendrocytes can be used. Exemplary methods of inducing differentiation of pluripotent stem cells into oligodendrocytes can be found in, e.g., Ehrlich et al., PNAS (2017), Vol. 114(11): E2243-E2252; Douvaras et al., Stem Cell Reports (2014), Vol. 3(2): 250-259; Stacpoole et al., Stem Cell Reports (2013), Vol. 1(5): 437-450; Wang et al., Cell Stem Cell (2013), Vol. 12(2): 252-264; and Piao et al., Cell Stem Cell (2015), Vol. 16(2): 198-210, the contents of which are hereby incorporated by reference in their entirety.

### A. Floor Plate Midbrain Progenitor Cells, Determined DA Neuron Progenitor Cells, and DA Neurons

Provided herein are methods of differentiating neural cells that comprises differentiating pluripotent stem cells, such as any of the cells produced by the methods as described, e.g., in Section II. The methods of differentiating neural cells are not limited and can be any available or known method for inducing the differentiation of pluripotent stem cells into floor plate midbrain progenitor cells, determined dopaminergic (DA) neuron progenitor cells, and/or dopaminergic (DA) neurons. Exemplary methods of differentiating neural cells can be found, e.g., in WO2013104752, WO2010096496, WO2013067362, WO2014176606, WO2016196661, WO2015143342, US20160348070, the contents of which are hereby incorporated by reference in their entirety.

Provided herein are methods of differentiating neural cells, involving (1) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

The provided methods of differentiating neural cells, such as by subjecting iPSCs to cell culture methods that induce their differentiation into floor plate midbrain progenitor cells, determined dopaminergic (DA) neuron progenitor cells, and/or, dopaminergic (DA) neurons.

As described herein, iPSCs were generated from fibroblasts of human patients with Parkinson's disease. In a first incubation, the iPSCs were then differentiated to midbrain floor plate precursors and grown as spheroids in a non-adherent culture by exposure to small molecules, such as LDN, SB, PUR, SHH, CHIR, and combinations thereof, beginning on day 0. The resulting spheroids were then transferred to an adherent culture as part of a second incubation, optionally following dissociation of the spheroid, before being exposed to additional small molecules (e.g., LDN, CHIR, BDNF, GDNF, ascorbic acid, dbcAMP, TGFβ3, DAPT, and combinations thereof) to induce further differentiation into engraftable determined DA neuron progenitor cells or DA neurons. The provided methods may include any of those described in PCT/US2021/013324, which is incorporated herein by reference in its entirety.

Also provided herein are methods of differentiating neural cells, comprising differentiating pluripotent stem cells, such as any of the cells produced by the methods as described, e.g., in Section II, using any of the methods disclosed in any one of WO2013104752, WO2010096496, WO2013067362, WO2014176606, WO2016196661, WO2015143342, and US20160348070.

Also provided are methods of differentiating neural cells, involving: exposing pluripotent stem cells to (a) an inhibitor of bone morphogenetic protein (BMP) signaling; (b) an inhibitor of TGF-β/activing-Nodal signaling; and (c) at least one activator of Sonic Hedgehog (SHH) signaling. In some embodiments, the method further comprising exposing the pluripotent stem cells to at least one inhibitor of GSK3β signaling. In some embodiments, the exposing to an inhibitor of BMP signaling and the inhibitor of TGF-β/activing-Nodal signaling occurs while the pluripotent stem cells are attached to a substrate. In some embodiments, the inhibitor of BMP signaling is any inhibitor of BMP signaling described herein, the inhibitor of TGF-β/activing-Nodal signaling is any inhibitor of TGF-β/activing-Nodal signaling described herein, and the at least one activator of SHH signaling is any activator of SHH signaling described herein. In some embodiments, during the exposing to the inhibitor of BMP signaling, the inhibitor of TGF-β/activing-Nodal signaling, and the at least one activator of SHH signaling, the pluripotent stem cells are attached to a substrate. In some embodiments, during the exposing to the at least one inhibitor of GSK3β signaling, the pluripotent stem cells are attached to a substrate. In some embodiments, during the exposing to the inhibitor of BMP signaling, the inhibitor of TGF-β/activing-Nodal signaling, and the at least one activator of SHH signaling, the pluripotent stem cells are in a non-adherent culture vessel under conditions to produce a cellular spheroid. In some embodiments, during the exposing to the at least one inhibitor of GSK3β signaling, the pluripotent stem cells are in a non-adherent culture vessel under conditions to produce a cellular spheroid.

### 1. Cells Selected for Differentiation

In some embodiments, the cells selected to undergo differentiation are pluripotent stem cells (PSCs), e.g., iPSCs, that underwent correction of one or more gene variants associated with PD, e.g., as described in Section II. In some embodiments, the cells selected to undergo differentiation are any cells corrected in accordance with the methods provided herein, e.g., in Section II. In some embodiments, the cells selected to undergo differentiation are any cells produced by the methods described herein, e.g., in Section II. In some embodiments, the cells selected to undergo differentiation are any cells selected by the methods described herein, e.g., in Section II.G.

### 2. Non-Adherent Culture

The provided methods include culturing PSCs (e.g. iPSCs) by incubation with certain molecules (e.g. small molecules) to induce their differentiation into floor plate midbrain progenitor cells, determined dopamine (DA) neuron progenitor cells, and/or, dopamine (DA) neurons. In particular, in some embodiments, the provided embodiments include a first incubation of PSCs under non-adherent conditions to produce spheroids, in the presence of certain molecules (e.g., small molecules), which can, in some aspects, improve the consistency of producing physiologically relevant cells for implantation. In some embodiments, the methods include performing a first incubation involving culturing pluripotent stem cells in a non-adherent culture vessel under conditions to produce a cell spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling.

In some embodiments, a non-adherent culture vessel is a culture vessel with a low or ultra-low attachment surface, such as to inhibit or reduce cell attachment. In some embodiments, culturing cells in a non-adherent culture vessel does not prevent all cells of the culture from attaching the surface of the culture vessel.

In some embodiments, a non-adherent culture vessel is a culture vessel with an ultra-low attachment surface. In some aspects, an ultra-low attachment surface may inhibit cell attachment for a period of time. In some embodiments, an ultra-low attachment surface may inhibit cell attachment for the period of time necessary to obtain confluent growth of the same cell type on an adherent surface. In some embodiments, the ultra-low attachment surface is coated or treated with a substance to prevent cell attachment, such as a hydrogel layer (e.g., a neutrally charged and/or hydrophilic hydrogel layer). In some embodiments, a non-adherent culture vessel is coated or treated with a surfactant prior to the first incubation. In some embodiments, the surfactant is pluronic acid.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a 6-well or 24-well plate. In some embodiments, the wells of the multi-well plate further include micro-wells. In some any of the provided embodiments, a non-adherent culture vessel, such as a multi-well plate, has round or concave wells and/or microwells. In any of the provided embodiments, a non-adherent culture vessel, such as a multi-well plate, does not have corners or seams.

In some embodiments, a non-adherent culture vessel allows for three-dimensional formation of cell aggregates. In some embodiments, iPSCs are cultured in a non-adherent culture vessel, such as a multi-well plate, to produce cell aggregates (e.g., spheroids). In some embodiments, iPSCs are cultured in a non-adherent culture vessel, such as a multi-well plate, to produce cell aggregates (e.g., spheroids) on about day 7 of the method. In some embodiments, the cell aggregate (e.g., spheroid) expresses at least one of PAX6 and OTX2 on or by about day 7 of the method.

In some embodiments, the first incubation includes culturing pluripotent stem cells in a non-adherent culture vessel under conditions to produce a cellular spheroid.

In some embodiments, the number of PSCs plated on day 0 of the method is between about between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the number of PSCs plated on day 0 of the method is between about 1××10⁵ pluripotent stem cells per well and about 20×10⁶ pluripotent stem cells per well, between about 1×10⁵ pluripotent stem cells per well and about 15×10⁶ pluripotent stem cells per well, between about 1×10⁵ pluripotent stem cells per well and about 10×10⁶ pluripotent stem cells per well, between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 20×10⁶ pluripotent stem cells per well, between about 5×10⁵ pluripotent stem cells per well and about 15×10⁶ pluripotent stem cells per well, between about 5×10⁵ pluripotent stem cells per well and about 10×10⁶ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 6-well plate on day 0 of the method is between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 24-well plate on day 0 of the method is between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, or between about 1×10⁶ pluripotent stem cells per well and about 5×10⁶ pluripotent stem cells per well.

In some days, the number of PSCs plated on day 0 of the method is a number of cells sufficient to produce a cellular spheroid containing between about 1,000 cells and about 5,000 cells, or between about 2,000 cells and about 3,000 cells. In some days, the number of PSCs plated on day 0 of the method is a number of cells sufficient to produce a cellular spheroid containing between about 1,000 cells and about 5,000 cells. In some days, the number of PSCs plated on day 0 of the method is a number of cells sufficient to produce a cellular spheroid containing between about 2,000 cells and about 3,000 cells. In some days, the number of PSCs plated on day 0 of the method is a number of cells sufficient to produce a cellular spheroid containing about 2,000 cells. In some days, the number of PSCs plated on day 0 of the method is a number of cells sufficient to produce a cellular spheroid containing about 3,000 cells. In some embodiments, the spheroids containing the desired number is produced by the method on or by about day 7.

In some embodiments of the method provided herein, the first incubation includes culturing pluripotent stem cells in a non-adherent culture vessel under conditions to produce a cellular spheroid. In some embodiments, the first incubation is from about day 0 through about day 6. In some embodiments, the first incubation comprises culturing pluripotent stem cells in a culture media (“media”). In some embodiments, the first incubation comprises culturing pluripotent stem cells in the media from about day 0 through about day 6. In some embodiments, the first incubation comprises culturing pluripotent stem cells in the media to induce differentiation of the PSCs into floor plate midbrain progenitor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on day 0 and day 1. In some embodiments, the serum replacement is provided in the media at 2% (v/v) for at least a portion of the first incubation. In some embodiments, the serum replacement is provided in the media at 2% (v/v) from day 2 through day 6. In some embodiments, the serum replacement is provided in the media at 5% (v/v) on day 0 and day 1, and at 2% (v/v) from day 2 through day 6.

In some embodiments, the media is further supplemented with small molecules, such as any described above. In some embodiments, the small molecules are selected from among the group consisting of: a Rho-associated protein kinase (ROCK) inhibitor, an inhibitor of TGF-β/activing-Nodal signaling, at least one activator of Sonic Hedgehog (SHH) signaling, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 0.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

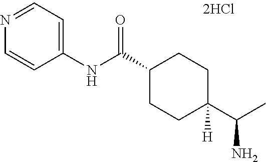 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 0.

In some embodiments the media is supplemented with an inhibitor of TGF-β/activing-Nodal signaling. In some embodiments the media is supplemented with an inhibitor of TGF-β/activing-Nodal signaling up to about day 7 (e.g. day 6 or day 7). In some embodiments the media is supplemented with an inhibitor of TGF-β/activing-Nodal signaling from about day 0 through day 6, each day inclusive.

In some embodiments, cells are exposed to the inhibitor of TGF-β/activing-Nodal signaling at a concentration of between about 1 μM and about 20 μM, between about 5 μM and about 15 μM, or between about 8 μM and about 12 μM. In some embodiments, cells are exposed to the inhibitor of TGF-β/activing-Nodal signaling at a concentration of between about 1 μM and about 20 μM. In some embodiments, cells are exposed to the inhibitor of TGF-β/activing-Nodal signaling at a concentration of between about 5 μM and about 15 μM. In some embodiments, cells are exposed to the inhibitor of TGF-β/activing-Nodal signaling at a concentration of between about 8 μM and about 12 μM. In some embodiments, cells are exposed to the inhibitor of TGF-β/activing-Nodal signaling at a concentration of about 10 μM.

In some embodiments, the inhibitor of TGF-β/activing-Nodal signaling is a small molecule. In some embodiments, the inhibitor of TGF-β/activing-Nodal signaling is capable of lowering or blocking transforming growth factor beta (TGFβ)/Activin-Nodal signaling. In some embodiments, the inhibitor of TGF-β/activing-Nodal signaling inhibits ALK4, ALK5, ALK7, or combinations thereof. In some embodiments, the inhibitor of TGF-β/activing-Nodal signaling inhibits ALK4, ALK5, and ALK7. In some embodiments, the inhibitor of TGF-β/activing-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activing-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

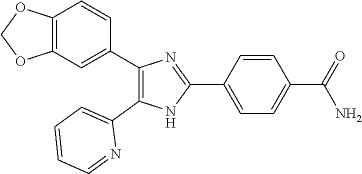 [SMILES: NC(=O)c1ccc(-c2nc(-c3ccc4c(c3)OCO4)c(-c3ccccn3)[nH]2)cc1]

In some embodiments, cells are exposed to SB431542 at a concentration of about 10 μM. In some embodiments, cells are exposed to SB431542 at a concentration of about 10 μM until about day 7. In some embodiments, cells are exposed to SB431542 at a concentration of about 10 μM from about day 0 through about day 6, inclusive of each day.

In some embodiments the media is supplemented with at least one activator of sonic hedghehog (SHH) signaling. SHH refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). In some embodiments the media is supplemented with the at least one activator of SHH signaling up to about day 7 (e.g., day 6 or day 7). In some embodiments the media is supplemented with the at least one activator of SHH signaling from about day 0 through day 6, each day inclusive.

In some embodiments, the at least one activator of SHH signaling is SHH protein. In some embodiments, the at least one activator of SHH signaling is recombinant SHH protein. In some embodiments, the at least one activator of SHH signaling is recombinant mouse SHH protein. In some embodiments, the at least one activator of SHH signaling is recombinant human SHH protein. In some embodiments, the least one activator of SHH signaling is a recombinant N-Terminal fragment of a full-length murine sonic hedgehog protein capable of binding to the SHH receptor for activating SHH. In some embodiments, the at least one activator of SHH signaling is C25II SHH protein.

In some embodiments, cells are exposed to the at least one activator of SHH signaling at a concentration of between about 10 ng/mL and about 500 ng/mL, between about 20 ng/mL and 400 g/mL, between about 30 ng/mL and about 300 ng/mL, between about about 40 ng/mL and about 200 ng/mL, or between about 50 ng/mL and about 100 ng/mL, each inclusive. In some embodiments, cells are exposed to the at least one activator of SHH signaling at a concentration of between about 50 ng/mL and about 100 ng/mL, each inclusive. In some embodiments, cells are exposed to the at least one activator of SHH signaling at a concentration of about 100 ng/mL. In some embodiments, the cells are exposed to SHH protein at about 100 ng/mL. In some embodiments, the cells are exposed to recombinant SHH protein at about 100 ng/mL. In some embodiments, the cells are exposed to recombinant mouse SHH protein at about 100 ng/mL. In some embodiments, the cells are exposed to C25II SHH protein at about 100 ng/mL.

In some embodiments, cells are exposed to recombinant SHH protein at a concentration of about 10 ng/mL. In some embodiments, cells are exposed to recombinant SHH protein at a concentration of about 10 ng/mL up to about day 7 (e.g., day 6 or day 7). In some embodiments, cells are exposed to recombinant SHH protein at a concentration of about 10 ng/mL from about day 0 through about day 6, inclusive of each day.

In some embodiments, cells are exposed to the at least one activator of SHH signaling at a concentration of between about 1 μM and about 20 μM, between about 5 μM and about 15 μM, or between about 8 μM and about 12 μM. In some embodiments, cells are exposed to the at least one activator of SHH signaling at a concentration of between about 1 μM and about 20 μM. In some embodiments, cells are exposed to the at least one activator of SHH signaling at a concentration of between about 5 μM and about 15 μM. In some embodiments, cells are exposed to the at least one activator of SHH signaling at a concentration of between about 8 μM and about 12 μM. In some embodiments, cells are exposed to the at least one activator of SHH signaling at a concentration of about 10 μM.

In some embodiments, the at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g. CAS 483367-10-8), having the formula below:

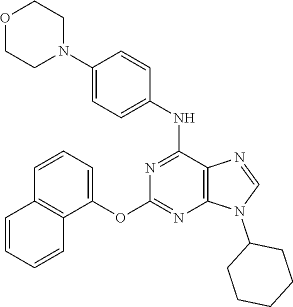 [SMILES: c1ccc2c(Oc3nc(Nc4ccc(N5CCOCC5)cc4)c4ncn(C5CCCCC5)c4n3)cccc2c1]

In some embodiments, cells are exposed to purmorphamine at a concentration of about 10 μM. In some embodiments, cells are exposed to purmorphamine at a concentration of about 10 μM up to day 7 (e.g., day 6 or day 7). In some embodiments, cells are exposed to purmorphamine at a concentration of about 10 μM from about day 0 through about day 6, inclusive of each day.

In some embodiments, the at least one activator of SHH signaling is SHH protein and purmorphamine. In some embodiments, cells are exposed to SHH protein and purmorphamine at a concentration up to about day 7 (e.g., day 6 or day 7). In some embodiments, cells are exposed to SHH protein and purpomorphamine from about day 0 through about day 6, inclusive of each day. In some embodiments, cells are exposed to 100 ng/mL SHH protein and 10 μM purmorphamine at a concentration up to about day 7 (e.g., day 6 or day 7). In some embodiments, cells are exposed to 100 ng/mL SHH protein and 10 μM purpomorphamine from about day 0 through about day 6, inclusive of each day.

In some embodiments the media is supplemented with an inhibitor of BMP signaling. In some embodiments the media is supplemented with an inhibitor of BMP signaling up to about day 7 (e.g., day 6 or day 7). In some embodiments the media is supplemented with an inhibitor of BMP signaling from about day 0 through day 6, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

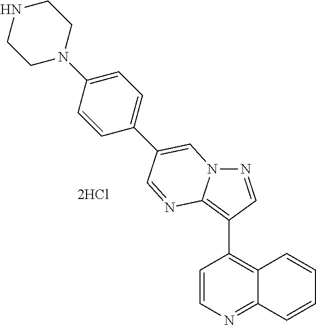 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM up to about day 7 (e.g., day 6 or day 7). In some embodiments, cells are exposed to LDN193189 at a concentration of about 0.1 μM from about day 0 through about day 6, inclusive of each day.

In some embodiments the media is supplemented with an inhibitor of GSK3β signaling. In some embodiments the media is supplemented with an inhibitor of GSK3β signaling up to about day 7 (e.g., day 6 or day 7). In some embodiments the media is supplemented with an inhibitor of GSK3β signaling from about day 0 through day 6, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of BMP signaling at a concentration of between about 1 μM and about 4 μM. In some embodiments, cells are exposed to the inhibitor of BMP signaling at a concentration of between about 2 μM and about 3 μM. In some embodiments, cells are exposed to the inhibitor of GSK3β signaling at a concentration of about 2 μM.

In some embodiments, the inhibitor of GSK3β signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3a. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

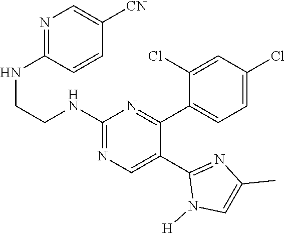 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM up to about day 7 (e.g., day 6 or day 7). In some embodiments, cells are exposed to CHIR99021 at a concentration of about 2.0 μM from about day 0 through about day 6, inclusive of each day.

In some embodiments, from day about 2 to about day 6, at least about 50% of the media is replaced daily. In some embodiments, from about day 2 to about day 6, about 50% of the media is replaced daily, every other day, or every third day. In some embodiments, from about day 2 to about day 6, about 50% of the media is replaced daily. In some embodiments, at least about 75% of the media is replaced on day 1. In some embodiments, about 100% of the media is replaced on day 1. In some embodiments, the replacement media contains small molecules about twice as concentrated as compared to the concentration of the small molecules in the media on day 0.

In some embodiments, the first incubation comprises culturing pluripotent stem cells in a “basal induction media.” In some embodiments, the first incubation comprises culturing pluripotent stem cells in the basal induction media from about day 0 through about day 6. In some embodiments, the first incubation comprises culturing pluripotent stem cells in the basal induction media to induce differentiation of the PSCs into floor plate midbrain progenitor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the small molecules as described above.

### 3. Transfer or Dissociation of Spheroids

In some embodiments, cell aggregates (e.g. spheroids) that are produced following the first incubation of culturing pluripotent stem cells in a non-adherent culture vessel are transferred or dissociated, prior to carrying out a second incubation of the cells on a substrate (adherent culture).

In some embodiments, the first incubation is carried out to produce a cell aggregate (e.g. a spheroid) that expresses at least one of PAX6 and OTX2. In some embodiments, the first incubation produces a cell aggregate (e.g. a spheroid) that expresses PAX6 and OTX2. In some embodiments, the first incubation produces a cell aggregate (e.g. a spheroid) on or by about day 7 of the methods provided herein. In some embodiments, the first incubation produces a cell aggregate (e.g. a spheroid) that expresses at least one of PAX6 and OTX2 on or by about day 7 of the methods provided herein. In some embodiments, the first incubation produces a cell aggregate (e.g. a spheroid) that expresses PAX6 and OTX2 on or by about day 7 of the methods provided herein.

In some embodiments, the cell aggregate (e.g. spheroid) produced by the first incubation is dissociated prior to the second incubation of the cells on a substrate. In some embodiments, the cell aggregate (e.g. spheroid) produced by the first incubation is dissociated to produce a cell suspension. In some embodiments, the cell suspension produced by the dissociation is a single cell suspension. In some embodiments, the dissociation is carried out at a time when the spheroid cells express at least one of PAX6 and OTX2. In some embodiments, the dissociation is carried out at a time when the spheroid cells express PAX6 and OTX2. In some embodiments, the dissociation is carried out on about day 7. In some embodiments, the cell aggregate (e.g. spheroid) is dissociated by enzymatic dissociation. In some embodiments, the enzyme is selected from among the group consisting of: accutase, dispase, collagenase, and combinations thereof. In some embodiments, the enzyme comprises accutase. In some embodiments, the enzyme is accutase. In some embodiments, the enzyme is dispase. In some embodiments, the enzyme is collagenase.

In some embodiments, the cell aggregate or cell suspension produced therefrom is transferred to a substrate-coated culture vessel for a second incubation. In some embodiments, the cell aggregate (e.g. spheroid) or cell suspension produced therefrom is transferred to a substrate-coated culture vessel following dissociation of the cell aggregate (e.g. spheroid). In some embodiments, the transferring is carried out immediately after the dissociating. In some embodiments, the transferring is carried out on about day 7.

In some embodiments, the cell aggregate (e.g., spheroid) is not dissociated prior to a second incubation. In some embodiments, a cell aggregate (e.g. spheroid) is transferred in its entirety to a substrate-coated culture vessel for a second incubation. In some embodiments, the transferring is carried out at a time when the spheroid cells express at least one of PAX6 and OTX2. In some embodiments, the transferring is carried out at a time when the spheroid cells express PAX6 and OTX2. In some embodiments, the transferring is carried out on about day 7.

In some embodiments, the transferring is to an adherent culture vessel. In some embodiments, the culture vessel is a plate, a dish, a flask, or a bioreactor. In some embodiments, the culture vessel is substrate-coated. In some embodiments, the substrate is a basement membrane protein. In some embodiments, the substrate is selected from laminin, collagen, entactin, heparin sulfate proteoglycans, and combinations thereof. In some embodiments, the substrate is laminin. In some embodiments, the substrate is recombinant. In some embodiments, the substrate is recombinant laminin. In some embodiments, the substrate-coated culture vessel is exposed to poly-L-ornithine, optionally prior to being used for culturing cells. In some embodiments, the substrate-coated culture vessel is a 6-well or 24-well plate. In some embodiments, the substrate-coated culture vessel is a 6-well plate. In some embodiments, the substrate-coated culture vessel is a 24-well plate.

### 4. Adherent Culture

In some embodiments, the methods include performing a second incubation of the spheroid cells transferred to the substrate-coated culture vessel. In some embodiments, culturing the cells of the spheroid in the substrate-coated culture vessel under adherent conditions induces their differentiation into floor plate midbrain progenitor cells, determined dopamine (DA) neuron progenitor cells, and/or, dopamine (DA) neurons.

In some embodiments, the second incubation involves culturing cells of the spheroid in a culture vessel coated with a substrate including laminin, collagen, entactin, heparin sulfate proteoglycans, or a combination thereof, wherein beginning on day 7, the cells are exposed to (i) an inhibitor of BMP signaling and (ii) an inhibitor of GSK3β signaling; and beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling. In some embodiments, the method further includes harvesting the differentiated cells.

In some embodiments, the substrate-coated culture vessel is a culture vessel with a surface to which cells can attach. In some embodiments, the substrate-coated culture vessel is a culture vessel with a surface to which a substantial number of cells attach. In some embodiments, the substrate is a basement membrane protein. In some embodiments, the substrate is laminin, collagen, entactin, heparin sulfate proteoglycans, or a combination thereof. In some embodiments, the substrate is laminin. In some embodiments, the substrate is collagen. In some embodiments, the substrate is entactin. In some embodiments, the substrate is heparin sulfate proteoglycans. In some embodiments, the substrate is a recombinant protein. In some embodiments, the substrate is recombinant laminin. In some embodiments, the substrate-coated culture vessel is exposed to poly-L-ornithine. In some embodiments, the substrate-coated culture vessel is exposed to poly-L-ornithine prior to being used for cell culture.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a plate. In some embodiments, the non-adherent culture vessel is a 6-well or 24-well plate. In some embodiments, the non-adherent culture vessel is a dish. In some embodiments, the non-adherent culture vessel is a flask. In some embodiments, the non-adherent culture vessel is a bioreactor.

In some embodiments, the substrate-coated culture vessel allows for a monolayer cell culture. In some embodiments, cells derived from the cell aggregate (e.g. spheroid) produced by the first incubation are cultured in a monolayer culture on the substrate-coated plates. In some embodiments, cells derived from the cell aggregate (e.g. spheroid) produced by the first incubation are cultured to produce a monolayer culture of cells positive for one or more of LMX1A, FOXA2, EN1, CORIN, and combinations thereof. In some embodiments, cells derived from the cell aggregate (e.g. spheroid) produced by the first incubation are cultured to produce a monolayer culture of cells, wherein at least some of the cells are positive for EN1 and CORIN. In some embodiments, cells derived from the cell aggregate (e.g. spheroid) produced by the first incubation are cultured to produce a monolayer culture of cells, wherein at least some of the cells are TH+. In some embodiments, at least some cells are TH+ by or on about day 25. In some embodiments, cells derived from the cell aggregate (e.g. spheroid) produced by the first incubation are cultured to produce a monolayer culture of cells, wherein at least some of the cells are TH+FOXA2+. In some embodiments, at least some cells are TH+FOXA2+ by or on about day 25.

In the methods provided herein, the second incubation involves culturing cells of the spheroid in a substrate-coated culture vessel under conditions to induce neural differentiation of the cells. In some embodiments, the cells of the spheroid are plated on the substrate-coated culture vessel on about day 7.

In some embodiments, the number of cells plated on the substrate-coated culture vessel is between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the second incubation is from about day 7 until harvesting of the cells. In some embodiments, the cells are harvested on about day 16 or later. In some embodiments, the cells are harvested between about day 16 and about day 30. In some embodiments, the cells are harvested between about day 18 and about day 25. In some embodiments, the cells are harvested on about day 18. In some embodiments, the cells are harvested on about day 25. In some embodiments, the second incubation is from about day 7 until about day 18. In some embodiments, the second incubation is from about day 7 until about day 25.

In some embodiments, the second incubation involves culturing cells derived from the cell aggregate (e.g. spheroid) in a culture media (“media”).

In some embodiments, the second incubation involves culturing the cells in the media from about day 7 until harvest or collection. In some embodiments, cells are cultured in the media to produce determined dopamine (DA) neuron progenitor cells, or dopamine (DA) neurons.

In some embodiments, the media is also supplemented with a serum replacement containing minimal non-human-derived components (e.g., KnockOut™ serum replacement). In some embodiments, the media is supplemented with the serum replacement from about day 7 through about day 10. In some embodiments, the media is supplemented with about 2% (v/v) of the serum replacement. In some embodiments, the media is supplemented with about 2% (v/v) of the serum replacement from about day 7 through about day 10.

In some embodiments, the media is further supplemented with small molecules. In some embodiments, the small molecules are selected from among the group consisting of: a Rho-associated protein kinase (ROCK) inhibitor, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 7, day 16, day 20, or a combination thereof. In some embodiments the media is supplemented with a ROCK inhibitor on day 7. In some embodiments the media is supplemented with a ROCK inhibitor on day 16. In some embodiments the media is supplemented with a ROCK inhibitor on day 20. In some embodiments the media is supplemented with a ROCK inhibitor on day 7 and day 16. In some embodiments the media is supplemented with a ROCK inhibitor on day 16 and day 20. In some embodiments the media is supplemented with a ROCK inhibitor on day 7, day 16, and day 20.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is Fasudil, Ripasudil, Netarsudil, RKI-1447, Y-27632, GSK429286A, Y-30141, or a combination thereof. In some embodiments, the ROCK inhibitor is a small molecule. In some embodiments, the ROCK inhibitor selectively inhibits p160ROCK. In some embodiments, the ROCK inhibitor is Y-27632, having the formula:

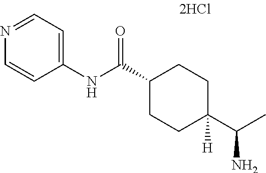 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 7, day 16, day 20, or a combination thereof. In some embodiments, cells are exposed to Y-27632 at a concentration of about 10 μM on day 7. In some embodiments, cells are exposed to Y-27632 at a concentration of about 10 μM on day 16. In some embodiments, cells are exposed to Y-27632 at a concentration of about 10 μM on day 20. In some embodiments, cells are exposed to Y-27632 at a concentration of about 10 μM on day 7 and day 16. In some embodiments, cells are exposed to Y-27632 at a concentration of about 10 μM on day 16 and day 20. In some embodiments, cells are exposed to Y-27632 at a concentration of about 10 μM on day 7, day 16, and day 20.

In some embodiments the media is supplemented with an inhibitor of BMP signaling. In some embodiments the media is supplemented with an inhibitor of BMP signaling from about day 7 up to about day 11 (e.g., day 10 or day 11). In some embodiments the media is supplemented with an inhibitor of BMP signaling from about day 7 through day 10, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is LDN193189 or K02288. In some embodiments, the inhibitor of BMP signaling is capable of inhibiting “Small Mothers Against Decapentaplegic” SMAD signaling. In In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

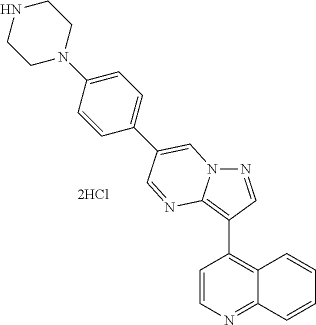 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM from about day 7 up to about day 11 (e.g., day 10 or day 11). In some embodiments, cells are exposed to LDN193189 at a concentration of about 0.1 μM from about day 7 through about day 10, inclusive of each day.

In some embodiments the media is supplemented with an inhibitor of GSK3β signaling. In some embodiments the media is supplemented with an inhibitor of GSK3β signaling from about day 7 up to about day 13 (e.g., day 12 or day 13). In some embodiments the media is supplemented with an inhibitor of GSK3β signaling from about day 7 through day 12, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3β signaling at a concentration of between about 1 μM and about 4 μM. In some embodiments, cells are exposed to the inhibitor of GSK3β signaling at a concentration of between about 2 μM and about 3 μM. In some embodiments, cells are exposed to the inhibitor of GSK3β signaling at a concentration of about 2 μM.

In some embodiments, the inhibitor of GSK3β signaling is selected from lithium ion, valproic acid, iodotubercidin, naproxen, famotidine, curcumin, olanzapine, CHIR99012, or a combination thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3α. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

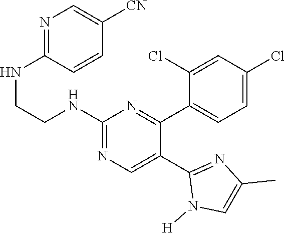 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM from about day 7 up to about day 13 (e.g., day 12 or day 13). In some embodiments, cells are exposed to CHIR99021 at a concentration of about 2.0 μM from about day 7 through about day 12, inclusive of each day.

In some embodiments the media is supplemented with brain-derived neurotrophic factor (BDNF). In some embodiments the media is supplemented with BDNF beginning on about day 11. In some embodiments the media is supplemented with BDNF from about day 11 until harvest or collection. In some embodiments the media is supplemented with BDNF from about day 11 through day 18. In some embodiments the media is supplemented with BDNF from about day 11 through day 25.

In some embodiments, cells are exposed to BDNF at a concentration of between about 1 ng/mL and 100 ng/mL, between about 5 ng/mL and about 50 ng/mL, between about 10 ng/mL and about 30 ng/mL. In some embodiments, cells are exposed to BDNF at a concentration of between about 10 ng/mL and about 30 ng/mL. In some embodiments, cells are exposed to BDNF at a concentration of about 20 ng/mL.

In some embodiments, the media is supplemented with about 20 ng/mL BDNF beginning on about day 11. In some embodiments the media is supplemented with 20 ng/mL BDNF from about day 11 until harvest or collection. In some embodiments the media is supplemented with about 20 ng/mL BDNF from about day 11 through day 18. In some embodiments the media is supplemented with about 20 ng/mL BDNF from about day 11 through day 25.

In some embodiments the media is supplemented with glial cell-derived neurotrophic factor (GDNF). In some embodiments the media is supplemented with GDNF beginning on about day 11. In some embodiments the media is supplemented with GDNF from about day 11 until harvest or collection. In some embodiments the media is supplemented with GDNF from about day 11 through day 18. In some embodiments the media is supplemented with GDNF from about day 11 through day 25.

In some embodiments, cells are exposed to GDNF at a concentration of between about 1 ng/mL and 100 ng/mL, between about 5 ng/mL and about 50 ng/mL, between about 10 ng/mL and about 30 ng/mL. In some embodiments, cells are exposed to GDNF at a concentration of between about 10 ng/mL and about 30 ng/mL. In some embodiments, cells are exposed to GDNF at a concentration of about 20 ng/mL.

In some embodiments, the media is supplemented with about 20 ng/mL GDNF beginning on about day 11. In some embodiments the media is supplemented with 20 ng/mL GDNF from about day 11 until harvest or collection. In some embodiments the media is supplemented with about 20 ng/mL GDNF from about day 11 through day 18. In some embodiments the media is supplemented with about 20 ng/mL GDNF from about day 11 through day 25.

In some embodiments the media is supplemented with ascorbic acid. In some embodiments the media is supplemented with ascorbic acid beginning on about day 11. In some embodiments the media is supplemented with ascorbic acid from about day 11 until harvest or collection. In some embodiments the media is supplemented with ascorbic acid from about day 11 through day 18. In some embodiments the media is supplemented with ascorbic acid from about day 11 through day 25.

In some embodiments, cells are exposed to ascorbic acid at a concentration of between about 0.05 mM and 5 mM, between about 0.1 mM and about 1 mM, between about 0.2 mM and about 0.5 mM, each inclusive. In some embodiments, cells are exposed to ascorbic acid at a concentration of between about 0.05 mM and about 5 mM, each inclusive. In some embodiments, cells are exposed to ascorbic acid at a concentration of between about 0.1 mM and about 1 mM, each inclusive. In some embodiments, cells are exposed to ascorbic acid at a concentration of about 0.2 mM.

In some embodiments, the media is supplemented with about 0.2 mM ascorbic acid beginning on about day 11. In some embodiments the media is supplemented with 0.2 mM ascorbic acid from about day 11 until harvest or collection. In some embodiments the media is supplemented with about 0.2 mM ascorbic acid from about day 11 through day 18. In some embodiments the media is supplemented with about 0.2 mM ascorbic acid from about day 11 through day 25.

In some embodiments the media is supplemented with dibutyryl cyclic AMP (dbcAMP). In some embodiments the media is supplemented with dbcAMP beginning on about day 11. In some embodiments the media is supplemented with dbcAMP from about day 11 until harvest or collection. In some embodiments the media is supplemented with dbcAMP from about day 11 through day 18. In some embodiments the media is supplemented with dbcAMP from about day 11 through day 25.

In some embodiments, cells are exposed to dbcAMP at a concentration of between about 0.05 mM and 5 mM, between about 0.1 mM and about 3 mM, between about 0.2 mM and about 1 mM, each inclusive. In some embodiments, cells are exposed to dbcAMP at a concentration of between about 0.1 mM and about 3 mM, each inclusive. In some embodiments, cells are exposed to dbcAMP at a concentration of between about 0.2 mM and about 1 mM, each inclusive. In some embodiments, cells are exposed to dbcAMP at a concentration of about 0.5 mM.

In some embodiments, the media is supplemented with about 0.5 mM dbcAMP beginning on about day 11. In some embodiments the media is supplemented with 0.5 mM dbcAMP from about day 11 until harvest or collection. In some embodiments the media is supplemented with about 0.5 mM dbcAMP from about day 11 through day 18. In some embodiments the media is supplemented with about 0.5 mM dbcAMP from about day 11 through day 25.

In some embodiments the media is supplemented with transforming growth factor beta 3 (TGFβ3). In some embodiments the media is supplemented with TGFβ3 beginning on about day 11. In some embodiments the media is supplemented with TGFβ3 from about day 11 until harvest or collection. In some embodiments the media is supplemented with TGFβ3 from about day 11 through day 18. In some embodiments the media is supplemented with TGFβ3 from about day 11 through day 25.

In some embodiments, cells are exposed to TGFβ3 at a concentration of between about 0.1 ng/mL and 10 ng/mL, between about 0.5 ng/mL and about 5 ng/mL, or between about 1.0 ng/mL and about 2.0 ng/mL. In some embodiments, cells are exposed to TGFβ3 at a concentration of between about 1.0 ng/mL and about 2.0 ng/mL, each inclusive. In some embodiments, cells are exposed to TGFβ3 at a concentration of about 1 ng/mL.

In some embodiments, the media is supplemented with about 1 ng/mL TGFβ3 beginning on about day 11. In some embodiments the media is supplemented with 1 ng/mL TGFβ3 from about day 11 until harvest or collection. In some embodiments the media is supplemented with about 1 ng/mL TGFβ3 from about day 11 through day 18. In some embodiments the media is supplemented with about 1 ng/mL TGFβ3 from about day 11 through day 25.

In some embodiments the media is supplemented with an inhibitor of Notch signaling. In some embodiments the media is supplemented with an inhibitor of Notch signaling beginning on about day 11. In some embodiments the media is supplemented with an inhibitor of Notch signaling from about day 11 until harvest or collection. In some embodiments the media is supplemented with an inhibitor of Notch signaling from about day 11 through day 18. In some embodiments the media is supplemented with an inhibitor of Notch signaling from about day 11 through day 25.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

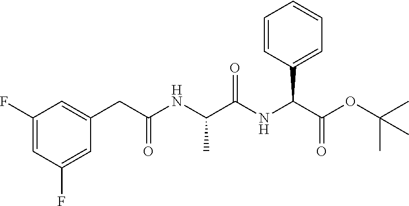 [SMILES: C[C@H](NC(=O)Cc1cc(F)cc(F)c1)C(=O)N[C@H](C(=O)OC(C)(C)C)c1ccccc1]

In some embodiments, cells are exposed to DAPT at a concentration of between about 1 μM and about 20 μM, between about 5 μM and about 15 μM, or between about 8 μM and about 12 μM. In some embodiments, cells are exposed to DAPT at a concentration of between about 1 μM and about 20 μM. In some embodiments, cells are exposed to DAPT at a concentration of between about 5 μM and about 15 μM. In some embodiments, cells are exposed to DAPT at a concentration of between about 8 μM and about 12 μM. In some embodiments, cells are exposed to DAPT at a concentration of about 10 μM.

In some embodiments, the media is supplemented with about 10 μM DAPT beginning on about day 11. In some embodiments the media is supplemented with 10 μM DAPT from about day 11 until harvest or collection. In some embodiments the media is supplemented with about 10 μM DAPT from about day 11 through day 18. In some embodiments the media is supplemented with about 10 μM DAPT from about day 11 through day 25.

In some embodiments, beginning on about day 11, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until harvest or collection, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 18, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 25, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT.

In some embodiments, a serum replacement is provided in the media from about day 7 through about day 10. In some embodiments, the serum replacement is provided at 2% (v/v) in the media on day 7 through day 10.

In some embodiments, from day about 7 to about day 16, at least about 50% of the media is replaced daily. In some embodiments, from about day 7 to about day 16, about 50% of the media is replaced daily, every other day, or every third day. In some embodiments, from about day 7 to about day 16, about 50% of the media is replaced daily. In some embodiments, beginning on about day 17, at least about 50% of the media is replaced daily, every other day, or every third day. In some embodiments, beginning on about day 17, at least about 50% of the media is replaced every other day. In some embodiments, beginning on about day 17, about 50% of the media is replaced daily, every other day, or every third day. In some embodiments, beginning on about day 17, about 50% of the media is replaced every other day. In some embodiments, the replacement media contains small molecules about twice as concentrated as compared to the concentration of the small molecules in the media on day 0.

In some embodiments, the second incubation involves culturing cells derived from the cell aggregate (e.g. spheroid) in a “basal induction media.” In some embodiments, the second incubation involves culturing cells derived from the cell aggregate (e.g. spheroid) in a “maturation media.” In some embodiments, the second incubation involves culturing cells derived from the cell aggregate (e.g. spheroid) in the basal induction media, and then in the maturation media.

In some embodiments, the second incubation involves culturing the cells in the basal induction media from about day 7 through about day 10. In some embodiments, the second incubation involves comprises culturing the cells in the maturation media beginning on about day 11. In some embodiments, the second incubation involves culturing the cells in the basal induction media from about day 7 through about day 10, and then in the maturation media beginning on about day 11. In some embodiments, cells are cultured in the maturation media to produce determined dopamine (DA) neuron progenitor cells, or dopamine (DA) neurons.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the molecules described in Section II.

In some embodiments, the maturation media is formulated to contain Neurobasal™ media, supplemented with N-2 and B27 supplements, non-essential amino acids (NEAA), and GlutaMAX™. In some embodiments, the maturation media is further supplemented with any of the molecules described in Section II.

In some embodiments, the cells are cultured in the basal induction media from about day 7 up to about day 11 (e.g., day 10 or day 11). In some embodiments, the cells are cultured in the basal induction media from about day 7 through day 10, each day inclusive. In some embodiments, the cells are cultured in the maturation media beginning on about day 11. In some embodiments, the cells are cultured in the basal induction media from about day 7 through about day 10, and then the cells are cultured in the maturation media beginning on about day 11. In some embodiments, the cells are cultured in the maturation media from about day 11 until harvest or collection of the cells. In some embodiments, cells are harvested between day 16 and 27. In some embodiments, cells are harvested between day 18 and day 25. In some embodiments, cells are harvested on day 18. In some embodiments, cells are harvested on day 25.

### 5. Harvesting, Collecting, and Formulating Differentiated Cells

In embodiments of the provided methods, neutrally differentiated cells produced by the methods provided herein can be harvested or collected, such as for formulation and use of the cells. In some embodiments, the provided methods for producing differentiated cells, such as for use as a cell therapy in the treatment of a neurodegenerative disease may include formulation of cells, such as formulation of differentiated cells resulting from the provided methods described herein. In some embodiments, the dose of cells comprising differentiated cells (e.g. determined DA neuron progenitor cells or DA neurons), is provided as a composition or formulation, such as a pharmaceutical composition or formulation. Such compositions can be used in accord with the provided methods, such as in the prevention or treatment of neurodegenerative disorders, including Parkinson's disease.

In some cases, the cells are processed in one or more steps for manufacturing, generating or producing a cell therapy and/or differentiated cells may include formulation of cells, such as formulation of differentiated cells resulting from the methods. In some cases, the cells can be formulated in an amount for dosage administration, such as for a single unit dosage administration or multiple dosage administration.

In certain embodiments, one or more compositions of differentiated cells are formulated. In particular embodiments, one or more compositions of differentiated cells are formulated after the one or more compositions have been produced. In some embodiments, the one or more compositions have been previously cryopreserved and stored, and are thawed prior to the administration.

In certain embodiments, the differentiated cells include determined DA neuron progenitor cells. In some embodiments, a formulated composition of differentiated cells is a composition enriched for determined DA neuron progenitor cells. In certain embodiments, the differentiated cells include DA neurons. In some embodiments, a formulated composition of differentiated cells is a composition enriched for DA neurons.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are differentiated for at least 16 days. In some embodiments, the cells are differentiated for between 16 day and 30 days. In some embodiments, the cells are differentiated for between 16 day and 27 days. In some embodiments, the cells are differentiated for between 18 and 25 day. In some embodiments, the cells are differentiated for about 18 days. In some embodiments, the cells are differentiated for about 25 days.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are harvested after at least 16 days of culture. In some embodiments, the cells are harvested between 16 days and 30 days of culture. In some embodiments, the cells are harvested between 16 days and 27 days of culture. In some embodiments, the cells are harvested between 18 days and 25 days of culture. In some embodiments, the cells are harvested after about 18 days of cluture. In some embodiments, the cells are harvested after about 25 days of culture.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the differentiated cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the neurodegenerative condition or disease (e.g. Parkinson's disease), such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the differentiated cells to replace the cells in a cryopreservative solution. In some embodiments, the cells are frozen, e.g., cryopreserved or cryoprotected, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of differentiated cells are formulated, cryopreserved, and then stored for an amount of time. In certain embodiments, the formulated, cryopreserved cells are stored until the cells are released for administration. In particular embodiments, the formulated cryopreserved cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryopreserved and stored for, for about, or for less than 1 days, 2 days, 3 days, 4 days, 5 days, 6 days, or 7 days. In certain embodiments, the cells are thawed and administered to a subject after the storage.

In some embodiments, the formulation is carried out using one or more processing step including washing, diluting or concentrating the cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to differentiated cells. In some embodiments, the volume of formulation buffer is from or from about 1 μL to 5000 μL, such as at least or about at least or about or 5 μL, 10 μL, 20 μL, 50 μL, 100 μL, 200 μL, 300 μL, 400 μL, 500 μL, 1000 μL, 2000 μL, 3000 μL, 4000 μL, or 5000 μL.

A container may generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, such cells produced by the method, or a composition comprising such cells, are administered to a subject for treating a neurodegenerative disease or condition.

### B. Exemplary Processes

As described by the methods provided herein, pluripotent stem cells may be differentiated into lineage specific cell populations, including determined DA progenitors cells and DA neurons. These cells may then be used in cell replacement therapy. As described by the methods here, in some embodiments, the pluripotent stem cells are differentiated into floor plate midbrain progenitor cells, and the resulting spheroid cells are further differentiated into determined dopamine (DA) neuron progenitor cells, and/or dopamine (DA) neurons. In some embodiments, the pluripotent stem cells are differentiated into determined DA neuron progenitor cells. In some embodiments, the pluripotent stem cells are differentiated into DA neurons. In some embodiments, pluripotent stem cells are embryonic stem cells. In some embodiments, pluripotent stem cells are induced pluripotent stem cells.

In some embodiments, embryonic stem cells are differentiated into floor plate midbrain progenitor cells, and then into determined dopamine (DA) neuron progenitor cells, and/or dopamine (DA) neurons. In some embodiments, embryonic stem cells are differentiated into determined DA neuron progenitor cells. In some embodiments, embryonic stem cells are differentiated into DA neurons.

In some embodiments, induced pluripotent stem cells are differentiated into floor plate midbrain progenitor cells, and then into determined dopamine (DA) neuron progenitor cells, and/or dopamine (DA) neurons. In some embodiments, induced pluripotent stem cells are differentiated into determined DA neuron progenitor cells. In some embodiments, induced pluripotent stem cells are differentiated into DA neurons.

In some embodiments, the method involves (a) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to induce neural differentiation the cells.

In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the method involves (a) performing a first incubation including culturing pluripotent stem cells in a plate having microwells under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; (iv) an inhibitor of glycogen synthase kinase 3β (GSK3β) signaling; and (v) a serum replacement; (b) dissociating the cells of the spheroid to produce a cell suspension; (c) transferring cells of the cell suspension to a laminin-coated culture vessel; (d) performing a second incubation including culturing cells of the spheroid in the laminin-coated culture vessel under conditions to induce neural differentiation of the cells; and (e) harvesting the neurally differentiated cells. In some embodiments, the second incubation involves culturing cells in the presence of a serum replacement. In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the cells are exposed to the inhibitor of TGF-β/activing-Nodal (e.g., SB431542 or “SB”) from day 0 up to about day 7 (e.g., day 6 or day 7). In some embodiments, the cells are exposed to the inhibitor of TGF-β/activing-Nodal (e.g., SB431542 or “SB”) from day 0 through day 6, inclusive of each day. In some embodiments, the cells are exposed to the at least one activator of SHH signaling (e.g., SHH protein and purmorphamine, collectively “SHH/PUR”) from day 0 up to about day 7 (e.g. day 6 or day 7). In some embodiments, the cells are exposed to the at least one activator of SHH signaling (e.g., SHH protein and purmorphamine, collectively “SHH/PUR”) from day 0 through day 6, inclusive of each day. In some embodiments, the cells are exposed to the inhibitor of BMP signaling (e.g., LDN193189 or “LDN”) from day 0 up to about day 11 (e.g., day 10 or day 11). In some embodiments, the cells are exposed to the inhibitor of BMP signaling (e.g., LDN193189 or “LDN”) from day 0 through day 10, inclusive of each day. In some embodiments, the cells are exposed to the inhibitor of GSK3β signaling (e.g., CHIR99021 or “CHIR”) from day 0 up to about day 13 (e.g., day 12 or day 13). In some embodiments, the cells are exposed to the inhibitor of GSK3β signaling (e.g., CHIR99021 or “CHIR”) from day 0 through day 12.

In some embodiments, the cells are exposed to (i) an inhibitor of TGF-β/activing-Nodal signaling from day 0 up to about day 7 (e.g., day 6 or day 7); (ii) at least one activator of Sonic Hedgehog (SHH) signaling from day 0 up to about day 7 (e.g., day 6 or day 7); (iii) an inhibitor of bone morphogenetic protein (BMP) signaling from day 0 up to about day 11 (e.g., day 10 or day 11); and (iv) an inhibitor of glycogen synthase kinase 3β (GSK3β) signaling from day 0 up to about day 13 (e.g., day 12 or day 13). In some embodiments, the cells are exposed to (i) SB from day 0 up to about day 7 (e.g., day 6 or day 7); (ii) SHH/PUR from day 0 up to about day 7 (e.g., day 6 or day 8); (iii) LDN from day 0 up to about day 11 (e.g. day 10 or day 11); and (iv) CHIR from day 0 up to about day 13 (e.g. day 12 or day 13). In some embodiments, the cells are exposed to (i) an inhibitor of TGF-β/activing-Nodal signaling from day 0 through day 6, each day inclusive; (ii) at least one activator of Sonic Hedgehog (SHH) signaling from day 0 through day 6, each day inclusive; (iii) an inhibitor of bone morphogenetic protein (BMP) signaling from day 0 through day 10, each day inclusive; and (iv) an inhibitor of glycogen synthase kinase 3β (GSK3β) signaling from day 0 through day 12, each day inclusive. In some embodiments, the cells are exposed to (i) SB from day 0 through day 6, each day inclusive; (ii) SHH/PUR from day 0 through day 6, each day inclusive; (iii) LDN from day 0 through day 10, each day inclusive; and (iv) CHIR from day 0 through day 12, each day inclusive.

In some embodiments, the cells are exposed to brain-derived neurotrophic factor (BDNF) beginning on day 11. In some embodiments, the cells are exposed to ascorbic acid. In some embodiments, the cells are exposed to glial cell-derived neurotrophic factor (GDNF) beginning on day 11. In some embodiments, the cells are exposed to dibutyryl cyclic AMP (dbcAMP) beginning on day 11. In some embodiments, the cells are exposed to transforming growth factor beta-3 (TGFβ3) beginning on day 11. In some embodiments, the cells are exposed to the inhibitor of Notch signaling (e.g., DAPT) beginning on day 11. In some embodiments, beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) the inhibitor of Notch signaling (e.g., DAPT) (collectively “BAGCT/DAPT”). In some embodiments, the cells are exposed to BAGCT/DAPT beginning on day 11 until harvest or collection. In some embodiments, the cells are exposed to BAGCT/DAPT from day 11 through day 18. In some embodiments, the cells are exposed to BAGCT/DAPT from day 11 through day 25.

In some embodiments, the cells are exposed to a Rho-associated protein kinase (ROCK) inhibitor on day 0. In some embodiments, the cells are exposed to a Rho-associated protein kinase (ROCK) inhibitor on day 7. In some embodiments, the cells are exposed to a Rho-associated protein kinase (ROCK) inhibitor on day 16. In some embodiments, the cells are exposed to a Rho-associated protein kinase (ROCK) inhibitor on day 20. In some embodiments, the cells are exposed to a Rho-associated protein kinase (ROCK) inhibitor on day 0, day 7, day 16, and day 20. In some embodiments, the cells are exposed to a ROCK inhibitor on the day on which the cells are passaged. In some embodiments, the cells are passaged on day 0, 7, 16, 20, or combinations thereof. In some embodiments, the cells are passaged on day 0, 7, 16, and 20.

In some embodiments, the cells are cultured in a basal induction medium comprising DMEM/F-12 and Neurobasal media (e.g., at a 1:1 ratio), supplemented with N2, B27, non-essential amino acids (NEAA), Glutamax, L-glutamine, β-mercaptoethanol, and insulin. In some embodiments, the cells are cultured in the basal induction media from about day 0 through about day 10. In some embodiments, the basal induction media is for differentiating pluripotent stem cells into floor plate midbrain progenitor cells.

In some embodiments, the cells are cultured in a maturation medium comprising Neurobasal media, supplemented with N2, B27, non-essential amino acids (NEAA), and Glutamax. In some embodiments, the cells are cultured in the basal induction media from about day 11 until harvest or collection. In some embodiments, the cells are cultured in the basal induction media from about day 11 through day 18. In some embodiments, the maturation media is for differentiating floor plate midbrain progenitor cells into determined dopamine (DA) neuron progenitor cells. In some embodiments, the cells are cultured in the basal induction media from about day 11 through day 25. In some embodiments, the maturation media is for differentiating floor plate midbrain progenitor cells into dopamine (DA) neurons.

In some embodiments, the media is supplemented with small molecules as described above, including SB, SHH/PUR, LDN, CHIR, BAGCT/DAPT, and ROCKi. In some embodiments, the media is changed every day or every other day. In some embodiments the media is changed every day. In some embodiments the media is changed every other day. In some embodiments, the media is changed every day from about day 0 up to about day 17 (e.g., day 16 or day 18). In some embodiments, the media is changed every other day from about day 18 until harvest or collection. In some embodiments, the media is changed every day from about day 0 up to about day 17 (e.g., day 16 or day 18), and then every other day from about day 18 until harvest or collection.

In some embodiments, a serum replacement is provided in the media from about day 0 up to about day 10 (e.g. day 9 or day 11). In some embodiments, the serum replacement is provided at 5% (v/v) in the media on day 0 and day 1. In some embodiments, the serum replacement is provided at 2% (v/v) in the media on day 2 through day 10. In some embodiments, the serum replacement is provided at 5% (v/v) in the media on day 0 and day 1 and at 2% (v/v) in the media on day 2 through day 10. In some embodiments, serum replacement is not provided in the media after day 10.

In some embodiments, at least about 50% or at least about 75% of the media is changed. In some embodiments, at least about 50% of the media is changed. In some embodiments, at least about 75% of the media is changed. In some embodiments about 100% of the media is changed.

In some embodiments, about 50% or about 75% of the media is changed. In some embodiments, about 50% of the media is changed. In some embodiments, about 75% of the media is changed. In some embodiments about 100% of the media is changed.

In some embodiments, the media is supplemented with small molecules selected from SB, SHH/PUR, LDN, CHIR, BAGCT/DAPT, ROCKi, or a combination thereof. In some embodiments, when about 50% of the media is changed, the concentration of each small molecule is doubled as compared to its concentration on day 0.

In some embodiments, cells are harvested between about day 16 and about day 30. In some embodiments, cells are harvested between about day 16 and about day 27. In some embodiments, cells are harvested between about day 18 and about day 25. In some embodiments, cells are harvested on about day 18. In some embodiments, cells are harvested on about day 25. In some embodiments, compositions comprising cells generated by the methods provided herein are used for the treatment of a neurodegenerative disease or condition, such as Parkinson's disease. In some embodiments, a composition of cells generated by any of the methods described herein are administered to a subject who has Parkinson's disease. In some embodiments, a composition of cells generated by any of the methods described herein are administered by stereotactic injection, such as with a catheter. In some embodiments, a composition of cells generated by any of the methods described herein are administered to the striatum of a subject with Parkinson's disease.

Also provided herein is an exemplary method of differentiating neural cells, the method comprising: exposing the pluripotent stem cells to: (a) an inhibitor of bone morphogenetic protein (BMP) signaling; (b) an inhibitor of TGF-β/activing-Nodal signaling; and (c) at least one activator of Sonic Hedgehog (SHH) signaling. In some embodiments, during the exposing, the pluripotent stem cells are attached to a substrate. In some embodiments, during the exposing, the pluripotent stem cells are in a non-adherent culture vessel under conditions to produce a cellular spheroid.

In some embodiments, the method further comprises exposing the pluripotent stem cells to at least one inhibitor of GSK3β signaling. In some embodiments, during the exposing to the at least one inhibitor of GSK3β signaling, the pluripotent stem cells are attached to a substrate. In some embodiments, during the exposing to the at least one inhibitor of GSK3β signaling, the pluripotent stem cells are in a non-adherent culture vessel under conditions to produce a cellular spheroid.

In some embodiments, the inhibitor of TGF-β/activing-Nodal signaling is SB431542.

In some embodiments, the at least one activator of SHH signaling is SHH or purmorphamine. In some embodiments, the inhibitor of BMP signaling is LDN193189. In some embodiments, the at least one inhibitor of GSK3β signaling is CHIR99021.

In some embodiments, the exposing results in a population of differentiated neural cells. In some embodiments, the differentiated neural cells are floor plate midbrain progenitor cells, determined dopamine (DA) neuron progenitor cells, and/or, dopamine (DA) neurons.

The differentiated neural cells produced by any of the methods described herein are sometimes referred to as “corrected and differentiated cells.”

### IV. COMPOSITIONS AND FORMULATIONS

Also provided herein are populations of engineered cells, compositions containing engineered cells, and compositions enriched for engineered cells. The engineered cells are cells, e.g., PSCs, such as iPSCs, and cells differentiated therefrom, that have been edited to correct a gene variant in accordance with any of the methods described in Section II. In some embodiments, the engineered cells, the compositions containing engineered cells, and compositions enriched for engineered cells, are produced by the methods described herein, e.g., as described in Section II and Section III. In some embodments, the population of engineered cells, the composition containing engineered cells, and the compositions enriched for engineered cells, include engineered cells that are differentiated neural cells, such as floor plate midbrain progenitor cells, determined dopamine (DA) neuron progenitor cells, and/or dopamine (DA) neurons, or glial cells, e.g., microglial cells, astrocytes, oligodendrocytes, or ependymocytes. In some embodiments, the provided population of engineered cells is a population of the cell produced by any the methods described herein, e.g., as described in Section II and Section III. Accordngly, also provided herein is a population of the cell produced by any the methods described herein, e.g., as described in Section II and Section III, as well as compositions comprising the cell produced by any the methods described herein, e.g., as described in Section II and Section III, and compositions enriched for the cell produced by any the methods described herein, e.g., as described in Section II and Section III.

In some embodiments, the provided population of engineered cells, composition containing engineered cells, or composition enriched for engineered cells, include a cell population comprising cells that have been engineered to correct a gene variant associated with PD, such as a gene variant associated with PD that is within the human GBA1 locus. In some embodiments, at least 75%, 80%, 85%, 90%, 95%, 96%, 97%, 98%, or 99%, or 100 of the cells in the population of engineered cells, composition containing engineered cells, or composition enriched for engineered cells have been engineered to correct a gene variant associated with PD, such as a gene variant associated with PD that is within the human GBA1 locus. In some embodiments, the cells in the population of engineered cells, composition containing engineered cells, or composition enriched for engineered cells have been engineered to include a corrected form of the gene variant, e.g., SNP. In some embodiments, the cells have been engineered to correct the gene variant associated with PD by the methods described herein. In some embodiments, the cells that have been engineered to correct the gene variant associated with PD are less susceptible to causing, or contributing to, PD than the cells would be without the engineering.

### A. Exemplary Features of Compositions

In some embodiments, the cells produced by any of the methods described herein comprise the corrected form of the SNP instead of the SNP. In some embodiments, at least 10%, at least 20%, at least 30%, at least 40%, or at least 50% of the cells of any of the compositions described herein comprise the corrected form of the SNP instead of the SNP. In some embodiments, at least 10% of the cells of any of the compositions described herein comprise the corrected form of the SNP instead of the SNP. In some embodiments, at least 20% of the cells of any of the compositions described herein comprise the corrected form of the SNP instead of the SNP. In some embodiments, at least 30% of the cells of any of the compositions described herein comprise the corrected form of the SNP instead of the SNP. In some embodiments, at least 40% of the cells of any of the compositions described herein comprise the corrected form of the SNP instead of the SNP. In some embodiments, at least 50% of the cells of any of the compositions described herein comprise the corrected form of the SNP instead of the SNP. In some embodiments, at least 60% of the cells of any of the compositions described herein comprise the corrected form of the SNP instead of the SNP. In some embodiments, at least 70% of the cells of any of the compositions described herein comprise the corrected form of the SNP instead of the SNP. In some embodiments, at least 80% of the cells of any of the compositions described herein comprise the corrected form of the SNP instead of the SNP. In some embodiments, at least 90% of the cells of any of the compositions described herein comprise the corrected form of the SNP instead of the SNP.

In some embodiments, the differentiated cells produced by any of the methods described herein are determined dopamine (DA) neuron progenitor cells.

In some embodiments, the determined dopamine (DA) neuron progenitor cells are homozygous for a GBA1 gene encoding an asparagine at amino acid position 370 in the expressed GCase enzyme. In some embodiments, the determined dopamine (DA) neuron progenitor cells are homozygous for a GBA1 gene encoding a leucine at position 444 in the expressed GCase enzyme. In some embodiments, the determined dopamine (DA) neuron progenitor cells are homozygous for a GBA1 gene encoding a glutamic acid at position 326 in the expressed GCase enzyme.

In some embodiments, the determined dopamine (DA) neuron progenitor cells are homozygous for a GBA1 gene that includes a thymine wildtype variant of the rs76763715 SNP. In some embodiments, the determined dopamine (DA) neuron progenitor cells are homozygous for a GBA1 gene that includes an adenine wildtype variant of the rs421016 SNP. In some embodiments, the determined dopamine (DA) neuron progenitor cells are homozygous for a GBA1 gene that includes a cytosine wildtype variant of the rs2230288 SNP.

In some embodiments, the differentiated cells produced by any of the methods described herein are capable of producing dopamine (DA). In some embodiments, the differentiated cells produced by any of the methods described herein do not produce or do not substantially produce norepinephrine (NE). Thus, in some embodiments, the differentiated cells produced by any of the methods described herein are capable of producing DA but do not produce or do not substantially produce NE

In some embodiments, the determined dopamine (DA) neuron progenitor cells express EN1. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1.

In some embodiments, the determined dopamine (DA) neuron progenitor cells express CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express CORIN.

In some embodiments, the determined dopamine (DA) neuron progenitor cells express EN1 and CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1 and CORIN.

In some embodiments, less than 10% of determined dopamine (DA) neuron progenitor cells express TH. In some embodiments, the determined dopamine (DA) neuron progenitor cells express low levels of TH. In some embodiments, the determined dopamine (DA) neuron progenitor cells do not express TH. In some embodiments, the determined dopamine (DA) neuron progenitor cells express TH at lower levels than cells harvested or collected on other days. In some embodiments, some of the determined dopamine (DA) neuron progenitor cells express EN1 and CORIN and less than 10% of the cells express TH. In some embodiments, less than 10% of the determined dopamine (DA) neuron progenitor cells express TH, and at least about 20% of the cells express EN1. In some embodiments, less than 10% of the determined dopamine (DA) neuron progenitor cells express TH, and at least about 20% of the cells express CORIN. In some embodiments, less than 10% of the total determined dopamine (DA) neuron progenitor cells express TH, and at least about 20% of the cells express EN1 and CORIN.

In some embodiments, the differentiated cells produced by any of the methods described herein are dopamine (DA) neurons (e.g., midbrain fate DA neurons). In some embodiments, the midbrain fate dopamine (DA) neurons are FOXA2+/TH+ at the time of harvest. In some embodiments, the midbrain fate dopamine (DA) neurons are FOXA2+/TH+ by or on about day 18. In some embodiments, the midbrain fate dopamine (DA) neurons are FOXA2+/TH+ by or on about day 25.

### B. Compositions and Formulations

In some embodiments, the dose of cells comprising cells produced by any of the methods disclosed herein, is provided as a composition or formulation, such as a pharmaceutical composition or formulation. In some embodiments, the dose of cells comprises corrected and differentiated cells. In some embodiments, the dose of cells comprises cells produced by any of the methods described in Section III. In some embodiments, the dose of cells comprises cells produced by a combination of (1) any of the methods described in Section II, and (2) any of the methods described in Section III. In some embodiments, the dose of cells comprises cells produced by a process comprising (1) any of the methods of correcting gene variants described in Section II, and (2) any of the methods for differentiating cells described in Section III.

Such compositions can be used in accord with the provided methods, articles of manufacture, and/or with the provided compositions, such as in the prevention or treatment of diseases, conditions, and disorders, such as neurodegenerative disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the striatum. In some embodiments, the agents or cells can be administered by stereotactic injection into the striatum, such as in the putamen.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g. the striatum).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### V. METHODS OF TREATMENT

The present disclosure relates to methods of correcting gene variants associated with Parkinson's Disease (PD), such as gene variants of human GBA1, and methods of lineage specific differentiation of pluripotent stem cells (PSCs), including embryonic stem (ES) cells and induced pluripotent stem cells (iPSCs), including those in which a gene variant(s) associated with PD has been corrected, for use in treating neurodegenerative diseases. Specifically, the methods, compositions, and uses thereof provided herein contemplate correction of one or more gene variants associated with PD, e.g., as described in Section II, and differentiation of pluripotent stem cells that include the corrected form of the gene variant, e.g., SNP, as described, e.g., in Section III, for administration to subjects exhibiting a loss of a certain type of neuron, e.g., dopamine (DA) neurons, including Parkinson's disease. The methods, compositions, and uses thereof provided herein contemplate correction of one or more gene variants associated with PD, e.g., as described in Section II, and differentiation of pluripotent stem cells that include the corrected form of the gene variant, e.g., SNP, as described, e.g., in Section III, for administration to subjects exhibiting the one or more gene variants associated with PD.

Specifically, provided herein is a method of treatment, comprising administering to a subject a therapeutically effective amount of a therapeutic composition, e.g., any composition as described in Section IV, wherein the subject has a gene variant, e.g., SNP, associated with PD, such as a gene variant in human GBA1. In some embodiments, the subject has a gene variant in the GBA1 gene, e.g., a rs76763715 SNP, that results in an N370S amino acid change due to the presence of a serine, rather than an asparagine, at amino acid position 370 in the expressed GCase enzyme. In some embodiments, the subject has a gene variant in the GBA1 gene, e.g., a rs421016 SNP, that results in an L444P amino acid change due to the presence of a proline, rather than a leucine, at position 444 in the expressed GCase enzyme. In some embodiments, the subject has a gene variant in the GBA1 gene, e.g., a rs2230288 SNP, that results in an E326K amino acid change due to the presence of a lysine, rather than a glutamic acid, at position 326 in the expressed GCase enzyme.

In some embodiments, a subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [18F]-L-DOPA PET scan. In some embodiments, the subject exhibits [18F]-DG-PET evidence for a Parkinson's Disease Related Pattern (PDRP).

In some embodiments, the neurodegenerative disease is Parkinsonism. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has Parkinson's Disease. In some embodiments, the subject has moderate or advanced Parkinson's disease. In some embodiments, the subject has mild Parkinson's disease. In some embodiments, the subject has a MDS-UPDRS motor score of between 33 and 60.

In some embodiments, the subject has a target gene, e.g., GBA1, that includes a gene variant associated with PD. In some embodiments, the target gene that includes a gene variant associated with PD is GBA1. In some embodiments, the target gene is GBA1 and the gene variant associated with PD is a gene variant that encodes a serine, rather than an asparagine, at position 370 (N370S). In some embodiments, the target gene is GBA1 and encodes an amino acid sequence comprising the amino acid sequence set forth in SEQ ID NO: 7. In some embodiments, the target gene is GBA1 and the gene variant associated with PD is a gene variant that encodes a proline, rather than a leucine, at position 444 (L444P). In some embodiments, the target gene is GBA1 and encodes an amino acid sequence comprising the amino acid sequence set forth in SEQ ID NO: 9. In some embodiments, the target gene is GBA1 and the gene variant associated with PD is a gene variant that encodes a lysine, rather than a glutamic acid, at position 326 (E326K). In some embodiments, the target gene is GBA1 and encodes an amino acid sequence comprising the amino acid sequence set forth in SEQ ID NO: 10. In some embodiments, the target gene is GBA1 and encodes an amino acid sequence comprising the amino acid sequence set forth in any one of SEQ ID NOs: 7, 9, and 10.

In some embodiments, thee subject has a gene variant associated with PD that is a variant of rs76763715. In some embodiments, the subject has a gene variant associated with PD that is a variant of rs76763715 that encodes a serine, rather than an asparagine, at position 370 (N370S). In some embodiments, the subject has a gene variant associated with PD that is a a cytosine variant of rs76763715.

In some embodiments, the subject has a gene variant associated with PD that is a variant of rs421016. In some embodiments, the subject has a gene variant associated with PD that is a variant of rs421016 that encodes a proline, rather than a leucine, at position 444 (L444P). In some embodiments, the subject has a gene variant associated with PD that is a guanine variant of rs421016.

In some embodiments, the subject has a gene variant associated with PD that is a variant of rs2230288. In some embodiments, the subject has a gene variant associated with PD that is a variant of rs2230288 that encodes a lysine, rather than a glutamic acid, at position 326 (E326K). In some embodiments, the subject has a gene variant associated with PD that is a thymine variant of rs2230288.

In some embodiments, the therapeutic composition comprising cells, e.g., iPSCs, having a corrected form of the gene variant, is administered to treat a neurodegenerative disease, e.g., PD, using cells that include a corrected form of the gene variant associated with PD. By administering a therapeutic composition comprising cells, e.g., iPSCs, having a corrected form of the gene variant associated with PD, the risk of recurrence of the neurodegenerative disease, e.g., PD, is reduced.

In some embodiments, a dose of cells comprising a corrected form of the gene variant, e.g., as described in Section II, that have been differentiated, e.g., as described in Section III, is administered to subjects in accord with the provided methods, and/or with the provided articles of manufacture, and/or with the provided compositions, e.g., as described in Section IV. The dose of cells is a dose of cells, e.g., PSCs, such as iPSCs, comprising a corrected form of the gene variant, e.g., as described in Section II, that have been differentiated, e.g., as described in Section III. In some embodiments, the dose of cells is a dose of a composition of cells, e.g., as described in Section IV.

In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the striatum (e.g. putamen) of the subject. In some embodiments, the dose of cells is administered to one hemisphere of the subject's striatum (e.g. putamen). In some embodiments, the dose of cells is administered to both hemispheres of the subject's striatum (e.g. putamen).

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about about 3×10⁶ cells/hemisphere and 15×10⁶ cells/hemisphere. In some embodiments, the dose of cells is about about 3×10⁶ cells/hemisphere. In some embodiments, the dose of cells is about about 4×10⁶ cells/hemisphere. In some embodiments, the dose of cells is about about 5×10⁶ cells/hemisphere. In some embodiments, the dose of cells is about about 6×10⁶ cells/hemisphere. In some embodiments, the dose of cells is about about 7×10⁶ cells/hemisphere. In some embodiments, the dose of cells is about about 8×10⁶ cells/hemisphere. In some embodiments, the dose of cells is about about 9×10⁶ cells/hemisphere. In some embodiments, the dose of cells is about about 10×10⁶ cells/hemisphere. In some embodiments, the dose of cells is about about 11×10⁶ cells/hemisphere. In some embodiments, the dose of cells is about about 12×10⁶ cells/hemisphere. In some embodiments, the dose of cells is about about 13×10⁶ cells/hemisphere. In some embodiments, the dose of cells is about about 14×10⁶ cells/hemisphere. In some embodiments, the dose of cells is about about 15×10⁶ cells/hemisphere.

In some embodiments, the dose of cells is about about 5×10⁶ cells in each putamen. In some embodiments, the dose of cells is about about 10×10⁶ cells in each putamen.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g. differentiated cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In particular embodiments, the numbers and/or concentrations of cells refer to the number of TH-positive cells. In other embodiments, the numbers and/or concentrations of cells refer to the number or concentration of all cells administered.

In some embodiments, the cells are administered at a desired dosage, which in some aspects includes a desired dose or number of cells or cell type(s) and/or a desired ratio of cell types. Thus, the dosage of cells in some embodiments is based on a total number of cells and a desired ratio of the individual populations or sub-types In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In particular embodiments, the numbers and/or concentrations of cells refer to the number of TH-positive cells. In other embodiments, the numbers and/or concentrations of cells refer to the number or concentration of all cells administered.

In some aspects, the size of the dose is determined based on one or more criteria such as response of the subject to prior treatment, e.g. disease type and/or stage, and/or likelihood or incidence of the subject developing toxic outcomes, e.g., dyskinesia.

### VI. ARTICLES OF MANUFACTURE AND KITS

Also provided are articles of manufacture, systems, apparatuses, and kits useful in performing the provided methods.

Also provided are articles of manufacture, including: (i) one or more agent(s) capable of inducing a double strand break (DSB), and a donor template; and (ii) instructions for use of the one or more agent(s) and the donor template for performing any methods described herein.

Also provided are articles of manufacture, including: (i) one or more agent(s) comprising a recombinant nuclease for inducing a DNA break; and a donor template; and (ii) instructions for use of the one or more agent(s) and the donor template for performing any methods described herein.

Also provided are articles of manufacture, including: (i) one or more agent(s) comprising a recombinant nuclease for inducing a DSB; and a donor template; and (ii) instructions for use of the one or more agent(s) and the donor template for performing any methods described herein.

Also provided are articles of manufacture, including: (i) one or more agent(s) comprising a recombinant nuclease for inducing a SSB; and a donor template; and (ii) instructions for use of the one or more agent(s) and the donor template for performing any methods described herein.

In some of any such embodiments, the one or more agent(s) comprises a recombinant nuclease, such as any of the suitable recombinant nucleases described herein, e.g., in Section II.C. In some of any such embodiments, the one or more agent(s) comprises a fusion protein comprising a DNA cleavage domain and a DNA binding domain. In some embodiments, the DNA cleavage domain is or comprises a recombinant nuclease. In some of any such embodiments, the one or more agent(s) comprises a fusion protein comprising a recombinant nuclease and a DNA binding domain. In some of any such embodiments, the recombinant nuclease is selected from the group consisting of Cas9, a transcription activator-like effector nuclease (TALEN), and a zinc finger nuclease (ZFN). In some of any such embodiments, the recombinant nuclease is Cas9. In some of any such embodiments, the recombinant nuclease is a modified version of Cas9, such as an enhanced specificity Cas9 (eSpCas9) or a high fidelity Cas9 (HiFi Cas9). In some of any such embodiments, the recombinant nuclease is a TALEN. In some of any such embodiments, the recombinant nuclease is a ZFN.

In some embodiments comprising one or more agent(s) comprising a recombinant nuclease for inducing a SSB, the recombinant nuclease comprises one or more mutations in the RuvC catalytic domain or the HNH catalytic domain. Examples of such recombinant nucleases are described, e.g., in Section II.C.

In some of any such embodiments, the one or more agent(s) comprises a recombinant nuclease and a guide RNA. In some embodiments, the guide RNA is a sgRNA. In some of any such embodiments, the one or more agent(s) comprises a recombinant nuclease and a sgRNA. In some of any such embodiments, the recombinant nuclease is Cas9. In some of any such embodiments, the recombinant nuclease is a modified version of Cas9, such as an enhanced specificity Cas9 (eSpCas9) or a high fidelity Cas9 (HiFi Cas9). In some of any such embodiments, the Cas9 and the sgRNA are in a complex. In some embodiments, the complex is a ribonucleoprotein (RNP) complex.

In some of any such embodiments, the one or more agent(s) comprises a recombinant nuclease; a first guide RNA; and a second guide RNA. In some embodiments, the first guide RNA is a first sgRNA, and the second guide RNA is a first sgRNA. In some of any such embodiments, the one or more agent(s) comprises a recombinant nuclease; a first sgRNA; and a second sgRNA. In some of any such embodiments, the recombinant nuclease is Cas9. In some of any such embodiments, the recombinant nuclease is a modified version of Cas9, such as an enhanced specificity Cas9 (eSpCas9) or a high fidelity Cas9 (HiFi Cas9). In some of any such embodiments, the Cas9 and the first sgRNA are in a complex. In some of any such embodiments, the Cas9 and the second sgRNA are in a complex. In some of any such embodiments, the Cas9 and the first sgRNA are in a complex, and the Cas9 and the second sgRNA are in a complex. In some embodiments, the complex is a ribonucleoprotein (RNP) complex.

In some of any such embodiments, the one or more agent(s) are in a protein form. In some of any such embodiments, the one or more agent(s) are in a nucleic acid form. In some of any such embodiments, the one or more agent(s) include one or more components in protein form, e.g., the recombinant nuclease, and one or more components in nucleic acid form, e.g., the sgRNA. In some embodments, the nucleic acid form is DNA. In some embodments, the nucleic acid form is RNA.

In some of any such embodiments, the donor template is a ssODN. In some embodiments, the ssODN comprises a corrected form of the gene variant, e.g., SNP, of a gene variant in GBA1 that is associated with PD. In some embodiments, the ssODN comprises a 5′ ssODN arm and a 3′ ssODN arm. In some embodiments, the 5′ ssODN arm comprises the nucleic acid sequence of SEQ ID NO: 1 or 4. In some embodiments, the 3′ ssODN arm comprises the nucleic acid sequence of SEQ ID NO: 2. In some embodiments, the 5′ ssODN arm comprises the nucleic acid sequence of SEQ ID NO: 1 or 4, and the 3′ ssODN arm comprises the nucleic acid sequence of SEQ ID NO: 2. In some of any such embodiments, the ssODN comprises the nucleic acid sequence as set forth in SEQ ID NO: 3 or 5. In some embodiments, the 5′ ssODN arm comprises the nucleic acid sequence of SEQ ID NO: 1. In some embodiments, the 3′ ssODN arm comprises the nucleic acid sequence of SEQ ID NO: 2. In some embodiments, the 5′ ssODN arm comprises the nucleic acid sequence of SEQ ID NO: 1, and the 3′ ssODN arm comprises the nucleic acid sequence of SEQ ID NO: 2. In some of any such embodiments, the ssODN comprises the nucleic acid sequence as set forth in SEQ ID NO: 3. In some embodiments, the 5′ ssODN arm comprises the nucleic acid sequence of SEQ ID NO: 1, the 3′ ssODN arm comprises the nucleic acid sequence of SEQ ID NO: 2, and the ssODN comprises the nucleic acid sequence as set forth in SEQ ID NO: 3.

n some embodiments, the 5′ ssODN arm comprises the nucleic acid sequence of SEQ ID NO: 4. In some embodiments, the 3′ ssODN arm comprises the nucleic acid sequence of SEQ ID NO: 2. In some embodiments, the 5′ ssODN arm comprises the nucleic acid sequence of SEQ ID NO: 4, and the 3′ ssODN arm comprises the nucleic acid sequence of SEQ ID NO: 2. In some of any such embodiments, the ssODN comprises the nucleic acid sequence as set forth in SEQ ID NO: 5. In some embodiments, the 5′ ssODN arm comprises the nucleic acid sequence of SEQ ID NO: 4, the 3′ ssODN arm comprises the nucleic acid sequence of SEQ ID NO: 2, and the ssODN comprises the nucleic acid sequence as set forth in SEQ ID NO: 5.

In some embodiments, the 5′ ssODN arm comprises the nucleic acid sequence of SEQ ID NO: 25. In some embodiments, the 3′ ssODN arm comprises the nucleic acid sequence of SEQ ID NO: 26. In some embodiments, the 5′ ssODN arm comprises the nucleic acid sequence of SEQ ID NO: 25, and the 3′ ssODN arm comprises the nucleic acid sequence of SEQ ID NO: 26. In some of any such embodiments, the ssODN comprises the nucleic acid sequence as set forth in SEQ ID NO: 27. In some embodiments, the 5′ ssODN arm comprises the nucleic acid sequence of SEQ ID NO: 25, the 3′ ssODN arm comprises the nucleic acid sequence of SEQ ID NO: 26, and the ssODN comprises the nucleic acid sequence as set forth in SEQ ID NO: 27.

In some embodiments, the 5′ ssODN arm comprises the nucleic acid sequence of SEQ ID NO: 28. In some embodiments, the 3′ ssODN arm comprises the nucleic acid sequence of SEQ ID NO: 29. In some embodiments, the 5′ ssODN arm comprises the nucleic acid sequence of SEQ ID NO: 28, and the 3′ ssODN arm comprises the nucleic acid sequence of SEQ ID NO: 29. In some of any such embodiments, the ssODN comprises the nucleic acid sequence as set forth in SEQ ID NO: 30. In some embodiments, the 5′ ssODN arm comprises the nucleic acid sequence of SEQ ID NO: 28, the 3′ ssODN arm comprises the nucleic acid sequence of SEQ ID NO: 29, and the ssODN comprises the nucleic acid sequence as set forth in SEQ ID NO: 30.

In some embodiments, the 5′ ssODN arm comprises the nucleic acid sequence of SEQ ID NO: 31. In some embodiments, the 3′ ssODN arm comprises the nucleic acid sequence of SEQ ID NO: 32. In some embodiments, the 5′ ssODN arm comprises the nucleic acid sequence of SEQ ID NO: 31, and the 3′ ssODN arm comprises the nucleic acid sequence of SEQ ID NO: 32. In some of any such embodiments, the ssODN comprises the nucleic acid sequence as set forth in SEQ ID NO: 33. In some embodiments, the 5′ ssODN arm comprises the nucleic acid sequence of SEQ ID NO: 31, the 3′ ssODN arm comprises the nucleic acid sequence of SEQ ID NO: 32, and the ssODN comprises the nucleic acid sequence as set forth in SEQ ID NO: 33.

In some embodiments, the 5′ ssODN arm comprises the nucleic acid sequence of SEQ ID NO: 34. In some embodiments, the 3′ ssODN arm comprises the nucleic acid sequence of SEQ ID NO: 35. In some embodiments, the 5′ ssODN arm comprises the nucleic acid sequence of SEQ ID NO: 34, and the 3′ ssODN arm comprises the nucleic acid sequence of SEQ ID NO: 35. In some of any such embodiments, the ssODN comprises the nucleic acid sequence as set forth in SEQ ID NO: 36. In some embodiments, the 5′ ssODN arm comprises the nucleic acid sequence of SEQ ID NO: 34, the 3′ ssODN arm comprises the nucleic acid sequence of SEQ ID NO: 35, and the ssODN comprises the nucleic acid sequence as set forth in SEQ ID NO: 36.

In some embodiments, the 5′ ssODN arm comprises the nucleic acid sequence of SEQ ID NO: 37. In some embodiments, the 3′ ssODN arm comprises the nucleic acid sequence of SEQ ID NO: 38. In some embodiments, the 5′ ssODN arm comprises the nucleic acid sequence of SEQ ID NO: 37, and the 3′ ssODN arm comprises the nucleic acid sequence of SEQ ID NO: 38. In some of any such embodiments, the ssODN comprises the nucleic acid sequence as set forth in SEQ ID NO: 39. In some embodiments, the 5′ ssODN arm comprises the nucleic acid sequence of SEQ ID NO: 37, the 3′ ssODN arm comprises the nucleic acid sequence of SEQ ID NO: 38, and the ssODN comprises the nucleic acid sequence as set forth in SEQ ID NO: 39.

In some embodiments, the 5′ ssODN arm comprises the nucleic acid sequence of SEQ ID NO: 40. In some embodiments, the 3′ ssODN arm comprises the nucleic acid sequence of SEQ ID NO: 41. In some embodiments, the 5′ ssODN arm comprises the nucleic acid sequence of SEQ ID NO: 40, and the 3′ ssODN arm comprises the nucleic acid sequence of SEQ ID NO: 41. In some of any such embodiments, the ssODN comprises the nucleic acid sequence as set forth in SEQ ID NO: 42. In some embodiments, the 5′ ssODN arm comprises the nucleic acid sequence of SEQ ID NO: 40, the 3′ ssODN arm comprises the nucleic acid sequence of SEQ ID NO: 41, and the ssODN comprises the nucleic acid sequence as set forth in SEQ ID NO: 42.

In some embodiments, the 5′ ssODN arm comprises the nucleic acid sequence of SEQ ID NO: 43. In some embodiments, the 3′ ssODN arm comprises the nucleic acid sequence of SEQ ID NO: 44. In some embodiments, the 5′ ssODN arm comprises the nucleic acid sequence of SEQ ID NO: 43, and the 3′ ssODN arm comprises the nucleic acid sequence of SEQ ID NO: 44. In some of any such embodiments, the ssODN comprises the nucleic acid sequence as set forth in SEQ ID NO: 45. In some embodiments, the 5′ ssODN arm comprises the nucleic acid sequence of SEQ ID NO: 43, the 3′ ssODN arm comprises the nucleic acid sequence of SEQ ID NO: 44, and the ssODN comprises the nucleic acid sequence as set forth in SEQ ID NO: 45.

In some embodiments, the 5′ ssODN arm comprises the nucleic acid sequence of SEQ ID NO: 46. In some embodiments, the 3′ ssODN arm comprises the nucleic acid sequence of SEQ ID NO: 47. In some embodiments, the 5′ ssODN arm comprises the nucleic acid sequence of SEQ ID NO: 46, and the 3′ ssODN arm comprises the nucleic acid sequence of SEQ ID NO: 47. In some of any such embodiments, the ssODN comprises the nucleic acid sequence as set forth in SEQ ID NO: 48. In some embodiments, the 5′ ssODN arm comprises the nucleic acid sequence of SEQ ID NO: 46, the 3′ ssODN arm comprises the nucleic acid sequence of SEQ ID NO: 47, and the ssODN comprises the nucleic acid sequence as set forth in SEQ ID NO: 48.

In some embodiments, the 5′ ssODN arm comprises the nucleic acid sequence of SEQ ID NO: 49. In some embodiments, the 3′ ssODN arm comprises the nucleic acid sequence of SEQ ID NO: 50. In some embodiments, the 5′ ssODN arm comprises the nucleic acid sequence of SEQ ID NO: 49, and the 3′ ssODN arm comprises the nucleic acid sequence of SEQ ID NO: 50. In some of any such embodiments, the ssODN comprises the nucleic acid sequence as set forth in SEQ ID NO: 51. In some embodiments, the 5′ ssODN arm comprises the nucleic acid sequence of SEQ ID NO: 49, the 3′ ssODN arm comprises the nucleic acid sequence of SEQ ID NO: 50, and the ssODN comprises the nucleic acid sequence as set forth in SEQ ID NO: 51.

In some embodiments, the 5′ ssODN arm comprises the nucleic acid sequence of SEQ ID NO: 52. In some embodiments, the 3′ ssODN arm comprises the nucleic acid sequence of SEQ ID NO: 53. In some embodiments, the 5′ ssODN arm comprises the nucleic acid sequence of SEQ ID NO: 52, and the 3′ ssODN arm comprises the nucleic acid sequence of SEQ ID NO: 53. In some of any such embodiments, the ssODN comprises the nucleic acid sequence as set forth in SEQ ID NO: 54. In some embodiments, the 5′ ssODN arm comprises the nucleic acid sequence of SEQ ID NO: 52, the 3′ ssODN arm comprises the nucleic acid sequence of SEQ ID NO: 53, and the ssODN comprises the nucleic acid sequence as set forth in SEQ ID NO: 54.

In some embodiments, the 5′ ssODN arm comprises the nucleic acid sequence of SEQ ID NO: 55. In some embodiments, the 3′ ssODN arm comprises the nucleic acid sequence of SEQ ID NO: 56. In some embodiments, the 5′ ssODN arm comprises the nucleic acid sequence of SEQ ID NO: 55, and the 3′ ssODN arm comprises the nucleic acid sequence of SEQ ID NO: 56. In some of any such embodiments, the ssODN comprises the nucleic acid sequence as set forth in SEQ ID NO: 57. In some embodiments, the 5′ ssODN arm comprises the nucleic acid sequence of SEQ ID NO: 55, the 3′ ssODN arm comprises the nucleic acid sequence of SEQ ID NO: 56, and the ssODN comprises the nucleic acid sequence as set forth in SEQ ID NO: 57.

In some embodiments, the 5′ ssODN arm comprises the nucleic acid sequence of SEQ ID NO: 58. In some embodiments, the 3′ ssODN arm comprises the nucleic acid sequence of SEQ ID NO: 59. In some embodiments, the 5′ ssODN arm comprises the nucleic acid sequence of SEQ ID NO: 58, and the 3′ ssODN arm comprises the nucleic acid sequence of SEQ ID NO: 59. In some of any such embodiments, the ssODN comprises the nucleic acid sequence as set forth in SEQ ID NO: 60. In some embodiments, the 5′ ssODN arm comprises the nucleic acid sequence of SEQ ID NO: 58, the 3′ ssODN arm comprises the nucleic acid sequence of SEQ ID NO: 59, and the ssODN comprises the nucleic acid sequence as set forth in SEQ ID NO: 60.

In some embodiments, the 5′ ssODN arm comprises the nucleic acid sequence of SEQ ID NO: 61. In some embodiments, the 3′ ssODN arm comprises the nucleic acid sequence of SEQ ID NO:62. In some embodiments, the 5′ ssODN arm comprises the nucleic acid sequence of SEQ ID NO: 61, and the 3′ ssODN arm comprises the nucleic acid sequence of SEQ ID NO: 62. In some of any such embodiments, the ssODN comprises the nucleic acid sequence as set forth in SEQ ID NO: 63. In some embodiments, the 5′ ssODN arm comprises the nucleic acid sequence of SEQ ID NO: 61, the 3′ ssODN arm comprises the nucleic acid sequence of SEQ ID NO: 62, and the ssODN comprises the nucleic acid sequence as set forth in SEQ ID NO: 63.

Also provided are articles of manufacture, including: (i) one or more reagents for differentiation of pluripotent stem cells into floor plate midbrain progenitor cells, determined dopaminergic (DA) neuron progenitor cells, and/or dopaminergic (DA) neurons; and (ii) instructions for use of the one or more reagents for performing any methods described herein.

Also provided are articles of manufacture, including: (i) one or more agent(s) capable of inducing a double strand break (DSB); and a donor template; (ii) one or more reagents for differentiation of pluripotent stem cells into floor plate midbrain progenitor cells, determined dopamine (DA) neuron progenitor cells, and/or dopamine (DA) neurons; and instructions for use of the one or more agent(s), the donor template, and the one or more reagents for performing any methods described herein.

Also provided are articles of manufacture, including: (i) one or more agent(s) comprising a recombinant nuclease for inducing a DNA break; and a donor template; (ii) one or more reagents for differentiation of pluripotent stem cells into floor plate midbrain progenitor cells, determined dopaminergic (DA) neuron progenitor cells, and/or dopaminergic (DA) neurons; and instructions for use of the one or more agent(s), the donor template, and the one or more reagents for performing any methods described herein.

Also provided are articles of manufacture, including: (i) one or more agent(s) comprising a recombinant nuclease for inducing a DSB; and a donor template; (ii) one or more reagents for differentiation of pluripotent stem cells into floor plate midbrain progenitor cells, determined dopaminergic (DA) neuron progenitor cells, and/or dopaminergic (DA) neurons; and instructions for use of the one or more agent(s), the donor template, and the one or more reagents for performing any methods described herein.

Also provided are articles of manufacture, including: (i) one or more agent(s) comprising a recombinant nuclease for inducing a SSB; and a donor template; (ii) one or more reagents for differentiation of pluripotent stem cells into floor plate midbrain progenitor cells, determined dopaminergic (DA) neuron progenitor cells, and/or dopaminergic (DA) neurons; and instructions for use of the one or more agent(s), the donor template, and the one or more reagents for performing any methods described herein.

In some of any such embodiments, the reagent for differentiation is or includes a small molecule, capable of inhibiting TGF-β/activing-Nodal signaling. In some of any such embodiments, the reagent for differentation is or includes SB431542. In some of any such embodiments, the reagent for differentiation is or includes a small molecule, capable of activating SHH signaling. In some of any such embodiments, the reagent for activating SHH signaling is or includes SHH. In some of any such embodiments, the reagent for activating SHH signaling is or includes purmorphamine. In some of any such embodiments, the reagent for activating SHH signaling is or includes SHH and purmorphamine. In some of any such embodiments, the reagent for differentiation is or includes a small molecule, capable of inhibiting BMP signaling. In some of any such embodiments, the reagent for inhibiting BMP signaling is LDN193189. In some of any such embodiments, the reagent for differentiation is or includes a small molecule, capable of inhibiting GSK3β signaling. In some of any of such embodiments, the reagent is or includes CHIR99021. In some of any of such embodiments, the reagent for differentiation is or includes one or more of BDNF, GDNF, dbcAMP, ascorbic acid, TGFβ3, and DAPT. The reagents in the kit in one embodiment may be in solution, may be frozen, or may be lyophilized.

Also provided are articles of manufacture, including (i) any composition described herein; and (ii) instructions for administering the composition to a subject.

In some embodiments, the articles of manufacture or kits include one or more containers, typically a plurality of containers, packaging material, and a label or package insert on or associated with the container or containers and/or packaging, generally including instructions for use, e.g., instructions for reagents for differentiation of pluripotent cells, e.g., differentiation of iPSCs into floor plate midbrain progenitor cells, determined dopamine (DA) neuron progenitor cells, and/or dopamine (DA) neurons, and instructions to carry out any of the methods provided herein. In some aspects, the provided articles of manufacture contain reagents for differentiation and/or maturation of cells, for example, at one or more steps of the manufacturing process, such as any reagents described in any steps of Sections III and IV.

Also provided are articles of manufacture and kits containing corrected and differentiated cells, such as those generated using the methods provided herein, and optionally instructions for use, for example, instructions for administering. In some embodiments, the instructions provide directions or specify methods for assessing if a subject, prior to receiving a cell therapy, is likely or suspected of being likely to respond and/or the degree or level of response following administration of differentiated cells for treating a disease or disorder. In some aspects, the articles of manufacture can contain a dose or a composition of corrected and differentiated cells.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the reagents and/or cell compositions are packaged separately. In some embodiments, each container can have a single compartment. In some embodiments, other components of the articles of manufacture or kits are packaged separately, or together in a single compartment.

### VII. EXEMPLARY EMBODIMENTS

Among the provided embodiments are:

1. A method of correcting a gene variant associated with Parkinson's Disease, the method comprising:
introducing, into a cell, one or more agents comprising a recombinant nuclease for inducing a DNA break within an endogenous target gene in the cell, wherein the target gene is human GBA1 and includes a single nucleotide polymorphism (SNP) that is associated with Parkinson's Disease; and
introducing, into the cell, a single-stranded DNA oligonucleotide (ssODN), wherein the ssODN is homologous to the target gene and comprises a corrected form of the SNP,
wherein the introducing of the one or more agents and the ssODN results in homology-directed repair (HDR) and integration of the ssODN into the target gene.

2. A method of correcting a gene variant associated with Parkinson's Disease, the method comprising:
introducing into an induced pluripotent stem cell (iPSC) one or more agents comprising a recombinant nuclease for inducing a DNA break within an endogenous target gene in the cell, wherein the target gene is human GBA1 and comprises a single nucleotide polymorphism (SNP) that is associated with Parkinson's Disease; and
introducing into the cell a single-stranded DNA oligonucleotide (ssODN), wherein the ssODN is homologous to the target gene and comprises a corrected form of the SNP,
wherein (i) the introducing of the one or more agents and the ssODN results in homology-directed repair (HDR) and integration of the ssODN into the target gene; and (ii) after the integration of the ssODN into the target gene, the target gene comprises the corrected form of the SNP instead of the SNP.

3. A method of correcting a gene variant associated with Parkinson's Disease, the method comprising:
introducing, into a cell, a single-stranded DNA oligonucleotide (ssODN);
wherein the cell comprises a DNA break within an endogenous target gene in the cell,
wherein the target gene is human GBA1 and includes a single nucleotide polymorphism (SNP) that is associated with Parkinson's Disease,
wherein the ssODN is homologous to the target gene and comprises a corrected form of the SNP, and
wherein the introducing results in HDR and integration of the ssODN into the target gene.

4. The method of embodiment 1 or embodiment 2, wherein the DNA break is a double strand break (DSB) at a cleavage site within the endogenous target gene.

5. The method of embodiment 3, wherein the DNA break is a DSB at a cleavage site within the endogenous target gene.

6. The method of embodiment 4, wherein the DSB is induced by one or more agents comprising a recombinant nuclease.

7. The method of any one of embodiments 1, 2, 4, and 6, wherein the recombinant nuclease is capable of cleaving both strands of double stranded DNA.

8. The method of any one of embodiments 1, 2, 4, 6, and 7, wherein the recombinant nuclease is selected from the group consisting of a Cas nuclease, a transcription activator-like effector nuclease (TALEN), and a zinc finger nuclease (ZFN).

9. The method of any one of embodiments 1, 2, 4, and 6-8, wherein the recombinant nuclease is a Cas nuclease.

10. The method of embodiment 8 or embodiment 9, wherein the one or more agents comprises the Cas nuclease and a single guide RNA (sgRNA).

11. The method embodiment 10, wherein the Cas nuclease and the sgRNA are in a complex when they are introduced into the cell.

12. The method of embodiment 10 or embodiment 11, wherein the Cas nuclease and the sgRNA are introduced as a ribonucleoprotein (RNP) complex.

13. The method of any one of embodiments 8-10, wherein the Cas nuclease is introduced into the cell by introducing a nucleic acid encoding the Cas nuclease into the cell.

14. The method of embodiment 13, wherein the nucleic acid encoding the Cas nuclease is DNA.

15. The method of embodiment 13, wherein the nucleic acid encoding the Cas nuclease is RNA.

16. The method of any one of embodiments 8-15, wherein the Cas nuclease is selected from the group consisting of Cas3, Cas9, Cas10, Cas12, and Cas13.

17. The method of embodiment 16, wherein the Cas nuclease is Cas9 or a variant thereof.

18. The method of embodiment 16 or embodiment 17, wherein the Cas9 or a variant thereof is from a bacteria selected from the group consisting of Streptococcus pyogenes, Staphylococcus aureus, Neisseria meningitides, Campylobacter jejuni, and Streptococcus thermophilis.

19. The method of embodiment 17 or embodiment 18, wherein the Cas9 or a variant thereof is a Cas9 variant that exhibits reduced off-target effector activity.

20. The method of any oen of embodiments 17-19, wherein the Cas9 variant is an enhanced specificity Cas 9 (eSpCas9) or a high fidelity Cas 9 (HiFiCas9).

20. The method of embodiment 18-20, wherein the Cas9 or a variant thereof is from Streptococcus pyogenes.

21. The method of any one of embodiments 1, 2, 4, and 6-8, wherein the recombinant nuclease is a TALEN.

22. The method of any one of embodiments 1, 2, 4, and 6-8, wherein the recombinant nuclease is a ZFN.

23. The method of any one of embodiments 1, 2, 4, and 6-85-7, wherein the recombinant nuclease is introduced into the cell by introducing a nucleic acid encoding the recombinant nuclease into the cell.

24. The method of embodiment 21, wherein the TALEN is introduced into the cell by introducing a nucleic acid encoding the TALEN into the cell.

25. The method of embodiment 22, wherein the ZFN is introduced into the cell by introducing a nucleic acid encoding the ZFN into the cell.

26. The method of any one of embodiments 1, 2, 4, and 6-8, wherein the recombinant nuclease is introduced into the cell as a protein.

27. The method of embodiment 21, wherein the TALEN is introduced into the cell as a protein.

28. The method of embodiment 21, wherein the ZFN is introduced into the cell as a protein.

29. The method of any one of embodiments 1-28, wherein the cleavage site is at a position that is less than 200, 180, 160, 140, 120, 100, 90, 80, 70, 60, 50, 40, 30, or 20 nucleotides from the SNP.

30. The method of any one of embodiments 1-29, wherein the ssODN comprises a nucleic acid sequence that is substantially homologous to a targeting sequence in the target gene, wherein the targeting sequence comprises the SNP.

31. The method of embodiment 30, wherein the ssODN comprises a nucleic acid sequence that has at least 80%, 81%, 82%, 83%, 84%, 85%, 86%, 87%, 88%, 89%, 90%, 91%, 92%, 93%, 94%, 95%, 96%, 97%, 98%, or 99% sequence identity to the target gene.

32. The method of embodiment 30 or embodiment 31, wherein the ssODN comprises a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence.

33. The method of any one of embodiments 30-32, wherein the ssODN comprises a nucleic acid sequence that is not homologous to the targeting sequence at the nucleotide of the SNP.

34. The method of any one of embodiments 30-33, wherein the targeting sequence is between about 50 and about 500 nucleotides in length, optionally between 50 and 450, 50 and 400, 50 and 350, 50 and 300, 50 and 250, 50 and 200, 50 and 175, 50 and 150, 50 and 125, 50 and 100, 75 and 450, 75 and 400, 75 and 350, 75 and 300, 75 and 250, 75 and 200, 75 and 175, 75 and 150, 75 and 125, 75 and 100, 100 and 450, 100 and 400, 100 and 350, 100 and 300, 100 and 250, 100 and 200, 100 and 175, 100 and 150, or 100 and 125 nucleotides in length.

35. The method of any one of embodiments 30-34, wherein the targeting sequence comprises the SNP and a protospacer adjacent motif (PAM) sequence.

36. The method of embodiment 35, wherein the ssODN comprises a nucleic acid sequence that comprises a PAM sequence that is homologous to the PAM sequence in the targeting sequence.

37. The method of embodiment 35, wherein the ssODN comprises a nucleic acid sequence that comprises a PAM sequence that is not homologous to the PAM sequence in the targeting sequence at one or more nucleotide positions, wherein the integration of the ssODN into the targeting sequence results in a silent mutation in the PAM sequence.

38. The method of any one of embodiments 30-37, wherein the ssODN comprises a nucleic acid sequence that comprises one or more nucleotides that are not homologous to corresponding nucleotides of the the targeting sequence, and wherein the one or more nucleotides comprises one or more nucleotides that introduce a restriction site into the target gene that is recognized by one or more restriction enzymes.

39. The method of any one of embodiments 1-38, wherein, after the integration of the ssODN into the target gene, the target gene comprises the corrected form of the SNP instead of the SNP.

40. The method of any one of embodiments 1-39, wherein the corrected form of the SNP is not associated with PD.

41. The method of any one of embodiments 1-40, wherein the corrected form of the SNP is a wildtype form of the SNP.

42. The method of any one of embodiments 1-41, wherein the target gene is human GBA1.

43. The method of embodiment 42, wherein the SNP is rs76763715.

44. The method of embodiment 43, wherein the rs76763715 is a cytosine variant.

45. The method of embodiment 43 or embodiment 44, wherein the GBA1 comprising the SNP encodes a serine, rather than an asparagine, at amino acid position 370 (N370S).

46. The method of any one of embodiments 43-45, wherein the ssODN comprises a 5′ ssODN arm and a 3′ ssODN arm, and wherein the 5′ ssODN arm comprises the nucleic acid sequence set forth in any one of SEQ ID NO: 1, 4, 25, 28, 31, 34, 37, 40, 43, 46, 49, 52, 55, 58, and 61; and/or the 3′ ssODN arm comprises the nucleic acid sequence set forth in any one of SEQ ID NOS: 2, 26, 29, 32, 35, 38, 41, 44, 47, 50, 53, 56, 59, and 62.

47. The method of any one of embodiments 43-46, wherein the ssODN comprises the nucleic acid sequence set forth in any one of SEQ ID NO: 3, 5, 27, 30, 33, 36, 39, 42, 45, 48, 51, 54, 57, 60, and 63

48. The method of any one of embodiments 43-47, wherein the corrected form of the SNP is a thymine wildtype variant.

49. The method of any one of embodiments 43-48, wherein, after the integration of the ssODN into the GBA1, the GBA1 comprises the corrected form of the SNP and encodes an asparagine at amino acid position 370.

50. The method of embodiment 42, wherein the SNP is rs421016.

51. The method of embodiment 50, wherein the rs421016 is a guanine variant.

52. The method of embodiment 50 or embodiment 51, wherein the GBA1 comprising the SNP encodes a proline, rather than a leucine, at amino acid position 444 (L444P).

53. The method of any one of embodiments 50-52, wherein the corrected form of the SNP is an adenine wildtype variant.

54. The method of any one of embodiments 50-53, wherein, after the integration of the ssODN into the GBA1, the GBA1 comprises the corrected form of the SNP and encodes a leucine at amino acid position 444.

55. The method of embodiment 42, wherein the SNP is rs2230288.

56. The method of embodiment 55, wherein the rs2230288 is a thymine variant.

57. The method of embodiment 55 or embodiment 56, wherein the GBA1 comprising the SNP encodes a lysine, rather than a glutamic acid, at position 326 (E326K).

58. The method of any one of embodiments 55-57, wherein the corrected form of the SNP is a cytosine wildtype variant.

59. The method of any one of embodiments 55-58, wherein, after the integration of the ssODN into the GBA1, the GBA1 comprises the corrected form of the SNP and encodes a glutamic acid at position 326.

60. The method of any one of embodiments 11-20 and 29-59, wherein the sgRNA comprises a CRISPR targeting RNA (crRNA) sequence that is homologous to a sequence in the target gene that includes the cleavage site, optionally wherein the crRNA sequence has 100% sequence identity to the sequence in the target gene that includes the cleavage site.

61. The method of embodiment 60, wherein the sequence in the target gene that includes the cleavage site is immediately upstream of the PAM sequence.

62. The method of embodiment 60 or embodiment 61, wherein the crRNA sequence comprises the nucleic acid sequence set forth in any one of SEQ ID NOS: 8 and 13-24.

63. The method of any of embodiments 60-62, wherein the crRNA sequence and the ssODN sequence comprise the nucleic acid sequences set forth in:
SEQ ID NOS: 8 and 3, respectively;
SEQ ID NOS: 8 and 5, respectively;
SEQ ID NOS: 8 and 33, respectively;
SEQ ID NOS: 13 and 27, respectively;
SEQ ID NOS: 14 and 30, respectively;
SEQ ID NOS: 15 and 36, respectively;
SEQ ID NOS: 16 and 39, respectively;
SEQ ID NOS: 17 and 42, respectively;
SEQ ID NOS: 18 and 45, respectively;
SEQ ID NOS: 19 and 48, respectively;
SEQ ID NOS: 20 and 51, respectively;
SEQ ID NOS: 21 and 54, respectively;
SEQ ID NOS: 22 and 57, respectively;
SEQ ID NOS: 23 and 60, respectively; or
SEQ ID NOS: 24 and 63, respectively.

64. The method of embodiment 1 or embodiment 2, wherein the endogenous target gene comprises a sense strand and an antisense strand, and the DNA break comprises a single strand break (SSB) at a cleavage site in the sense strand or the antisense strand.

65. The method of embodiment 1 or embodiment 2, wherein the endogenous target gene comprises a sense strand and an antisense strand, and the DNA break comprises a SSB at a cleavage site in the sense strand, and a SSB at a cleavage site in the antisense strand, thereby resulting in a DSB.

66. The method of embodiment 3, wherein the endogenous target gene comprises a sense strand and an antisense strand, and the DNA break comprises a single strand break (SSB) at a cleavage site within the endogenous target gene.

67. The method of embodiment 3, wherein the endogenous target gene comprises a sense strand and an antisense strand, and the DNA break comprises a SSB at a cleavage site in the sense strand, and a SSB at a cleavage site in the antisense strand, thereby resulting in a DSB.

68. The method of embodiment 66, wherein the SSB is induced by one or more agents comprising a recombinant nuclease.

69. The method of embodiment 67, wherein the SSB in the sense strand and the SSB in the antisense strand are induced by one or more agents comprising a recombinant nuclease.

70. The method of any one of embodiments 1, 3, 64, 66, 68, and 69, wherein the recombinant nuclease lacks the ability to induce a DSB by cleaving both strands of double stranded DNA.

71. The method of any one of embodiments 1, 3, 64, 65, and 68-70, wherein the one or more agents comprises a recombinant nuclease, a first sgRNA, and a second sgRNA.

72. The method of any one of embodiments 1, 3, 64, 65, and 68-71, wherein the recombinant nuclease is selected from the group consisting of a Cas nuclease, a transcription activator-like effector nuclease (TALEN), and a zinc finger nuclease (ZFN).

73. The method of embodiment 72, wherein the recombinant nuclease is a Cas nuclease.

74. The method embodiment 73, wherein (i) the Cas nuclease and the first sgRNA are in a complex when they are introduced into the cell; and/or (ii) the Cas nuclease and the second sgRNA are in a complex when they are introduced into the cell.

75. The method of embodiment 73 or embodiment 74, wherein (i) the Cas nuclease and the first sgRNA are introduced into the cell as a ribonucleoprotein (RNP) complex; and/or (ii) the Cas nuclease and the second sgRNA are introduced into the cell as a RNP complex.

76. The method of embodiment 72 or embodiment 73, wherein the Cas nuclease is introduced into the cell by introducing a nucleic acid encoding the Cas nuclease into the cell.

77. The method of embodiment 76, wherein the nucleic acid encoding the Cas nuclease is DNA.

78. The method of embodiment 76, wherein the nucleic acid encoding the Cas nuclease is RNA.

79. The method of any one of embodiments 72-78, wherein the Cas nuclease comprises one or more mutations such that the Cas nuclease is converted into a nickase that lacks the ability to cleave both strands of a double stranded DNA molecule.

80. The method of any one of embodiments 72-78, wherein the Cas nuclease comprises one or more mutations such that the Cas nuclease is converted into a nickase that is able to cleave only one strand of a double stranded DNA molecule.

81. The method of any one of embodiments 72-80, wherein the Cas nuclease is selected from the group consisting of Cas3, Cas9, Cas10, Cas12, and Cas13.

82. The method of embodiment 81, wherein the Cas nuclease is Cas9.

83. The method of embodiment 81 or embodiment 82, wherein the Cas9 is from a bacteria selected from the group consisting of Streptococcus pyogenes, Staphylococcus aureus, Neisseria meningitides, Campylobacter jejuni, and Streptococcus thermophilis.

84. The method of embodiment 83, wherein the Cas9 is from Streptococcus pyogenes.

85. The method of embodiment 84, wherein the Cas9 comprises one or more mutations in the RuvC catalytic domain, optionally wherein the one or more mutations is in one or more of the RuvC I, RuvC II, or RuvC III motifs.

86. The method of embodiment 85, wherein the one or more mutations comprises a D10A mutation in the RuvC I motif.

87. The method of embodiment 84, wherein the Cas9 comprises one or more mutations in the HNH catalytic domain.

88. The method of embodiment 87, wherein the one or more mutations in the HNH catalytic domain is selected from the group consisting of H840A, H854A, and H863A.

89. The method of embodiment 87 or embodiment 88, wherein the one or more mutations in the HNH catalytic domain comprises a H840A mutation.

90. The method of embodiment 84, wherein the Cas9 comprises a mutation selected from the group consisting of D10A, H840A, H854A, and H863A.

91. The method of embodiment 72, wherein the recombinant nuclease is a TALEN.

92. The method of embodiment 91, wherein the TALEN is introduced into the cell by introducing a nucleic acid encoding the TALEN into the cell.

93. The method of embodiment 91, wherein the TALEN is introduced into the cell as a protein.

94. The method of any one of embodiments 91-93, wherein the TALEN comprises one or more mutations such that the TALEN is converted into a nickase that lacks the ability to cleave both strands of a double stranded DNA molecule.

95. The method of any one of embodiments 91-93, wherein the TALEN comprises one or more mutations such that the TALEN is converted into a nickase that is able to cleave only one strand of a double stranded DNA molecule.

96. The method of embodiment 72, wherein the recombinant nuclease is a ZFN.

97. The method of embodiment 96, wherein the ZFN is introduced into the cell by introducing a nucleic acid encoding the ZFN into the cell.

98. The method of embodiment 96, wherein the ZFN is introduced into the cell as a protein.

99. The method of any one of embodiments 64 and 66-98, wherein the cleavage site is at a position that is less than 200, 180, 160, 140, 120, 100, 90, 80, 70, 60, 50, 40, 30, or 20 nucleotides from the SNP.

100. The method of any one of embodiments 65-98, wherein the cleavage site in the sense strand is at a position that is less than 200, 180, 160, 140, 120, 100, 90, 80, 70, 60, 50, 40, 30, or 20 nucleotides from the SNP; and/or the cleavage site in the antisense strand is at a position that is less than 200, 180, 160, 140, 120, 100, 90, 80, 70, 60, 50, 40, 30, or 20 nucleotides from the SNP.

101. The method of any one of embodiments 64-100, wherein the ssODN comprises a nucleic acid sequence that is substantially homologous to a targeting sequence in the target gene that includes the SNP.

102. The method of embodiment 101, wherein the nucleic acid sequence has at least 80%, 81%, 82%, 83%, 84%, 85%, 86%, 87%, 88%, 89%, 90%, 91%, 92%, 93%, 94%, 95%, 96%, 97%, 98%, or 99% sequence identity to the target gene.

103. The method of embodiment 101 or embodiment 102, wherein the nucleic acid sequence is at least 80%, 81%, 82%, 83%, 84%, 85%, 86%, 87%, 88%, 89%, 90%, 91%, 92%, 93%, 94%, 95%, 96%, 97%, 98%, or 99% homologous to the targeting sequence.

104. The method of any one of embodiments 101-103, wherein the nucleic acid sequence is not homologous to the targeting sequence at the SNP.

105. The method of any one of embodiments 101-104, wherein the targeting sequence has a length that is between 50 and 500 nucleotides in length, optionally between 50 and 450, 50 and 400, 50 and 350, 50 and 300, 50 and 250, 50 and 200, 50 and 175, 50 and 150, 50 and 125, 50 and 100, 75 and 450, 75 and 400, 75 and 350, 75 and 300, 75 and 250, 75 and 200, 75 and 175, 75 and 150, 75 and 125, 75 and 100, 100 and 450, 100 and 400, 100 and 350, 100 and 300, 100 and 250, 100 and 200, 100 and 175, 100 and 150, or 100 and 125 nucleotides in length.

106. The method of any one of embodiments 101-105, wherein the sense strand comprises the targeting sequence, and wherein the targeting sequence includes the SNP and a protospacer adjacent motif (PAM) sequence.

107. The method of embodiment 106, wherein the antisense strand comprises a sequence that is complementary to the targeting sequence and includes a PAM sequence.

108. The method of any one of embodiments 101-105, wherein the antisense strand comprises the targeting sequence, and wherein the targeting sequence includes the SNP and a PAM sequence.

109. The method of embodiment 108, wherein the sense strand comprises a sequence that is complementary to the targeting sequence and includes a PAM sequence.

110. The method of any one of embodiments 106-109, wherein the nucleic acid sequence comprises a PAM sequence that is homologous to the PAM sequence in the targeting sequence.

111. The method of any one of embodiments 106-109, wherein the nucleic acid sequence comprises a PAM sequence that is not homologous to the PAM sequence in the targeting sequence at one or more positions that result in a silent mutation.

112. The method of any one of 101-111, wherein the nucleic acid sequence comprises one or more nucleotides that are not homologous to the targeting sequence, and wherein the one or more nucleotides comprises one or more nucleotides that introduce a restriction site that is recognized by one or more restriction enzymes.

113. The method of any one of embodiments 1-112, wherein, after the integration of the ssODN into the target gene, the target gene comprises the corrected form of the SNP instead of the SNP.

114. The method of any one of embodiments 1-113, wherein the corrected form of the SNP is not associated with PD.

115. The method of any one of embodiments 1-114, wherein the corrected form of the SNP is a wildtype form of the SNP.

116. The method of any one of embodiments 1-115, wherein the target gene is human GBA1.

117. The method of embodiment 116, wherein the SNP is rs76763715.

118. The method of embodiment 117, wherein the rs76763715 is a cytosine variant.

119. The method of embodiment 116 or embodiment 117, wherein the GBA1 comprising the SNP encodes a serine, rather than an asparagine, at amino acid position 370 (N370S).

120. The method of any one of embodiments 117-119, wherein the corrected form of the SNP is a thymine wildtype variant.

121. The method of any one of embodiments 117-120, wherein, after the integration of the ssODN into the GBA1, the GBA1 comprises the corrected form of the SNP and encodes an asparagine at amino acid position 370.

122. The method of embodiment 116, wherein the SNP is rs421016.

123. The method of embodiment 122, wherein the rs421016 is a guanine variant.

124. The method of embodiment 122 or embodiment 123, wherein the GBA1 comprising the SNP encodes a proline, rather than a leucine, at amino acid position 444 (L444P).

125. The method of any one of embodiments 119-121, wherein the corrected form of the SNP is an adenine wildtype variant.

126. The method of any one of embodiments 119-122, wherein, after the integration of the ssODN into the GBA1, the GBA1 comprises the corrected form of the SNP and encodes a leucine at amino acid position 444.

127. The method of embodiment 116, wherein the SNP is rs2230288.

128. The method of embodiment 127, wherein the rs2230288 is a thymine variant.

129. The method of embodiment 127 or embodiment 128, wherein the GBA1 comprising the SNP encodes a lysine, rather than a glutamic acid, at position 326 (E326K).

130. The method of any one of embodiments 127-129, wherein the corrected form of the SNP is a cytosine wildtype variant.

131. The method of any one of embodiments 127-130, wherein, after the integration of the ssODN into the GBA1, the GBA1 comprises the corrected form of the SNP and encodes a glutamic acid at position 326.

132. The method of any one of embodiments 71-131, wherein the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site.

133. The method of embodiment 132, wherein the crRNA sequence of the first sgRNA has 100% sequence identity to the sequence in the sense strand of the target gene that includes the cleavage site; and/or the crRNA sequence of the second sgRNA has 100% sequence identity to the sequence in the antisense strand of the target gene that includes the cleavage site.

134. The method of embodiment 132 or embodiment 133, wherein the sequence in the sense strand of the target gene that includes the cleavage site is immediately upstream of the PAM sequence; and/or the sequence in the antisense strand of the target gene that includes the cleavage site is immediately upstream of the PAM sequence.

135. The method of any one of embodiments 1-134, wherein the cell is an induced pluripotent stem cell (iPSC).

136. The method of embodiment 135, wherein the iPSC is artificially derived from a non-pluripotent cell from a subject.

137. The method of embodiment 136, wherein the non-pluripotent cell is a fibroblast.

138. The method of embodiment 136 or embodiment 137, wherein the subject has Parkinson's Disease.

139. The method of any one of embodiments 39-63 and 112-138, wherein, after the integration of the ssODN into the target gene, the method further comprises contacting DNA isolated from the cell with the one or more restriction enzymes.

140. The method of embodiment 139, wherein, after the contacting, the method further comprises determining whether the DNA isolated from the cell has been cleaved at the restriction site.

141. The method of embodiment 140, wherein, if the DNA has been cleaved, the cell is identified as a cell comprising an integrated ssODN.

142. The method of any one of embodiments 1-141, wherein, after integration of the ssODN into the target gene, the method further comprises determining whether the cell comprises an integrated ssODN, optionally by one or more of of CIRCLE-seq, genomic qPCR, whole genome sequencing (WGS), targeted Sanger sequencing, and deep exome sequencing.

143. A complex for correcting a gene variant associated with Parkinson's Disease, comprising:
a Cas nuclease; and
a sgRNA comprising a CRISPR targeting RNA (crRNA) sequence that is homologous to a sequence in a target gene that includes a cleavage site,
wherein the target gene is human GBA1 and includes a single nucleotide polymorphism (SNP) that is associated with Parkinson's Disease.

144. The complex of embodiment 143, wherein the Cas nuclease is selected from the group consisting of Cas3, Cas9, Cas10, Cas12, and Cas13.

145. The complex of embodiment 143 or embodiment 144, wherein the Cas nuclease is Cas9 or a variant thereof.

146. The complex of embodiment 144 or embodiment 145, wherein the Cas9 is from a bacteria selected from the group consisting of Streptococcus pyogenes, Staphylococcus aureus, Neisseria meningitides, Campylobacter jejuni, and Streptococcus thermophilis.

147. The complex of embodiment 145 or embodiment 146, wherein the Cas9 or a variant thereof is a Cas9 variant that exhibits reduced off-target effector activity, optionally wherein the Cas9 variant is an enhanced specificity Cas 9 (eSpCas9) or a high fidelity Cas 9 (HiFiCas9).

148. The complex of embodiment 146, wherein the Cas9 is from Streptococcus pyogenes.

149. The complex of any one of embodiments 143-148, wherein the sgRNA comprises a CRISPR targeting RNA (crRNA) sequence that is homologous to a sequence in the target gene that includes the cleavage site.

150. The complex of embodiment 149, wherein the crRNA sequence has 100% sequence identity to the sequence in the target gene that includes the cleavage site.

151. The complex of embodiment 149 or embodiment 150, wherein the crRNA sequence comprises the nucleic acid sequence set forth in any one of SEQ ID NOS: 8 and 13-24.

152. The complex of any one of embodiments 143-151, wherein the Cas nuclease and the sgRNA form a ribonucleoprotein (RNP) complex.

153. A combination for correcting a gene variant associated with Parkinson's Disease, comprising:
a Cas nuclease;
a sgRNA comprising a CRISPR targeting RNA (crRNA) sequence that is homologous to a sequence in a target gene that includes a cleavage site, wherein the target gene is human GBA1 and includes a single nucleotide polymorphism (SNP) that is associated with Parkinson's Disease; and
a single-stranded DNA oligonucleotide (ssODN), wherein the ssODN is homologous to the target gene and comprises a corrected form of the SNP.

154. The combination of embodiment 153, wherein the Cas nuclease is Cas9 or a variant thereof.

155. The combination of embodiment 154, wherein the Cas9 or a variant thereof is from Streptococcus pyogenes.

156. The combination of embodiment 154 or embodiment 155, wherein the Cas9 or a variant thereof is a Cas9 variant that exhibits reduced off-target effector activity, optionally wherein the Cas9 variant is an enhanced specificity Cas 9 (eSpCas9) or a high fidelity Cas 9 (HiFiCas9).

157. The combination of any one of embodiments 153-156, wherein the crRNA sequence has 100% sequence identity to the sequence in the target gene that includes the cleavage site.

158 The combination of any one of embodiments 153-157, wherein the crRNA sequence comprises the nucleic acid sequence set forth in any one of SEQ ID NOS: 8 and 13-24.

159. The combination of any one of embodiments 153-158, wherein the Cas nuclease and the sgRNA form a ribonucleoprotein (RNP) complex.

160. The combination of any one of embodiments 153-159, wherein the ssODN sequence comprises the nucleic acid sequence set forth in any one of SEQ ID NOS: 3, 5, 27, 30, 33, 36, 39, 42, 45, 48, 51, 54, 57, 60, and 63.

161. The combination of any one of embodiments 153-160, wherein the crRNA sequence and the ssODN sequence comprise the nucleic acid sequences set forth in:
SEQ ID NOS: 8 and 3, respectively;
SEQ ID NOS: 8 and 5, respectively;
SEQ ID NOS: 8 and 33, respectively;
SEQ ID NOS: 13 and 27, respectively;
SEQ ID NOS: 14 and 30, respectively;
SEQ ID NOS: 15 and 36, respectively;
SEQ ID NOS: 16 and 39, respectively;
SEQ ID NOS: 17 and 42, respectively;
SEQ ID NOS: 18 and 45, respectively;
SEQ ID NOS: 19 and 48, respectively;
SEQ ID NOS: 20 and 51, respectively;
SEQ ID NOS: 21 and 54, respectively;
SEQ ID NOS: 22 and 57, respectively;
SEQ ID NOS: 23 and 60, respectively; or
SEQ ID NOS: 24 and 63, respectively.

162. A complex for correcting a gene variant associated with Parkinson's Disease, comprising:
a Cas nuclease; and
a first sgRNA comprising a CRISPR targeting RNA (crRNA) sequence that is homologous to a sequence in a target gene;
wherein the target gene is human GBA1 and includes a single nucleotide polymorphism (SNP) that is associated with Parkinson's Disease.

163. The complex of embodiment 162, wherein the target gene comprises a sense strand and an antisense strand, and (i) the crRNA sequence is homologous to a sequence in the sense strand that includes a cleavage site.

164. The complex of embodiment 162, wherein the target gene comprises a sense strand and an antisense strand, and (i) the crRNA sequence is homologous to a sequence in the antisense strand that includes a cleavage site.

165. The complex of embodiment 162 or embodiment 163, wherein the crRNA sequence has 100% sequence identity to the sequence in the sense strand that includes the cleavage site.

166. The complex of embodiment 162 or embodiment 164, wherein the crRNA sequence has 100% sequence identity to the sequence in the antisense strand that includes the cleavage site.

167. The complex of any one of embodiments 162-166, wherein the Cas nuclease comprises one or more mutations such that the Cas nuclease is converted into a nickase that lacks the ability to cleave both strands of a double stranded DNA molecule.

168. The complex of any one of embodiments 162-167, wherein the Cas nuclease comprises one or more mutations such that the Cas nuclease is converted into a nickase that is able to cleave only one strand of a double stranded DNA molecule.

169. The complex of any one of embodiments 162-168, wherein the Cas nuclease is selected from the group consisting of Cas3, Cas9, Cas10, Cas12, and Cas13.

170. The complex of embodiment 169, wherein the Cas nuclease is Cas9.

171. The complex of embodiment 169 or embodiment 170, wherein the Cas9 is from a bacteria selected from the group consisting of Streptococcus pyogenes, Staphylococcus aureus, Neisseria meningitides, Campylobacter jejuni, and Streptococcus thermophilis.

172. The complex of embodiment 171, wherein the Cas9 is from Streptococcus pyogenes.

173. The complex of embodiment 172, wherein the Cas9 comprises one or more mutations in the RuvC I, RuvC II, or RuvC III motifs.

174. The complex of embodiment 173, wherein the one or more mutations comprises a D10A mutation in the RuvC I motif.

175. The complex of embodiment 172, wherein the Cas9 comprises one or more mutations in the HNH catalytic domain.

176. The complex of embodiment 175, wherein the one or more mutations in the HNH catalytic domain is selected from the group consisting of H840A, H854A, and H863A.

177. The complex of embodiment 175 or embodiment 176, wherein the one or more mutations in the HNH catalytic domain comprises a H840A mutation.

178. The complex of embodiment 172, wherein the Cas9 comprises a mutation selected from the group consisting of D10A, H840A, H854A, and H863A.

179. The complex of any one of embodiments 162-178, wherein the Cas nuclease and the first sgRNA form a ribonucleoprotein (RNP) complex.

180. A pair of complexes for correcting a gene variant associated with Parkinson's Disease, comprising:
(1) a first Cas nuclease; and
a first sgRNA comprising a CRISPR targeting RNA (crRNA) sequence that is homologous to a sequence in a target gene; and
(2) a second Cas nuclease; and
a second sgRNA comprising a crRNA sequence that is homologous to a sequence in the target gene;
wherein the target gene comprises a sense strand and an antisense strand;
wherein the crRNA sequence of the first sgRNA is homologous to a sequence in the sense strand that includes a cleavage site, and the crRNA sequence of the second sgRNA is homologous to a sequence in the antisense strand that includes a cleavage site; and
wherein the target gene is human GBA1 and includes a single nucleotide polymorphism (SNP) that is associated with Parkinson's Disease.

181. The pair of complexes of embodiment 180, wheren the SNP is situated between the cleavage site of the sense strand and the cleavage site of the antisense strand.

182. The pair of complexes of embodiment 180 or embodiment 181, wherein the first Cas nuclease and the second Cas nuclease comprise one or more mutations such that the first Cas nuclease and the second Cas nuclease are each converted into a nickase that lacks the ability to cleave both strands of a double stranded DNA molecule.

183. The pair of complexes of embodiment 180 or embodiment 181, wherein the first Cas nuclease and the second Cas nuclease comprise one or more mutations such that the first Cas nuclease and the second Cas nuclease are each converted into a nickase that is able to cleave only one strand of a double stranded DNA molecule.

184. The pair of complexes of any one of embodiments 180-183, wherein the first Cas nuclease and the second Cas nuclease is selected from the group consisting of Cas3, Cas9, Cas10, Cas12, and Cas13.

185. The pair of complexes of embodiment 184, wherein the first Cas nuclease and the second Cas nuclease is Cas9.

186. The pair of complexes of embodiment 184 or embodiment 185, wherein the first Cas nuclease and the second Cas nuclease is from a bacteria selected from the group consisting of Streptococcus pyogenes, Staphylococcus aureus, Neisseria meningitides, Campylobacter jejuni, and Streptococcus thermophilis.

187. The pair of complexes of embodiment 186, wherein the first Cas nuclease and the second Cas nuclease is from Streptococcus pyogenes.

188. The pair of complexes of embodiment 187, wherein the first Cas nuclease and the second Cas nuclease comprises one or more mutations in the RuvC I, RuvC II, or RuvC III motifs.

189. The pair of complexes of embodiment 188, wherein the one or more mutations comprises a D10A mutation in the RuvC I motif.

190. The pair of complexes of embodiment 189, wherein the first Cas nuclease and the second Cas nuclease comprises one or more mutations in the HNH catalytic domain.

191. The pair of complexes of embodiment 190, wherein the one or more mutations in the HNH catalytic domain is selected from the group consisting of H840A, H854A, and H863A.

192. The pair of complexes of embodiment 190 or embodiment 191, wherein the one or more mutations in the HNH catalytic domain comprises a H840A mutation.

193. The pair of complexes of embodiment 192, wherein the first Cas nuclease and the second Cas nuclease comprises a mutation selected from the group consisting of D10A, H840A, H854A, and H863A.

194. The pair of complexes of any one of embodiments 180-193, wherein the crRNA sequence of the first sgRNA has 100% sequence identity to the sequence in the sense strand that includes the cleavage site.

195. The pair of complexes of any one of embodiments 180-194, wherein the crRNA sequence of the second sgRNA has 100% sequence identity to the sequence in the antisense strand that includes the cleavage site.

196. The pair of complexes of any one of embodiments 180-195, wherein (i) the first Cas nuclease and the first sgRNA form a ribonucleoprotein (RNP) complex; and/or (ii) the second Cas nuclease and the second sgRNA form a RNP complex.

197. A nucleic acid, comprising the nucleic acid sequence set forth in any one of SEQ ID NOS: 8 and 13-24.

198. A nucleic acid, comprising the nucleic acid sequence set forth in any one of SEQ ID NOS: 1, 4, 25, 28, 31, 34, 37, 40, 43, 46, 49, 52, 55, 58, and 61.

199. A nucleic acid, comprising the nucleic acid sequence set forth in any one of SEQ ID NOS: 2, 26, 29, 32, 35, 38, 41, 44, 47, 50, 53, 56, 59, and 62.

200. A nucleic acid, comprising the nucleic acid sequence set forth in any one of SEQ ID NOS: 3, 5, 27, 30, 33, 36, 39, 42, 45, 48, 51, 54, 57, 60, and 63.

201. A cell produced by the method of any one of embodiments 1-138.

202. A cell identified by the method of embodiment 141.

203. A method for selecting for a cell comprising an integrated ssODN, comprising
contacting DNA isolated from a cell derived from the cell of any one of embodiments 23-63 and 112-138 with the one or more restriction enzymes; and
determining whether the DNA isolated from the cell has been cleaved at the restriction site,
wherein, if the DNA has been cleaved, the cell is identified as a cell comprising an integrated ssODN.

204. A method for selecting for a cell comprising a corrected SNP, comprising
sequencing DNA isolated from a cell derived from the cell of any one of embodiments 1-138; and
determining whether the target gene comprises a corrected form of the SNP,
wherein, if the target gene comprises a corrected form of the SNP, the cell is identified as a cell comprising a corrected SNP.

205. The method of embodiment 204, wherein the sequencing comprises one or more of whole genome sequencing (WGS), targeted Sanger sequencing, and deep exome sequencing.

206. A population of the cell of embodiment 201 or embodiment 202.

207. The population of embodiment 206, wherein the population is a population of pluripotent stem cells.

208. An induced pluripotent stem cell (iPSC) comprising a single-strand DNA oligonucleotide (ssODN) integrated into a target gene, wherein:
the target gene is human GBA1 and comprises a corrected single nucleotide polymorphism (SNP), wherein the non-corrected SNP is associated with Parkinson's Disease;
the integrated ssODN comprises the corrected SNP instead of the non-corrected SNP; and
(i) the ssODN comprises a protospacer adjacent motif (PAM) sequence that differs from a PAM sequence in the GBA1 target gene by at least one nucleotide position, wherein the integrated ssODN introduces a silent mutation in the PAM sequence of the target gene; and/or (ii) the ssODN comprises one or more nucleotides that are not homologous to the corresponding nucleotides of the GBA1 target gene, wherein the integrated ssODN introduces a restriction site in the target gene.

209. The iPSC of embodiment 208, wherein the ssODN comprises the nucleic acid sequence set forth in any one of SEQ ID NOS: 3, 5, 27, 30, 33, 36, 39, 42, 45, 48, 51, 54, 57, 60, and 63.

210. A method of differentiating neural cells, the method comprising:
(a) performing a first incubation comprising culturing the pluripotent stem cell(s) of any one of embodiments 207-209 in a non-adherent culture vessel under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and
(b) performing a second incubation comprising culturing cells of the spheroid in a substrate-coated culture vessel under conditions to neurally differentiate the cells.

211. The method of embodiment 210, wherein the cells are exposed to the inhibitor of TGF-β/activing-Nodal signaling up to a day at or before day 7.

212. The method of embodiment 210 or embodiment 211, wherein the cells are exposed to the inhibitor of TGF-β/activing-Nodal beginning at day 0 and through day 6, inclusive of each day.

213. The method of any one of embodiments 210-212, wherein the cells are exposed to the at least one activator of SHH signaling up to a day at or before day 7.

214. The method of any one of embodiments 210-213, wherein the cells are exposed to the at least one activator of SHH signaling beginning at day 0 and through day 6, inclusive of each day.

215. The method of any one of embodiments 210-214, wherein the cells are exposed to the inhibitor of BMP signaling up to a day at or before day 11.

216. The method of any one of embodiments 210-215, wherein the cells are exposed to the inhibitor of BMP signaling beginning at day 0 and through day 10, inclusive of each day.

217. The method of any one of embodiments 210-216, wherein the cells are exposed to the inhibitor of GSK3β signaling up to a day at or before day 13.

218. The method of any one of embodiments 210-217, wherein the cells are exposed to the inhibitor of GSK3b signaling beginning at day 0 and through day 12, inclusive of each day.

219. The method of any one of embodiments 210-218, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

220. The method of any one of embodiments 210-219, wherein the cells are exposed to BAGCT and the inhibitor of Notch signaling beginning on day 11.

221. The method of any one of embodiments 210-220, wherein the cells are exposed to BAGCT and the inhibitor of Notch signaling beginning at day 11 and until harvest of the neurally differentiated cells, optionally until day 18, optionally until day 25.

222. The method of any one of embodiments 210-221, wherein the inhibitor of TGF-β/activing-Nodal signaling is SB431542.

223. The method of any one of embodiments 210-222, wherein the at least one activator of SHH signaling is SHH or purmorphamine.

224. The method of any one of embodiments 210-223, wherein the inhibitor of BMP signaling is LDN193189.

225. The method of any one of embodiments 210-224, wherein the inhibitor of GSK3β signaling is CHIR99021.

226. A method of differentiating neural cells, the method comprising:
exposing the pluripotent stem cells of embodiment 194 to:
   (a) an inhibitor of bone morphogenetic protein (BMP) signaling;
   (b) an inhibitor of TGF-β/activing-Nodal signaling;
   (c) at least one activator of Sonic Hedgehog (SHH) signaling; and
   (d) at least one inhibitor of GSK3β signaling.

227. The method of embodiment 226, wherein, during the exposing, the pluripotent stem cells are attached to a substrate.

228. The method of embodiment 226, wherein, during the exposing, the pluripotent stem cells are in a non-adherent culture vessel under conditions to produce a cellular spheroid.

229. The method of any one of embodiments 226-228, wherein the inhibitor of TGF-β/activing-Nodal signaling is SB431542.

230. The method of any one of embodiments 226-229, wherein the at least one activator of SHH signaling is SHH or purmorphamine.

231. The method of any one of embodiments 226-230, wherein the inhibitor of BMP signaling is LDN193189.

232. The method of any one of embodiments 226-231, wherein the at least one inhibitor of GSK3β signaling is CHIR99021.

233. The method of any one of embodiments 226-232, wherein the exposing results in a population of differentiated neural cells.

234. The method of embodiment 233, wherein the differentiated neural cells are floor plate midbrain progenitor cells, determined dopamine (DA) neuron progenitor cells, and/or, dopamine (DA) neurons.

235. A therapeutic composition of cells produced by the method of any one of embodiments 210-225.

236. The therapeutic composition of embodiment 235, wherein cells of the composition express EN1 and CORIN and less than 10% of the total cells in the composition express TH.

237. The therapeutic composition of embodiment 236, wherein less than 5% of the total cells in the composition express TH.

238. A therapeutic composition of cells produced by the method of any one of embodiments 226-234.

239. The therapeutic composition of any one of claims 235-239, wherein at least 10%, at least 20%, at least 30%, at least 40%, or at least 50% of the cells of the composition comprise the corrected form of the SNP instead of the SNP.

240. The therapeutic composition of any one of embodiments 235-240, wherein at least 30% of the cells of the composition comprise the corrected form of the SNP instead of the SNP.

241. A method of treatment, comprising administering to a subject a therapeutically effective amount of the therapeutic composition of any one of embodiments 235-240.

242. The method of embodiment 241, wherein the cells of the therapeutic composition are autologous to the subject.

243. The method of embodiment 241 or embodiment 242, wherein the subject has Parkinson's disease.

244. The method of any one of embodiments 241-243, wherein the administering comprises delivering cells of a composition by stereotactic injection.

245. The method of any one of embodiments 241-244, wherein the administering comprises delivering cells of a composition through a catheter.

246. The method of embodiment 244 or embodiment 245, wherein the cells are delivered to the striatum of the subject.

247. Use of the composition of any one of embodiments 235-240, for the treatment of Parkinson's Disease.

### VIII. EXAMPLES

The following examples are included for illustrative purposes only and are not intended to limit the scope of the invention.

### Example 1: Genomic Correction of Parkinson Disease Risk Variants

### A. Analysis of Donor Fibroblasts

Fibroblasts from a human donor (“Donor 1”) having Parkinson Disease (PD) were obtained, and single nucleotide polymorphism (SNP) analysis was performed to confirm the donor carries a PD risk variant identified as SNP rs76763715 caused by the presence of a cytosine in place of a thymine (C>T), which causes an amino acid substitution of asparagine to serine at position 370 (N370S) in the beta-glucocerebrosidase enzyme encoded by the beta-glucocerebrosidase (GBA1) gene.

A primary fibroblast cell line derived from Donor 1 (“Donor 1 cell line”) was analyzed by 30× Whole Genome Sequencing (WGS). Germline variants were identified throughout the genome, which included the presence of the wildtype (C) and variant (T) copy of the rs76763715 SNP.

The genomic sequence of the Donor 1 cell line was further analyzed to determine the presence of any other known SNP variant(s) that might contribute to PD, other than the rs76763715 SNP. No other known SNP variant(s) that might contribute to PD were identified in this donor cell line.

The presence of other PD-associated SNPs identified in available genome-wide association studies (GWAS) (See Iwaki, H., et al., Genetic risk of Parkinson disease and progression: An analysis of 13 longitudinal cohorts, Neurol. Genet. (2019), Vol. 5(4): p. e348; Nalls, M. A., et al., Expanding Parkinson's disease genetics: novel risk loci, genomic context, causal insights and heritable risk, bioRxiv (2019): p. 388165), were assessed in this Donor 1 cell line, as well as cells from nine (9) other humans with PD (termed PD1, PD2, PD3, PD4, PD5, PD6, PD7, PD8, and PD9), and cells from two (2) humans without PD as controls (Control 1 and Control 2). The top PD-associated GWAS hits were ranked based on the effect size of the genetic risk score (GRS) for each SNP variant, and the presence of the SNP variants in each of the donor samples was assessed. FIGS. 1A and 1B together depict results from this GWAS analysis. In the heatmaps of FIGS. 1A and 1B, white indicates the absence of the variant allele for the particular PD-associated SNP, light grey indicates the heterozygous presence of the variant allele for the particular PD-associated SNP, and dark grey indicates the homozygous presence of the variant allele for the particular PD-associated SNP. Duplicate testing of the cells from donors with PD (PD3, PD4, and PD5) were included in the heatmaps.

As shown in FIG. 1A, the SNP variant rs76763715 in the GBA1 gene, discussed above, had the second highest effect size among the plotted SNP variants.

As shown in FIGS. 1A and 1B, there were several PD-associated GWAS hits for the Donor 1 cell line, but these SNP variants have a very small effect size and almost all of them were also present in one or both of the controls. In contrast, the PD-associated GWAS hits with larger effect size, such as variants of SNP rs76763715 and rs2230288 of the GBA1 gene, were only present in the sequences derived from donors with PD. As discussed above, the SNP variant rs76763715 includes a cytosine in place of a thymine, which results in a N370S mutation in the amino acid sequence. The SNP variant rs2230288 is caused by the presence of a guanine in place of an adenine (A>G), which causes an amino acid substitution of glutamic acid to lysine at position 365 (E365K). The SNP variant rs76763715 was present in the Donor 1 cell line, as expected, but was absent from the control donors who were not affected by PD.

B. Generation and Quality Control of iPSCs

The Donor 1 cell line was reprogrammed into induced pluripotent stem cells (iPSCs) using CytoTune™-iPS 2.0 Sendai Reprogramming Kit (ThermoFisher). Nine (9) clones were obtained at passage 15. These iPSC clones derived from the Donor 1 cell line were termed Clone 1, Clone 2, Clone 3, Clone 4, Clone 5, Clone 6, Clone 7, Clone 8, and Clone 9.

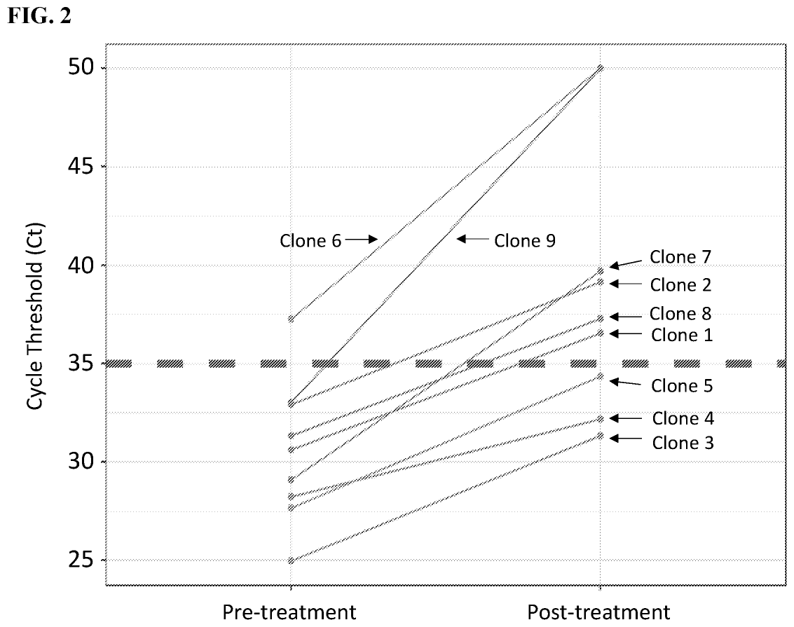

Although the Sendai virus does not integrate into the genome, a Sendai clearance test was carried out by quantitative PCR (qPCR) to ensure that the virus was cleared from the cells. To pass the Sendai clearance test, a standard for quality control was set such that there must be fewer than 8 copies of Sendai virus per 500 cells of the clone, as determined by qPCR. Each clone was tested by qPCR before (pre-treatment) and after (post-treatment) treatment intended to kill the Sendai virus. The treatment involved culturing the cells of each clone at 39 degrees Celsius for 5 days, which kills the heat-sensitive Sendai virus. Results from the Sendai clearance test are shown in FIG. 2. As shown in FIG. 2, six of the nine clones passed the Sendai clearance test: Clone 1, Clone 2, Clone 6, Clone 7, Clone 8, and Clone 9, as determined by having a cycle threshold (Ct) above 35 in the post-treatment group.

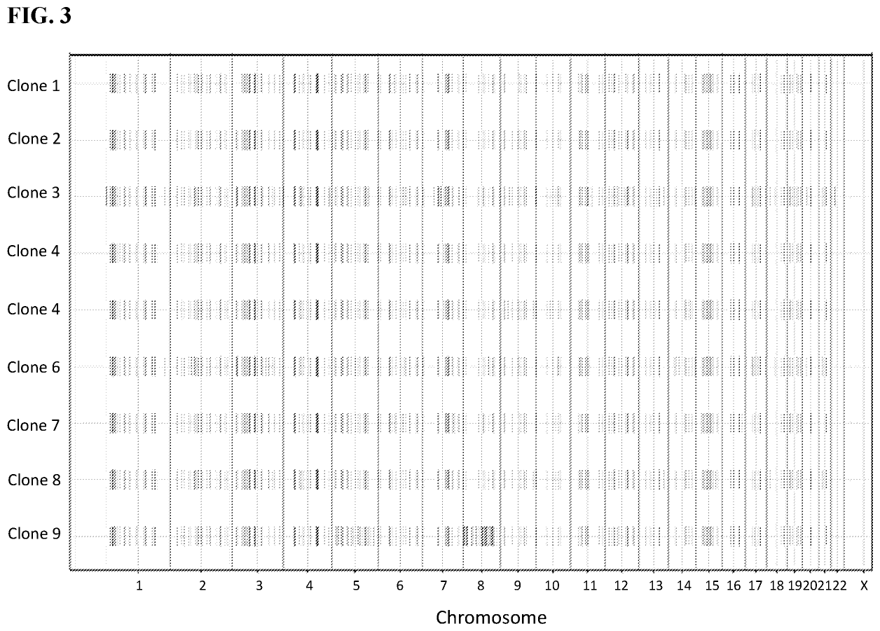

The clones were then subjected to genomic quality control, which included performing SNP analysis. The SNP genotyping analysis was carried out on all nine clones. Copy number variation (CNV) analysis was performed using a 300,000 SNP array (Infinium Core 300k Microarray; Illumina). Results from the SNP analysis of the clones are shown in FIG. 3. As shown in FIG. 3, each of the iPSC clones showed similar SNP genotypes with no major genomic deletions, duplications, or loss of heterozygosity. The SNP profile confirmed that all nine clones were derived from the same individual. Although small differences in genomic sequences among the iPSC clones were observed, this was expected, since each iPSC clone was derived from a single fibroblast. It is known that somatic cells, such as fibroblasts, from the same individual will include small differences in their genomic sequence.

WGS analysis for genomic integrity and RNA sequencing also are carried out on the iPSC clones. The RNA sequencing information is used to confirm the pluripotency of the clone using PluriTest™. Following further quality control analysis, including WGS analysis for genomic integrity, and RNA sequencing, two iPSC clones are selected for genomic editing of the rs76763715 variant (N370S in GBA).

### C. Genomic Correction of GBA N370S

A strategy was designed to correct the N370S mutation in GBA (the rs76763715 variant), caused by the presence of a cytosine in place of a thymine, using CRISPR/Cas9 gene editing.

The human GBA1 locus has high sequence similarity to its expressed pseudogene, GBAP1, which is thought to function as a regulator of GBA transcript level. Whole genome sequencing revealed that the GBA1 locus in the iPSC clones derived from the Donor 1 cell line (Clones 1, 2, 3, 4, 5, 6, 7, 8, and 9) encodes the cytosine (C) variant of rs76763715 associated with PD. Clones 1 through 9 include the wildtype thymine (T) variant of rs76763715 in the GBAP1 psuedogene.

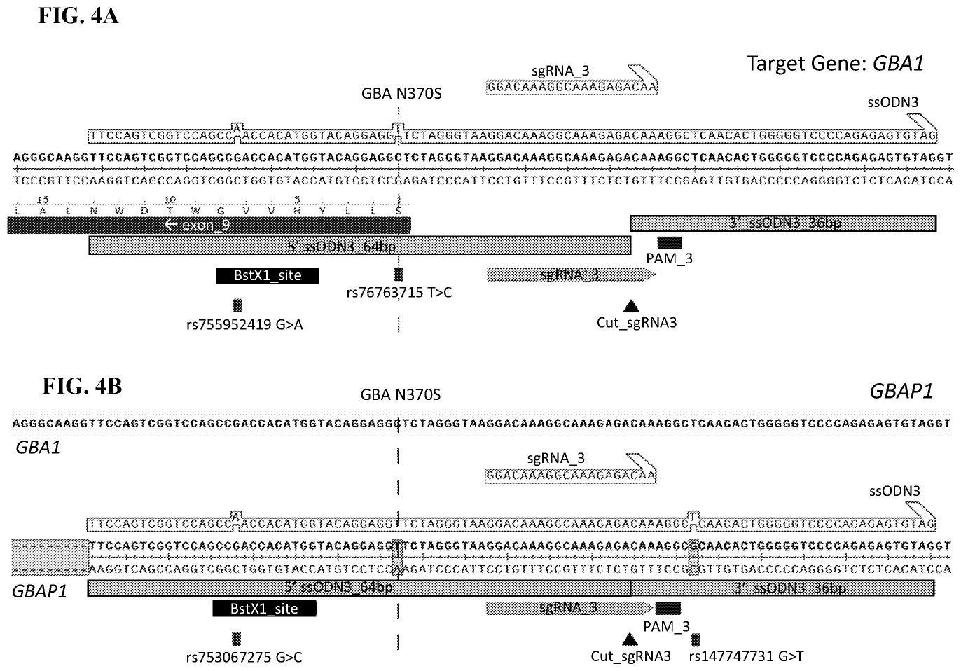

A sequence of the targeting sequence for GBA and GBAP1, including the rs76763715 position, are shown in FIGS. 4A and 4B. The similarity in sequence of the GBA1 gene and the GBAP1 pseudogene means that strategies to target the GBA1 gene may not only target the wildtype GBA1 allele, but also the two alleles of the GBAP1 psuedogene. Therefore, a strategy was developed that converts the cytosine variant of rs76763715 to a wild type thymine variant, while maximally preserving the sequence of the wild type allege of GBA1 and both wild type alleles of the GBAP1 psuedogene.

The sequence homology of GBA1 and GBAP1 in Donor 1 cell line was examined and three major differences at the targeting sequence were identified: (1) upstream of the correction site, GBAP1 contains deletions; (2) the homologous site of the GBA1 mutation in GBAP1 is the wild type sequence; and (3) an additional nucleotide differs between GBA1 and GBAP1 at the GBAP1 SNP variant rs147747731 downstream of the Protospacer Adjacent Moif (PAM). GBA1 has a thymine (T) at its homology site of rs147747731, and GBAP1 has a guanine (G) in place of thymine (G>T) at rs147747731.

For an efficient correction design, a length of ˜100-200 bp of recombinant template is generally required. An example of an exemplary targeting design is depicted in FIGS. 4A and 4B. Single-stranded DNA oligonucleotides (ssODNs) were designed, with arms designated as 5′ ssODN3 and 3′ ssODN3, to act as templates to convert the C variant of rs76763715 to a wild type T variant in GBA1. The nucleotide sequence of the 5′ ssODN arm template is as follows: TTCCAGTCGGTCCAGCCAACCACATGGTACAGGAGGTTCTAGGGTAAGGACAAAGGCAAAG AGA (SEQ ID NO: 1). The nucleotide sequence of the 3′ ssODN arm template is as follows: CAAAGGCTCAACACTGGGGGTCCCCAGAGAGTGTAG (SEQ ID NO: 2). The nucleotide sequence of the full length ssODN is as follows: TTCCAGTCGGTCCAGCCAACCACATGGTACAGGAGGTTCTAGGGTAAGGACAAAGGCAAAG AGACAAAGGCTCAACACTGGGGGTCCCCAGAGAGTGTAG (SEQ ID NO: 3). The ssODNs were designed to be delivered with a CRISPR/Cas9 (sgRNA and Cas9), generated from a plasmid expression vector or provided as a ribonucleoprotein (RNP) complex, to initiate DNA cleavage at a site surrounding the mutant base for correction by the ssODN. The nucleotide sequence of the CRISPR RNA (crRNA) portion of the single guide RNA (sgRNA) that is homologous to GBA1 is as follows: GGACAAAGGCAAAGAGACAA (SEQ ID NO: 8).

This targeting approach was also designed to introduce the SNP variant rs755952419 with a guanine in place of an adenine (G>A) in order to introduce a restriction site for the BstXI enzyme that could be used for screening purposes. Importantly, this SNP variant is a naturally occurring SNP and is not known to contribute to PD.

This targeting approach might edit the rs147747731 SNP in one or both GBAP1 alleles from a guanine (G) variant to a thymine (T). Importantly, the rs147747731 guanine variant (T>G) in GBAP1 is a naturally occurring SNP and does not have any known clinical significance. Thus, this strategy is expected to correct the N370S mutation in GBA1 without introducing any clinically significant changes in the GBAP1 pseudogene. Some designs, including the exemplary design depicted in FIGS. 4A and 4B, further include a synomymous restriction site, such as at rs753067275 by converting a guanine into an adenine, which does not result in any change in amino acid, to use as an internal marker for characterizing the genomic targeting.

Clones that were previously found to pass the Sendai clearance test (Clone 1, Clone 2, Clone 6, Clone 7, Clone 8, and Clone 9) are genetically edited using CRISPR/Cas9 with the targeting approach described above and depicted in FIGS. 4A and 4B. Clones derived from one or more of the genetically engineered Clones 1, 2, 6, 7, 8, and 9 are obtained and are assessed to confirm the presence of the wild type T variant of the rs76763715 SNP in both GBA1 alleles. The rs147747731 SNP is also assessed to determine whether one or both GBAP1 alleles were edited from the G variant to a T variant. Since it is possible that base pair substitutions may occur in addition to the targeted base due to the constraints of the CRISPR/Cas9 gene editing systems, all changes in sequence attributable to CRISPR editing will be identified by WGS, targeted Sanger sequencing, and deep exome sequencing.

iPSC clones that are confirmed to have been correctly edited at the rs76763715 SNP to include a wild type T variant in both alleles are then differentiated into dopaminergic (DA) neuron progenitors as described in Example 3.

### Example 2: Optimization of Cas9 Nuclease and sgRNAs for Gene Editing

A strategy similar to that described above in Example 1 was designed to correct the N370S mutation in GBA (the rs76763715 variant), caused by the presence of a cytosine (C) in place of a thymine (T), using CRISPR/Cas9 gene editing of iPSCs. In particular, the efficacy and accuracy of various combinations of Cas9 nucleases and sgRNAs was assessed in iPSCs derived from Donor 1, substantially as described in Example 1.

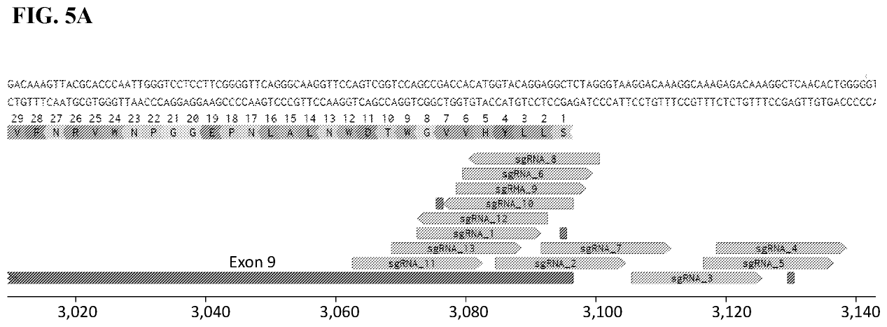

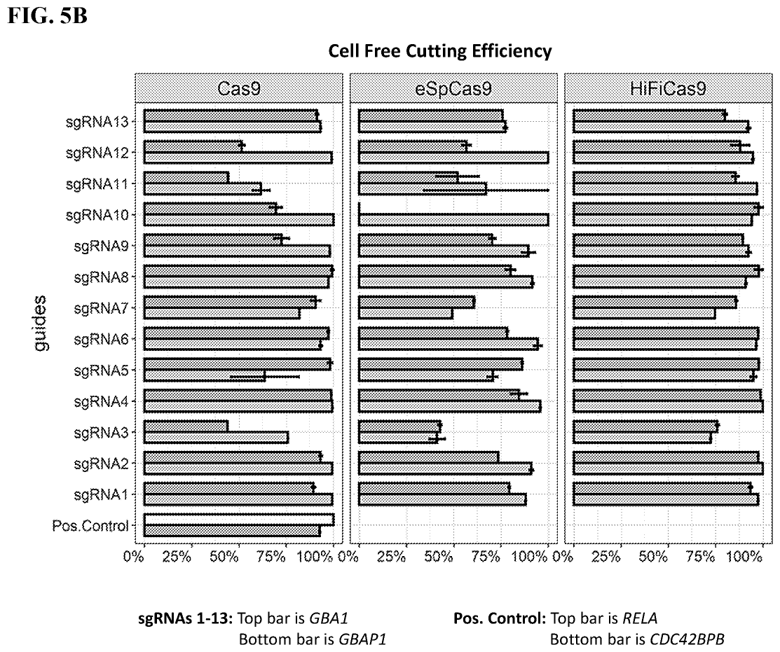

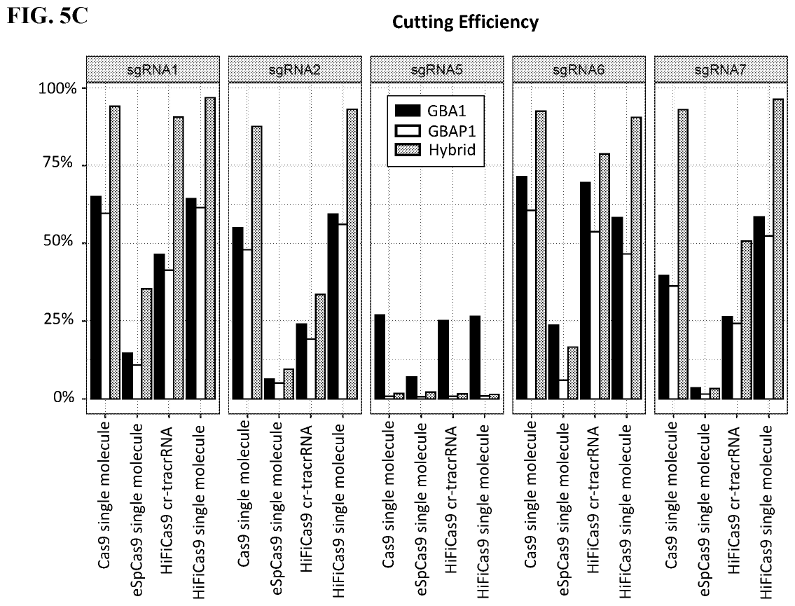

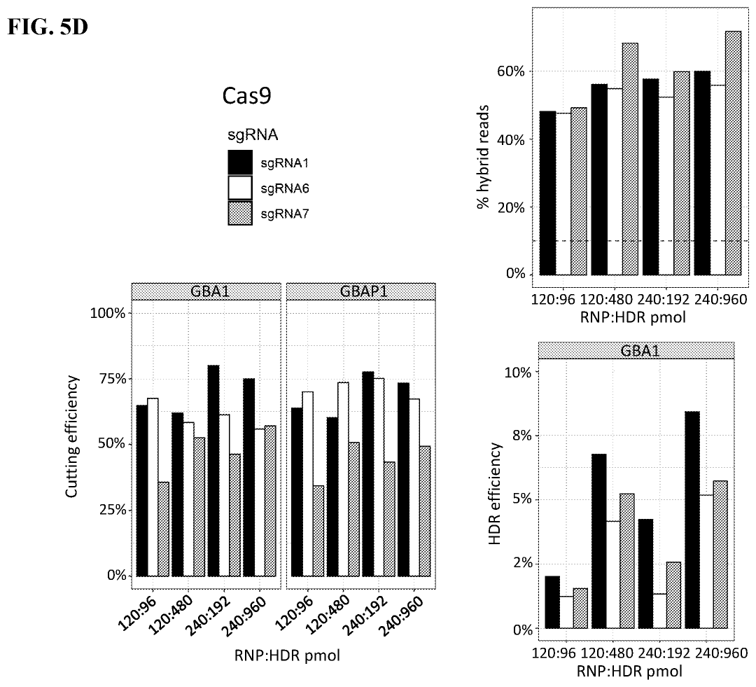

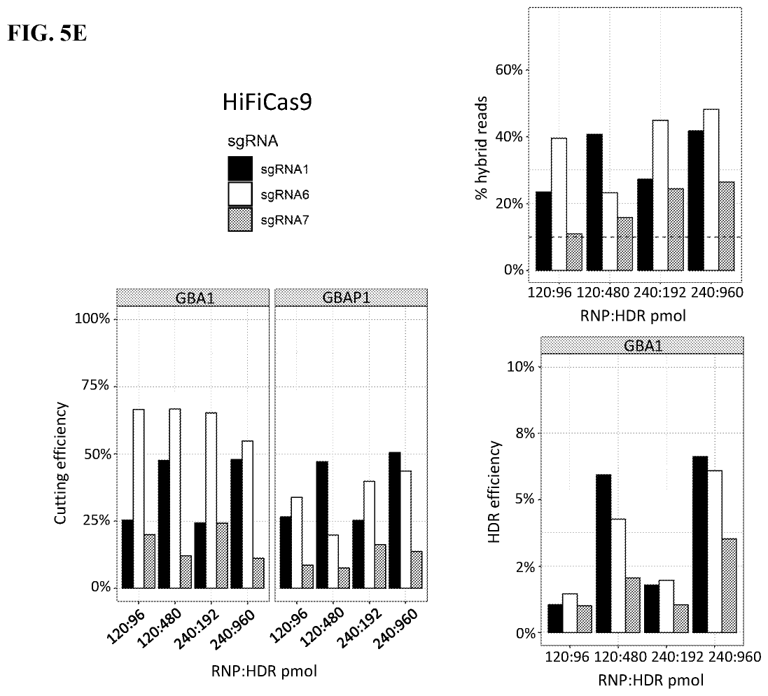

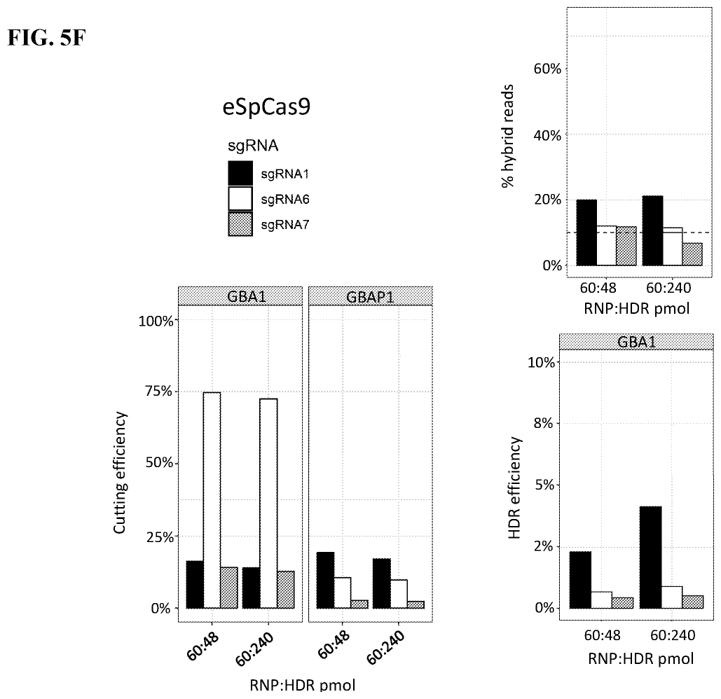

Three different Cas9 nucleases were assessed in combination with 13 different crRNA sequences of sgRNAs (“sgRNA 1” to “sgRNA 13”) and the respective ssODNS used with each crRNA sequence (FIG. 5A, showing sgRNA 1-13). The sequences of the crRNAs and the respective ssODNs used with each crRNA sequence are given by Table E1, below. The Cas9 nucleases assessed were (1) wildtype Cas9; (2) a high fidelity version of Cas9 (HiFiCas9); and (3) an enhanced version of Cas9 (eSpCas9).

TABLE E1
crRNA and ssODN Sequences

| sgRNA | crRNA | 5′ ssODN | 3′ ssODN | ssODN |
|---|---|---|---|---|
| No. | SEQ ID NO | SEQ ID NO | SEQ ID NO | SEQ ID NO |
| 1 | 13 | 25 | 26 | 27 |
| 2 | 14 | 28 | 29 | 30 |
| 3 | 8 | 1 | 2 | 3 |
| 3 | 8 | 4 | 2 | 5 |
| 3 | 8 | 31 | 32 | 33 |
| 4 | 15 | 34 | 35 | 36 |
| 5 | 16 | 37 | 38 | 39 |
| 6 | 17 | 40 | 41 | 42 |
| 7 | 18 | 43 | 44 | 45 |
| 8 | 19 | 46 | 47 | 48 |
| 9 | 20 | 49 | 50 | 51 |
| 10 | 21 | 52 | 53 | 54 |
| 11 | 22 | 55 | 56 | 57 |
| 12 | 23 | 58 | 59 | 60 |
| 13 | 24 | 61 | 62 | 63 |

As shown in FIG. 5B, the cutting efficiency of each of the crRNA sequences in combination with each of the three Cas9 nucleases was assessed in a cell-free setting in which purified DNA (500 basepair GBA1 or GBAP1 amplicon flanking the target site) was present. A positive Cas9 control was included using purified RELA or CDC42BPB DNA. Briefly, the recombinant nucleases were incubated with gRNA at room temperature for 20 minutes to yield a RNP. The RNP was then resuspended in solution with the iPSCs prior to electroporation. The results showed high cutting efficiency for several crRNA sequences in combination with different Cas9 enzymes.

The cutting efficiency of crRNA sequences of sgRNAs 1, 2, 5, 6, and 7 was further assessed in iPSCs from Donor 1. Each of Cas9, eSpCas9, and HiFiCas9 were provided with a sgRNA, while HiFiCas9 was also provided with a cr-tracrRNA. For cr-tracrRNA assembly. crRNA and tracrRNA were denatured at 95 Celsius for 5 minutes followed by a 20-minute cool to room temperature. For RNP assembly, the recombinant nucleases were incubated with the sgRNA or cr-trcrRNA at room temperature for 20 minutes, followed by resuspension and electroporation in solution with iPSCs. As GBAP1 is located approximately 16 kilobases (kb) upstream of GBA1 in cells, it was hypothesized that cutting at both loci may occur and create a large deletion of approximately the same size (i.e. 16 kb). If no large deletion occurs, both the GBA1 and GBAP1 amplicons will be detected by sequencing. If a large deletion occurs, only a hybrid amplicon is detected by sequencing. The percent of cutting events that created large deletions were approximated by the equation: Percent deletions=hybrid/((GBA1+GBAP1)/2+hybrids)*100%. The cutting efficiency of the five crRNA sequences selected for further assessment is shown in FIG. 5C. Among the five crRNA sequences assessed, crRNA sequences of sgRNAs 1, 6, and 7 were observed to have the highest cutting efficiencies, and were selected for further assessment.

The frequency of deletion events, cutting efficiency, and editing efficiency were compared in Donor 1 iPSCs using the crRNA sequences of sgRNAs 1, 6, and 7 and each of the three Cas9 nucleases with varying ratios of picomoles (pmol) Cas9/crRNA ribunocleoprotein (RNP) to pmol ssODN, to assess impacts on deletion frequency (given by % hybrid reads) and cutting and editing efficiency. Results for wildtype Cas9, HiFiCas9, and eSpCas9 are shown in FIGS. 5D-5F, respectively.

Based on these results, the following Cas9-crRNA combination and conditions were chosen for gene editing of iPSCs and subsequent clonal isolation: (1) wildtype Cas9 with crRNA sequence of sgRNA 1 (RNP:ssODN of 240:960 pmol); (2) HiFiCas9 with crRNA sequence of sgRNA 6 (RNP:ssODN 120:480 pmol); (3) HiFiCas9 with crRNA sequence of sgRNA 7 (RNP:ssODN of 240:960 pmol); and (4) eSpCas9 with crRNA sequence of sgRNA 6 (RNP:ssODN of 60:240 pmol).

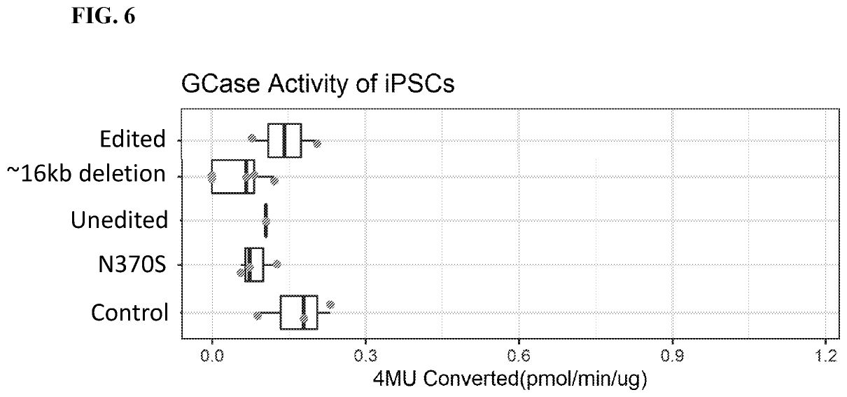

For each set of Cas9-sgRNA combination and conditions, clones of the edited iPSCs were isolated, expanded, and collected for DNA isolation, PCR, and Sanger sequencing. The activity of the GBA1 protein encoded by GBA1 in the edited iPSC clones (“edited”) was compared to that of cells having the large deletion (“˜16 kb deletion”), the parent N307S clone (“unedited”), three clones of the parent harboring the N370S variant (“N370S”), and healthy cells not harboring the N370S variant (“control”). In general, protein activity of edited iPSCs was found to be increased as compared to cells having the ˜16 kb deletion, the unedited cells, and the N370S cells (FIG. 6). iPSC clones that were confirmed to have been correctly edited at the rs76763715 SNP to include a wild type T variant in both alleles were differentiated into dopaminergic neuron progenitors by the methods described in Example 3. The differentiated, genome edited cells were observed to express both tyrosine hydroxylase (TH) and FOXA2, indicating their commitment to DA neuron fate.

### Example 3: Differentiation of Genomically Corrected Target Clones into Dopaminergic Neuron Progenitors

iPSC clones that are confirmed to have been correctly edited at the rs76763715 SNP (from Examples 1 and 2) to include a wild type T variant in both alleles are subjected to an exemplary dopaminergic (DA) neuronal differentiation protocol. Expression of various midbrain markers is assessed.

iPSCs from the human donors are maintained by plating in Geltrex™-coated 6-well plates at 200,000 cells per cm². The cells are cultured without feeder cells in mTeSR™1-based media until they reach approximately 90% confluence (day 0). The iPSCs are then washed with sterile PBS and detached from the 6-well plates by enzymatic dissociation with Accutase™. The collected iPSCs are then used in the subsequent differentiation protocol.

### A. Differentiation Protocol

The collected iPSCs are re-suspended in “basal induction media” (see below) and are seeded under non-adherent conditions using 6-well or 24-well AggreWell™ plates. The cells are seeded under conditions to achieve the following concentrations: 500 cells/spheroid; 1,000 cells/spheroid, 2,000 cells/spheroid; 3,000 cells/spheroid; 10,000 cells/spheroid; or 15,000 cells/spheroid. Following seeding of the cells, the 6-well or 24-well plates are immediately centrifuged at 200×g or 100×g for 3 minutes, respectively. Beginning at day 0, the media is supplemented with various small molecules as described below. The cells are cultured for 7 days, with media replacement as detailed below, to form spheroids. On day 7, the resulting spheroids are dissociated into single cells by enzymatic dissociation with Accutase™, and the cells are plated as monolayers at a concentration of 600,000 cells/cm² on substrate-coated 6-well plates (Geltrex™) for the remainder of culture, and are further supplemented with nutrients and small molecules as described below.

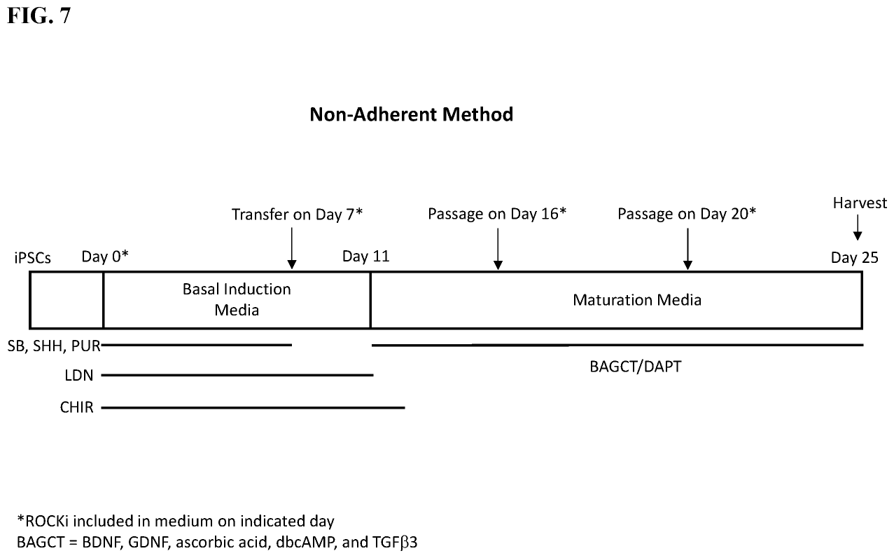

A schematic of the exemplary non-adherent differentiation protocol is shown in FIG. 7 and Table E2, which depict the small molecule compounds that are added at various days during the differentiation method. From days 0 to 10, cells are cultured in the basal induction media, which is formulated to contain Neurobasal™ media and DMEM/F12 media at a 1:1 ratio (and with N-2 and B27 supplements, non-essential amino acids (NEAA), GlutaMAX™, L-glutamine, β-mercaptoethanol, and insulin), and is supplemented with the appropriate small molecule compound(s). From days 11 to 25, cells are cultured in a “maturation media” (Neurobasal™ media containing N-2 and B27 supplements, non-essential amino acids (NEAA), and GlutaMAX™), and are supplemented with the appropriate small molecule compound(s). The basal induction media also includes a serum replacement.

TABLE E2
Differentiation Protocol

| Day | Media | Small Molecules | | | | | | | |
|---|---|---|---|---|---|---|---|---|---|
| 0* | Basal | 5% | LDN | SB | SHH | PUR | CHIR | ROCKi | |
| | Induction | S | | | | | | | |
| 1 | Basal | 5% | LDN | SB | SHH | PUR | CHIR | | |
| | Induction | S | | | | | | | |
| 2 | Basal | 2% | LDN | SB | SHH | PUR | CHIR | | |
| | Induction | S | | | | | | | |
| 3 | Basal | 2% | LDN | SB | SHH | PUR | CHIR | | |
| | Induction | S | | | | | | | |
| 4 | Basal | 2% | LDN | SB | SHH | PUR | CHIR | | |
| | Induction | S | | | | | | | |
| 5 | Basal | 2% | LDN | SB | SHH | PUR | CHIR | | |
| | Induction | S | | | | | | | |
| 6 | Basal | 2% | LDN | SB | SHH | PUR | CHIR | | |
| | Induction | S | | | | | | | |
| 7* | Basal | 2% | LDN | | | | CHIR | ROCKi | |
| | Induction | S | | | | | | | |
| 8 | Basal | 2% | LDN | | | | CHIR | | |
| | Induction | S | | | | | | | |
| 9 | Basal | 2% | LDN | | | | CHIR | | |
| | Induction | S | | | | | | | |
| 10 | Basal | 2% | LDN | | | | CHIR | | |
| | Induction | S | | | | | | | |
| 11 | Maturation | | BDNF | GDNF | ascorbic | dbcAMP | CHIR | TGFβ3 | DAPT |
| 12 | Maturation | | BDNF | GDNF | ascorbic | dbcAMP | CHIR | TGFβ3 | DAPT |
| 13 | Maturation | | BDNF | GDNF | ascorbic | dbcAMP | | TGFβ3 | DAPT |
| 14 | Maturation | | BDNF | GDNF | ascorbic | dbcAMP | | TGFβ3 | DAPT |
| 15 | Maturation | | BDNF | GDNF | ascorbic | dbcAMP | | TGFβ3 | DAPT |

Day 16: 1^{st} Passage

| | | | | | | | |
|---|---|---|---|---|---|---|---|
| 16* | Maturation | BDNF | GDNF | ascorbic | dbcAMP | TGFβ3 | DAPT |
| 17 | Maturation | BDNF | GDNF | ascorbic | dbcAMP | TGFβ3 | DAPT |
| 18 | Maturation | BDNF | GDNF | ascorbic | dbcAMP | TGFβ3 | DAPT |
| 19 | Maturation | BDNF | GDNF | ascorbic | dbcAMP | TGFβ3 | DAPT |

Day 20: 2^{nd} Passage

| | | | | | | | |
|---|---|---|---|---|---|---|---|
| 20* | Maturation | BDNF | GDNF | ascorbic | dbcAMP | TGFβ3 | DAPT |
| 21 | Maturation | BDNF | GDNF | ascorbic | dbcAMP | TGFβ3 | DAPT |
| 22 | Maturation | BDNF | GDNF | ascorbic | dbcAMP | TGFβ3 | DAPT |
| 23 | Maturation | BDNF | GDNF | ascorbic | dbcAMP | TGFβ3 | DAPT |
| 24 | Maturation | BDNF | GDNF | ascorbic | dbcAMP | TGFβ3 | DAPT |
| 25 | Maturation | BDNF | GDNF | ascorbic | dbcAMP | TGFβ3 | DAPT |

S: Serum replacement;
LDN: LDN193189;
SB: SB431542;
SHH: recombinant mouse Sonic Hedgehog (rmSHH);
PUR: Purmorphamine;
CHIR: CHI99021;
ROCKi: Y-27632;
BDNF: recombinant human brain-derived neurotrophic factor (rhBDNF);
GDNF: recombinant human glial cell-derived neurotrophic factor (rhGDNF);
TGFβ3: recombinant human transforming growth factor beta 3 (rhTGFβ3);
dbcAMP: dibutyryl cyclic AMP;
Ascorbic: ascorbic acid;
*Indicates media supplemented with ROCK inhibitor (Y-27632)

Specifically, on day 0, the basal induction media is formulated to contain: 5% serum replacement, 0.1 μM LDN, 10 μM SB, 0.1 μg/mL SHH, 2 μM PUR, 2 μM of the GSK3β inhibitor CHIR99021, and 10 μM of the ROCK inhibitor Y-27632. The media is completely replaced on day 1 to provide the same concentration of the small molecule compounds as on day 0, except that no ROCK inhibitor is added. From days 2 to 6, the same concentration of the small molecule compounds as on day 1 is provided daily but by 50% media exchange; the concentrations of small molecules in the basal induction media are doubled on days 2 to 6, to ensure the same total concentration of compounds is added to the cell cultures. Also, the media on days 2 to 6 is formulated with 2% serum replacement.

On day 7 when the cells are transferred to substrate-coated plates, the basal induction media is formulated to contain: 2% serum replacement, 0.1 μM LDN, 10 μM SB, 2 μM CHIR99021, and 10 μM Y-27632. The media is replaced daily from days 8 to 10, with basal induction media formulated to contain 2% serum replacement, 0.1 μM LDN and 2 μM CHIR99021.

Starting on day 11, the media is switched to maturation media formulated to contain: 20 ng/mL BDNF, 0.2 mM ascorbic acid, 20 ng/mL GDNF, 0.5 mM dbcAMP, and 1 ng/mL TGFβ3 (collectively, “BAGCT”), 10 μM DAPT, and 2 μM CHIR99021. The media is replaced on day 12 with the same media formulation containing the same concentrations of small molecule compounds as on day 11. From day 13 until harvest, the media is replaced either every day (days 13-17) or every other day (after day 17) with maturation media formulated to contain BAGCT and DAPT (collectively, “BAGCT/DAPT”) at the same concentrations as on days 11 and 12.

On days 16 and 20, the cells are passaged by enzymatic dissociation with dispase and collagenase. Cells are re-suspended as small clumps and are re-plated. On passaging days 16 and 20, cells are re-plated in maturation media that is further supplemented with the ROCK inhibitor.

### B. Differentiation Status

On day 25, the differentiated cells are analyzed by immunohistochemistry for markers of midbrain DA neurons, including FOXA2 and tyrosine hydroxylase (TH), or are harvested by enzymatic dissociation and cryofrozen for downstream use or analysis. Nuclei are identified by DAPI staining.

In some studies, to compare differentiation carried out in the presence of serum replacement versus in the absence of any serum, cells are grown under the same conditions, but in the absence of serum replacement from days 0-10.

In another experiment, cells are treated as described in the previous section, but the conditions under which the CHIR inhibitor is added from days 7 to 12 are modified by the addition of fibroblast growth factor 8 (FGF8) and with different concentrations of CHIR. All other aspects of media formulation and small molecule supplementation are maintained as shown in Table E1. Specifically, from days 7 to 12, either 2 μM, 4 μM, or 6 μM of CHIR is added, with or without 100 ng/mL fibroblast growth factor 8 (FGF8). At day 26 of the differentiation protocol, the number of TH+ neurons is assessed.

### C. Neuronal Differentiation Marker Expression

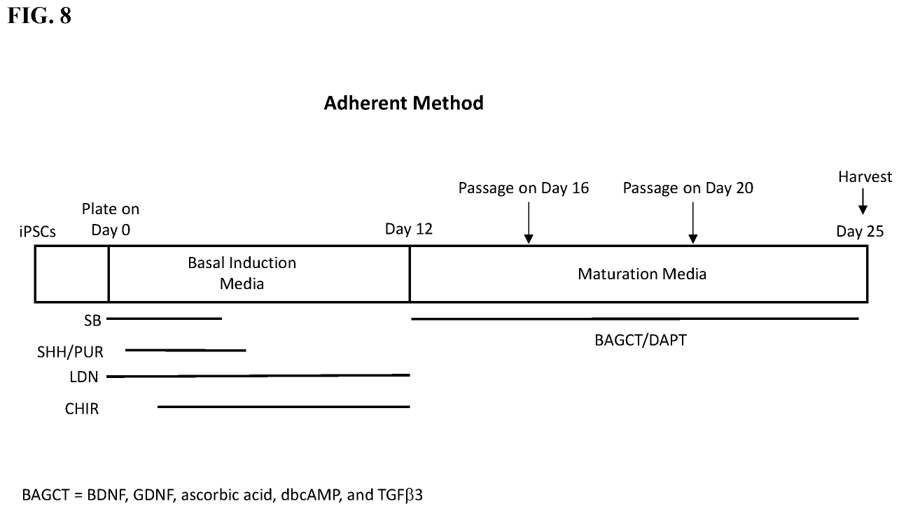

Expression levels of neuronal differentiation markers are compared between cells generated from the exemplary non-adherent method described above and cells generated by a different method, in which the cells are initially plated in Geltrex™-coated 6-well plates on day 0 and remained plated for the duration of the differentiation protocol (“adherent method”). The adherent method also differs from the non-adherent method, in that the small molecules are added on different schedules (FIG. 8). For all experimental conditions, cells are derived from the same human donor.

Cells from the adherent and non-adherent methods are harvested on day 25 of differentiation and assessed for the presence of FOXA2+TH+ neurons. Nuclei are identified by staining with DAPI.

### D. Tyrosine Hydroxylase (TH) Expression

The expression of tyrosine hydroxylase is compared between cells that are generated by the exemplary non-adherent method described above. Cells are collected at various time points, e.g., at either day 18 or day 25.

### E. Dopamine (DA) Production

The production of dopamine (DA) by cells that are generated by the exemplary non-adherent method described above is assessed. Cells are collected at various time points, e.g., at either day 18 or day 25.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### SEQUENCES

| SEQ | | |
|---|---|---|
| ID NO | Sequence | Description |
| 1 | TTCCAGTCGGTCCAGCCAACCACATGGTACAGGAGGTTCTAGGGT | 5′ ssODN |
| | AAGGACAAAGGCAAAGAGA | (rs76763715) |
| 2 | CAAAGGCTCAACACTGGGGGTCCCCAGAGAGTGTAG | 3′ ssODN |
| | | (rs76763715) |
| 3 | TTCCAGTCGGTCCAGCCAACCACATGGTACAGGAGGTTCTAGGGT | ssODN |
| | AAGGACAAAGGCAAAGAGACAAAGGCTCAACACTGGGGGTCCCC | (rs76763715) |
| | AGAGAGTGTAG | |
| 4 | TTCCAGTCGGTCCAGCCGACCACATGGTACAGGAGGTTCTAGGGT | 5′ ssODN |
| | AAGGACAAAGGCAAAGAGA | (rs76763715) |
| 5 | TTCCAGTCGGTCCAGCCGACCACATGGTACAGGAGGTTCTAGGGT | ssODN |
| | AAGGACAAAGGCAAAGAGACAAAGGCTCAACACTGGGGGTCCCC | (rs76763715) |
| | AGAGAGTGTAG | |
| 6 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (wildtype) |
| | LHNFSLPEEDTKLKIPLIHRALQLAQRPVSLLASPWTSPTWLKINGAV | amino acid |
| | NGKGSLKGQPGDIYHQTWARYFVKFLDAYAEHKLQFWAVTAENEPS | sequence |
| | AGLLSGYPFQCLGFTPEHQRDFIARDLGPTLANSTHHNVRLLMLDDQ | |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 7 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (N370S |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITSLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 8 | GGACAAAGGCAAAGAGACAA | crRNA |
| | | (rs76763715) |
| 9 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (L444P |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDPDAVALMHPDGSAVVVVLNRSSKDVPLT | |
| | IKDPAVGFLETISPGYSIHTYLWRRQ | |
| 10 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (E326K |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGKTHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 11 | ACAGAAGTACAATGCTGATTATGATCTGTCAGCTCAGCAAGGGGC | Human |
| | AGACACCCTGGCCTTCATGTCTCTCCTGGAGGAGAAGTTGATCCCG | GBA1 |
| | GTGCTGGTGAGTGTGCCCAGACCTCCCAGCATCCATGGCCAGCCG | (wildtype) |
| | GGGAGGGGACGGGAACACACAGACCCACACAGAGACTCAGGAGA | nucleotide |
| | GCATGGAGGTCAGAAGCCCACCTTGAATCAGACAGGTGCACTGGC | sequence |
| | TCAGACCTGCCTGTTTCTTCCTGCCCACCCAATCCAGGTACATACT | |
| | TTTTGGATAGACACCAAGAACTACGTAGAAGTGACCCGGAAGTGG | |
| | TATGCAGAGGCTATGCCCTTTCCCCTCAACTTCTTCCTGCCTGGCC | |
| | GCATGCAGCGGCAGTACATGGAACGGCTACAGCTGCTGACTGGGG | |
| | AGCACAGGCCTGAGGACGAGGAAGAGCTGGAGAAGGAGGTAGCT | |
| | CTGAGACCGGGGGCTATTGTATGAGATGAGCCCCAAGGATGCTGG | |
| | CCAGGAATGGGAGTGCTTAGGTGCGGAGGTGGCACTGTTCCCGCA | |
| | GCTGCAAGCCTACCTGTGTCGCCCCTACAGCTGTACCGAGAGGCTC | |
| | GGGAGTGTCTGACCCTGCTCTCTCAGCGCCTGGGCTCTCAAAAGTT | |
| | CTTCTTTGGAGATGCGTGAGTCTGACTCCAAGAGGGTAATGGGTG | |
| | GCTTGGAAGAAGATACAGGTTCAGATGGAGCAGCTGGAGCTGGGG | |
| | CTGGGGCTGGGGCTGGCTCAGGCTCTGGATAGGAGGTCCCTGAGA | |
| | CAGATACTGGCCCTGGTGACAGTGGGGCTGTGCGTGGGGCCAGAG | |
| | CCTTCTCAGAGGTACAAAAGGGTAGGGTGGGAGGGCAGCCAGGCA | |
| | CAGGAAGGGCCTGAAGAGCTGTGGGGCACTGAGTGTGCCCTTTAT | |
| | GCAGCCCTGGGATAGAGCCCTATTCAGGGCCAGGCTGGCGCCACC | |
| | TGGGGATCTCTCCCCATACCAGGTCTAGAACTGTGTGTCCTGTCCT | |
| | TCCCTGGTGGCCGCCTGCTGCCCAGAGCCCACCTCCCAAGGCTGAC | |
| | TCTTCCTCCAGCTCCATCTTTACCCCTTCTACCCCAGTGGTTCTCCT | |
| | CCATCCCACCCTTCTCTCTCTGCTCCAGCCCTGCCTCCTTGGACGCC | |
| | TTCGTCTTCAGCTACTTGGCCCTGCTGCTGCAGGCAAAGCTGCCCA | |
| | GTGGGAAGCTGCAGGTCCACCTGCGTGGGCTGCACAACCTCTGTG | |
| | CCTATTGTACCCACATTCTCAGTCTCTACTTCCCCTGGGATGGAGG | |
| | TAAGGGGCAGATGGGAGGGGCAGCCCTGGGGAGAGTGGGCAGGG | |
| | ATCCAAGAACTAGTTCTCCTAACACACCTTCCTTCCTTGACCCTCA | |
| | GCTGAGGTACCACCGCAACGCCAGACACCAGCAGGCCCAGAGACT | |
| | GAGGAGGAGCCATACCGGCGCCGGAACCAGATCCTATCTGTGCTG | |
| | GCAGGACTGGCAGCCATGGTGGGCTACGCCTTGCTCAGCGGCATT | |
| | GTCTCCATCCAGCGGGCAACGCCTGCTCGGGCCCCAGGCACCCGG | |
| | ACCCTGGGCATGGCTGAGGAGGATGAAGAGGAATGATTTGTCCTC | |
| | ACGCTCCCAAGACTGGTTTTTCTACTCTCATGCATTCCAGAGGCCC | |
| | CCGTGCCTCCTCGTTGTTGGTACAGCCGGACACGGGGTGCTGCCAC | |
| | CCAGAATAAAGCCACTCACACTGACTGGGCTCAAACATTTTCTCCT | |
| | TTAAGAGCTGCCATTTTTCCTGGCTGGTGCCATAGGAATCATCTGG | |
| | GTGCCTGGGCACACCCGCTGCTGCTTTAAGGCTTCCGCCCTGATGC | |
| | TGACACTGCTGCTCCACGGGCCCAGTTCTGCATCTCCAGGAAAGAC | |
| | AAACAGTCTCCAGTTTTGGGCCCAGCTTTCCTAGTCTCTTCTTTTCC | |
| | TTACCCTCAGCCCTGATCTTGTGTTTGTACGGACAGTGAGCTCACC | |
| | CTAGGCCTGGACCCAGGCCCAGTTTCCAAAGCAAGCAGCACACAG | |
| | CGCATGTTCACATAAGCATGGGGGCTGGGGGGACACTGGGGCTTA | |
| | CTGATCTTTTTCTAGGGGCCTCCAGCCCCTGGCACCACCTAGAGGG | |
| | GAAAGTGAGTCACCCAAACCATTGCCCCTGGGCTTACGTCGCTGTA | |
| | AGCTCACACTGGCCCTGCTGTGCCCTCTTTAGTCACAGACAGCGTG | |
| | TGAGCTGACTCTGTCCCTTTAATGCCCAGGCTGAGCCCAGTGCCTC | |
| | CTTGAGTATCTGCTCCATCACTGGCGACGCCACAGGTAGGTGTGAA | |
| | TGGAGTAGCCAGGTGAGATTGTCTCCAGGAAGCCCACAGCAGGAT | |
| | CCTTGATGGTAAGAGGCACATCCTTAGAGGAGCTAGGGAGCAGGG | |
| | AGGAGAAGCTGAGAGTGTGATCCTGCCAAGGCCCCCAACGCTGTC | |
| | TTCAGCCCACTTCCCAGACCTCACCATTGCCCTCACCGGTTTAGCA | |
| | CGACCACAACAGCAGAGCCATCGGGATGCATCAGTGCCACTGCGT | |
| | CCAGGTCGTTCTTCTGACTGGCAACCAGCCCCACTCTCTGGGAGCC | |
| | CTCAGGAATGAACTTGCTGAATGTGGGAACAGATGGTCAGAGTCC | |
| | CTCGGGGTACCTCCCATGAAACCCTCATCTAAGAAGTCACCCACCC | |
| | ACGGACCCACCCCATAACTCCTGCAGAGGCTCTGCCCTGGCTCTCT | |
| | AGGCCTGGAGCCATGCTGCTGGGCACTGACCCTGCTTTTCTGCATC | |
| | GCAGTCCAGCCTCAGGCATTGGGGTTTTCTGTTGCTACCTAGTCAC | |
| | TTCCTGCCTCCATGGTGCAAAAGGGGATGGGTGTGCCTCTTCCGAG | |
| | GTTCCACCCTGAACACCTTCCTGCTCCCTCGTGGTGTAGAGTGATG | |
| | TAAGCCATCCGATGTAGGAGATGATAGGCCTGGTATGGAATGGGG | |
| | GTGCCCGCCCTCCACTCACCTGAAGTGGCCAAGGTGGTAGAACAT | |
| | GGGCTGTTTGTAAAACGTGTCCTTGGTGATGTCTACAATGATGGGA | |
| | CTGTCGACAAAGTTACGCACCCAATTGGGTCCTCCTTCGGGGTTCA | |
| | GGGCAAGGTTCCAGTCGGTCCAGCCGACCACATGGTACAGGAGGT | |
| | TCTAGGGTAAGGACAAAGGCAAAGAGACAAAGGCGCAACACTGG | |
| | GGGTCCCCAGAGAGTGTAGGTAAGGGTCACATGTGGGAGAGGCAG | |
| | CTGTGGGTAGGTCAGCCCTGTGAGGGGCACATTCCTTAGTAGCTAA | |
| | GGAGTTGGGGGTGTGAAGATCCAGGCATCTCAAGGGGAGCTGAGA | |
| | AGTCTGAGGCAGCTGCAAGTGCCTCAGTAGTTGCAAAAGGGGCAA | |
| | TGAGGTGTGCAGACCTGTGAAGGAAAGGGAAGATAGGGAATCAT | |
| | GGTTCCCCAGAGTTGCTCAAAAGGGCAGGCTAGCTGGGGAAAGCT | |
| | GGACAGGAAGGGCTTCTGTCAGTCTTTGGTGAAACTAGTAAGAGG | |
| | TCTGAGGTCTGCTTTGCAGGAAGGGAGACTGGGGTGGCTTACCGT | |
| | GATGATGCTGTGGCTGTACTGCATCCCTCGATCCCAGGAGCCTAGC | |
| | CGCACACTCTGCTCCCAGAACTTGGAGCCCACACAGGCCTCTGAG | |
| | GCAAAGAGCATGGTGTTGGGGAACAGGCGGTGTGTCTCCCCTAGG | |
| | GTGGCTTTGGCTGGAGCCAGAAAGTCCAGGTACCAATGTACAGCA | |
| | ATGCCATGAACATATTTAGCTGCTTCTGGGTCTGTCAGTACCTGCA | |
| | AAGGAAGAGCAACTGATCCTGGACCTTGCACACAGGCTTCTGGAA | |
| | CTTCTAGTTCCTGTTGTAGGAATCCTGGAGTTGGGTGACGGGAAGA | |
| | ATGCAACTAGAGAGGTTTGGGGAGATTTTTTTTTGTTTTTGAGACA | |
| | GGATGTCACTCTGTCACTCGGGCTGGAGTACAGTGGCGCAATCAC | |
| | GACTCACTGCAGCCTTGACTTCCTAGGGTCAACTGATCCTCCCACA | |
| | TTAGCCTCCTGAGTAGCTGGGACTACACGGGTGCCACACCCAGCT | |
| | AATTTGTGTGTGTATGTGTGTGTATGTATGTGTGTGTGTGTATATAT | |
| | ATATACATATAAACACATATATATGTATATACACATACATACATGA | |
| | ACCACCACCCCCAGCCTAGATAGTTTTGTTTTGTTTTGTTTCGAGAT | |
| | GGAGTCTCGCTCAGCCTCCCAGGCTGGAGTGCAGTGGCGCGATCT | |
| | CGGCTCACCGCAACCACCATCTCCCGGGTTCAACTGATTCTCCCTT | |
| | CTCAGCCTCCTGAGTAGCTGGGATTACAGACACCCACCATCATGCC | |
| | CAGATAATTTTTTTTTTTTGTATTTTAGTAGACACAGGGTTTCAACA | |
| | CGTTGGCCAGGGTGGTCTTAAACTCCTGACCTCAGGTGATCACCCG | |
| | CCTCGGCTTCCCAAAGTGCTGGGTTTGCATGAGTGAGCCACCTCGC | |
| | CCAGCCCCTAGAAAGGTTTCAAGCGACAACTGTGGGATCCATGGC | |
| | ACCCTGGAGGTCCAGGGGAATGGTGCTCTAGGAATCCATAGTTGG | |
| | GTAGAGAAATCGCTCTAAGTTTGGGAGCCAGTCATTTGGATGCTG | |
| | GATTTGAAGGTCACTGGAGCACCATGGAGGTCCAGGCCTTACCAC | |
| | CTTTGCCCAGTGGGGCAGCAGCAAGCGTTGGTCATCCAGCATGAG | |
| | TAGGCGGACATTGTGGTGAGTACTGTTGGCGAGGGTAGGACCTAG | |
| | GTCACGGGCAATGAAGTCTCGCTGATGTTCAGGGGTGAAGCCCAG | |
| | GCACTGGAAGGGGTATCCACTCAACAGCCCAGCAGAAGGCTCATT | |
| | TTCAGCTGTCACTGCCCAGAACTGTAACTTGTGCTCAGCATAGGCA | |
| | TCCAGGAACCTGGCAAGAGAAAGGTCATGAATGATCCGGCCAAGA | |
| | AAGTGGACCAGACCAGCTGGGTGTGGTGGCTCACACCTGTAATCC | |
| | CAGCACTTTGGGAAGCCGAGGCAGGTGGATCACTTGAGTTCAGGA | |
| | GTTCGAGAACAGCCTGGCGAAACCCCGTCTCTACTAAAAATAGAA | |
| | AAATCAGCTGGGCCTGGTGGCAGGCGCCTATAATCCCAGCTACTT | |
| | GGAAGGCTGAGGCAGGAGAATTGCTTGAACTCAGGAGGCAGAGG | |
| | TTACAGTGAGTGAAGATGGCGCCACAGCACTCCAGCCTGGGTGAC | |
| | AGAGAGAGAGACTCCTTCTCAAAAAAAAAAAAAAGAAGAAAAAT | |
| | AAAAAGAAAGTGGGCCAGACCGAGAGAACAGGAAGCCTGATGGA | |
| | GTGGGCAAGATTGACAGGCCCAAGGCTGAAAGGCCCAGAAGGTA | |
| | GAAAGGTGAGCTGAGGACAGGCAGATCTGGAAGTGGAACTAGGTT | |
| | GAGGGTTGGGACACAGATCAGCATGGCTAAATGGGAGGCCAGTCC | |
| | TGATCCCACATCCTTGCTGATCCCTTACTTCACAAAGTATCTGGCC | |
| | CAGGTCTGGTGGTAGATGTCTCCGGGCTGTCCCTTGAGTGACCCCT | |
| | TCCCATTCACCGCTCCATTGGTCTTGAGCCAAGTGGGTGATGTCCA | |
| | GGGGCTGGCAAGGAGTGAAACGGGACGCTGGGCCAACTGCAGGG | |
| | CTCGGTGAATCAGGGGTATCTAGAGACAAAGGTAGTGAAGAGAGA | |
| | AGCACCCAGAGTTGGAACACATACTAGCCCAACCAGTGCATCCGG | |
| | TTCAGCCATTAGCCTCCACCCTCCCACCCCCAGGACAAAACAGCA | |
| | GGGGACAAAATGTCTGTACAAGCAGACCTACCCTACAGTTTCTCA | |
| | ACCCCCAGACATCAGGGCCCTCAGGGCCTGAAAAAGCTAGAATGC | |
| | CTACCTTGAGCTTGGTATCTTCCTCTGGGAGGCTGAAGTTGTGCAA | |
| | CTGGAAATCATCAGGGGTGTCTGCATAGGTGTAGGTGCGGATGGA | |
| | GAAGTCACAGCTGGCCATGGGTACCCGGATGATGTTATATCCGATT | |
| | CCTACAGAAAAGGATGATCAAGATATGGTAGTCCGAGTCAATAGG | |
| | AGAGTATGGGACTCTGCTTATCACTTGCCAGTCCTAATAGTGTCTG | |
| | AGTCAGGGCCAAAGGGAACTTGGGCTCCTGGGTTGGAACCTGTGG | |
| | AGGCTGGCACCTGGGTGAAGCGCAGGCCTTTCTGAGCCTGAGTCC | |
| | GTAGCAGTTAGCAGATGATAGGCGGTGAAATCTTATTTCACAGGG | |
| | CATTAAAACAGGAACCAAATGTCAGGGATGGGCAGAAGTCAGGGT | |
| | CCAAAGAAAGGGCAAAGAAAAGTGTCAGTGGCTCACGCATGTAAT | |
| | CCCAGCACTTTGGGAGGCCGATGTGGGCAGATCACGAGGTCAGGA | |
| | GTTCGCAATCATCCTGGCCAACATAGTGAAACCCTGTCTCTACTAA | |
| | AAATACAAAAAATTAGCCGGGCGTGGTGGCAGGCACCTGTAATCC | |
| | CAGGTACTCGAGAGGCTGAGACAGGAGAATCACCTGAACCCGGGA | |
| | GGTGGAGGTTGCAGTGAGCTGAGATTGTGCCACTGCACTCCAGCC | |
| | TGGGTGACAGTGCGAGACTCTGTCTCAAAAAAAAAAAAAGAAAA | |
| | AAGAAAAGTGTCTGCTGGGCTCGGTGGCTCACACTTGTAATTCCAG | |
| | CACTTCGGGAGGCCAAGGCAGGTATATCATTTGAGGTCAGGAGTT | |
| | TAAGACTAGCCTGGTCAACATGATGAAACCCTGTCTCTACTAAAA | |
| | ATACAAAAATTAGCCAGGTGGTAGTGGCGCACGCCTATAATTCCA | |
| | GCTACTCAGGAGGCTGAGGCAGGAGAATCACTATAGCCTGGGAGG | |
| | CAGAGGTTGCTGTGAGGGGAGATCACACCACTGCACTCCTGTCTC | |
| | GCCGACAGAATGGGCAGAGTGAGATTCTGCCTCAAAAAAATTTTA | |
| | AAAAAAGAAAAGAAAAACGAAAAGTTTCAATGGCTCTATGTCATC | |
| | TTGTCCCCTTCCTCCTCACCTTCTTCAGAGAAGTACGATTTAAGTA | |
| | GCAAATTTTGGGCAGGGGGTGACAGGGCAAGGATGTTGAGAGCAG | |
| | CAGCATCTGTCATGGCCCCTCCAAATCCCTTCACTTTCTGGAACTT | |
| | CTGTTCTGGCTGCAGGGTCAGTAGCAGGCCTGAGGACATCCACAG | |
| | GGAATAAGGGTATCAGTACCCAGCGGGAAACTCCATGGTGATCAC | |
| | TGACACCATTTACCTCTAGGAGGACCCAGCCTGGCCCAGGGGGTG | |
| | AGGGGTGTAATGGTTACCTGTGCCCGTGTGATTAGCCTGGATGGGC | |
| | CCCATACTCAGCTCCATCCGTCGCCCACTGCGTGTACTCTCATAGC | |
| | GGCTGAAGGTACCAAGGGCAGGAAAGGTCGGGGGGTCAAAGGAG | |
| | TCACAGTATGTGGCATTGCAGACACACACCACCGAGCTGTAGCCG | |
| | AAGCTTTTAGGGATGCAGGGGGGGGCACCTGGGAGGGAGGGAGT | |
| | ACAAGCAGAGTGAGGTCTGATGAAGACATGGAGAATGGACACATC | |
| | TGCTAGGAGAGACTGAACACGGTTTCAAAATTCCTCACCCCTTGGC | |
| | CGGGCGCAGGGGCTCACACCTGTAATCCTAGCACTTTAGGAGGCC | |
| | GAGGTGGGCGGATCACCTGAGGTGAGGAGTTTGAGACCTGCCTTG | |
| | CCAGCATGGTGAAACCCCGTCTCTACTAAAAATACAAAAATTAGC | |
| | TGGGCGTAGTGGTGGCCGCCTGTAATCCCAGCTACCTGGGAAGCT | |
| | GAAGCAAGAGAATCGCTTGAATCTGGGAGGCAGAGGTTGGAATGA | |
| | GCCAAAATTGCACCACTGCACTCCAGCCCAGGCAACAGAGTAAGA | |
| | CTCTGTTTCAAAAGAAAAAAAACAAAATCCTCACCCCAAAGTTGG | |
| | TCTCAGTCACTCAAAAGGATTTATTGAGCACCTACTAAAAGTCTAC | |
| | CACCAGCTTACTGGAAGGCTACCAAAGGACTATGAGGCAGAAGGG | |
| | AGGCTCTGTGCTACCTCCCCACTGCCTTGACTCACTCACCTGATGC | |
| | CCACGACACTGCCTGAAGTAGAAGCAATCCTGTGAGGCTGCCAGC | |
| | CATGATGCTTACCCTACTCAAAGGCTTGGGACATTCCTGAGGACAG | |
| | AATGAGGAATGACTGAAAAGCAAGCCCCTCTCCACCCCTCAACTA | |
| | CTCTCCTGGGCAGGGCTTAGCTGCCTTTGGGTGCCCATGGCCCGGT | |
| | CTCCCACATTCATTAGGACAAGGCCCACAGAAACCTGGGTGCAGC | |
| | TCTCCCTGCAACCCTTCTGATGACAACTCTCTGCCCACCCCAAATC | |
| | AGGATGCTCCCCACCACTCTTCCCACCCATTTCAACTTCGACCCCT | |
| | CCCTCCATCTGTGCCTTGCTCAAAGAGCCATGATGGCCCTGGATTC | |
| | AAAGAGAGTCTGTCATTCATTAAATTCCAGTGCCAGGATTCCAGA | |
| | AGCACTGTGACAATGCTGATTGGGAGCTCTCTCTCTTACCTCTCTG | |
| | GAAGGACTTGAAAACTCCATCCCCTCAGGGTCATTAGATGAAGAG | |
| | AAGACCACAGGGGTTCCAGAGTCTCTGAAGGATAGAGGATCCACT | |
| | AAACAAAAACAAGGATGCAGGTACCTGCCTTAGCTATAGGCACTA | |
| | GGTTAGCCCTGCAAGTAATTCCGGCTTCCCGATGTGGATGGGTCAT | |
| | GTGATGACTAGGAGCGTCACATGACACAGGAAGTGAGGCAATCAC | |
| | AGCCATATTTCTAAAGGGCAATTGGCTTCCTCTCATCTGTTACAGA | |
| | TTATATGCCCTATAAAACTCTGGAGGGCATGTATGGGTGACAACTT | |
| | TAGGAAGAGCCTAGAACACAGATTTGCATGGAGAATGGGGTAGAG | |
| | GCTCCTAAATCCCAGAGGATGGGAACCACAGCAGGCACACTTCTT | |
| | TTTTGATAGAAATGTCAAAAAGGTACAAATAAAGCTGATATAATT | |
| | TAAAAAAAAAAACTTCACTGTGCAAAATACTGACACTACCAAGAT | |
| | GTTTAAGACAGTCTTTGCCCTATAGAGGTGTGTGAGGCATGGAAGT | |
| | CAGACACACAGATATTTACAGATAAAGACAGAAACGGTAACAGGT | |
| | GTGCATGGAGAGCTCTCTGAGATGAGGAGGGACCATTCGTGGGTG | |
| | GGGAATTATCCAGGATGGCTTCACAGAGGAAAAAGTGAGGGGGGT | |
| | TATTAGCCAGTGAAGTGCAGGGCACAAGAGGGTGGGACACTGGCA | |
| | GTGGGATGACAGGACTGGAGGGAGGGAGTGGTCAGTGCGGCTCCT | |
| | CTGCAGCGTCCCTTGTTTCATCATCAGATGCAGATGGTAATAACTG | |
| | TTCTTCCTTCCTCACAAGACAGGGAGGTTTGTAAAGTCGTTCAAAA | |
| | ACCAAAGTGTTGTACAAAGCCATATCCTCAGTGGACACAAGGAGG | |
| | AAGCTGTCCATGGTGTGGCCTCATGAACCACATCAAATGAGATTTA | |
| | GCGGGAGTGGCACACACAGTCATGACCTGACTAATCCCAGCTCTC | |
| | AGCCCATTTCCTTGCCTGGAAAATGGAGGCAATGCCACAACCTCA | |
| | AAGGGTGGTTACTGCAGTCAGTGAGGTAAGTGCAGTGCCTGACCA | |
| | CTTGGTAGGCACCTGGGAACTACTTGTCTCTTGTTTGTATTTTTTGT | |
| | TGTTGTTGTTTTTTGAGACAGAGTCTCACTCTGTTGCCCAGGCTGG | |
| | AGTGCAGTGGCGTGATCTTGGCTCACTGCAACCTCCACCTCCTGGG | |
| | TTCGAGTGATTCTCCTGCCTCAGCCTCCCAAGTAGCTGGGATTACA | |
| | GGTGCCCTCCAACATGCTCAGCTAATTTTTTTATTTTTAGTAGAGAT | |
| | GGGGTTTCACTATGTTGGCCAGGCTGGTCTGAAACTCTGGACCTCA | |
| | GGTGATCTGCTGCCTTGGTATCCGAAAGTGCTGGGATTACAGGCAT | |
| | GAGCCACTGCGCTGGACCCCAGCCATCCTTTTTGTTCTTTAAATGA | |
| | TCTCAGTGAAGTCTTTCTAGATACACTCCAAGCAAAATTGATCATT | |
| | TCTTCTTCTAGGTTCCTCCAGTAATTTTTTTTTTTTTGGTTTTGAGAC | |
| | AGAGTCTTGCCCTGTTGCCCAGGCTGGAGTGCAGTGTGATCTCGGC | |
| | TCAAGCAATTGCCCTGCCTCAGCCTCCTGAGTAGCTGGGATTACAG | |
| | GAGCCCACCACCATGCCCAGCTAATTTTTGTATTTTTAGTAGAGAT | |
| | GGGGTTTCACCATGTTGGCCAGGCTGGTCTCGAACCCCTGACCTCA | |
| | GGTGATCCACCAGCCTCAGCCTCCCAAGGTGTTGGGATTATAGCA | |
| | ATGAGCCACCACACCCGGTCCCTCCAGTAATTAAGTACAGTCTCAG | |
| | TGAGACAGCAAGTTTGGAATCCTGGCTACACCATTTACTGGCTGTT | |
| | TGACTTTCAATAAATCAATCACTCTAAGCCTCTGTTTCATCTATAA | |
| | AAGGGGAGTGATAACTCCTACCTCACAGAATTGTTGTGAGGTTTG | |
| | AGTGTGATAATGTGTCTCTAGTACACTGCTTGACACTAAACATTGC | |
| | AACATGTCAGGCCTCTGTTCCTGGAGTTCCTTGAAGGAATGTCTTA | |
| | TGCATTCTAAGTATCCTCGTAGATAGCCTGGCACAGGGGTAGCTGT | |
| | CAAGTTGTAGAATTGAACTAGTTGCTTCACTATGTCAGTAGCCACC | |
| | CCTTCCAGACTTCTCACTTTTCAAGGAAACATTGCACAGGATTTGT | |
| | TCTGGGTAGGGCAGGTAATATCTAGTACCTTACTTCCCTCAAGTTC | |
| | ATTCATCTCACAGATATTTCCTGAGCACATTCTACATTTGCCTCTCC | |
| | TGCTCTATTGAGTTTAGAAGTCCAAACATCTCCTTCCACTTCCCCTC | |
| | TGCATAGTGAGCCTCTTCTTTTTTGGCCAGGTACTGAGCAATTTTTT | |
| | GTTTGTTTGTTTGAGACAGGGTCTCCGTATGTTGCCCTGGATGGAG | |
| | TGTAGTAGCTTGATTTCGGCTCAGTGCAACCTCTGCCTCCAGAATA | |
| | AGCAATCCTCCCACCTCAGCCTCCAGAATAGCTGGGACTATAGGC | |
| | GCACACCACCACACCTGGCTAATTTCTGTATTTTTTGTAGAGACGG | |
| | GGTCTTACTGTGTTGCGCAGGTTGGTCTCAAACTCCTAAACTCAAA | |
| | GGATCCTCCCGCCTTGGCTTTGCAAAGTGCTGGGATTACAGGGTGA | |
| | GGCACTGCGCCCAGTCCGCTTGTTTTTTGTTTTGTTTTTTTTTTTGA | |
| | GACGGAGTATCTCTGTCACCCAGGCTGGAGTGCAGTGGCACGATC | |
| | TCGGTTCACTGCAACCTCCGCCTCCTAGGTTCAAGCGGAGGGTAGG | |
| | GACCAGTCCATCCTGGCACCCATCTGCAGCTCCAGTGCGAATCCCA | |
| | ACCCCGACGCTCGTCGCCGGGCTCCGTGAATGTTTGTCACATGTCT | |
| | GAAGAACGTATGAATTACATAACCTTCTTCCCACTCCACCCCTCAA | |
| | AAAGCAAGTGGATATAAAGACTTGAAGATTTTATAATCTCTTCTTC | |
| | ATTAGTAAAATCTGACCATCCTTACCGTTAAAAATAATAATGATGG | |
| | TTGGCCGGGCGCGGTGGCTCACGCCTATAATCACAGCTGTTTGGCA | |
| | GGCCGAGGCGGGCGGATCACGAGGTCAGGAATTGGAGACCAGCCT | |
| | GACCAACATGGTGAAACCCCGTCTCTACTAAAAGATACAAAAAAT | |
| | TAGCCGTGCGTGGTGGCGGGTGCCTGTAATCCCACGTACTTGGGA | |
| | GGCTGAGGTCGGAGAATCGCCTGAACCCGGGAGGCGGAGGTTGCA | |
| | GTGAGCCAAGAAGGCGCCATTACACTCCAGCCTGGGCGACAGGGC | |
| | GAGACTCCCTCAAAAAATAATAATAATAACAATAATAATAATAAT | |
| | GGTGAAAAAGGTTAAGTGCGAACGCAGGGAGGGGACAGCTAAGA | |
| | TCCAAAGGTCGAAATATTCATTACGCCTACCGTCGGCGAAGAGAA | |
| | ACAGCAGCCCCAACCGGAGGCAAAACGAAATCCCACCGCAGCCTG | |
| | CAAAGGCGCCTGGGCGGGACTGGAGACTGGGGCCCCGCGCAGTAA | |
| | GACTCTGAAGGCAGGATGCAGCCCGACCACCCGCAGCCGCGAGAA | |
| | AAGCAGCCCTGGGGAGTCGGGGGGGGACCTGGATTGGAAAAGAG | |
| | ACGGTCACTCATGCAGAGGCGGGACTCAGAGCCCTTCCTCAAGTC | |
| | TCATTGGTCAAGTTGAACGAACAAGTGTCGCTGGCGAGCCGGAGA | |
| | GAGAGAGAGAGCGAGAGCGAGAGAGCGAGCGAGAGAGAGCGAG | |
| | AGAGCGAGAGAGCGAGCGAGAGAGAGAGAGAGAGAGAGAGGAG | |
| | CCGGCGCGAGAACTACGCATGCGTGTCGGCGTTTTCCCGCCAGCA | |
| | CACTGTTGGTGGATGGGGGCGATTGAATTCCCACAGTGAGTCCAG | |
| | CCCACCGAAGCTCAGAGGATTCCTAACCTTCCTCTTTAGAGAGCCT | |
| | CAGGTTAGGGAACGTCCAGTGCGCAGAAGCTGCCCCGTGGGAATC | |
| | CCATTGTCCATCGCCTCCTAATGTTTCGTCCTGGCCGTGCCCTATCC | |
| | CTTCCTGAGGCTGGGTTGTTATGATGCTGAATTATTCAAGAAGTCT | |
| | TGCAGCCTGACGCCATCTCTGGGCAGTGCTCCTCCCACCTCCCTGT | |
| | CTTCCTTGGAGGGCACCACGTTGCCCCTACAAGCACAGGGTCCTGA | |
| | AGCTGTTTACAGGGCCCCACCCTGCCACTTTAGTATCTTAACGAAT | |
| | GTTTGTTGAGGACCTACTGAGTGTTAGACCCACTGCAATGAGCAA | |
| | GTTTCTGATCTTGGGACCTTAACATTCTATGGGACGACACAGATAT | |
| | ATAAACAAAAATAATAATTTTAGATAGTGATACATGCTGAAACCA | |
| | TGGGTATGTGGCCAGGCGCGGTGAGTCACACCTGTAATCCCAGCA | |
| | CTTTGGGAGACCGAGGCAGGCGGATTACTTGAGGTCAGGAGTTTG | |
| | AGACCCGCCTGGCCAACATGGTGAAACCCTATCTCTTCTAAAAATA | |
| | CAAAATTTAGCTGGGCGGGCATGGGGACGGGCGCCTGTAATCCCA | |
| | GCTACTCGGGAGGCTGAGGCAGGAGAATTGCTTGAACCCAGGAGG | |
| | TGGAAGGTTGCAGTGAGCCCAAATCACACCACTGCACTCCAGCCT | |
| | GGGTAACAAAGCAAGACCCTGTCTCAAAAAAAGACAAAATGAAA | |
| | CAAACAAACAAAAAAAAACATGGCTATGTGATAGAATGACTGCGA | |
| | TGGGGATTGGGTGCTCACTGAAATGGTGAGAATGGAGCTAAAACC | |
| | TGATATTAGGTGATAGCACCAGATATAGGGGCCAAGGGTTTCATG | |
| | CTTGGAAAACAGCAGGTGCAAACGTCCTTGTGCTGTGAGGAAATT | |
| | TGGCTGGAACAGAAGGTGGGCCAGATTGTAGCTGACCATAGTCTC | |
| | TGGGAGTTTGGAGATGATTCTAAATACAATGGGAAGCCATTAAAT | |
| | TGAGGCAGAGCCTTGATGTGACGTGCTTTATCATCATTTTGCTGAG | |
| | TGGAGAATGGATTGCTGGGAAAAGTGAAAGACTGATTCAGAGGTT | |
| | GTGGAGAGAGGATGGCTGGAGCTAGGTAGCTGGGGAGGTGGTGG | |
| | GATGTGGATGAATTCAGGATAGGATTGGTTGTGTTGGATATGGGA | |
| | AAAGAAGTCACCATGGTTCCTGAGCTTTTGGAATGTGCAAATGGG | |
| | TAATGGAGGTGCCATTGAGATGGGAACAGTGGAAAAATAACCGGT | |
| | GCTGGGGTGGGAATCAAGAGTTCAGTTGTGGCCATGTTGAATCTG | |
| | AAATGCCCATTAGACATCCAAGCAGACATGCTGAGTTGGATGGCG | |
| | CTCAT | |
| 12 | ACAGAAGTACAATGCTGATTATGATCTGTCAGCTCAGCAAGGGGC | Human |
| | AGACACCCTGGCCTTCATGTCTCTCCTGGAGGAGAAGTTGATCCCG | GBA1 |
| | GTGCTGGTGAGTGTGCCCAGACCTCCCAGCATCCATGGCCAGCCG | (N370S |
| | GGGAGGGGACGGGAACACACAGACCCACACAGAGACTCAGGAGA | mutant) |
| | GCATGGAGGTCAGAAGCCCACCTTGAATCAGACAGGTGCACTGGC | nucleotide |
| | TCAGACCTGCCTGTTTCTTCCTGCCCACCCAATCCAGGTACATACT | sequence |
| | TTTTGGATAGACACCAAGAACTACGTAGAAGTGACCCGGAAGTGG | |
| | TATGCAGAGGCTATGCCCTTTCCCCTCAACTTCTTCCTGCCTGGCC | |
| | GCATGCAGCGGCAGTACATGGAACGGCTACAGCTGCTGACTGGGG | |
| | AGCACAGGCCTGAGGACGAGGAAGAGCTGGAGAAGGAGGTAGCT | |
| | CTGAGACCGGGGGCTATTGTATGAGATGAGCCCCAAGGATGCTGG | |
| | CCAGGAATGGGAGTGCTTAGGTGCGGAGGTGGCACTGTTCCCGCA | |
| | GCTGCAAGCCTACCTGTGTCGCCCCTACAGCTGTACCGAGAGGCTC | |
| | GGGAGTGTCTGACCCTGCTCTCTCAGCGCCTGGGCTCTCAAAAGTT | |
| | CTTCTTTGGAGATGCGTGAGTCTGACTCCAAGAGGGTAATGGGTG | |
| | GCTTGGAAGAAGATACAGGTTCAGATGGAGCAGCTGGAGCTGGGG | |
| | CTGGGGCTGGGGCTGGCTCAGGCTCTGGATAGGAGGTCCCTGAGA | |
| | CAGATACTGGCCCTGGTGACAGTGGGGCTGTGCGTGGGGCCAGAG | |
| | CCTTCTCAGAGGTACAAAAGGGTAGGGTGGGAGGGCAGCCAGGCA | |
| | CAGGAAGGGCCTGAAGAGCTGTGGGGCACTGAGTGTGCCCTTTAT | |
| | GCAGCCCTGGGATAGAGCCCTATTCAGGGCCAGGCTGGCGCCACC | |
| | TGGGGATCTCTCCCCATACCAGGTCTAGAACTGTGTGTCCTGTCCT | |
| | TCCCTGGTGGCCGCCTGCTGCCCAGAGCCCACCTCCCAAGGCTGAC | |
| | TCTTCCTCCAGCTCCATCTTTACCCCTTCTACCCCAGTGGTTCTCCT | |
| | CCATCCCACCCTTCTCTCTCTGCTCCAGCCCTGCCTCCTTGGACGCC | |
| | TTCGTCTTCAGCTACTTGGCCCTGCTGCTGCAGGCAAAGCTGCCCA | |
| | GTGGGAAGCTGCAGGTCCACCTGCGTGGGCTGCACAACCTCTGTG | |
| | CCTATTGTACCCACATTCTCAGTCTCTACTTCCCCTGGGATGGAGG | |
| | TAAGGGGCAGATGGGAGGGGCAGCCCTGGGGAGAGTGGGCAGGG | |
| | ATCCAAGAACTAGTTCTCCTAACACACCTTCCTTCCTTGACCCTCA | |
| | GCTGAGGTACCACCGCAACGCCAGACACCAGCAGGCCCAGAGACT | |
| | GAGGAGGAGCCATACCGGCGCCGGAACCAGATCCTATCTGTGCTG | |
| | GCAGGACTGGCAGCCATGGTGGGCTACGCCTTGCTCAGCGGCATT | |
| | GTCTCCATCCAGCGGGCAACGCCTGCTCGGGCCCCAGGCACCCGG | |
| | ACCCTGGGCATGGCTGAGGAGGATGAAGAGGAATGATTTGTCCTC | |
| | ACGCTCCCAAGACTGGTTTTTCTACTCTCATGCATTCCAGAGGCCC | |
| | CCGTGCCTCCTCGTTGTTGGTACAGCCGGACACGGGGTGCTGCCAC | |
| | CCAGAATAAAGCCACTCACACTGACTGGGCTCAAACATTTTCTCCT | |
| | TTAAGAGCTGCCATTTTTCCTGGCTGGTGCCATAGGAATCATCTGG | |
| | GTGCCTGGGCACACCCGCTGCTGCTTTAAGGCTTCCGCCCTGATGC | |
| | TGACACTGCTGCTCCACGGGCCCAGTTCTGCATCTCCAGGAAAGAC | |
| | AAACAGTCTCCAGTTTTGGGCCCAGCTTTCCTAGTCTCTTCTTTTCC | |
| | TTACCCTCAGCCCTGATCTTGTGTTTGTACGGACAGTGAGCTCACC | |
| | CTAGGCCTGGACCCAGGCCCAGTTTCCAAAGCAAGCAGCACACAG | |
| | CGCATGTTCACATAAGCATGGGGGCTGGGGGGACACTGGGGCTTA | |
| | CTGATCTTTTTCTAGGGGCCTCCAGCCCCTGGCACCACCTAGAGGG | |
| | GAAAGTGAGTCACCCAAACCATTGCCCCTGGGCTTACGTCGCTGTA | |
| | AGCTCACACTGGCCCTGCTGTGCCCTCTTTAGTCACAGACAGCGTG | |
| | TGAGCTGACTCTGTCCCTTTAATGCCCAGGCTGAGCCCAGTGCCTC | |
| | CTTGAGTATCTGCTCCATCACTGGCGACGCCACAGGTAGGTGTGAA | |
| | TGGAGTAGCCAGGTGAGATTGTCTCCAGGAAGCCCACAGCAGGAT | |
| | CCTTGATGGTAAGAGGCACATCCTTAGAGGAGCTAGGGAGCAGGG | |
| | AGGAGAAGCTGAGAGTGTGATCCTGCCAAGGCCCCCAACGCTGTC | |
| | TTCAGCCCACTTCCCAGACCTCACCATTGCCCTCACCGGTTTAGCA | |
| | CGACCACAACAGCAGAGCCATCGGGATGCATCAGTGCCACTGCGT | |
| | CCAGGTCGTTCTTCTGACTGGCAACCAGCCCCACTCTCTGGGAGCC | |
| | CTCAGGAATGAACTTGCTGAATGTGGGAACAGATGGTCAGAGTCC | |
| | CTCGGGGTACCTCCCATGAAACCCTCATCTAAGAAGTCACCCACCC | |
| | ACGGACCCACCCCATAACTCCTGCAGAGGCTCTGCCCTGGCTCTCT | |
| | AGGCCTGGAGCCATGCTGCTGGGCACTGACCCTGCTTTTCTGCATC | |
| | GCAGTCCAGCCTCAGGCATTGGGGTTTTCTGTTGCTACCTAGTCAC | |
| | TTCCTGCCTCCATGGTGCAAAAGGGGATGGGTGTGCCTCTTCCGAG | |
| | GTTCCACCCTGAACACCTTCCTGCTCCCTCGTGGTGTAGAGTGATG | |
| | TAAGCCATCCGATGTAGGAGATGATAGGCCTGGTATGGAATGGGG | |
| | GTGCCCGCCCTCCACTCACCTGAAGTGGCCAAGGTGGTAGAACAT | |
| | GGGCTGTTTGTAAAACGTGTCCTTGGTGATGTCTACAATGATGGGA | |
| | CTGTCGACAAAGTTACGCACCCAATTGGGTCCTCCTTCGGGGTTCA | |
| | GGGCAAGGTTCCAGTCGGTCCAGCCGACCACATGGTACAGGAGGC | |
| | TCTAGGGTAAGGACAAAGGCAAAGAGACAAAGGCTCAACACTGG | |
| | GGGTCCCCAGAGAGTGTAGGTAAGGGTCACATGTGGGAGAGGCAG | |
| | CTGTGGGTAGGTCAGCCCTGTGAGGGGCACATTCCTTAGTAGCTAA | |
| | GGAGTTGGGGGTGTGAAGATCCAGGCATCTCAAGGGGAGCTGAGA | |
| | AGTCTGAGGCAGCTGCAAGTGCCTCAGTAGTTGCAAAAGGGGCAA | |
| | TGAGGTGTGCAGACCTGTGAAGGAAAGGGAAGATAGGGAATCAT | |
| | GGTTCCCCAGAGTTGCTCAAAAGGGCAGGCTAGCTGGGGAAAGCT | |
| | GGACAGGAAGGGCTTCTGTCAGTCTTTGGTGAAACTAGTAAGAGG | |
| | TCTGAGGTCTGCTTTGCAGGAAGGGAGACTGGGGTGGCTTACCGT | |
| | GATGATGCTGTGGCTGTACTGCATCCCTCGATCCCAGGAGCCTAGC | |
| | CGCACACTCTGCTCCCAGAACTTGGAGCCCACACAGGCCTCTGAG | |
| | GCAAAGAGCATGGTGTTGGGGAACAGGCGGTGTGTCTCCCCTAGG | |
| | GTGGCTTTGGCTGGAGCCAGAAAGTCCAGGTACCAATGTACAGCA | |
| | ATGCCATGAACATATTTAGCTGCTTCTGGGTCTGTCAGTACCTGCA | |
| | AAGGAAGAGCAACTGATCCTGGACCTTGCACACAGGCTTCTGGAA | |
| | CTTCTAGTTCCTGTTGTAGGAATCCTGGAGTTGGGTGACGGGAAGA | |
| | ATGCAACTAGAGAGGTTTGGGGAGATTTTTTTTTGTTTTTGAGACA | |
| | GGATGTCACTCTGTCACTCGGGCTGGAGTACAGTGGCGCAATCAC | |
| | GACTCACTGCAGCCTTGACTTCCTAGGGTCAACTGATCCTCCCACA | |
| | TTAGCCTCCTGAGTAGCTGGGACTACACGGGTGCCACACCCAGCT | |
| | AATTTGTGTGTGTATGTGTGTGTATGTATGTGTGTGTGTGTATATAT | |
| | ATATACATATAAACACATATATATGTATATACACATACATACATGA | |
| | ACCACCACCCCCAGCCTAGATAGTTTTGTTTTGTTTTGTTTCGAGAT | |
| | GGAGTCTCGCTCAGCCTCCCAGGCTGGAGTGCAGTGGCGCGATCT | |
| | CGGCTCACCGCAACCACCATCTCCCGGGTTCAACTGATTCTCCCTT | |
| | CTCAGCCTCCTGAGTAGCTGGGATTACAGACACCCACCATCATGCC | |
| | CAGATAATTTTTTTTTTTTGTATTTTAGTAGACACAGGGTTTCAACA | |
| | CGTTGGCCAGGGTGGTCTTAAACTCCTGACCTCAGGTGATCACCCG | |
| | CCTCGGCTTCCCAAAGTGCTGGGTTTGCATGAGTGAGCCACCTCGC | |
| | CCAGCCCCTAGAAAGGTTTCAAGCGACAACTGTGGGATCCATGGC | |
| | ACCCTGGAGGTCCAGGGGAATGGTGCTCTAGGAATCCATAGTTGG | |
| | GTAGAGAAATCGCTCTAAGTTTGGGAGCCAGTCATTTGGATGCTG | |
| | GATTTGAAGGTCACTGGAGCACCATGGAGGTCCAGGCCTTACCAC | |
| | CTTTGCCCAGTGGGGCAGCAGCAAGCGTTGGTCATCCAGCATGAG | |
| | TAGGCGGACATTGTGGTGAGTACTGTTGGCGAGGGTAGGACCTAG | |
| | GTCACGGGCAATGAAGTCTCGCTGATGTTCAGGGGTGAAGCCCAG | |
| | GCACTGGAAGGGGTATCCACTCAACAGCCCAGCAGAAGGCTCATT | |
| | TTCAGCTGTCACTGCCCAGAACTGTAACTTGTGCTCAGCATAGGCA | |
| | TCCAGGAACCTGGCAAGAGAAAGGTCATGAATGATCCGGCCAAGA | |
| | AAGTGGACCAGACCAGCTGGGTGTGGTGGCTCACACCTGTAATCC | |
| | CAGCACTTTGGGAAGCCGAGGCAGGTGGATCACTTGAGTTCAGGA | |
| | GTTCGAGAACAGCCTGGCGAAACCCCGTCTCTACTAAAAATAGAA | |
| | AAATCAGCTGGGCCTGGTGGCAGGCGCCTATAATCCCAGCTACTT | |
| | GGAAGGCTGAGGCAGGAGAATTGCTTGAACTCAGGAGGCAGAGG | |
| | TTACAGTGAGTGAAGATGGCGCCACAGCACTCCAGCCTGGGTGAC | |
| | AGAGAGAGAGACTCCTTCTCAAAAAAAAAAAAAAGAAGAAAAAT | |
| | AAAAAGAAAGTGGGCCAGACCGAGAGAACAGGAAGCCTGATGGA | |
| | GTGGGCAAGATTGACAGGCCCAAGGCTGAAAGGCCCAGAAGGTA | |
| | GAAAGGTGAGCTGAGGACAGGCAGATCTGGAAGTGGAACTAGGTT | |
| | GAGGGTTGGGACACAGATCAGCATGGCTAAATGGGAGGCCAGTCC | |
| | TGATCCCACATCCTTGCTGATCCCTTACTTCACAAAGTATCTGGCC | |
| | CAGGTCTGGTGGTAGATGTCTCCGGGCTGTCCCTTGAGTGACCCCT | |
| | TCCCATTCACCGCTCCATTGGTCTTGAGCCAAGTGGGTGATGTCCA | |
| | GGGGCTGGCAAGGAGTGAAACGGGACGCTGGGCCAACTGCAGGG | |
| | CTCGGTGAATCAGGGGTATCTAGAGACAAAGGTAGTGAAGAGAGA | |
| | AGCACCCAGAGTTGGAACACATACTAGCCCAACCAGTGCATCCGG | |
| | TTCAGCCATTAGCCTCCACCCTCCCACCCCCAGGACAAAACAGCA | |
| | GGGGACAAAATGTCTGTACAAGCAGACCTACCCTACAGTTTCTCA | |
| | ACCCCCAGACATCAGGGCCCTCAGGGCCTGAAAAAGCTAGAATGC | |
| | CTACCTTGAGCTTGGTATCTTCCTCTGGGAGGCTGAAGTTGTGCAA | |
| | CTGGAAATCATCAGGGGTGTCTGCATAGGTGTAGGTGCGGATGGA | |
| | GAAGTCACAGCTGGCCATGGGTACCCGGATGATGTTATATCCGATT | |
| | CCTACAGAAAAGGATGATCAAGATATGGTAGTCCGAGTCAATAGG | |
| | AGAGTATGGGACTCTGCTTATCACTTGCCAGTCCTAATAGTGTCTG | |
| | AGTCAGGGCCAAAGGGAACTTGGGCTCCTGGGTTGGAACCTGTGG | |
| | AGGCTGGCACCTGGGTGAAGCGCAGGCCTTTCTGAGCCTGAGTCC | |
| | GTAGCAGTTAGCAGATGATAGGCGGCGAAATCTTATTTCACAGGG | |
| | CATTAAAACAGGAACCAAATGTCAGGGATGGGCAGAAGTCAGGGT | |
| | CCAAAGAAAGGGCAAAGAAAAGTGTCAGTGGCTCACGCATGTAAT | |
| | CCCAGCACTTTGGGAGGCCGATGTGGGCAGATCACGAGGTCAGGA | |
| | GTTCGCAATCATCCTGGCCAACATAGTGAAACCCTGTCTCTACTAA | |
| | AAATACAAAAAATTAGCCGGGCGTGGTGGCAGGCACCTGTAATCC | |
| | CAGGTACTCGAGAGGCTGAGACAGGAGAATCACCTGAACCCGGGA | |
| | GGTGGAGGTTGCAGTGAGCTGAGATTGTGCCACTGCACTCCAGCC | |
| | TGGGTGACAGTGCGAGACTCTGTCTCAAAAAAAAAAAAAGAAAA | |
| | AAGAAAAGTGTCTGCTGGGCTCGGTGGCTCACACTTGTAATTCCAG | |
| | CACTTCGGGAGGCCAAGGCAGGTATATCATTTGAGGTCAGGAGTT | |
| | TAAGACTAGCCTGGTCAACATGATGAAACCCTGTCTCTACTAAAA | |
| | ATACAAAAATTAGCCAGGTGGTAGTGGCGCACGCCTATAATTCCA | |
| | GCTACTCAGGAGGCTGAGGCAGGAGAATCACTATAGCCTGGGAGG | |
| | CAGAGGTTGCTGTGAGGGGAGATCACACCACTGCACTCCTGTCTC | |
| | GCCGACAGAATGGGCAGAGTGAGATTCTGCCTCAAAAAAATTTTA | |
| | AAAAAAGAAAAGAAAAACGAAAAGTTTCAATGGCTCTATGTCATC | |
| | TTGTCCCCTTCCTCCTCACCTTCTTCAGAGAAGTACGATTTAAGTA | |
| | GCAAATTTTGGGCAGGGGGTGACAGGGCAAGGATGTTGAGAGCAG | |
| | CAGCATCTGTCATGGCCCCTCCAAATCCCTTCACTTTCTGGAACTT | |
| | CTGTTCTGGCTGCAGGGTCAGTAGCAGGCCTGAGGACATCCACAG | |
| | GGAATAAGGGTATCAGTACCCAGCGGGAAACTCCATGGTGATCAC | |
| | TGACACCATTTACCTCTAGGAGGACCCAGCCTGGCCCAGGGGGTG | |
| | AGGGGTGTAATGGTTACCTGTGCCCGTGTGATTAGCCTGGATGGGC | |
| | CCCATACTCAGCTCCATCCGTCGCCCACTGCGTGTACTCTCATAGC | |
| | GGCTGAAGGTACCAAGGGCAGGAAAGGTCGGGGGGTCAAAGGAG | |
| | TCACAGTATGTGGCATTGCAGACACACACCACCGAGCTGTAGCCG | |
| | AAGCTTTTAGGGATGCAGGGGCGGGCACCTGGGAGGGAGGGAGT | |
| | ACAAGCAGAGTGAGGTCTGATGAAGACATGGAGAATGGACACATC | |
| | TGCTAGGAGAGACTGAACACGGTTTCAAAATTCCTCACCCCTTGGC | |
| | CGGGCGCAGGGGCTCACACCTGTAATCCTAGCACTTTAGGAGGCC | |
| | GAGGTGGGCGGATCACCTGAGGTGAGGAGTTTGAGACCTGCCTTG | |
| | CCAGCATGGTGAAACCCCGTCTCTACTAAAAATACAAAAATTAGC | |
| | TGGGCGTAGTGGTGGCCGCCTGTAATCCCAGCTACCTGGGAAGCT | |
| | GAAGCAAGAGAATCGCTTGAATCTGGGAGGCAGAGGTTGGAATGA | |
| | GCCAAAATTGCACCACTGCACTCCAGCCCAGGCAACAGAGTAAGA | |
| | CTCTGTTTCAAAAGAAAAAAAACAAAATCCTCACCCCAAAGTTGG | |
| | TCTCAGTCACTCAAAAGGATTTATTGAGCACCTACTAAAAGTCTAC | |
| | CACCAGCTTACTGGAAGGCTACCAAAGGACTATGAGGCAGAAGGG | |
| | AGGCTCTGTGCTACCTCCCCACTGCCTTGACTCACTCACCTGATGC | |
| | CCACGACACTGCCTGAAGTAGAAGCAATCCTGTGAGGCTGCCAGC | |
| | CATGATGCTTACCCTACTCAAAGGCTTGGGACATTCCTGAGGACAG | |
| | AATGAGGAATGACTGAAAAGCAAGCCCCTCTCCACCCCTCAACTA | |
| | CTCTCCTGGGCAGGGCTTAGCTGCCTTTGGGTGCCCATGGCCCGGT | |
| | CTCCCACATTCATTAGGACAAGGCCCACAGAAACCTGGGTGCAGC | |
| | TCTCCCTGCAACCCTTCTGATGACAACTCTCTGCCCACCCCAAATC | |
| | AGGATGCTCCCCACCACTCTTCCCACCCATTTCAACTTCGACCCCT | |
| | CCCTCCATCTGTGCCTTGCTCAAAGAGCCATGATGGCCCTGGATTC | |
| | AAAGAGAGTCTGTCATTCATTAAATTCCAGTGCCAGGATTCCAGA | |
| | AGCACTGTGACAATGCTGATTGGGAGCTCTCTCTCTTACCTCTCTG | |
| | GAAGGACTTGAAAACTCCATCCCCTCAGGGTCATTAGATGAAGAG | |
| | AAGACCACAGGGGTTCCAGAGTCTCTGAAGGATAGAGGATCCACT | |
| | AAACAAAAACAAGGATGCAGGTACCTGCCTTAGCTATAGGCACTA | |
| | GGTTAGCCCTGCAAGTAATTCCGGCTTCCCGATGTGGATGGGTCAT | |
| | GTGATGACTAGGAGCGTCACATGACACAGGAAGTGAGGCAATCAC | |
| | AGCCATATTTCTAAAGGGCAATTGGCTTCCTCTCATCTGTTACAGA | |
| | TTATATGCCCTATAAAACTCTGGAGGGCATGTATGGGTGACAACTT | |
| | TAGGAAGAGCCTAGAACACAGATTTGCATGGAGAATGGGGTAGAG | |
| | GCTCCTAAATCCCAGAGGATGGGAACCACAGCAGGCACACTTCTT | |
| | TTTTGATAGAAATGTCAAAAAGGTACAAATAAAGCTGATATAATT | |
| | TAAAAAAAAAAACTTCACTGTGCAAAATACTGACACTACCAAGAT | |
| | GTTTAAGACAGTCTTTGCCCTATAGAGGTGTGTGAGGCATGGAAGT | |
| | CAGACACACAGATATTTACAGATAAAGACAGAAACGGTAACAGGT | |
| | GTGCATGGAGAGCTCTCTGAGATGAGGAGGGACCATTCGTGGGTG | |
| | GGGAATTATCCAGGATGGCTTCACAGAGGAAAAAGTGAGGGGGGT | |
| | TATTAGCCAGTGAAGTGCAGGGCACAAGAGGGTGGGACACTGGCA | |
| | GTGGGATGACAGGACTGGAGGGAGGGAGTGGTCAGTGCGGCTCCT | |
| | CTGCAGCGTCCCTTGTTTCATCATCAGATGCAGATGGTAATAACTG | |
| | TTCTTCCTTCCTCACAAGACAGGGAGGTTTGTAAAGTCGTTCAAAA | |
| | ACCAAAGTGTTGTACAAAGCCATATCCTCAGTGGACACAAGGAGG | |
| | AAGCTGTCCATGGTGTGGCCTCATGAACCACATCAAATGAGATTTA | |
| | GCGGGAGTGGCACACACAGTCATGACCTGACTAATCCCAGCTCTC | |
| | AGCCCATTTCCTTGCCTGGAAAATGGAGGCAATGCCACAACCTCA | |
| | AAGGGTGGTTACTGCAGTCAGTGAGGTAAGTGCAGTGCCTGACCA | |
| | CTTGGTAGGCACCTGGGAACTACTTGTCTCTTGTTTGTATTTTTTGT | |
| | TGTTGTTGTTTTTTGAGACAGAGTCTCACTCTGTTGCCCAGGCTGG | |
| | AGTGCAGTGGCGTGATCTTGGCTCACTGCAACCTCCACCTCCTGGG | |
| | TTCGAGTGATTCTCCTGCCTCAGCCTCCCAAGTAGCTGGGATTACA | |
| | GGTGCCCTCCAACATGCTCAGCTAATTTTTTTATTTTTAGTAGAGAT | |
| | GGGGTTTCACTATGTTGGCCAGGCTGGTCTGAAACTCTGGACCTCA | |
| | GGTGATCTGCTGCCTTGGTATCCGAAAGTGCTGGGATTACAGGCAT | |
| | GAGCCACTGCGCTGGACCCCAGCCATCCTTTTTGTTCTTTAAATGA | |
| | TCTCAGTGAAGTCTTTCTAGATACACTCCAAGCAAAATTGATCATT | |
| | TCTTCTTCTAGGTTCCTCCAGTAATTTTTTTTTTTTTGGTTTTGAGAC | |
| | AGAGTCTTGCCCTGTTGCCCAGGCTGGAGTGCAGTGTGATCTCGGC | |
| | TCAAGCAATTGCCCTGCCTCAGCCTCCTGAGTAGCTGGGATTACAG | |
| | GAGCCCACCACCATGCCCAGCTAATTTTTGTATTTTTAGTAGAGAT | |
| | GGGGTTTCACCATGTTGGCCAGGCTGGTCTCGAACCCCTGACCTCA | |
| | GGTGATCCACCAGCCTCAGCCTCCCAAGGTGTTGGGATTATAGCA | |
| | ATGAGCCACCACACCCGGTCCCTCCAGTAATTAAGTACAGTCTCAG | |
| | TGAGACAGCAAGTTTGGAATCCTGGCTACACCATTTACTGGCTGTT | |
| | TGACTTTCAATAAATCAATCACTCTAAGCCTCTGTTTCATCTATAA | |
| | AAGGGGAGTGATAACTCCTACCTCACAGAATTGTTGTGAGGTTTG | |
| | AGTGTGATAATGTGTCTCTAGTACACTGCTTGACACTAAACATTGC | |
| | AACATGTCAGGCCTCTGTTCCTGGAGTTCCTTGAAGGAATGTCTTA | |
| | TGCATTCTAAGTATCCTCGTAGATAGCCTGGCACAGGGGTAGCTGT | |
| | CAAGTTGTAGAATTGAACTAGTTGCTTCACTATGTCAGTAGCCACC | |
| | CCTTCCAGACTTCTCACTTTTCAAGGAAACATTGCACAGGATTTGT | |
| | TCTGGGTAGGGCAGGTAATATCTAGTACCTTACTTCCCTCAAGTTC | |
| | ATTCATCTCACAGATATTTCCTGAGCACATTCTACATTTGCCTCTCC | |
| | TGCTCTATTGAGTTTAGAAGTCCAAACATCTCCTTCCACTTCCCCTC | |
| | TGCATAGTGAGCCTCTTCTTTTTTGGCCAGGTACTGAGCAATTTTTT | |
| | GTTTGTTTGTTTGAGACAGGGTCTCCGTATGTTGCCCTGGATGGAG | |
| | TGTAGTAGCTTGATTTCGGCTCAGTGCAACCTCTGCCTCCAGAATA | |
| | AGCAATCCTCCCACCTCAGCCTCCAGAATAGCTGGGACTATAGGC | |
| | GCACACCACCACACCTGGCTAATTTCTGTATTTTTTGTAGAGACGG | |
| | GGTCTTACTGTGTTGCGCAGGTTGGTCTCAAACTCCTAAACTCAAA | |
| | GGATCCTCCCGCCTTGGCTTTGCAAAGTGCTGGGATTACAGGGTGA | |
| | GGCACTGCGCCCAGTCCGCTTGTTTTTTGTTTTGTTTTTTTTTTTGA | |
| | GACGGAGTATCTCTGTCACCCAGGCTGGAGTGCAGTGGCACGATC | |
| | TCGGTTCACTGCAACCTCCGCCTCCTAGGTTCAAGCGGAGGGTAGG | |
| | GACCAGTCCATCCTGGCACCCATCTGCAGCTCCAGTGCGAATCCCA | |
| | ACCCCGACGCTCGTCGCCGGGCTCCGTGAATGTTTGTCACATGTCT | |
| | GAAGAACGTATGAATTACATAACCTTCTTCCCACTCCACCCCTCAA | |
| | AAAGCAAGTGGATATAAAGACTTGAAGATTTTATAATCTCTTCTTC | |
| | ATTAGTAAAATCTGACCATCCTTACCGTTAAAAATAATAATGATGG | |
| | TTGGCCGGGCGCGGTGGCTCACGCCTATAATCACAGCTGTTTGGCA | |
| | GGCCGAGGCGGGCGGATCACGAGGTCAGGAATTGGAGACCAGCCT | |
| | GACCAACATGGTGAAACCCCGTCTCTACTAAAAGATACAAAAAAT | |
| | TAGCCGTGCGTGGTGGCGGGTGCCTGTAATCCCACGTACTTGGGA | |
| | GGCTGAGGTCGGAGAATCGCCTGAACCCGGGAGGCGGAGGTTGCA | |
| | GTGAGCCAAGAAGGCGCCATTACACTCCAGCCTGGGCGACAGGGC | |
| | GAGACTCCCTCAAAAAATAATAATAATAACAATAATAATAATAAT | |
| | GGTGAAAAAGGTTAAGTGCGAACGCAGGGAGGGGACAGCTAAGA | |
| | TCCAAAGGTCGAAATATTCATTACGCCTACCGTCGGCGAAGAGAA | |
| | ACAGCAGCCCCAACCGGAGGCAAAACGAAATCCCACCGCAGCCTG | |
| | CAAAGGCGCCTGGGCGGGACTGGAGACTGGGGCCCCGCGCAGTAA | |
| | GACTCTGAAGGCAGGATGCAGCCCGACCACCCGCAGCCGCGAGAA | |
| | AAGCAGCCCTGGGGAGTCGGGGGGGGACCTGGATTGGAAAAGAG | |
| | ACGGTCACTCATGCAGAGGCGGGACTCAGAGCCCTTCCTCAAGTC | |
| | TCATTGGTCAAGTTGAACGAACAAGTGTCGCTGGCGAGCCGGAGA | |
| | GAGAGAGAGAGCGAGAGCGAGAGAGCGAGCGAGAGAGAGCGAG | |
| | AGAGCGAGAGAGCGAGCGAGAGAGAGAGAGAGAGAGAGAGGAG | |
| | CCGGCGCGAGAACTACGCATGCGTGTCGGCGTTTTCCCGCCAGCA | |
| | CACTGTTGGTGGATGGGGGCGATTGAATTCCCACAGTGAGTCCAG | |
| | CCCACCGAAGCTCAGAGGATTCCTAACCTTCCTCTTTAGAGAGCCT | |
| | CAGGTTAGGGAACGTCCAGTGCGCAGAAGCTGCCCCGTGGGAATC | |
| | CCATTGTCCATCGCCTCCTAATGTTTCGTCCTGGCCGTGCCCTATCC | |
| | CTTCCTGAGGCTGGGTTGTTATGATGCTGAATTATTCAAGAAGTCT | |
| | TGCAGCCTGACGCCATCTCTGGGCAGTGCTCCTCCCACCTCCCTGT | |
| | CTTCCTTGGAGGGCACCACGTTGCCCCTACAAGCACAGGGTCCTGA | |
| | AGCTGTTTACAGGGCCCCACCCTGCCACTTTAGTATCTTAACGAAT | |
| | GTTTGTTGAGGACCTACTGAGTGTTAGACCCACTGCAATGAGCAA | |
| | GTTTCTGATCTTGGGACCTTAACATTCTATGGGACGACACAGATAT | |
| | ATAAACAAAAATAATAATTTTAGATAGTGATACATGCTGAAACCA | |
| | TGGGTATGTGGCCAGGCGCGGTGAGTCACACCTGTAATCCCAGCA | |
| | CTTTGGGAGACCGAGGCAGGCGGATTACTTGAGGTCAGGAGTTTG | |
| | AGACCCGCCTGGCCAACATGGTGAAACCCTATCTCTTCTAAAAATA | |
| | CAAAATTTAGCTGGGCGGGCATGGGGACGGGCGCCTGTAATCCCA | |
| | GCTACTCGGGAGGCTGAGGCAGGAGAATTGCTTGAACCCAGGAGG | |
| | TGGAAGGTTGCAGTGAGCCCAAATCACACCACTGCACTCCAGCCT | |
| | GGGTAACAAAGCAAGACCCTGTCTCAAAAAAAGACAAAATGAAA | |
| | CAAACAAACAAAAAAAAACATGGCTATGTGATAGAATGACTGCGA | |
| | TGGGGATTGGGTGCTCACTGAAATGGTGAGAATGGAGCTAAAACC | |
| | TGATATTAGGTGATAGCACCAGATATAGGGGCCAAGGGTTTCATG | |
| | CTTGGAAAACAGCAGGTGCAAACGTCCTTGTGCTGTGAGGAAATT | |
| | TGGCTGGAACAGAAGGTGGGCCAGATTGTAGCTGACCATAGTCTC | |
| | TGGGAGTTTGGAGATGATTCTAAATACAATGGGAAGCCATTAAAT | |
| | TGAGGCAGAGCCTTGATGTGACGTGCTTTATCATCATTTTGCTGAG | |
| | TGGAGAATGGATTGCTGGGAAAAGTGAAAGACTGATTCAGAGGTT | |
| | GTGGAGAGAGGATGGCTGGAGCTAGGTAGCTGGGGAGGTGGTGG | |
| | GATGTGGATGAATTCAGGATAGGATTGGTTGTGTTGGATATGGGA | |
| | AAAGAAGTCACCATGGTTCCTGAGCTTTTGGAATGTGCAAATGGG | |
| | TAATGGAGGTGCCATTGAGATGGGAACAGTGGAAAAATAACCGGT | |
| | GCTGGGGTGGGAATCAAGAGTTCAGTTGTGGCCATGTTGAATCTG | |
| | AAATGCCCATTAGACATCCAAGCAGACATGCTGAGTTGGATGGCG | |
| | CTCAT | |
| 13 | AGCCGACCACATGGTACAGG | crRNA |
| | | (rs76763715) |
| 14 | GTACAGGAGGCTCTAGGGTA | crRNA |
| | | (rs76763715) |
| 15 | GAGACAAAGGCTCAACACTG | crRNA |
| | | (rs76763715) |
| 16 | AAGAGACAAAGGCTCAACAC | crRNA |
| | | (rs76763715) |
| 17 | ACATGGTACAGGAGGCTCTA | crRNA |
| | | (rs76763715) |
| 18 | AGGCTCTAGGGTAAGGACAA | crRNA |
| | | (rs76763715) |
| 19 | CTAGAGCCTCCTGTACCATG | crRNA |
| | | (rs76763715) |
| 20 | CACATGGTACAGGAGGCTCT | crRNA |
| | | (rs76763715) |
| 21 | AGCCTCCTGTACCATGTGGT | crRNA |
| | | (rs76763715) |
| 22 | AGTCGGTCCAGCCGACCACA | crRNA |
| | | (rs76763715) |
| 23 | ATGTGGTCGGCTGGACCGAC | crRNA |
| | | (rs76763715) |
| 24 | TCCAGCCGACCACATGGTAC | crRNA |
| | | (rs76763715) |
| 25 | CAGGGCAAGGTTCCAGTCGGTCCAGCCGACCACATGGTAC | 5′ sSODN |
| | | (rs76763715) |
| 26 | AGCAAGTTCTAGGGTAAGGACAAAGGCAAAGAGACAAAGGCTCA | 3′ ssODN |
| | A | (rs76763715) |
| 27 | CAGGGCAAGGTTCCAGTCGGTCCAGCCGACCACATGGTACAGCAA | ssODN |
| | GTTCTAGGGTAAGGACAAAGGCAAAGAGACAAAGGCTCAA | (rs76763715) |
| 28 | CAAGGTTCCAGTCGGTCCAGCCGACCACATGGTACAGGAGGTTCT | 5′ ssODN |
| | AG | (rs76763715) |
| 29 | GGTAAGCACAAAGGCAAAGAGACAAAGGCGCAACACTGGGG | 3′ ssODN |
| | | (rs76763715) |
| 30 | CAAGGTTCCAGTCGGTCCAGCCGACCACATGGTACAGGAGGTTCT | ssODN |
| | AGGGTAAGCACAAAGGCAAAGAGACAAAGGCGCAACACTGGGG | (rs76763715) |
| 31 | AAGGTTCCAGTCGGTCCAGCCGACCACATGGTACAGGAGGTTCTA | 5′ ssODN |
| | GGGTAAGGACAAAGGCAAAGAGA | (rs76763715) |
| 32 | CAAACGCTCAACACTGGGGGTCCCCAGAGAGTGTAGGTAA | 3′ ssODN |
| | | (rs76763715) |
| 33 | AAGGTTCCAGTCGGTCCAGCCGACCACATGGTACAGGAGGTTCTA | ssODN |
| | GGGTAAGGACAAAGGCAAAGAGACAAACGCTCAACACTGGGGGT | (rs76763715) |
| | CCCCAGAGAGTGTAGGTAA | |
| 34 | AAGGTTCCAGTCGGTCCAGCCGACCACATGGTACAGGAGGTTCTA | 5′ ssODN |
| | GGGTAAGGACAAAGGCAAAGAGACAAAGGCTCAACA | (rs76763715) |
| 35 | CTGGCCGTCCCCAGAGAGTGTAGGTAAGGGTCACATGTGG | 3′ ssODN |
| | | (rs76763715) |
| 36 | AAGGTTCCAGTCGGTCCAGCCGACCACATGGTACAGGAGGTTCTA | ssODN |
| | GGGTAAGGACAAAGGCAAAGAGACAAAGGCTCAACACTGGCCGT | (rs76763715) |
| | CCCCAGAGAGTGTAGGTAAGGGTCACATGTGG | |
| 37 | CAAGGTTCCAGTCGGTCCAGCCGACCACATGGTACAGGAGGTTCT | 5′ ssODN |
| | AGGGTAAGGACAAAGGCAAAGAGACAAAGGCTCAA | (rs76763715) |
| 38 | CACTCCGGGTCCCCAGAGAGTGTAGGTAAGGGTCACATGT | 3′ ssODN |
| | | (rs76763715) |
| 39 | CAAGGTTCCAGTCGGTCCAGCCGACCACATGGTACAGGAGGTTCT | ssODN |
| | AGGGTAAGGACAAAGGCAAAGAGACAAAGGCTCAACACTCCGGG | (rs76763715) |
| | TCCCCAGAGAGTGTAGGTAAGGGTCACATGT | |
| 40 | CAAGGTTCCAGTCGGTCCAGCCGACCACATGGTACAGGAGGTT | 5′ ssODN |
| | | (rs76763715) |
| 41 | CTAGGCTAAGGACAAAGGCAAAGAGACAAAGGCTCAACAC | 3′ ssODN |
| | | (rs76763715) |
| 42 | CAAGGTTCCAGTCGGTCCAGCCGACCACATGGTACAGGAGGTTCT | ssODN |
| | AGGCTAAGGACAAAGGCAAAGAGACAAAGGCTCAACAC | (rs76763715) |
| 43 | CAAGGTTCCAGTCGGTCCAGCCGACCACATGGTACAGGAGGTTCT | 5′ ssODN |
| | AGGGTAAGGA | (rs76763715) |
| 44 | CAAAGCCAAAGAGACAAAGGCTCAACACTGGGGGTCCCCA | 3′ ssODN |
| | | (rs76763715) |
| 45 | CAAGGTTCCAGTCGGTCCAGCCGACCACATGGTACAGGAGGTTCT | ssODN |
| | AGGGTAAGGACAAAGCCAAAGAGACAAAGGCTCAACACTGGGGG | (rs76763715) |
| | TCCCCA | |
| 46 | AAGGTTCCAGTCGGTCCAGCCGACGACAT | 5′ ssODN |
| | | (rs76763715) |
| 47 | GGTACAGGAGGTTCTAGGGTAAGGACAAAGGCAAAGAGACAAAG | 3′ ssODN |
| | GCTCAACA | (rs76763715) |
| 48 | AAGGTTCCAGTCGGTCCAGCCGACGACATGGTACAGGAGGTTCTA | ssODN |
| | GGGTAAGGACAAAGGCAAAGAGACAAAGGCTCAACA | (rs76763715) |
| 49 | AAGGTTCCAGTCGGTCCAGCCGACCACATGGTACAGGAGGT | 5′ ssODN |
| | | (rs76763715) |
| 50 | TCTACCGTAAGGACAAAGGCAAAGAGACAAAGGCTCAACA | 3′ ssODN |
| | | (rs76763715) |
| 51 | AAGGTTCCAGTCGGTCCAGCCGACCACATGGTACAGGAGGTTCTA | ssODN |
| | CCGTAAGGACAAAGGCAAAGAGACAAAGGCTCAACA | (rs76763715) |
| 52 | AAGGTTCCAGTCGGTCCAGCCGACC | 5′ ssODN |
| | | (rs76763715) |
| 53 | ACATGGTACAGGAGGTTCTAGGGTAAGGACAAAGGCAAAGAGAC | 3′ ssODN |
| | AAAGGCTCAACA | (rs76763715) |
| 54 | AAGGTTCCAGTCGGTCCAGCCGACCACATGGTACAGGAGGTTCTA | ssODN |
| | GGGTAAGGACAAAGGCAAAGAGACAAAGGCTCAACA | (rs76763715) |
| 55 | TTCGGGGTTCAGGGCAAGGTTCCAGTCGGTCCAGCCGACC | 5′ ssODN |
| | | (rs76763715) |
| 56 | ACATGATACAGGAGGTTCTAGGGTAAGGACAAAGGCAAAGAGAC | 3′ ssODN |
| | AAAGGCTCAACA | (rs76763715) |
| 57 | TTCGGGGTTCAGGGCAAGGTTCCAGTCGGTCCAGCCGACCACATG | ssODN |
| | ATACAGGAGGTTCTAGGGTAAGGACAAAGGCAAAGAGACAAAGG | (rs76763715) |
| | CTCAACA | |
| 58 | AATTGGGTCCTCCTTCGGGGTTCAGGGCAAGGTTCCAGTCGGTCCA | 5′ ssODN |
| | GCC | (rs76763715) |
| 59 | GACCACATGGTACAGGAGGTTCTAGGGTAAGGACAAAGGCAAAG | 3′ ssODN |
| | AGACAAAGGCTCAACA | (rs76763715) |
| 60 | AATTGGGTCCTCCTTCGGGGTTCAGGGCAAGGTTCCAGTCGGTCCA | ssODN |
| | GCCGACCACATGGTACAGGAGGTTCTAGGGTAAGGACAAAGGCAA | (rs76763715) |
| | AGAGACAAAGGCTCAACA | |
| 61 | GTTCAGGGCAAGGTTCCAGTCGGTCCAGCCGACCACATGG | 5′ ssODN |
| | | (rs76763715) |
| 62 | TACAGAAGGTTCTAGGGTAAGGACAAAGGCAAAGAGACAAAGGC | 3′ ssODN |
| | TCAACA | (rs76763715) |
| 63 | GTTCAGGGCAAGGTTCCAGTCGGTCCAGCCGACCACATGGTACAG | ssODN |
| | AAGGTTCTAGGGTAAGGACAAAGGCAAAGAGACAAAGGCTCAAC | (rs76763715) |
| | A | |
| 64 | NGG, where N is A, G, T, or C | PAM |
| | | sequence |
| 65 | NGRRT, where N is A, G, T, or C and R is G or A | PAM |
| | | sequence |
| 66 | NGRRN, where N is A, G, T, or C and R is G or A | PAM |
| | | sequence |
| 67 | NNNNGATT, where N is A, G, T, or C | PAM |
| | | sequence |
| 68 | NNNNRYAC, where N is A, G, T, or C; R is G or A; and Y is C | PAM |
| | or T | sequence |
| 69 | NNAGAAW, where N is A, G, T, or C and W is A or T | PAM |
| | | sequence |

1. A method of correcting a gene variant associated with Parkinson's Disease, the method comprising:
introducing into an induced pluripotent stem cell (iPSC) one or more agents comprising a recombinant nuclease for inducing a DNA break within an endogenous target gene in the cell, wherein the target gene is human GBA1 and comprises a single nucleotide polymorphism (SNP) that is associated with Parkinson's Disease; and
introducing into the cell a single-stranded DNA oligonucleotide (ssODN), wherein the ssODN is homologous to the target gene and comprises a corrected form of the SNP,
wherein (i) the introducing of the one or more agents and the ssODN results in homology-directed repair (HDR) and integration of the ssODN into the target gene; and (ii) after the integration of the ssODN into the target gene, the target gene comprises the corrected form of the SNP instead of the SNP.

2. The method of claim 1, wherein the DNA break is a double strand break (DSB) at a cleavage site within the endogenous target gene.

3. (canceled)

4. The method of claim 1, wherein the recombinant nuclease is selected from the group consisting of a Cas nuclease, a transcription activator-like effector nuclease (TALEN), and a zinc finger nuclease (ZFN).

5. The method of claim 1, wherein the recombinant nuclease is a Cas nuclease.

6. The method of claim 5, wherein the one or more agents comprises the Cas nuclease and a single guide RNA (sgRNA).

7. (canceled)

8. (canceled)

9. The method of claim 5, wherein the Cas nuclease is selected from the group consisting of Cas3, Cas9, Cas10, Cas12, and Cas13.

10. The method of claim 9, wherein the Cas nuclease is Cas9 or a variant thereof.

11. (canceled)

12. The method of claim 10, wherein the Cas9 or a variant thereof is a Cas9 variant that exhibits reduced off-target effector activity, optionally wherein the Cas9 variant is an enhanced specificity Cas 9 (eSpCas9) or a high fidelity Cas 9 (HiFiCas9).

13-15. (canceled)

16. The method of claim 1, wherein the ssODN comprises a nucleic acid sequence that (i) is at least 80% homologous to a targeting sequence in the target gene, wherein the targeting sequence comprises the SNP, and (ii) is not homologous to the targeting sequence at the nucleotide of the SNP.

17-19. (canceled)

20. The method of claim 16, wherein the targeting sequence comprises a protospacer adjacent motif (PAM) sequence.

21. (canceled)

22. (canceled)

23. The method of claim 16, wherein the ssODN comprises a nucleic acid sequence that comprises one or more nucleotides that are not homologous to the corresponding nucleotides of the targeting sequence, and wherein the one or more nucleotides comprises one or more nucleotides that introduce a restriction site into the target gene that is recognized by one or more restriction enzymes.

24. The method of claim 1, wherein the corrected form of the SNP is not associated with PD and/or is a wildtype form of the SNP.

25. (canceled)

26. The method of claim 1, wherein the SNP is rs76763715.

27. (canceled)

28. The method of claim 26, wherein the GBA1 comprising the SNP encodes a serine, rather than an asparagine, at amino acid position 370 (N370S).

29. (canceled)

30. (canceled)

31. The method of claim 26, wherein the corrected form of the SNP is a thymine wildtype variant.

32. The method of claim 28, wherein, after the integration of the ssODN into the GBA1, the GBA1 comprises the corrected form of the SNP and encodes an asparagine at amino acid position 370.

33. The method of claim 1, wherein the SNP is rs421016.

34. (canceled)

35. The method of claim 33, wherein the GBA1 comprising the SNP encodes a proline, rather than a leucine, at amino acid position 444 (L444P).

36. The method of claim 33, wherein the corrected form of the SNP is an adenine wildtype variant.

37. The method of claim 35, wherein, after the integration of the ssODN into the GBA1, the GBA1 comprises the corrected form of the SNP and encodes a leucine at amino acid position 444.

38. The method of claim 1, wherein the SNP is rs2230288.

39. (canceled)

40. The method of claim 38, wherein the GBA1 comprising the SNP encodes a lysine, rather than a glutamic acid, at position 326 (E326K).

41. The method of claim 38, wherein the corrected form of the SNP is a cytosine wildtype variant.

42. The method of claim 40, wherein, after the integration of the ssODN into the GBA1, the GBA1 comprises the corrected form of the SNP and encodes a glutamic acid at position 326.

43. The method of claim 6, wherein the sgRNA comprises a CRISPR targeting RNA (crRNA) sequence that is homologous to a sequence in the target gene that includes a cleavage site.

44. The method of claim 43, wherein the sequence in the target gene that includes the cleavage site is immediately upstream of the PAM sequence.

45. (canceled)

46. The method of claim 43, wherein the crRNA sequence and the ssODN sequence comprise the nucleic acid sequences set forth in:
SEQ ID NOS: 8 and 3, respectively;
SEQ ID NOS: 8 and 5, respectively;
SEQ ID NOS: 8 and 33, respectively;
SEQ ID NOS: 13 and 27, respectively;
SEQ ID NOS: 14 and 30, respectively;
SEQ ID NOS: 15 and 36, respectively;
SEQ ID NOS: 16 and 39, respectively;
SEQ ID NOS: 17 and 42, respectively;
SEQ ID NOS: 18 and 45, respectively;
SEQ ID NOS: 19 and 48, respectively;
SEQ ID NOS: 20 and 51, respectively;
SEQ ID NOS: 21 and 54, respectively;
SEQ ID NOS: 22 and 57, respectively;
SEQ ID NOS: 23 and 60, respectively; or
SEQ ID NOS: 24 and 63, respectively.

47. The method of claim 1, wherein the recombinant nuclease lacks the ability to induce a DSB by cleaving both strands of double stranded DNA.

48. (canceled)

49. The method of claim 47, wherein (a) the recombinant nuclease is a Cas nuclease comprising one or more mutations such that the Cas nuclease is converted into a nickase that lacks the ability to cleave both strands of a double stranded DNA molecule; and/or (b) the recombinant nuclease is a Cas nuclease comprising one or more mutations such that the Cas nuclease is converted into a nickase that is able to cleave only one strand of a double stranded DNA molecule.

50. The method of claim 1, wherein the iPSC is artificially derived from a non-pluripotent cell from a subject.

51. The method of claim 50, wherein the subject has Parkinson's Disease.

52. The method of claim 23, wherein, after the integration of the ssODN into the target gene, the method further comprises:
contacting DNA isolated from the cell with the one or more restriction enzymes; and
determining whether the DNA isolated from the cell has been cleaved at the restriction site, wherein, if the DNA has been cleaved, the cell is identified as comprising an integrated ssODN.

53. (canceled)

54. (canceled)

55. The method of claim 1, wherein, after integration of the ssODN into the target gene, the method further comprises determining whether the cell comprises an integrated ssODN.

56. A complex for correcting a gene variant associated with Parkinson's Disease, comprising:
a Cas nuclease; and
a sgRNA comprising a CRISPR targeting RNA (crRNA) sequence that is homologous to a sequence in a target gene that includes a cleavage site,
wherein the target gene is human GBA1 and includes a single nucleotide polymorphism (SNP) that is associated with Parkinson's Disease.

57-63. (canceled)

64. A combination for correcting a gene variant associated with Parkinson's Disease, comprising:
a Cas nuclease;
a sgRNA comprising a CRISPR targeting RNA (crRNA) sequence that is homologous to a sequence in a target gene that includes a cleavage site, wherein the target gene is human GBA1 and includes a single nucleotide polymorphism (SNP) that is associated with Parkinson's Disease; and
a single-stranded DNA oligonucleotide (ssODN), wherein the ssODN is homologous to the target gene and comprises a corrected form of the SNP.

65-72. (canceled)

73. A complex for correcting a gene variant associated with Parkinson's Disease, comprising:
a Cas nuclease; and
a first sgRNA comprising a CRISPR targeting RNA (crRNA) sequence that is homologous to a sequence in a target gene;
wherein the target gene is human GBA1 and includes a single nucleotide polymorphism (SNP) that is associated with Parkinson's Disease.

74. A nucleic acid, comprising:
the nucleic acid sequence set forth in any one of SEQ ID NOS: 8 and 13-24;
the nucleic acid sequence set forth in any one of SEQ ID NOS: 1, 4, 25, 28, 31, 34, 37, 40, 43, 46, 49, 52, 55, 58, and 61:
the nucleic acid sequence set forth in any one of SEQ ID NOS: 2, 26, 29, 32, 35, 38, 41, 44, 47, 50, 53, 56, 59, and 62; or
the nucleic acid sequence set forth in any one of SEQ ID NOS: 3, 5, 27, 30, 33, 36, 39, 42, 45, 48, 51, 54, 57, 60, and 63.

75-77. (canceled)

78. A cell produced by the method of claim 1.

79. A cell identified by the method of claim 52.

80. A method for selecting for a cell comprising an integrated ssODN, comprising
contacting DNA isolated from a cell derived from the cell of claim 23 with the one or more restriction enzymes; and
determining whether the DNA isolated from the cell has been cleaved at the restriction site,
wherein, if the DNA has been cleaved, the cell is identified as comprising an integrated ssODN.

81. A method for selecting for a cell comprising a corrected SNP, comprising
sequencing DNA isolated from a cell derived from the cell of claim 1; and
determining whether the target gene comprises a corrected form of the SNP,
wherein, if the target gene comprises a corrected form of the SNP, the cell is identified as a cell comprising a corrected SNP.

82. A population of the cell of claim 78.

83. The population of claim 82, wherein the population is a population of pluripotent stem cells.

84. An induced pluripotent stem cell (iPSC) comprising a single-strand DNA oligonucleotide (ssODN) integrated into a target gene, wherein:
the target gene is human GBA1 and comprises a corrected single nucleotide polymorphism (SNP), wherein the non-corrected SNP is associated with Parkinson's Disease;
the integrated ssODN comprises the corrected SNP instead of the non-corrected SNP; and
(i) the ssODN comprises a protospacer adjacent motif (PAM) sequence that differs from a PAM sequence in the GBA1 target gene by at least one nucleotide position, wherein the integrated ssODN introduces a silent mutation in the PAM sequence of the target gene; and/or (ii) the ssODN comprises one or more nucleotides that are not homologous to the corresponding nucleotides of the GBA1 target gene, wherein the integrated ssODN introduces a restriction site in the target gene.

85. (canceled)

86. A method of differentiating neural cells, the method comprising:
(a) performing a first incubation comprising culturing the pluripotent stem cell(s) of claim 83 in a non-adherent culture vessel under conditions to produce a cellular spheroid, wherein beginning at the initiation of the first incubation (day 0) the cells are exposed to (i) an inhibitor of TGF-β/activing-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and
(b) performing a second incubation comprising culturing cells of the spheroid in a substrate-coated culture vessel under conditions to neurally differentiate the cells.

87-90. (canceled)

91. A method of differentiating neural cells, the method comprising:
exposing the pluripotent stem cell(s) of claim 83 to:
(a) an inhibitor of bone morphogenetic protein (BMP) signaling;
(b) an inhibitor of TGF-β/activing-Nodal signaling;
(c) at least one activator of Sonic Hedgehog (SHH) signaling; and
(d) at least one inhibitor of GSK3β signaling.

92. (canceled)

93. A therapeutic composition of cells produced by the method of claim 86.

94. A therapeutic composition of cells produced by the method of claim 91.

95. (canceled)

96. (canceled)

97. A method of treatment, comprising administering to a subject a therapeutically effective amount of the therapeutic composition of claim 93.

98-100. (canceled)
